(19) United States
(12) Patent Application Publication
(10) Pub. No.: US 2026/0139017 A1
(43) Pub. Date: May 21, 2026

(54) COMPOSITIONS AND METHODS USING REELIN IN ALZHEIMER'S DISEASE

(71) Applicants: The General Hospital Corporation, Boston, MA (US); Massachusetts Eye and Ear Infirmary, Boston, MA (US); Banner Health

(72) Inventors: Yakeel Tatiana Quiroz-Gaviria, Boston, MA (US); Joseph F. Arboleda-Velasquez, Newton, MA (US); Francisco Lopera, Medellin (CO); Eric Reiman, Phoenix, AZ (US)

(21) Appl. No.: 19/120,688

(22) PCT Filed: Oct. 13, 2023
(86) PCT No.: PCT/US2023/076914

§ 371 Date: Apr. 11, 2025

Related U.S. Application Data
(60) Provisional application No. 63/379,393, filed on Oct. 13, 2022.
(60) Provisional application No. 63/419,574, filed on Oct. 26, 2022.
(60) Provisional application No. 63/502,038, filed on May 12, 2023.

(51) Int. Cl. C07K 14/47; A61K 38/00; A61P 25/28

(52) U.S. Cl. CPC C07K 14/47; A61P 25/28; A61K 38/00

(57) ABSTRACT

Described herein are methods and compositions for treating Alzheimer's Disease (AD), as well as compositions comprising a reelin-derived peptide and methods of use thereof.

### CLAIM OF PRIORITY

This application is the U.S. National Phase Application under 35 U.S.C. § 371 of International Patent Application No. PCT/US2023/076914, filed on Oct. 13, 2023, which claims the benefit of U.S. Provisional Patent Application Serial Nos. 63/379,393, filed on Oct. 13, 2022; 63/419,574, filed on Oct. 26, 2022; and 63/502,038, filed on May 12, 2023. The entire contents of the foregoing are hereby incorporated by reference.

### FEDERALLY SPONSORED RESEARCH OR DEVELOPMENT

This invention was made with Government support under Grant Nos. OD019833, AG054671, NS100121, and NS110048 awarded by the National Institutes of Health. The Government has certain rights in the invention.

### SEQUENCE LISTING

This application contains a Sequence Listing that has been submitted electronically as an XML file named “29539-0549WO1.xml.” The XML file, created on Dec. 6, 2023 is 153,661 bytes in size. The material in the XML file is hereby incorporated by reference in its entirety.

### TECHNICAL FIELD

Described herein are methods and compositions for treating Alzheimer's Disease (AD), as well as compositions comprising a reelin-derived peptide or nucleic acid encoding the same, and methods of use thereof.

### BACKGROUND

Alzheimer's Disease (AD) is a progressive, neurodegenerative disorder that currently affects around 6.2 million people in the US. Currently, there are no effective treatments to forestall or reverse disease progression.

### SUMMARY

Provided herein are methods for treating or preventing (as used herein, “preventing” means reducing risk of developing) a neurodegenerative disease in a subject, e.g., Alzheimer's disease. The methods comprise administering to the subject an effective amount of a reelin protein or a nucleic acid encoding a reelin protein. Also provided herein are the reelin proteins for use in a method of treating or preventing a neurodegenerative disease. Preferably, the reelin protein comprises a H3447R or H3447K mutation and/or a R3454A mutation, optionally H3447R or H3447K in combination with an R3454A mutation. In some embodiments, the subject is a mammal, e.g., a human or non-human veterinary subject. In addition to Alzheimer's disease, the present methods may be used to treat other neurodegenerative diseases, disorders or conditions, including frontotemporal dementia, various types of memory loss, cognitive impairment including but not limited to mild cognitive impairment (MCI), or other conditions associated with accumulation of β amyloid or accumulation of tau or other proteopathies like frontotemporal dementia, or amyotrophic lateral sclerosis (ALS), or cognitive decline associated with aging. Neurodegenerative diseases can also include diseases of the eye like age-related macular degeneration, glaucoma, diabetic retinopathy or inherited retinal degeneration, stroke, brain trauma or concussion, retinal trauma, small vessel disease like cerebral autosomal dominant arteriopathy with subcortical infarcts and leukoencephalopathy (CADASIL), and aberrant angiogenesis like wet-age related macular degeneration. Other applications include conditions genetically or epigenetically associated with RELN loss-of-function including lateral temporal epilepsy, autism, schizophrenia, and bipolar disease, as well as cortex lamination defects, abnormal neuronal migration, and cerebellar hypotrophy.

In some embodiments, the reelin protein comprises full length reelin (or a sequence that is at least 80%, 85%, 90%, 95%, or 99% identical to human reelin), or a mini-reelin comprising A) a signal peptide; (B) an oligomerization, e.g., dimerization, domain, optionally reelin CR-50 Domain; (C) a receptor binding domain, optionally reelin domains (repeats) 5 and 6 (R5-6); and (D) a GAG binding domain, optionally a C-terminus from reelin (CTR), e.g., comprising a signal peptide, a CR-50 Domain, reelin domains 5 and 6 (R5-6), and a C-terminus from reelin.

In some embodiments, the methods comprise administering a nucleic acid encoding a reelin protein, wherein the nucleic acid is a naked mRNA or DNA encoding the reelin, or is in a viral vector, e.g., an AAV vector.

In some embodiments, reelin protein or a nucleic acid encoding a reelin protein are preferentially administered in or around the entorhinal cortex of the brain, e.g., to increase efficacy and or reduce side effects.

Also provided herein are compositions comprising a reelin protein or a nucleic acid encoding a reelin protein, preferably wherein the reelin protein comprises a H3447R or H3447K mutation and/or a R3454A mutation, optionally H3447R or H3447K in combination with an R3454A mutation, as described herein.

In some embodiments, the reelin protein comprises full length reelin, or a mini-reelin comprising (a mini-reelin comprising: (A) a signal peptide; (B) an oligomerization, e.g., dimerization, domain, e.g., reelin CR-50 Domain, Fc fragment of IgG, or FKBP; (C) a APOER2/VLDLR binding domain, e.g., reelin domains (repeats) 5 and 6 (R5-6), or Receptor binding domain of APOE, RAP, urokinase-type plasminogen activator (uPA), thrombospondin, f-spondin, or SEPP1; and (D) a glycosaminoglycan (GAG) binding domain, e.g., a C-terminus from reelin (CTR), TAT peptide, or P21, e.g., as described herein, e.g., in Table A. Exemplary signal peptides for secretion include IL2 signal peptide, human albumin signal peptide, human alpha 1-antitrypsin signal peptide, or human factor VIII signal peptide. Exemplary constructs include those in Table 5.

In some embodiments, the compositions comprise a nucleic acid encoding a reelin protein, optionally wherein the nucleic acid is a naked mRNA or DNA encoding the reelin, optionally with a human codon-optimized sequence, or is in a viral vector, e.g., an AAV vector.

Additionally, provided herein are compositions comprising or consisting of a reelin C-terminal region (CTR), and optionally a carrier, preferably wherein the CTR comprises a H3447R or H3447K mutation and/or a R3454A mutation, optionally H3447R or H3447K in combination with an R3454A mutation, optionally comprising or consisting of a sequence as shown in Table 1. In some embodiments, the compositions further comprise a non-reelin nucleic acid, e.g., mRNA, optionally wherein the mRNA encodes a therapeutic peptide. In some embodiments, the compositions further comprise an isolated non-reelin protein, e.g., complexed with or fused to the CTR. Also provided herein are methods of delivering a nucleic acid or protein to a cell, the method comprising administering to the cell an effective amount of the compositions comprising a CTR.

Also provided herein are methods of treating or preventing a neurodegenerative disease in a subject, e.g., Alzheimer's disease, the method comprising administering to the subject an effective amount of an agent that reduces methylation of the RELN promoter in an amount sufficient to increase RELN expression in the subject, wherein the agent that reduces methylation promoter is: (i) a fusion protein comprising a catalytically inactive CRISPR/Cas protein fused to a demethylation domain, and a guide RNA directing the fusion protein to demethylate a cytosine in the RELN promoter, optionally administered as a RNP; or (ii) a nucleic acid encoding a fusion protein comprising a catalytically inactive CRISPR/Cas protein fused to a demethylation domain, and a guide RNA directing the fusion protein to demethylate a cytosine in the RELN promoter, optionally administered as mRNA or in one or more vectors, optionally viral vectors, optionally adeno associated viral AAV vectors.

Further, provided herein are methods of treating or preventing a neurodegenerative disease in a subject, e.g., Alzheimer's disease. The methods comprise administering to the subject an effective amount of (i) a CRISPR/Cas protein, a guide RNA directing the Cas protein to the region of a RELN allele comprising a H3447, and an ssODN comprising a sequence comprising a H3447R or H3447K mutation and/or a R3454A mutation, optionally H3447R or H3447K in combination with an R3454A mutation, or one or more ssODNs comprising a sequence comprising a H3447R or H3447K in combination with an R3454A mutation, for insertion into the RELN allele, optionally administered as a RNP; or (ii) a nucleic acid encoding CRISPR/Cas protein, a guide RNA directing the Cas protein to the region of a RELN allele comprising H3447 and/or R3454, and an ssODN or plurality of ssODNs comprising a sequence comprising a H3447R or H3447K mutation and/or a R3454A mutation, optionally H3447R or H3447K in combination with an R3454A mutation for insertion into the RELN allele, optionally administered as mRNA or in one or more vectors, optionally viral vectors, optionally adeno associated viral AAV vectors.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-C. PET imaging of RELN-COLBOS (H3447R) carrier. (A) Representative PiB PET amyloid (top left row) and Fluortaucipir tau PET (bottom left row) imaging of (PSEN1 E280A; RELN-COLBOS) case (left panel) as compared to a PSEN1 E280A mutation carrier with MCI at typical age (right panel). For both measurements, specific binding of the tracer is represented using a scale with the lowest (0.8 DVR or SUVR) and the highest (2.00 DVR or SUVR) degree of binding. The right side of Panel A shows a representative ¹⁸F-fludeoxyglucose (FDG) PET precuneus cerebral metabolic rate for glucose (CMRgI) of the (PSEN1 E280A; RELN-COLBOS) case (left panel) as compared to a PSEN1 E280A mutation carrier with MCI at typical age (right panel). Binding affinity of the dye is represented using a scale with the lowest (0.5 SUVR) and the highest (2.1 SUVR) degree of binding. (B) Dot plot analysis of brain imaging measurements shown in Panel A for amyloid burden, tau burden, glucose metabolism and hippocampal volume. Brain imaging measurements of the (PSEN1 E280A; RELN-COLBOS) case compared to the previously published (PSEN1 E280A; APOEch) homozygote case, unimpaired PSEN1 E280A carriers (n=13-18) and younger MCI PSEN1 E280A carriers (n=7-11) (2). Some previously published data points are included in the figures presented here because they are the only available data for comparison (2). Mean cortical-to-cerebellar distribution volume ratios (DVR) was used to quantify the amyloid burden across individuals; entorhinal-to-cerebellar SUVRs was used to quantify tau burden; hippocampal-to-whole brain volume ratios is used to compare changes in hippocampal volume; precuneus-to whole-brain CMRgl ratios is used to compare changes in glucose metabolism between different cohorts. Data expressed as individual values with mean±s. e. m. (C) Anatomic details of tau burden in temporal lobe cortex. Flat-map representations of right hemisphere temporal lobe cortex for regions of interest (top-left, PPC=posterior parahippocampal cortex, ERC=entorhinal cortex), with tau PET (Fluortaucipir, FTP) overlay for four cases. Asymptomatic PSEN1 E280A mutation carrier was 38 years old. PSEN1 E280A mutation carrier with typical MCI was 44 years old. The PSEN1 carrier with RELN-COLBOS mutation was notable for having a relatively lower tau burden in medial temporal regions (ERC and PPC), compared to an asymptomatic PSEN1 E280A mutation carrier and a PSEN1 E280A mutation carrier with MCI at typical age of onset.

FIGS. 2A-M. (SEQ ID NO:64 and 65) The RELN H3448R variant enhances Dab1 signaling and the affinity of C-terminus region of RELN (CTR-RELN) to heparin, reduced tau hyperphosphorylation and preserved motor functions in mice. (A) Representative western blotting band of pDAB1 levels (top) and total protein staining (bottom) levels in primary mouse cortical neuron cells treated five min. at 37° C. with 4 μg/ml of full-length RELN WT or RELN H3448R, mouse orthologue of H3447R RELN. (MOCK; P<0.0029), and RELN WT (P=0.0246). Data is presented as mean±s. e. m. and analyzed by Kruskal-Wallis (Dunn's post-hoc analysis for multiple comparisons of n=4 independent experiments. (B) Spectroscopic analysis of heparin chromatography fractions of the CTR-RELN WT and CTR-RELN H3447R variant eluted in an increasing gradient of NaCl (0.05 M NaCl step gradient) in 20 mM Tris HCl buffer and detected via UV absorbance at 280 nm. Data is expressed as percentage of input over NaCl gradient fractions from 0.4 M to 5 M NaCl. Data is showing that 0.55 M NaCl can displace CTR-RELN WT binding from a heparin column. The affinity for heparin of the CTR-RELN increases in presence of the H3447R mutation, as suggested by the shift of the peak with maximum height of eluted fraction from 0.55 M to 0.7 M NaCl. N=3 independent experiments. Error bars represent s. e. m. (C) Representative sensorgrams of the binding analysis between chip sensors coated with heparin and increasing concentrations of CTR-RELN variants at concentrations ranging from 0 to 25 nM. Data expressed as response units over time in seconds. Equilibrium dissociation constants (K_{D}) for each SPR analysis are shown inside the graph and support the difference in affinity binding between heparin and CTR-RELN variants: H3447R (right plot, K_{D}=3·75e-9 M-1s-1)>H3347 (left plot, K_{D}=6·53e-9 M-1s-1). Sensorgrams of CTR-RELN with the H3447K and H3447D control variants are reported in FIG. 7A-B for comparison. (D) Representative WB of pDab1 levels (top blots) and GAPDH levels (bottom blots) detected in the cerebellum of both females (left) and males (right) either wild type (RELN WT/WT), heterozygous (RELN WT/H3448R) or homozygous (RELN H3448R/H3448R) for the mRELN H3448R mutation. Levels were detected in 6-12 months old mice. (E,F) Quantifications of pDab1 levels normalized to GAPDH and expressed as fold change of RELNWT showing a genotype effect in pDab1 levels in male mice (F. p=0.0284 for WT/WT vs. H3448R/H3448R; p=0.0037 for WT/H3448R vs. H3448R/H3448R, One-way ANOVA) and not in female mice (E). G, (SEQ ID NO:64)Isothermal calorimetry measurements of short variants CTR-RELN WT (left plot) and CTR-RELN H3447R (right plot) titrated with 5 μM heparin. Affinity calculations are reported on top of each plots. H, Binding analysis via BLI between Fc-fusion CTR-RELN WT or H3447R and a heparin coated biosensor. Association (kₐ) and dissociation constants (k_{d}) are used to calculate the equilibrium disassociation constant (K_{D}) that is displayed in the plot. I,(SEQ ID NO:65) Docking of CTR-RELN WT (purple) with a representative heparin molecule (cyan). AAs in the RELN CTR that have polar contacts with heparin are highlighted in magenta. J, Representative IHC images from the hippocampus of WT/WT, WT/RELN H3448R, hTau tg/WT and hTau tg/RELN H3448R mice stained with ptau T205 antibody. hTau tg/WT mice showed neurofibrillary tangles and neuropil threads in CA1 and dentate gyrus, while hTau tg/RELN H3448R showed tau pathology to a lesser degree (soma of an affected neuron depicted with a dotted line). Bar=100 μm. K, Bar graph for ptau T205 signal intensity values in hTau tg/WT (n=3 mice) and hTau tg/RELN H3448R mice (n=3 mice). The latter showed significantly less signal intensity values. p=0.022 *, two sided Student's T. Test. Error bars represent standard deviation from mean. L, Representative phenotype observed during the tail elevation test and the relative score (0=severely impaired, 1: 50% impaired; 2=normal). M, Tail elevation score recorded on RELNWT/Tau-P301L (n=13 male mice) and RLN-H3448R H3448R/Tau-P301L crossed male mice (n=11 male mice) shows a significantly improved tail elevation score in the presence of the RLN-H3448R variant as compared to Tau-P301L mice expressing RLN WT (*p=0.0305, two-tailed unpaired T Test, t=2.313, df=22). Box plots expressed as minimum to maximum values around average.

FIGS. 3A-E. Neuropathological characterization of the (PSEN1 E280A; RELN H3447R) case. (A) is reported amyloid beta (Aβ) and hyperphosphorylated tau (ptau) pathologies in CA1 and EC. Both pathologies present wide distribution and intensity, Aβ pathology shows diffuse plaques with varied distribution and size in both structures (panels and insets). ptau pathology shows varied density of neurofibrillary tangles and diffuse tau pathology. Scale bar=500 mm. (B) shows representative images of neurons stained with Kluver-Barrera in CA1 and EC of the PSEN1 E280A RELN-COLBOS (RELN-COLBOS) case, the PSEN1 E280A/APOE Christchurch (APOEch) case, an average onset PSEN1E280A familial Alzheimer's (FAD) case and a sporadic Alzheimer's (SAD) case. Scale bar=125 mm. (C) 3D scatter plot graph for Aβ, ptau and neuronal density for EC and CA1 from RELN-COLBOS, APOEch, FAD and SAD cases. The EC in the RELN-COLBOS case shows the highest neuronal density while presenting with low Aβ and ptau pathologies. In contrast, CA1 in the same case shows low neuronal density together with high levels of Aβ and ptau pathologies. For all other cases, neuronal density in both areas is low while presenting with varied levels of Aβ or ptau pathology. (D) Representative images of RELN-CT and ApoE staining of the RELN-COLBOS, APOEch, FAD and SAD cases in EC and CA1. The RELN-COLBOS case shows stronger background signal in both structures with lesser intraneuronal signal for RELN-CT in EC. Similarly, the APOEch case shows lower intraneuronal signal in EC with the RELN-CT antibody and very low intraneuronal signal in both structures with the ApoE antibody (magnified right panel). Finally, ApoE staining shows noticeable plaque-like and tangle-like signals in FAD and SAD cases in both structures, EC and CA1. Scale bars=100 mm and 25 mm in the magnified panel. (E) Representative images of Kluver Barrera staining of whole hippocampal and parahippocampal sections (above), together with representative magnified images of parahippocampal subcortical white matter stained with RELN-CT antibody in the RELN-COLBOS, APOEch, FAD and SAD cases. The RELN-COLBOS case showed increased white matter luxol fast blue signal intensity, while RELN-COLBOS and SAD cases showed increased intracellular RELN-CT signal in white matter. Scale bars=2-5 mm for above and 25 μm below panels.

FIGS. 4A-D. A putative model for ADAD pathology modifications APOEch and RELNH3447R PS1E280A cases. In normal conditions (A), RELN and ApoE can bind to VLDLr/APOER2 receptors complex aided by GAG; the modulation of this signaling pathway maintains physiological Dab1 activation (phosphorylation indicated by a circle), which keeps basal phosphorylation levels of GSK3β and tau phosphorylation (left panel). In the APOE Christchurch case, ApoE decreased binding to GAG allows increased RELN binding to the receptor complex, increasing Dab1 activation and inhibiting GSK3β and tau phosphorylation (middle panel B). In the presence of the RELN H3447R variant, increased RELN binding to GAG leads to enhanced Dab1 activation and subsequent inhibition of GSK3β and tau phosphorylation (right panel C). (D) Model of protective state wherein increased Reelin signaling and or reduced ApoE signaling have a beneficial effect.

FIGS. 5A-B. (SEQ ID NO:66 and 67) Subject's genealogy and Sanger validation of the C-terminus RELN H3447R variant. (A) Subject's genealogy. Circles representing females, squares representing males, diamonds representing individuals whose gender has been masked for privacy. Arrowhead depicts proband case. Deceased individuals are marked with a crossed bar. Black shapes indicate affected individuals and white shapes indicate unaffected individuals. (B) (SEQ ID NO:66 and 67) Representative DNA Sanger sequencing of amplicon in RELN gene from a carrier that does not carry the mutation (RELN/RELN, top panel), as compared to the individual carrying the variant (RELN/RELN H3447R, bottom panel). Region of the H3447R mutation is highlighted by the black circle.

FIGS. 6A-B. Treatments with RELN H3448R reduces tau phosphorylation in vitro. (A) Representative western blotting of ptau (Ser396, top blot), tau (Tau5, middle blot) and GAPDH (bottom blot) detected in primary mouse cortical neurons treated one h with either vehicle, RELN H3448 wild type (RELN WT) or RELN H3448R. (B) Quantification of normalized intensities of n=3 independent experiments showing a significant increase in ptau/tau ratio in neurons treated with RELN H3448R as compared to vehicle (p=0.03, unpaired T-test).

FIGS. 7A-B. Mutations at position 3447 in the CTR domain of RELN impact heparin binding. (A, B) Representative sensorgrams of the binding analysis between chip sensors coated with heparin and increasing concentrations of CTR-reelin variants ranging from 0 to 25 nM. Sensorgrams for RELN H3447K (A) and RELN H3447D (B). Data expressed as response units over time in seconds. Equilibrium dissociation constants (k_{D}) for each SPR analysis are shown inside the graph and supports the difference binding between heparin and reelin variants in the order H3447R (FIG. 2C, K_{D}=3·75e-9 M-1s-1)>WT (FIG. 2C, K_{D}=6·53e-9 M-1s-1)>H3447K (A, K_{D}=7·33e-9 M-1s-1)>H3447D (B, K_{D}=1·64e-7 M-1s-1) when compared to data presented in FIG. 2.

FIG. 8. Reelin C-terminal consensus across 128 mammalian species. Analysis of Reelin sequences across mammalian species shows that the CTR is highly conserved. Basic AAs might play a role in binding GAGs or lipoprotein receptors.

FIG. 9. (SEQ ID NO:68) Orientation of select basic amino acids in the heparin-binding motif. Analysis of Reelin sequences across mammalian species shows that the CTR is highly conserved. Basic AAs might play a role in binding GAGs or lipoprotein receptors. Position 3447 (arrow) orient in the same direction as a majority of other arginines. Arginines in 3446 position and 3453 may also interact with heparin as part of the heparin-binding motif but are oriented differently from most basic AAs. R3452 and R3457 are part of the heparin-binding motif but are unlikely to contribute to heparin interaction since they are each oriented differently from the other basic amino acids in the potential binding site.

FIG. 10. Twenty lowest-energy structures of Reelin CTR produced by 2D NMR. Structures show that there is a region of flexibility towards the c-terminal of the peptide, including the H3447R mutation.

FIGS. 11A-D. (SEQ ID NO:69-76)Representative HPLC chromatograms of Reelin-peptide variants. (A) (SEQ ID NO:69-76)Zero (R3446H), one (WT), or two (H3447R) basic amino acids in positions 3446-3447 show increased interaction with heparin, indicated by later peak retention time in isocratic 1M KCl elution. (B) This pattern held for short and long peptides. n=2 repeats, within <0.5 min of representative peaks. (C) Short reelin variants had earlier peak retention times in comparison to long reelin variants (D); however, both short and long peptides are similarly affected by AA substitutions at position 3447. H3447D had an earlier peak retention time in comparison to WT. Basic substitutions H3447K and H3447R had increased later peak retention times, therefore indicating increased interaction with heparin. n=2 repeats, within <0.5 min of representative peaks.

FIGS. 12A-B. Surface plasmon resonance to assay Reelin CTR-Heparin binding kinetics. Binding between H3447 (A) or H3447R (B) and a heparin coated metal film. Association (kₐ) and dissociation constants (k_{d}) were used to calculate the equilibrium disassociation constant (K_{D}).

FIG. 13. BLI of Heparin-Reelin interaction shows H3447R has ˜2-fold greater interaction in comparison to WT Reelin. Binding between WT Reelin (A) and a heparin coated biosensor. Association (kₐ) and dissociation constants (k_{d}) are used to calculate the equilibrium disassociation constant (K_{D}).

FIGS. 14A-B. RELN modulation of Aβ aggregation. (A) Aβ aggregation Thioflavin T assay (ThT) with RELN CTR WT and H3447R long (left) and short (right) showing that RELN CTR reduces Aβ aggregation. (B) ThT Assay of Aβ aggregation alone or in the presence of RELN 3431HH-R3446H-3451HH long or RELN 3431HH-R3446H short peptides, showing that in the presence of RELN 3431HH-R3446H short variant the antiaggregating effect is significantly reduced. For both D and E panels, data is expressed as percentage of maximum ThT emission of Aβ at 120 min aggregation kinetic. Comparisons were made between aggregation kinetics of Aβ alone or in the presence of RELN variants using 2-way ANOVA followed by Tukey's test for multiple comparisons. P-values were calculated for samples at 30 min and 40-45 min (*p<0.05; **p<0.001;***p<0.0001;****p<0.00001).

FIG. 15. RELN CTR modulation of Aβ aggregation. Aβ aggregation Thioflavin T assay (ThT) with RELN CTR H3447K and H3447D long (left) and short (right) showing that RELN CTR reduces Aβ aggregation. Data is expressed as percentage of maximum ThT emission of Aβ at 120 min of kinetic aggregation. Comparisons were made between aggregation kinetics of Aβ alone or in the presence of RELN variants using 2-way ANOVA followed by Tukey's test for multiple comparisons. P-values were calculated for samples at 30 min and 40-45 min (*p<0.05; **p<0.001;***p<0.0001;****p<0.00001).

FIG. 16. RELN CTR facilitates cellular uptake of a mRNA cargo. Green fluorescence protein (GFP) expression in human retinal endothelial cells treated with 5 μg of encoding—mRNA. mRNA was bonded with to a cationic peptide at three different ratios. Ratios expressed as mRNA:Peptide mass ratio.

FIGS. 17A-J. In vitro screening of mini-RELN constructs on human retinal endothelial cells (HRECs). Panels showing representative bright field acquisition, using 10× magnification, of HREC cells either untreated, treated with Lipofectamine, mini-RELN constructs 225Q and 225Xf (17A), 225T and 225S (17B), 225F and 225SW (19C), 225ZZ and 233C (19D), 22WT and 225Z (E), 225SV (F), 225RR and 225D (G), 225E (H), 225SU and 233F (I), or 233A (J). Acquisitions made either prior transfection (Pr. t., top row), 5 h post-transfection (5H p.t., middle row) and 24H post-transfection (24H p.t., bottom row). Scale bar=500 μm.

FIGS. 18A-E. In vitro screening of mini-RELN constructs on HRECs. A. Representative western blotting of HREC lysates upon 24 h overexpression of 225Q, 225Xf, 225T, and 225S (A), 225Yf (n=2), 225SW (n=2), 225ZZ (n=2) and 233C (n=2) (B), 225SW (n=2), 225Z (n=2), and 225SV (n=2) (C), 225RR (n=2), 233D (n=2), and 233E (n=2) (D), or 225SU (n=2), 233F (n=2), and 233A (n=2) (E) plasmids using lipofectamine as the transfection agent. As control, untreated and lipofectamine treated cells were also tested. WB was used to detect total DAB1 and β-actin to normalize DAB1 levels.

FIGS. 19A-E. Quantification of total DAB1 levels expressed as normalized intensities to β-actin and control (lipofectamine treatment, Lp), showing reduced levels of DAB1 in the presence of the mini-RELN constructs as compared to lipofectamine (Lp).

FIGS. 20A-F. In vitro screening of constructs on HRECs cells. Representative western blotting of HREC lysates upon 5-minute treatment with culture media containing different mini RELN constructs 225Q, 225R (A); 225S, 225T (B); 225S, 225T, 225Z (C), 225FX, 225FY, 225SU, 225SV, 225SW, FL-RELN WT, FL-RELN Mut, and 225Z (D); 225ZZ, 225Z (E); or Fc-RELN WT (184I), Fc-RELN H3447R (184J), and RELN (F). Constructs were obtained from Innovagen or Creative Bio. Recombinant mouse reelin protein was obtained from R&D systems at 4 μg/mL. Phosphorylated DAB1 (pDAB1, 20A-F) and total DAB1 (20F) levels were measured

FIGS. 21A-F. Quantification of pDAB1 levels expressed as normalized intensities to GAPDH and control (media).

FIG. 22. Quantification of total DAB levels expressed as normalized intensities to GAPDH and control upon 5-minute overexpression of different mini RELN constructs Fc-RELN WT (184I), Fc-RELN H3447R (184J), and RELN from Innovagen.

FIG. 23. Reduced oligomeric Tau-induced cytotoxicity in the presence of 184I mini-RELN peptide.

FIGS. 24A-C. C-terminus RELN domain modulates Aβ aggregation. A, B Thioflavin T (ThT) assay of Aβ aggregation either alone or in the presence of C-terminal RELN WT (either uncleaved or long, or furin-cleaved or short), panel A, or C-terminal RELN H3447R (either uncleaved or long, or furin-cleaved or short), panel B, or in the presence of vehicle or morin used as positive control for aggregation inhibition. Aggregation kinetic conducted up to 40 min showing that mini-RELN reduces Aβ aggregation. C. Analyses of changes in ThT fluorescence percentage of Aβ at different time points and under different treatment-conditions, showing the significant inhibition of Aβ aggregation. p-values were calculated for samples at 30 min and 40 min.

FIGS. 25A-B. Western blotting validation of mini-RELN overexpression in HEK cells. Representative western blotting of both cell lysates and culture media from HEK cells transfected to overexpress mini-RELN constructs as compared to controls (UT, untreated and LP, lipofectamine) showing the anti Fc-tag positive bands when overexpressing Fc-tagged mini-RELN constructs.

FIGS. 26A-E. In vitro screening of constructs on HRECs cells. A. Representative western blotting of HREC lysates upon 5-minute treatment with culture medium containing 225Xf and 225Yf (A); 225ZZ and 233C (B); 225SU and 225SV (C); 225SW and 233F (D); or 225RR and 233A (E) miniRELN constructs or controls at different concentrations as shown.

FIGS. 27A-E. Quantification of pDAB1 levels from FIGS. 26A-E expressed as normalized intensities to b-actin and control (lipofectamine treatment, Lp).

FIGS. 28A-K. In vivo drug delivery and activity of mini-RELN peptide 225S (A-F), 225T and 225Z (G-K). A. anti human IgG-Fc ELISA showing that optical density levels in the hippocampus both 24 h and 72 h post injection trended to be increased in the hippocampus 72 h post injection of mini-RELN-Fc tagged peptide as compared to the PBS vehicle. B. representative WB of pDAB1 levels in the hippocampus 24 h and 72 h post-trans-nasal drug delivery. C. quantification of B. D. Representative WB of pDAB1 levels in the hippocampus and entorhinal cortex of Tau P301S mice 72 h post-trans nasal drug delivery. GAPDH was detected as loading control (bottom blot). E, F. Quantification of the pDAB1-positive bands normalized to GAPDH and PBS treated control in the hippocampus (E) and in the entorhinal cortex (F) showing that levels of pDAB1 were increased in the entorhinal cortex 72 H post-injection. G. Representative WB of pDAB1 levels (top blot) in the hippocampus 72 h post-trans-nasal drug delivery of mini-RELN peptides 225Z and 225T on WT male mice. We used βActin as loading control (bottom blot). H, I. Representative WB of pDAB1 levels in the hippocampus 72 H post drug delivery of 225T (H) and 225Z (I) showing that levels of pDAB1 were increased in the hippocampus. J. Representative WB of pDAB1 levels (top blot) in the midbrain 72 h post-trans-nasal drug delivery of mini-RELN peptides 225Z and 225T on WT male mice. We used βActin as loading control (bottom blot). K. Representative WB of pDAB1 levels in the midbrain 72 H post drug delivery of 225T and 225Z showing that levels of pDAB1 were increased in the midbrain.

FIGS. 29A-B. In vivo characterization of pDab1 expression in Wildtype animals treated with vehicle (pluronic) or Mini Reelin 225S via trans nasal brain delivery. A. Entorhinal-hippocampal analysis of pDAB1 expression 24 hours after Mini Reelin administration. Positive pDab1 cells are surrounded by white dashed lines. Mini Reelin was detected using FC antibody. DAPI is also shown. B. Segmentation was done manually to show area of positive pDab1 staining of the MiniReelin compared to the Pluronic. Scale bar: 25 μm. Data demonstrates increased levels of RELN signaling measured via pDab1 in animals treated with miniRELN. Direct correlation between signal of miniRELN peptide and increased pDab1 signal is shown.

FIG. 30. Quantification of pTau Ser396 expression in MAPT P301S mice intraperitoneally injected with either PBS or mini-RELN peptide 225T, evaluated using immunofluorescence microscopy. pTau S396 fluorescence intensity was used as a hallmark of tau pathology. Data demonstrates decreased levels of pTau S396 signaling, which was indicative of reduced tau pathology in the hippocampal and entorhinal regions when treated systemically with mini-RELN.

### DETAILED DESCRIPTION

Efforts to develop treatments for Alzheimer's disease (AD) have focused on removing amyloid, a neuropathological hallmark. The present inventors have characterized over 5,000 individuals from a Colombian kindred with autosomal-dominant AD (ADAD) due to the E280A mutation in Presenilin-1 (PSEN1) of which about 1,200 are mutation carriers. PSEN1-E280A carriers usually develop cognitive impairment in their forties. E280A carriers develop mild cognitive impairment (MCI) by the median age of 44 years (95% CI, 43-45) and dementia by 49 years (95% CI, 49-50), (1) with rare exceptions (2). We reported a female PSEN1 E280A carrier with two copies of the APOE3 Christchurch (APOEch, R136S) mutation who remained cognitively unimpaired for nearly thirty years after the expected age at clinical onset (2). The present disclosure is based in part on the clinical, in vivo neuroimaging, genetic and neuropathological characteristics of a male case from the same population with the PSEN1 E280A mutation, who also presented with an extreme phenotype of delayed age at clinical onset of ADAD.

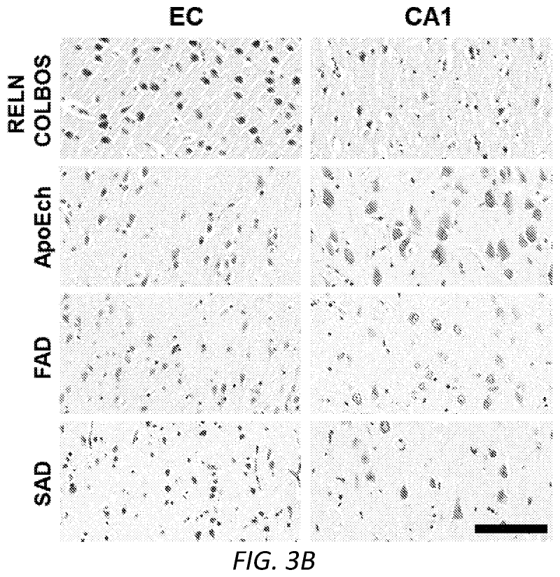

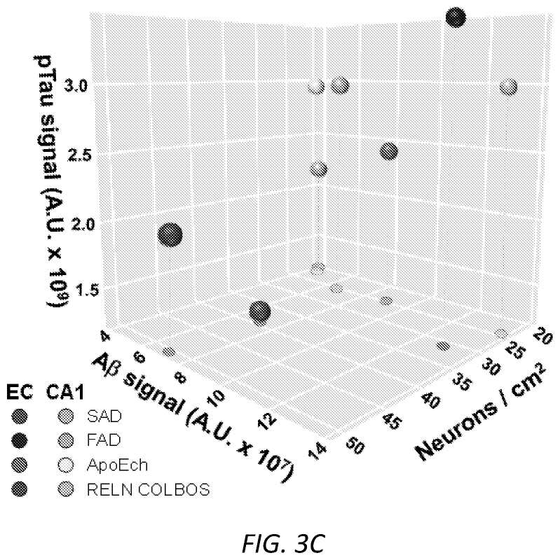

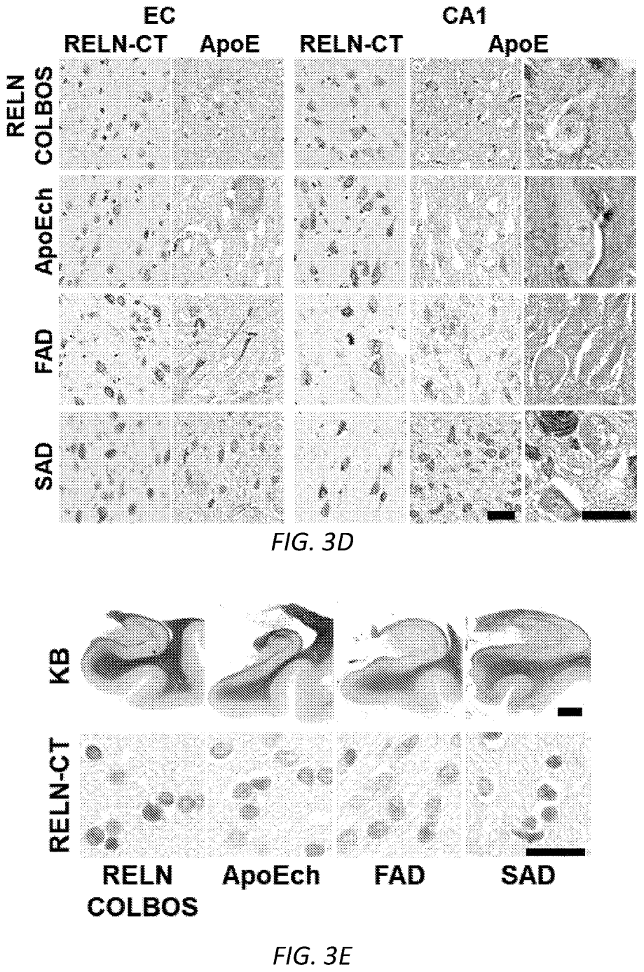

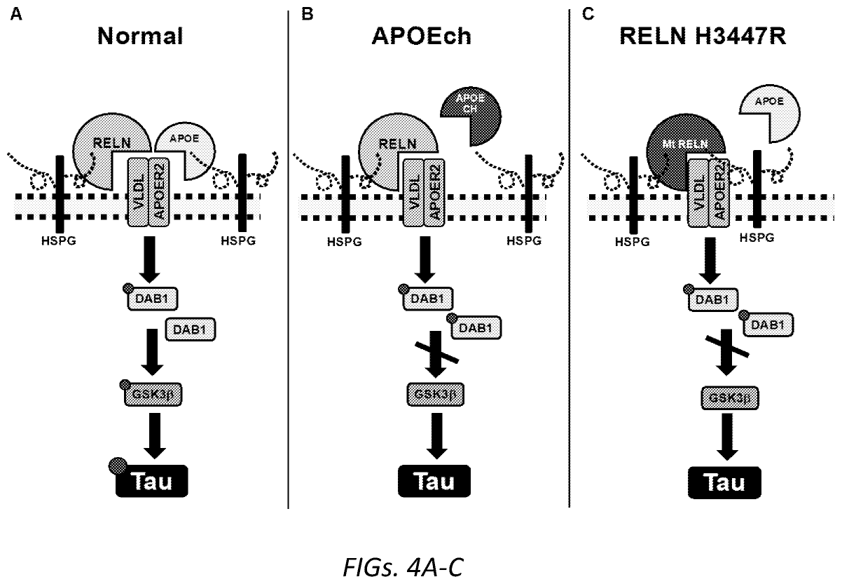

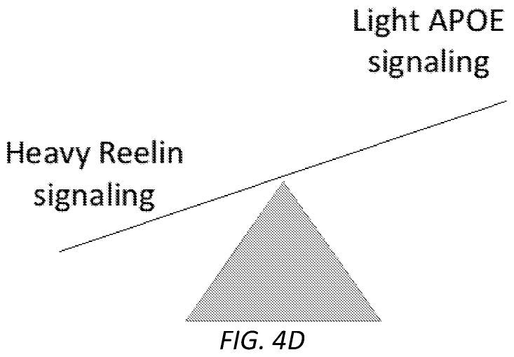

We characterized a male heterozygous for the RELN-COLBOS variant who was resilient to the cognitive impairment associated with the PSEN1 E280A mutation until age 67. The observation of low tau pathology and increased neuronal density in the entorhinal cortex as compared to other AD cases implicates this brain region in RELN-mediated mechanisms relevant to protection against AD (Table 2 & FIGS. 3A-E). A female sibling carrier of the RELN-COLBOS and PSEN1 E280A variants also presented with delayed age at onset of cognitive decline, though less optimal protection compared to her brother, and prolonged end-stage disease. RELN-specific sexual dimorphism may have contributed to her distinct features. We cannot rule out the possibility that other factors may have contributed to the AD resilience phenotype in the RELN-COLBOS carriers. Others identified RELN as a candidate gene associated with AD pathology in cognitively healthy individuals (26) and DAB1 variants were linked to AD risk in APOE4 homozygotes further linking the RELN/DAB1 pathway to Alzheimer's.(27).

The present inventors previously reported a female case homozygous for APOE3 Christchurch who was resistant to ADAD-related dementia, had widespread amyloid pathology and low tau pathology in the entorhinal cortex (2). Tauopathy was more extensive in the RELN-COLBOS case compared to the APOE3 Christchurch homozygote, except for the entorhinal cortex, which was largely spared in both, suggesting resilience in the RELN-COLBOS case.

The hypermorphic effect of RELN is mild. This is the first known report of a RELN hypermorph, and a stronger effect may not support proper development in this critical signaling process. The experimental evidence of a gain-of-function mechanism for the RELN-COLBOS variant, and the fact that a patient with extreme protection against ADAD has it, establish a rationale for genetic implication in the observed phenotypes. Without wishing to be bound by theory, it is hypothesized that RELN-COLBOS is not a neutral variant and is likely to contribute to the resilience phenotype of the subject.

The APOE Christchurch mutation impairs ApoE binding to GAG and the ApoE receptors (2, 28). Conversely, as shown herein, the RELN-COLBOS variant enhances RELN binding to GAG and (Neurophilin 1) NRP1, possibly giving it a competitive advantage for binding to its receptors (4). RELN-COLBOS binding to GAGs or heparan sulphate proteoglycans may increase local concentrations of RELN leading to enhanced signaling. The present analyses of the RELN-COLBOS case revealed a converging mechanism potentially linking ApoE and RELN interactions via GAG or other receptors to the protection against AD. RELN-COLBOS is a gain-of-function variant showing stronger ability to activate its canonical protein target Dab1 and reduce human tau phosphorylation in a knock-in mouse. Regulation of this APOE-RELN protective pathway, particularly in the entorhinal cortex, may have a profound therapeutic impact on the resistance to tau pathology and neurodegeneration, and resilience against cognitive decline and dementia in Alzheimer's disease.

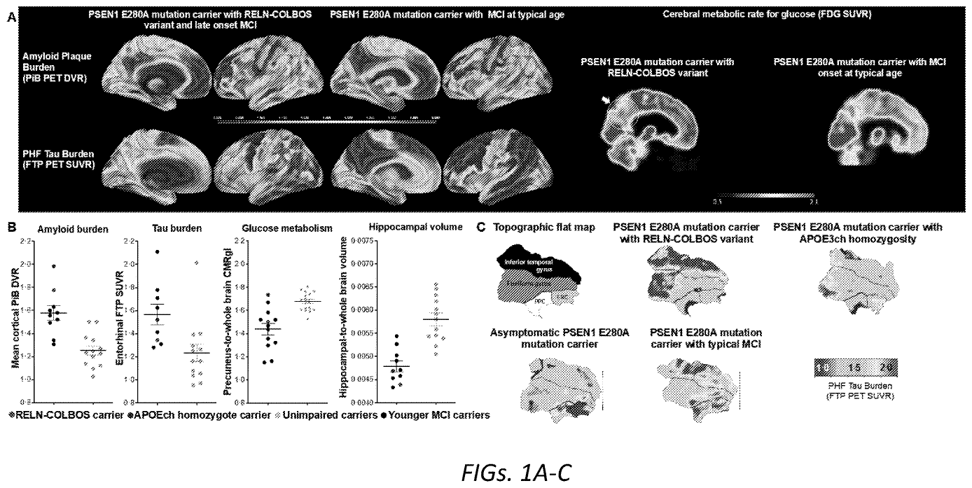

Measurements of metabolic rate for glucose in the precuneus-to-whole brain region using fluorodeoxyglucose (FDG) PET showed a slightly higher level of glucose metabolism compared to the mean levels of typical MCI carriers from the kindred, who were of much younger ages (FIG. 1a-b).

### Methods of Treating and Reducing Risk of Cognitive Decline and Dementia

Provided herein are methods for treating or reducing risk of developing or worsening cognitive decline and dementia. The methods can include administering a RELN protein, e.g., a full-length RELN recombinant protein or a mini-RELN recombinant protein or variant thereof as described herein (see, e.g., Table A), optionally wherein the protein comprises a variant described herein, e.g., an H3447R or H3447K mutation and/or a R3454A mutation, optionally H3447R or H3447K in combination with an R3454A mutation, or a nucleic acid encoding the RELN protein. R3455A provides resistance to Furin cleavage of the CTR of Reelin (Kohno et al., J Neurosci. 2015 Mar. 18; 35(11): 4776-4787). Alternatively or in addition, the methods can include administering a gene editing agent that alters at least one allele in a cell to include a H3447R or H3447K mutation and/or a R3454A mutation, optionally H3447R or H3447K in combination with an R3454A mutation, to an agent that increases expression or activity of reelin, e.g., an agent that reduces methylation of the reelin (e.g., a crispr/cas-demethylase fusion). In some embodiments, the methods include administering the proteins (and guide RNAs/ssODNs as needed) directly; administering nucleic acids encoding the proteins (and guide RNAs/ssODNs as needed), e.g., as naked DNA or mRNA or in expression vectors, e.g., viral vectors, or administering cells that express the proteins (and guide RNAs/ssODNs as needed). For example, naked DNA can be administered without a vector using electroporation devices (e.g. in the ciliary body of the eye).

### Alzheimer's Disease

The methods described herein may be used to treat or reduce the risk of developing subjects with all types of Alzheimer's disease including, but not limited to, familial and sporadic Alzheimer's disease, early onset or late onset Alzheimer's disease. In some embodiments, the present methods may be used to treat or reduce the risk of development of early onset familial form of Alzheimer's disease (AD) or cognitive decline associated with aging.

Typically, increasing forgetfulness or mild confusion are early symptoms of Alzheimer's disease. Gradually, cognitive impairment associated with Alzheimer's disease leads to memory loss, especially recent memories, disorientation and misinterpreting spatial relationships, difficulty in speaking, writing, thinking, reasoning, changes in personality and behavior resulting in depression, anxiety, social withdrawal, mood swings, distrust in others, irritability and aggressiveness, changes in sleeping habits, wandering, loss of inhibitions, delusions, and eventually death.

### Other Neurodegenerative Diseases, Disorders or Conditions

In addition to Alzheimer's disease, the present methods may be used to treat other neurodegenerative diseases, disorders or conditions, including frontotemporal dementia, various types of memory loss, cognitive impairment including but not limited to mild cognitive impairment (MCI), or other conditions associated with accumulation of β amyloid or accumulation of tau or other proteopathies like frontotemporal dementia, or amyotrophic lateral sclerosis (ALS), or cognitive decline associated with aging. Neurodegenerative diseases can also include diseases of the eye like age-related macular degeneration, glaucoma, diabetic retinopathy or inherited retinal degeneration, stroke, brain trauma or concussion, retinal trauma, inherited retinal degenerations, small vessel disease like cerebral autosomal dominant arteriopathy with subcortical infarcts and leukoencephalopathy (CADASIL) or aberrant angiogenesis. Other applications include conditions genetically associated with RELN loss-of-function including lateral temporal epilepsy, autism, Attention Deficit Hyperactivity Disorder (ADHD) schizophrenia, and bipolar disease, as well as cortex lamination defects, abnormal neuronal migration, and cerebellar hypotrophy). See, e.g. Shifman et al., 2008; Ober et al., 2008; Goes et al., 2010; Seripa et al., 2008; Feher et al., 2015; Kelemenova et al., 2009; and Abo El Fotoh et al., 2020.

### Reelin and Mini-Reelin

Reelin useful in the methods and compositions described herein can include full-length wild type reelin, as well as truncated and deletion variants thereof that retain function of the full-length protein, i.e., the ability to bind HSPGs and/or bind to receptors like APOER2 or VLDLR, and/or lead to the activation of downstream targets like Dab1 or promote resilience of neurons or glial cells, and/or bind to NRP1.

Exemplary full length human reelin sequences include the following:

| Nucleic acid | Protein | Variant/Isoform |
|---|---|---|
| NM_005045.4 | NP_005036.2 | Variant 1/reelin isoform a precursor* |
| NM_173054.3 | NP_774959.1 | Variant 2/reelin isoform b precursor** |

*Variant 1 represents the longer transcript, and encodes the longer isoform a.
**Variant 2 lacks an in-frame, 6 nt microexon in the coding region compared to variant 1, resulting in an isoform b that is 2 aa shorter than isoform a.

* Variant 1 represents the longer transcript, and encodes the longer isoform a.
**Variant 2 lacks an in-frame, 6 nt microexon in the coding region compared to variant 1, resulting in an isoform b that is 2 aa shorter than isoform a.
In some embodiments, the methods and compositions described herein us a sequence that is at least 80%, 85%, 90%, 95%, or 99% identical to human reelin.

In some embodiments, a mini-RELN (minimum RELN) is used. An exemplary mini-RELN can include a sequence as described herein; the mini RELN constructs can optionally include protein linkers between these domains, e.g., comprising amino acids between the domains that don't affect function such as Gly-Ser linkers; many others are known in the art, see, e.g., Chen et al., 2012. In some embodiments, the mini-RELN comprises: A) a signal peptide, (B) an oligomerizationdomain, e.g., a dimerization domain, e.g., a CR-50 Domain, (C) an APOER2/VLDLR binding domain, e.g., reelin domains 5 and 6 (R5-6), and (D) GAG binding domain/cell penetrating peptide, e.g., a C-terminus from reelin, e.g., as described herein, e.g., in Table A.

TABLE A
elements of Mini RELN

| ELE- | | |
|---|---|---|
| MENT | FUNCTION | EXAMPLES |
| A | Signal | RELN signal peptide, IL2 signal peptide, |
| | peptide for | human albumin signal peptide, human alpha |
| | secretion | 1-antitrypsin signal peptide, or human factor |
| | | VIII signal peptide |
| B | Oligo- | CR50 region of RELN with or without the |
| | merization | Fsp domain of RELN or Fc fragment of IgG |
| | domain | or FKBP, see, e.g., Dang et al., Front Chem. |
| | | 2022 Feb. 8; 10: 829312 |
| C | APOER2/ | RELN domains including 5 and 6 (R5-6) or |
| | VLDLR | two R6 in tandem (R6-6) or R3 through 6 |
| | binding | (R3-6) or R3 and R6 (R3,6) or Receptor |
| | domain | binding domain of APOE, RAP, urokinase- |
| | | type plasminogen activator (uPA), |
| | | thrombospondin, f-spondin, or SEPP1, see |
| | | Reddy et al., Mol Neurodegener. 2011 May |
| | | 9; 6: 30 |
| D | GAG binding | C-terminus from RELN, TAT peptide, or |
| | domain/cell | P21; see Dixon et al., Proc Natl Acad Sci |
| | penetrating | USA. 2016 Jan. 19; 113(3): E291-9 |
| | peptide | |

Additional sequences, e.g., from RELN, can also be included, but the mini-RELN is not the same as the full length RELN. Mini-RELN may be preferable because it is easier to administer, cheaper to produce, and feasible to deliver for expression in standard AAV vectors or via mRNA or as a recombinant protein. Preferably the RELN sequences, when used, are from human RELN.

In some embodiments a mini-RELN can include: a signal peptide, RAP, IgG Fc, and C-terminus RELN including the H3447R or H3447K mutation and/or a R3454A mutation, optionally H3447R or H3447K in combination with an R3454A mutation; a signal peptide, IgG Fc, RAP, and C-terminus RELN including the H3447R or H3447K mutation and/or a R3454A mutation, optionally H3447R or H3447K in combination with an R3454A mutation; or a signal peptide, R3-6, and C-terminus RELN including the H3447R or H3447K mutation and/or a R3454A mutation, optionally H3447R or H3447K in combination with an R3454A mutation. In some embodiments, the C-terminus RELN including the H3447R or H3447K mutation and/or a R3454A mutation, optionally H3447R or H3447K in combination with an R3454A mutation, can be longer than 30 amino acids (e.g., R8 to CTR—amino acids 3051-3460) but is less than the full length reelin, e.g., up to 100, 105, 110, 120, 130, 150, 200, 300, 400, or 410 amino acids.

In some embodiments, at the N terminus, the mini-RELN includes a signal peptide from RELN, or a signal peptide from another protein (e.g., IL2 signal peptide, human albumin signal peptide, human alpha 1-antitrypsin signal peptide, or human factor VIII signal peptide, not shown in SEQ ID NO:1 below); a CR-50 Domain (e.g., amino acids 229 to 345 of full length RELN, shown in lower case below); a linker (e.g., as shown in lower case italics); R5-6 of RELN (e.g., amino acids 1918 to 2664); and C-terminus (e.g., amino acids 3429 to 3460, shown in italics, with bold font indicating the position of the H3447R variant). SEQ ID NO:1 is the exemplary 225Z construct; other constructs including those shown herein can also be used.

(SEQ ID NO: 1)
eqcgaimhgnavtfcepygprelittglntttasvlqfsigsgscrfsys
dpsiivlyaknnsadwiqlekirapsnvstiihilylpedakgenvqfqw
kqenlrvgevyeacwalgsrggQTNATRFRLWQPYNNGKKEEIWIVDDFI
IDGNNVNNPVMLLDTFDFGPREDNWFFYPGGNIGLYCPYSSKGAPEEDSA
MVFVSNEVGEHSITTRDLNVNENTIIQFEINVGCSTDSSSADPVRLEFSR
DFGATWHLLLPLCYHSSSHVSSLCSTEHHPSSTYYAGTMQGWRREVVHFG
KLHLCGSVRFRWYQGFYPAGSQPVTWAIDNVYIGPQCEEMCNGQGSCING
TKCICDPGYSGPTCKISTKNPDFLKDDFEGQLESDRFLLMSGGKPSRKCG
ILSSGNNLFFNEDGLRMLMTRDLDLSHARFVQFFMRLGCGKGVPDPRSQP
VLLQYSLNGGLSWSLLQEFLFSNSSNVGRYIALEIPLKARSGSTRLRWWQ
PSENGHFYSPWVIDQILIGGNISGNMVLEDDFTTLDSRKWLLHPGGTKMP
VCGSTGDALVFIEKASTRYVVSTDVAVNEDSFLQIDFAVSCSVTDSCYAI
ELEYSVDLGLSWHPLVRDCLPTNVECSRYHLQRILVSDTFNKWTRITLPL
PPYTRSQATRFRWHQPAPFDKQQTWAIDNVYIGDGCIDMCSGHGRCIQGN
CVCDEQWGGLYCDDPETSLPTQLKDNFNRAPSSQNWLTVNGGKLSTVCGA
VASGMALHFSGGCSRLLVTVDLNLTNAEFIQFYFMYGCLITPNNRNQGVL
LEYSVNGGITWNLLMEIFYDQYSKPGFVNILLPPDAKEIATRFRWWQPRH
DGLDQNDWAIDNVLISGSASTRKQNYMMNFSRQHGLRHFYNRRRRSLRRY
P

Additional exemplary sequences of miniRELN constructs include the following (the key of font and style below indicates the order of the different modules):

184I pfcn-huIgG2-C-termH3447WT (nucleotide sequence)
IL2SS-igg2-fc-LINKER-c-terminus reln h3447WT
(SEQ ID NO: 2)
ATGTACAGGATGCAACTCCTGTCTTGCATTGCACTAAGTCTTGCACTT
GTCACGAATTCGgcacctctcgagcgcaaatctagtgtcgagtgcccaccgtgcccagcaccacctgtggca
ggaccgtcagtcttcctcttccccccaaaacccaaggacaccctcatgatctcccggacccctgaggtcacgtgcgtggtg
gtggacgtgagccacgaagaccccgaggtccagttcaactggtacgtggacggcgtggaggtgcataatgccaagacaa
agccacgggaggagcagttcaacagcacgttccgtgtggtcagcgtcctcaccgttgtgcaccaggactggctgaacgg
caaggagtacaagtgcaaggtctccaacaaaggcctcccagcccccatcgagaaaaccatctccaaaaccaaagggca
gccccgagaaccacaggtgtacaccctgcccccatcccgggaggagatgaccaagaaccaggtcagcctgacctgcct
ggtcaaaggcttctaccccagcgacatcgccgtggagtgggagagcaatgggcagccggagaacaactacaagaccac
gcctcccatgctggactccgacggctccttcttcctctacagcaagctcaccgtggacaagagcaggtggcagcagggga
acgtcttctcatgctccgtgatgcatgaggctctgcacaaccactacacgcagaagagcctctccctgtctccgggtgcaC
GTACGagcactcgcaaacaaaattacatgatgaatttttcacgacaacatgggctcagacatttctacaacaga
agacgaaggtcacttaggcgatacccat
184I pfcn-huIgG2-C-termH3447WT (amino acid sequence-translation of the
sequence by domains))
IL2SS-igg2-fc-LINKER-c-terminus reln h3447WT
(SEQ ID NO: 3)
MYRMQLLSCIALSLALVTNSaplerkssvecppcpappvagpsvflfppkpkdtlmisrtpevtcvv
vdvshedpevqfnwyvdgvevhnaktkpreeqfnstfrvvsvltvvhqdwlngkeykckvsnkglpapiektiskt
kgqprepqvytlppsre
Emtknqvsltclvkgfypsdiavewesngqpennykttppmldsdgsfflyskltvdksrwqqgnvfscsvmheal
hnhytqkslslspgaRTstrkqnymmnfsrqhglrhfynrrrrslrryp
184J pfcn-huIgG2-C-termH3447H3447R (nucleotide sequence)
IL2SS-igg2-fc-LINKER-c-terminus reln h3447r
(SEQ ID NO: 4)
ATGTACAGGATGCAACTCCTGTCTTGCATTGCACTAAGTCTTGCACTT
GTCACGAATTCGgcacctctcgagcgcaaatctagtgtcgagtgcccaccgtgcccagcaccacctgtggca
ggaccgtcagtcttcctcttccccccaaaacccaaggacaccctcatgatctcccggacccctgaggtcacgtgcgtggtg
gtggacgtgagccacgaagaccccgaggtccagttcaactggtacgtggacggcgtggaggtgcataatgccaagacaa
agccacgggaggagcagttcaacagcacgttccgtgtggtcagcgtcctcaccgttgtgcaccaggactggctgaacgg
caaggagtacaagtgcaaggtctccaacaaaggcctcccagcccccatcgagaaaaccatctccaaaaccaaagggca
gccccgagaaccacaggtgtacaccctgcccccatcccgggaggagatgaccaagaaccaggtcagcctgacctgcct
ggtcaaaggcttctaccccagcgacatcgccgtggagtgggagagcaatgggcagccggagaacaactacaagaccac
gcctcccatgctggactccgacggctccttcttcctctacagcaagctcaccgtggacaagagcaggtggcagcagggga
acgtcttctcatgctccgtgatgcatgaggctctgcacaaccactacacgcagaagagcctctccctgtctccgggtgcaC
GTACGagcactcgcaaacaaaattacatgatgaatttttcacgacaacatgggctcagacgtttctacaacaga
agacgaaggtcacttaggcgatacccat
184J pfcn-huIgG2-C-termH3447R (amino acid sequence-translation of the
sequence by domains))
IL2SS-igg2-fc-LINKER-c-terminus reln h3447r
(SEQ ID NO: 5)
MYRMQLLSCIALSLALVTNSaplerkssvecppcpappvagpsvflfppkpkdtlmisrtpevtcvv
vdvshedpevqfnwyvdgvevhnaktkpreeqfnstfrvvsvltvvhqdwlngkeykckvsnkglpapiektiskt
kgqprepqvytlppsre
Emtknqvsltclvkgfypsdiavewesngqpennykttppmldsdgsfflyskltvdksrwqqgnvfscsvmheal
hnhytqkslslspgaRTstrkqnymmnfsrqhglrrfynrrrrslrryp
225Q pFUSEN-hG2Fc-R5-6-C-termH3447R (nucleotide sequence)
IL2SS-igg2fc-LINKER-r5-6 of reln-c-terminus reln h3447r
(SEQ ID NO: 6)
ATGTACAGGATGCAACTCCTGTCTTGCATTGCACTAAGTCTTGCACTT
GTCACGAATTCGgcacctctcgagcgcaaatctagtgtcgagtgcccaccgtgcccagcaccacctgtggca
ggaccgtcagtcttcctcttccccccaaaacccaaggacaccctcatgatctcccggacccctgaggtcacgtgcgtggtg
gtggacgtgagccacgaagaccccgaggtccagttcaactggtacgtggacggcgtggaggtgcataatgccaagacaa
agccacgggaggagcagttcaacagcacgttccgtgtggtcagcgtcctcaccgttgtgcaccaggactggctgaacgg
caaggagtacaagtgcaaggtctccaacaaaggcctcccagcccccatcgagaaaaccatctccaaaaccaaagggca
gccccgagaaccacaggtgtacaccctgcccccatcccgggaggagatgaccaagaaccaggtcagcctgacctgcct
ggtcaaaggcttctaccccagcgacatcgccgtggagtgggagagcaatgggcagccggagaacaactacaagaccac
gcctcccatgctggactccgacggctccttcttcctctacagcaagctcaccgtggacaagagcaggtggcagcagggga
acgtcttctcatgctccgtgatgcatgaggctctgcacaaccactacacgcagaagagcctctccctgtctccgggtgcaC
GTACGGGTGGTGGCGGTAGCGGTGGTGGCGGATCCcaaaccaatgctacaagattcag
actctggcaaccttataataacggtaagaaagaagaaatctggattgttgatgacttcattatcgatggaaataatgtaa
acaaccctgtgatgctcttggatacatttgattttgggcccagagaagacaattggtttttctatcctggtggtaacatcggt
ctttattgtccatattcttcaaagggggcacctgaagaagattcagctatggtgtttgtttcaaatgaagttggtgagcattc
cattaccacccgtgacctaaatgtgaatgagaacaccatcatacaatttgagatcaacgttggctgttcgactgatagct
catccgcggatccagtgagactggaattttcaagggacttcggggcgacctggcaccttctgctgcccctctgctaccac
agcagcagccacgtcagctctttatgctccaccgagcaccaccccagcagcacctactacgcaggaaccatgcaggg
ctggaggagggaggtcgtgcactttgggaagctgcacctttgtggatctgtccgtttcagatggtaccagggattttaccc
tgccggctctcagccagtgacatgggccattgataatgtctacatcggtccccagtgtgaggagatgtgtaatggacag
gggagctgtatcaatggaaccaaatgtatatgtgaccctggctactcaggtccaacctgtaaaataagcaccaaaaat
cctgattttctcaaagatgatttcgaaggtcagctagaatctgatagattcttattaatgagtggtgggaaaccatctcgaa
agtgtggaatcctttctagtggaaacaacctctttttcaatgaagatggcttgcgcatgttgatgacacgagacctggattt
atcacatgctagatttgtgcagttcttcatgagactgggatgtggtaaaggcgttcctgaccccaggagtcaacccgtgct
cctacagtattctctcaacggtggcctctcgtggagtcttcttcaggagttccttttcagcaattccagcaatgtgggcaggt
acattgccctggagatacccttgaaagcccgttctggttctactcgccttcgctggtggcaaccgtctgagaatgggcact
tctacagcccctgggttatcgatcagattcttattggaggaaatatttctggtaatatggtcttggaagatgatttcacaacc
cttgatagtaggaaatggctgcttcacccaggaggcaccaagatgcccgtgtgtggctctactggtgatgccctggtctt
cattgaaaaggccagcacccgttacgtggtcagcacagacgtcgccgtgaatgaggattccttcctacagatagacttc
gctgtctcctgctcagtcacagactcttgttatgcgattgaattggaatactcagtagatcttggattgtcatggcacccatt
ggtaagggactgtctgcctaccaatgtggaatgcagtcgctatcatctgcaacggatcctggtgtcagacactttcaaca
agtggactagaatcactctgcctctccctccttataccaggtcccaagccactcgtttccgttggcatcaaccagctcctttt
gacaagcagcagacatgggcaatagataatgtctatatcggggatggctgcatagacatgtgcagtggccatgggag
atgcatccagggaaactgcgtctgtgatgaacagtggggtggcctgtactgtgatgaccccgagacctetettccaacc
caactcaaagacaacttcaatcgagctccatccagtcagaactggctgactgtgaacggagggaaattgagtacagt
gtgtggagccgtggcgtcgggaatggctctccatttcagtgggggttgtagtcgattattagtcactgtggatctaaacctc
actaatgctgagttcatccaattttacttcatgtatgggtgcctgattacaccaaacaaccgtaaccaaggtgttctcttgg
aatattctgtcaatggaggcattacctggaacctgctcatggagattttctatgaccagtacagtaagcccggatttgtga
atatccttctccctcctgatgctaaagagattgccactcgcttccgctggtggcagccaagacatgacggcctggatcag
aacgactgggccattgacaatgtcctcatctcaggctctgctagcactcgcaaacaaaattacatgatgaatttttcac
gacaacatgggctcagacgtttctacaacagaagacgaaggtcacttaggcgatacccat
225Q pFUSEN-hG2Fc-R5-6-C-termH3447R (amino acid sequence-translation
of the sequence by domains)
IL2SS-igg2fc-LINKER-r5-6 of reln-c-terminus reln h3447r
(SEQ ID NO: 7)
MYRMQLLSCIALSLALVTNSaplerkssvecppcpappvagpsvflfppkpkdtlmisrtpevtcvv
vdvshedpevqfnwyvdgvevhnaktkpreeqfnstfrvvsvltvvhqdwlngkeykckvsnkglpapiektiskt
kgqprepqvytlppsreemtknqvsltclvkgfypsdiavewesngqpennykttppmldsdgsfflyskltvdksr
wqqgnvfscsvmhealhnhytqkslslspgaRTGGGGSGGGGSqtnatrfrlwqpynngkkeeiwivddfi
idgnnvnnpvmlldtfdfgprednwffypggniglycpysskgapeedsamvfvsnevgehsittrdlnvnentiiqfei
nvgcstdsssadpvrlefsrdfgatwhlllplcyhssshvsslcstehhpsstyyagtmqgwrrevvhfgklhlcgsvrfr
wyqgfypagsqpvtwaidnvyigpqceemcngqgscingtkcicdpgysgptckistknpdflkddfegqlesdrfll
msggkpsrkcgilssgnnlffnedglrmlmtrdldlsharfvqffmrlgcgkgvpdprsqpvllqyslngglswsllqefl
fsnssnvgryialeiplkarsgstrlrwwqpsenghfyspwvidqiliggnisgnmvleddfttldsrkwllhpggtkmp
vcgstgdalvfiekastryvvstdvavnedsflqidfavscsvtdscyaieleysvdlglswhplvrdclptnvecsryhlqr
ilvsdtfnkwtritlplppytrsqatrfrwhqpapfdkqqtavaidnvyigdgcidmcsghgrciqgncvcdeqvgglyc
ddpetslptqlkdnfnrapssqnwltvnggklstvcgavasgmalhfsggcsrllvtvdlnltnaefiqfyfmygclitpnn
rnqgvlleysvnggitwnllmeifydqyskpgfvnillppdakeiatrfrwwqprhdgldqndwaidnvlisgsastrk
qnymmnfsrqhglrrfynrrrrslrryp
225R pFUSEN-flag-CR-50-R5-6-CtermH3447R (nucleotide sequence)
IL2SS-LINKER-flag tag-LINKER-cr50 of reln-LINKER-R5-6 OF RELN-c-
terminus reln H3447R
(SEQ ID NO: 8)
ATGTACAGGATGCAACTCCTGTCTTGCATTGCACTAAGTCTTGCACTT
GTCACGAATTCGGGTGGTgactacaaagacgatgacgacaagGGAGGTgaacagtgtggcgcg
attatgcatggcaatgccgtcaccttctgtgaaccatatggcccacgagaactgattaccacaggccttaatacaacaa
cagcttctgtcctccaattttccattgggtcaggttcatgtcgctttagttattcagaccccagcatcatcgtgttatatgccaa
gaataactctgcggactggattcagctagagaaaattagagccccttccaatgtcagcacaatcatccatatcctctacc
ttcctgaggacgccaaaggggagaatgtccaatttcagtggaagcaggaaaatcttcgtgtaggtgaagtgtatgaag
cctgctgggccttaGGAGGACAAACCAATGCTACAAGATTCAGACTCTGGCAACCT
TATAATAACGGTAAGAAAGAAGAAATCTGGATTGTTGATGACTTCATTATCG
ATGGAAATAATGTAAACAACCCTGTGATGCTCTTGGATACATTTGATTTTGG
GCCCAGAGAAGACAATTGGTTTTTCTATCCTGGTGGTAACATCGGTCTTTAT
TGTCCATATTCTTCAAAGGGGGCACCTGAAGAAGATTCAGCTATGGTGTTT
GTTTCAAATGAAGTTGGTGAGCATTCCATTACCACCCGTGACCTAAATGTG
AATGAGAACACCATCATACAATTTGAGATCAACGTTGGCTGTTCGACTGAT
AGCTCATCCGCGGATCCAGTGAGACTGGAATTTTCAAGGGACTTCGGGGC
GACCTGGCACCTTCTGCTGCCCCTCTGCTACCACAGCAGCAGCCACGTCAG
CTCTTTATGCTCCACCGAGCACCACCCCAGCAGCACCTACTACGCAGGAAC
CATGCAGGGCTGGAGGAGGGAGGTCGTGCACTTTGGGAAGCTGCACCTTT
GTGGATCTGTCCGTTTCAGATGGTACCAGGGATTTTACCCTGCCGGCTCTCA
GCCAGTGACATGGGCCATTGATAATGTCTACATCGGTCCCCAGTGTGAGGA
GATGTGTAATGGACAGGGGAGCTGTATCAATGGAACCAAATGTATATGTGA
CCCTGGCTACTCAGGTCCAACCTGTAAAATAAGCACCAAAAATCCTGATTT
TCTCAAAGATGATTTCGAAGGTCAGCTAGAATCTGATAGATTCTTATTAATG
AGTGGTGGGAAACCATCTCGAAAGTGTGGAATCCTTTCTAGTGGAAACAA
CCTCTTTTTCAATGAAGATGGCTTGCGCATGTTGATGACACGAGACCTGGA
TTTATCACATGCTAGATTTGTGCAGTTCTTCATGAGACTGGGATGTGGTAAA
GGCGTTCCTGACCCCAGGAGTCAACCCGTGCTCCTACAGTATTCTCTCAAC
GGTGGCCTCTCGTGGAGTCTTCTTCAGGAGTTCCTTTTCAGCAATTCCAGC
AATGTGGGCAGGTACATTGCCCTGGAGATACCCTTGAAAGCCCGTTCTGGT
TCTACTCGCCTTCGCTGGTGGCAACCGTCTGAGAATGGGCACTTCTACAGC
CCCTGGGTTATCGATCAGATTCTTATTGGAGGAAATATTTCTGGTAATATGGT
CTTGGAAGATGATTTCACAACCCTTGATAGTAGGAAATGGCTGCTTCACCC
AGGAGGCACCAAGATGCCCGTGTGTGGCTCTACTGGTGATGCCCTGGTCTT
CATTGAAAAGGCCAGCACCCGTTACGTGGTCAGCACAGACGTCGCCGTGA
ATGAGGATTCCTTCCTACAGATAGACTTCGCTGTCTCCTGCTCAGTCACAGA
CTCTTGTTATGCGATTGAATTGGAATACTCAGTAGATCTTGGATTGTCATGGC
ACCCATTGGTAAGGGACTGTCTGCCTACCAATGTGGAATGCAGTCGCTATCA
TCTGCAACGGATCCTGGTGTCAGACACTTTCAACAAGTGGACTAGAATCAC
TCTGCCTCTCCCTCCTTATACCAGGTCCCAAGCCACTCGTTTCCGTTGGCAT
CAACCAGCTCCTTTTGACAAGCAGCAGACATGGGCAATAGATAATGTCTATA
TCGGGGATGGCTGCATAGACATGTGCAGTGGCCATGGGAGATGCATCCAGG
GAAACTGCGTCTGTGATGAACAGTGGGGTGGCCTGTACTGTGATGACCCCG
AGACCTCTCTTCCAACCCAACTCAAAGACAACTTCAATCGAGCTCCATCCA
GTCAGAACTGGCTGACTGTGAACGGAGGGAAATTGAGTACAGTGTGTGGA
GCCGTGGCGTCGGGAATGGCTCTCCATTTCAGTGGGGGTTGTAGTCGATTA
TTAGTCACTGTGGATCTAAACCTCACTAATGCTGAGTTCATCCAATTTTACT
TCATGTATGGGTGCCTGATTACACCAAACAACCGTAACCAAGGTGTTCTCTT
GGAATATTCTGTCAATGGAGGCATTACCTGGAACCTGCTCATGGAGATTTTC
TATGACCAGTACAGTAAGCCCGGaTTTGTGAATATCCTTCTCCCTCCTGATG
CTAAAGAGATTGCCACTCGCTTCCGCTGGTGGCAGCCAAGACATGACGGC
CTGGATCAGAACGACTGGGCCATTGACAATGTCCTCATCTCAGGCTCTGCT
agcactcgcaaacaaaattacatgatgaatttttcacgacaacatgggctcagacgtttctacaacagaagacgaa
ggtcacttaggcgatacccat
225R pFUSEN-flag-CR-50-R5-6-CtermH3447R (amino acid sequence-
translation of the sequence by domains)
IL2SS-LINKER-flag tag-LINKER-cr50 of reln-LINKER-R5-6 OF RELN-c-
terminus reln H3447R
(SEQ ID NO: 9)
MYRMQLLSCIALSLALVTNSGGdykddddkGGeqcgaimhgnavtfcepygprelittglnttta
svlqfsigsgscrfsysdpsiivlyaknnsadwiglekirapsnvstiihilylpedakgenvqfqwkqenlrvgevyeac
walGGQTNATRFRLWQPYNNGKKEEIWIVDDFIIDGNNVNNPVMLLDTFDFGP
REDNWFFYPGGNIGLYCPYSSKGAPEEDSAMVFVSNEVGEHSITTRDLNVNE
NTIIQFEINVGCSTDSSSADPVRLEFSRDFGATWHLLLPLCYHSSSHVSSLCSTE
HHPSSTYYAGTMQGWRREVVHFGKLHLCGSVRFRWYQGFYPAGSQPVTWA
IDNVYIGPQCEEMCNGQGSCINGTKCICDPGYSGPTCKISTKNPDFLKDDFEG
QLESDRFLLMSGGKPSRKCGILSSGNNLFFNEDGLRMLMTRDLDLSHARFVQ
FFMRLGCGKGVPDPRSQPVLLQYSLNGGLSWSLLQEFLFSNSSNVGRYIALEI
PLKARSGSTRLRWWQPSENGHFYSPWVIDQILIGGNISGNMVLEDDFTTLDSR
KWLLHPGGTKMPVCGSTGDALVFIEKASTRYVVSTDVAVNEDSFLQIDFAVS
CSVTDSCYAIELEYSVDLGLSWHPLVRDCLPTNVECSRYHLQRILVSDTFNK
WTRITLPLPPYTRSQATRFRWHQPAPFDKQQTWAIDNVYIGDGCIDMCSGHG
RCIQGNCVCDEQWGGLYCDDPETSLPTQLKDNFNRAPSSQNWLTVNGGKLS
TVCGAVASGMALHFSGGCSRLLVTVDLNLTNAEFIQFYFMYGCLITPNNRNQ
GVLLEYSVNGGITWNLLMEIFYDQYSKPGFVNILLPPDAKEIATRFRWWQPR
HDGLDQNDWAIDNVLISGSAstrkqnymmnfsrqhglrrfynrrrrslrryp
225S pFUSEN-FcIgG-RAP-CTRH3447R (nucleotide sequence)
IL2SS-igg2-fc-LINKER-rap-LINKER-c-terminus reln H3447R
(SEQ ID NO: 10)
ATGTACAGGATGCAACTCCTGTCTTGCATTGCACTAAGTCTTGCACTT
GTCACGAATTCGgcacctctcgagcgcaaatctagtgtcgagtgcccaccgtgcccagcaccacctgtggca
ggaccgtcagtcttcctcttccccccaaaacccaaggacaccctcatgatctcccggacccctgaggtcacgtgcgtggtg
gtggacgtgagccacgaagaccccgaggtccagttcaactggtacgtggacggcgtggaggtgcataatgccaagacaa
agccacgggaggagcagttcaacagcacgttccgtgtggtcagcgtcctcaccgttgtgcaccaggactggctgaacgg
caaggagtacaagtgcaaggtctccaacaaaggcctcccagcccccatcgagaaaaccatctccaaaaccaaagggca
gccccgagaaccacaggtgtacaccctgcccccatcccgggaggagatgaccaagaaccaggtcagcctgacctgcct
ggtcaaaggcttctaccccagcgacatcgccgtggagtgggagagcaatgggcagccggagaacaactacaagaccac
gcctcccatgctggactccgacggctccttcttcctctacagcaagctcaccgtggacaagagcaggtggcagcagggga
acgtcttctcatgctccgtgatgcatgaggctctgcacaaccactacacgcagaagagcctctccctgtctccgggtgcaC
GTACGGGTGGTGGCGGTAGCGGTGGTGGCGGATCCatggcgccgcggagggtcaggtc
gtttctgcgcgggctcccggcgctgctactgctgctgctcttcctcgggccctggcccgctgcgagccacggcggcaagt
actcgcgggagaagaaccagcccaagccgtccccgaaacgcgagtccggagaggagttccgcatggagaagttga
accagctgtgggagaaggcccagcgactgcatcttcctcccgtgaggctggccgagctccacgctgatctgaagatac
aggagagggacgaactcgcctggaagaaactaaagcttgacggcttggacgaagatggggagaaggaagcgaga
ctcatacgcaacctcaatgtcatcttggccaagtatggtctggacggaaagaaggacgctcggcaggtgaccagcaa
ctccctcagtggcacccaggaagacgggctggatgaccccaggctggaaaagctgtggcacaaggcgaagacctct
gggaaattctccggcgaagaactggacaagctctggcgggagttcctgcatcacaaagagaaagttcacgagtaca
acgtcctgctggagaccctgagcaggaccgaagaaatccacgagaacgtcattagcccctcggacctgagcgacat
caagggcagcgtcctgcacagcaggcacacggagctgaaggagaagctgcgcagcatcaaccagggcctggacc
gcctgcgcagggtcagccaccagggctacagcactgaggctgagttcgaggagcccagggtgattgacctgtggga
cctggcgcagtccgccaacctcacggacaaggagctggaggcgttccgggaggagctcaagcacttcgaagccaa
aatcgagaagcacaaccactaccagaagcagctggagattgcgcacgagaagctgaggcacgcagagagcgtgg
gcgacggcgagcgtgtgagccgcagccgcgagaagcacgccctgctggaggggcggaccaaggagctgggctac
acggtgaagaagcatctgcaggacctgtccggcaggatctccagagctcggcacaacgaactcACCGGTGGA
TCTGGAGGGagcactcgcaaacaaaattacatgatgaatttttcacgacaacatgggctcagacgtttctac
aacagaagacgaaggtcacttaggcgatacccat
225S pFUSEN-FcIgG-RAP-CTRH3447R (amino acid sequence-translation of
the sequence by domains)
IL2SS-igg2-fc-LINKER-rap-LINKER-c-terminus reln H3447R
(SEQ ID NO: 11)
MYRMQLLSCIALSLALVTNSaplerkssvecppcpappvagpsvflfppkpkdtlmisrtpevtcvv
vdvshedpevqfnwyvdgvevhnaktkpreeqfnstfrvvsvltvvhqdwlngkeykckvsnkglpapiektiskt
kgqprepqvytlppsreemtknqvsltclvkgfypsdiavewesngqpennykttppmldsdgsfflyskltvdksr
wqqgnvfscsvmhealhnhytqkslslspgaRTGGGGSGGGGSmaprrvrsflrglpallllllflgpwpaas
hggkysreknqpkpspkresgeefrmeklnqlwekaqrlhlppvrlaelhadlkiqerdelawkklkldgldedgeke
arlirnlnvilakygldgkkdarqvtsnslsgtqedglddprleklwhkaktsgkfsgeeldklwreflhhkekvheynvll
etlsrteeihenvispsdlsdikgsvlhsrhtelkeklrsinqgldrlrrvshqgysteaefeeprvidlwdlaqsanltdkel
eafreelkhfeakiekhnhyqkqleiaheklrhaesvgdgervsrsrekhallegrtkelgytvkkhlqdlsgrisrarhne
lTGGSGGstrkqnymmnfsrqhglrrfynrrrrslrryp
225T pFUSEN-RAP-FcIgG-CTRH3447R (nucleotide sequence)
IL2SS-rap-LINKER-igg2-fc-LINKER-c-terminus reln H3447R
(SEQ ID NO: 12)
ATGTACAGGATGCAACTCCTGTCTTGCATTGCACTAAGTCTTGCACTT
GTCACGAATTCCatggcgccgcggagggtcaggtcgtttctgcgcgggctcccggcgctgctactgctgctg
ctcttcctcgggccctggcccgctgcgagccacggcggcaagtactcgcgggagaagaaccagcccaagccgtccc
cgaaacgcgagtccggagaggagttccgcatggagaagttgaaccagctgtgggagaaggcccagcgactgcatct
tcctcccgtgaggctggccgagctccacgctgatctgaagatacaggagagggacgaactcgcctggaagaaacta
aagcttgacggcttggacgaagatggggagaaggaagcgagactcatacgcaacctcaatgtcatcttggccaagta
tggtctggacggaaagaaggacgctcggcaggtgaccagcaactccctcagtggcacccaggaagacgggctgga
tgaccccaggctggaaaagctgtggcacaaggcgaagacctctgggaaattctccggcgaagaactggacaagctc
tggcgggagttcctgcatcacaaagagaaagttcacgagtacaacgtcctgctggagaccctgagcaggaccgaag
aaatccacgagaacgtcattagcccctcggacctgagcgacatcaagggcagcgtcctgcacagcaggcacacgga
gctgaaggagaagctgcgcagcatcaaccagggcctggaccgcctgcgcagggtcagccaccagggctacagcac
tgaggctgagttcgaggagcccagggtgattgacctgtgggacctggcgcagtccgccaacctcacggacaaggag
ctggaggcgttccgggaggagctcaagcacttcgaagccaaaatcgagaagcacaaccactaccagaagcagctg
gagattgcgcacgagaagctgaggcacgcagagagcgtgggcgacggcgagcgtgtgagccgcagccgcgaga
agcacgccctgctggaggggcggaccaaggagctgggctacacggtgaagaagcatctgcaggacctgtccggca
ggatctccagagctcggcacaacgaactcACCGGTGGATCTGGAGGGAATTCGgcacctctcgag
cgcaaatctagtgtcgagtgcccaccgtgcccagcaccacctgtggcaggaccgtcagtcttcctcttccccccaaaaccc
aaggacaccctcatgatctcccggacccctgaggtcacgtgcgtggtggtggacgtgagccacgaagaccccgaggtcc
agttcaactggtacgtggacggcgtggaggtgcataatgccaagacaaagccacgggaggagcagttcaacagcacgtt
ccgtgtggtcagcgtcctcaccgttgtgcaccaggactggctgaacggcaaggagtacaagtgcaaggtctccaacaaag
gcctcccagcccccatcgagaaaaccatctccaaaaccaaagggcagccccgagaaccacaggtgtacaccctgcccc
catcccgggaggagatgaccaagaaccaggtcagcctgacctgcctggtcaaaggcttctaccccagcgacatcgccgt
ggagtgggagagcaatgggcagccggagaacaactacaagaccacgcctcccatgctggactccgacggctccttcttc
ctctacagcaagctcaccgtggacaagagcaggtggcagcaggggaacgtcttctcatgctccgtgatgcatgaggctct
gcacaaccactacacgcagaagagcctctccctgtctccgggtgcaCGTACGGGTGGTGGCGGTAGC
GGTGGTGGCGGATCCagcactcgcaaacaaaattacatgatgaatttttcacgacaacatgggctcaga
cgtttctacaacagaagacgaaggtcacttaggcgatacccat
225T pFUSEN-RAP-FcIgG-CTRH3447R (amino acid sequence-translation of
the sequence by domains)
IL2SS-rap-LINKER-igg2-fc-LINKER-c-terminus reln H3447R
(SEQ ID NO: 13)
MYRMQLLSCIALSLALVTNSmaprrvrsflrglpallllllflgpwpaashggkysreknqpkpspkre
sgeefrmeklnqlwekaqrlhlppvrlaelhadlkiqerdelawkklkldgldedgekearlirnlnvilakygldgkkd
arqvtsnslsgtqedglddprleklwhkaktsgkfsgeeldklwreflhhkekvheynvlletlsrteeihenvispsdlsdi
kgsvlhsrhtelkeklrsinqgldrlrrvshqgysteaefeeprvidlwdlaqsanltdkeleafreelkhfeakiekhnhyq
kqleiaheklrhaesvgdgervsrsrekhallegrtkelgytvkkhlqdlsgrisrarhnelTGGSGGNSaplerkss
vecppcpappvagpsvflfppkpkdtlmisrtpevtcvvvdvshedpevqfnwyvdgvevhnaktkpreeqfnstf
rvvsvltvvhqdwlngkeykckvsnkglpapiektisktkgqprepqvytlppsreemtknqvsltclvkgfypsdia
vewesngqpennykttppmldsdgsfflyskltvdksrwqqgnvfscsvmhealhnhytqkslslspgaRTGGG
GSGGGGSstrkqnymmnfsrqhglrrfynrrrrslrryp
225Xf pFUSEN-IL2ss-FcigG-APOE-R154S-CTRH3447R (nucleotide sequence)
IL2SS-igg2-fc-LINKER-apoe r154s christchurch-LINKER-c-terminus reln
H3447R
(SEQ ID NO: 14)
ATGTACAGGATGCAACTCCTGTCTTGCATTGCACTAAGTCTTGCACTT
GTCACGAATTCGgcacctctcgagcgcaaatctagtgtcgagtgcccaccgtgcccagcaccacctgtggca
ggaccgtcagtcttcctcttccccccaaaacccaaggacaccctcatgatctcccggacccctgaggtcacgtgcgtggtg
gtggacgtgagccacgaagaccccgaggtccagttcaactggtacgtggacggcgtggaggtgcataatgccaagacaa
agccacgggaggagcagttcaacagcacgttccgtgtggtcagcgtcctcaccgttgtgcaccaggactggctgaacgg
caaggagtacaagtgcaaggtctccaacaaaggcctcccagcccccatcgagaaaaccatctccaaaaccaaagggca
gccccgagaaccacaggtgtacaccctgcccccatcccgggaggagatgaccaagaaccaggtcagcctgacctgcct
ggtcaaaggcttctaccccagcgacatcgccgtggagtgggagagcaatgggcagccggagaacaactacaagaccac
gcctcccatgctggactccgacggctccttcttcctctacagcaagctcaccgtggacaagagcaggtggcagcagggga
acgtcttctcatgctccgtgatgcatgaggctctgcacaaccactacacgcagaagagcctctccctgtctccgggtgcaC
GTACGGGTGGTGGCGGTAGCGGTGGTGGCGGATCGatgaaggttctgtgggctgcgttgc
tggtcacattcctggcaggatgccaggccaaggtggagcaagcggtggagacagagccggagcccgagctgcgcc
agcagaccgagtggcagagcggccagcgctgggaactggcactgggtcgcttttgggattacctgcgctgggtgcag
acactgtctgagcaggtgcaggaggagctgctcagctcccaggtcacccaggaactgagggcgctgatggacgaga
ccatgaaggagttgaaggcctacaaatcggaactggaggaacaactgaccccggtggcggaggagacgcgggca
cggctgtccaaggagctgcaggcggcgcaggcccggctgggcgcggacatggaggacgtgtgcggccgcctggtg
cagtaccgcggcgaggtgcaggccatgctcggccagagcaccgaggagctgcgggtgagcctcgcctcccacctgc
gcaagctgcgtaagcggctcctccgcgatgccgatgacctgcagaagcgcctggcagtgtaccaggccggggcccg
cgagggcgccgagcgcggcctcagcgccatccgcgagcgcctggggcccctggtggaacagggccgcgtgcgggc
cgccactgtgggctccctggccggccagccgctacaggagcgggcccaggcctggggcgagcggctgcgcgcgcg
gatggaggagatgggcagccggacccgcgaccgcctggacgaggtgaaggagcaggtggcggaggtgcgcgcc
aagctggaggagcaggcccagcagatacgcctgcaggccgaggccttccaggcccgcctcaagagctggttcgagc
ccctggtggaagacatgcagcgccagtgggccgggctggtggagaaggtgcaggctgccgtgggcaccagcgccg
cccctgtgcccagcgacaatcacACCGGTGGATCTGGAGGGagcactcgcaaacaaaattacatgat
gaatttttcacgacaacatgggctcagacgtttctacaacagaagacgaaggtcacttaggcgatacccat
225Xf pFUSEN-IL2ss-FcigG-APOE-R154S-CTRH3447R (amino acid sequence-
translation of the sequence by domains)
IL2SS-igg2-fc-LINKER-apoe r154s christchurch-LINKER-c-terminus reln
H3447R
(SEQ ID NO: 15)
MYRMQLLSCIALSLALVTNSaplerkssvecppcpappvagpsvflfppkpkdtlmisrtpevtcvv
vdvshedpevqfnwyvdgvevhnaktkpreeqfnstfrvvsvltvvhqdwlngkeykckvsnkglpapiektiskt
kgqprepqvytlppsreemtknqvsltclvkgfypsdiavewesngqpennykttppmldsdgsfflyskltvdksr
wqqgnvfscsvmhealhnhytqkslslspgaRTGGGGSGGGGSmkvlwaallvtflagcqakveqavetep
epelrqqtewqsgqrwelalgrfwdylrwvqtlseqvqeellssqvtqelralmdetmkelkaykseleeqltpvaeetr
arlskelqaaqarlgadmedvcgrlvqyrgevqamlgqsteelrvslashlrklrkrllrdaddlqkrlavyqagareg
aerglsairerlgplveqgrvraatvgslagqplqeraqawgerlrarmeemgsrtrdrldevkeqvaevrakleeqa
qqirlqaeafqarlkswfeplvedmqrqwaglvekvqaavgtsaapvpsdnhTGGSGGstrkqnymmnfsr
qhglrrfynrrrrslrryp
225Yf pFUSEN-IL2ss-FcigG-APOE-CTRH3447R (nucleotide sequence)
IL2SS-igg2-fc-LINKER-apoe-LINKER-c-terminus reln H3447R
(SEQ ID NO: 16)
ATGTACAGGATGCAACTCCTGTCTTGCATTGCACTAAGTCTTGCACTT
GTCACGAATTCGgcacctctcgagcgcaaatctagtgtcgagtgcccaccgtgcccagcaccacctgtggca
ggaccgtcagtcttcctcttccccccaaaacccaaggacaccctcatgatctcccggacccctgaggtcacgtgcgtggtg
gtggacgtgagccacgaagaccccgaggtccagttcaactggtacgtggacggcgtggaggtgcataatgccaagacaa
agccacgggaggagcagttcaacagcacgttccgtgtggtcagcgtcctcaccgttgtgcaccaggactggctgaacgg
caaggagtacaagtgcaaggtctccaacaaaggcctcccagcccccatcgagaaaaccatctccaaaaccaaagggca
gccccgagaaccacaggtgtacaccctgcccccatcccgggaggagatgaccaagaaccaggtcagcctgacctgcct
ggtcaaaggcttctaccccagcgacatcgccgtggagtgggagagcaatgggcagccggagaacaactacaagaccac
gcctcccatgctggactccgacggctccttcttcctctacagcaagctcaccgtggacaagagcaggtggcagcagggga
acgtcttctcatgctccgtgatgcatgaggctctgcacaaccactacacgcagaagagcctctccctgtctccgggtgcaC
GTACGGGTGGTGGCGGTAGCGGTGGTGGCGGATCGatgaaggttctgtgggctgcgttgc
tggtcacattcctggcaggatgccaggccaaggtggagcaagcggtggagacagagccggagcccgagctgcgcc
agcagaccgagtggcagagcggccagcgctgggaactggcactgggtcgcttttgggattacctgcgctgggtgcag
acactgtctgagcaggtgcaggaggagctgctcagctcccaggtcacccaggaactgagggcgctgatggacgaga
ccatgaaggagttgaaggcctacaaatcggaactggaggaacaactgaccccggtggcggaggagacgcgggca
cggctgtccaaggagctgcaggcggcgcaggcccggctgggcgcggacatggaggacgtgtgcggccgcctggtg
cagtaccgcggcgaggtgcaggccatgctcggccagagcaccgaggagctgcgggtgcgcctcgcctcccacctgc
gcaagctgcgtaagcggctcctccgcgatgccgatgacctgcagaagcgcctggcagtgtaccaggccggggcccg
cgagggcgccgagcgcggcctcagcgccatccgcgagcgcctggggcccctggtggaacagggccgcgtgcgggc
cgccactgtgggctccctggccggccagccgctacaggagcgggcccaggcctggggcgagcggctgcgcgcgcg
gatggaggagatgggcagccggacccgcgaccgcctggacgaggtgaaggagcaggtggcggaggtgcgcgcc
aagctggaggagcaggcccagcagatacgcctgcaggccgaggccttccaggcccgcctcaagagctggttcgagc
ccctggtggaagacatgcagcgccagtgggccgggctggtggagaaggtgcaggctgccgtgggcaccagcgccg
cccctgtgcccagcgacaatcacACCGGTGGATCTGGAGGGagcactcgcaaacaaaattacatgat
gaatttttcacgacaacatgggctcagacgtttctacaacagaagacgaaggtcacttaggcgatacccat
225Yf pFUSEN-IL2ss-FcigG-APOE-CTRH3447R
(amino acid sequence-translation of the sequence by domains)
IL2SS-igg2-fc-LINKER-apoe-LINKER-c-terminus reln H3447R
(SEQ ID NO: 17)
MYRMQLLSCIALSLALVTNSaplerkssvecppcpappvagpsvflfppkpkdtlmisrtpevtcvv
vdvshedpevqfnwyvdgvevhnaktkpreeqfnstfrvvsvltvvhqdwlngkeykckvsnkglpapiektiskt
kgqprepqvytlppsreemtknqvsltclvkgfypsdiavewesngqpennykttppmldsdgsfflyskltvdksr
wqqgnvfscsvmhealhnhytqkslslspgaRTGGGGSGGGGSmkvlwaallvtflagcqakveqavetep
epelrqqtewqsgqrwelalgrfwdylrwvqtlseqvqeellssqvtqelralmdetmkelkaykseleeqltpvaeetr
arlskelqaaqarlgadmedvcgrlvqyrgevqamlgqsteelrvrlashlrklrkrllrdaddlqkrlavyqagareg
aerglsairerlgplveqgrvraatvgslagqplqeraqawgerlrarmeemgsrtrdrldevkeqvaevrakleeqa
qqirlqaeafqarlkswfeplvedmqrqwaglvekvqaavgtsaapvpsdnHTGGSGGstrkqnymmnfsr
qhglrrfynrrrrslrryp
225Z pFUSEN-6xHISCR-50-R5-6-CtermH3447R (nucleotide sequence)
IL2SS-LINKER-6Xhis-tag(SEQ ID NO: 62)-cr50 of reln-LINKER-R5-6 OF RELN-c-
terminus reln H3447R
(SEQ ID NO: 18)
atgtacaggatgcaactcctgtcttgcattgcactaagtcttgcacttgtcacgaattcgGGTGGTcatcatcatca
ccatcacggaggtgaacagtgtggcgcgattatgcatggcaatgccgtcaccttctgtgaaccatatggcccacgaga
actgattaccacaggccttaatacaacaacagcttctgtcctccaattttccattgggtcaggttcatgtcgctttagttattc
agaccccagcatcatcgtgttatatgccaagaataactctgcggactggattcagctagagaaaattagagccccttcc
aatgtcagcacaatcatccatatcctctaccttcctgaggacgccaaaggggagaatgtccaatttcagtggaagcagg
aaaatcttcgtgtaggtgaagtgtatgaagcctgctgggccttaGGTTCTAGAGGAGGACAAACCAA
TGCTACAAGATTCAGACTCTGGCAACCTTATAATAACGGTAAGAAAGAAGA
AATCTGGATTGTTGATGACTTCATTATCGATGGAAATAATGTAAACAACCCT
GTGATGCTCTTGGATACATTTGATTTTGGGCCCAGAGAAGACAATTGGTTTT
TCTATCCTGGTGGTAACATCGGTCTTTATTGTCCATATTCTTCAAAGGGGGC
ACCTGAAGAAGATTCAGCTATGGTGTTTGTTTCAAATGAAGTTGGTGAGCA
TTCCATTACCACCCGTGACCTAAATGTGAATGAGAACACCATCATACAATTT
GAGATCAACGTTGGCTGTTCGACTGATAGCTCATCCGCGGATCCAGTGAGA
CTGGAATTTTCAAGGGACTTCGGGGCGACCTGGCACCTTCTGCTGCCCCTC
TGCTACCACAGCAGCAGCCACGTCAGCTCTTTATGCTCCACCGAGCACCAC
CCCAGCAGCACCTACTACGCAGGAACCATGCAGGGCTGGAGGAGGGAGGT
CGTGCACTTTGGGAAGCTGCACCTTTGTGGATCTGTCCGTTTCAGATGGTA
CCAGGGATTTTACCCTGCCGGCTCTCAGCCAGTGACATGGGCCATTGATAAT
GTCTACATCGGTCCCCAGTGTGAGGAGATGTGTAATGGACAGGGGAGCTGT
ATCAATGGAACCAAATGTATATGTGACCCTGGCTACTCAGGTCCAACCTGTA
AAATAAGCACCAAAAATCCTGATTTTCTCAAAGATGATTTCGAAGGTCAGC
TAGAATCTGATAGATTCTTATTAATGAGTGGTGGGAAACCATCTCGAAAGTG
TGGAATCCTTTCTAGTGGAAACAACCTCTTTTTCAATGAAGATGGCTTGCG
CATGTTGATGACACGAGACCTGGATTTATCACATGCTAGATTTGTGCAGTTC
TTCATGAGACTGGGATGTGGTAAAGGCGTTCCTGACCCCAGGAGTCAACCC
GTGCTCCTACAGTATTCTCTCAACGGTGGCCTCTCGTGGAGTCTTCTTCAGG
AGTTCCTTTTCAGCAATTCCAGCAATGTGGGCAGGTACATTGCCCTGGAGA
TACCCTTGAAAGCCCGTTCTGGTTCTACTCGCCTTCGCTGGTGGCAACCGT
CTGAGAATGGGCACTTCTACAGCCCCTGGGTTATCGATCAGATTCTTATTGG
AGGAAATATTTCTGGTAATATGGTCTTGGAAGATGATTTCACAACCCTTGAT
AGTAGGAAATGGCTGCTTCACCCAGGAGGCACCAAGATGCCCGTGTGTGG
CTCTACTGGTGATGCCCTGGTCTTCATTGAAAAGGCCAGCACCCGTTACGT
GGTCAGCACAGACGTCGCCGTGAATGAGGATTCCTTCCTACAGATAGACTT
CGCTGTCTCCTGCTCAGTCACAGACTCTTGTTATGCGATTGAATTGGAATAC
TCAGTAGATCTTGGATTGTCATGGCACCCATTGGTAAGGGACTGTCTGCCTA
CCAATGTGGAATGCAGTCGCTATCATCTGCAACGGATCCTGGTGTCAGACA
CTTTCAACAAGTGGACTAGAATCACTCTGCCTCTCCCTCCTTATACCAGGTC
CCAAGCCACTCGTTTCCGTTGGCATCAACCAGCTCCTTTTGACAAGCAGCA
GACATGGGCAATAGATAATGTCTATATCGGGGATGGCTGCATAGACATGTGC
AGTGGCCATGGGAGATGCATCCAGGGAAACTGCGTCTGTGATGAACAGTG
GGGTGGCCTGTACTGTGATGACCCCGAGACCTCTCTTCCAACCCAACTCAA
AGACAACTTCAATCGAGCTCCATCCAGTCAGAACTGGCTGACTGTGAACG
GAGGGAAATTGAGTACAGTGTGTGGAGCCGTGGCGTCGGGAATGGCTCTC
CATTTCAGTGGGGGTTGTAGTCGATTATTAGTCACTGTGGATCTAAACCTCA
CTAATGCTGAGTTCATCCAATTTTACTTCATGTATGGGTGCCTGATTACACCA
AACAACCGTAACCAAGGTGTTCTCTTGGAATATTCTGTCAATGGAGGCATT
ACCTGGAACCTGCTCATGGAGATTTTCTATGACCAGTACAGTAAGCCCGGA
TTTGTGAATATCCTTCTCCCTCCTGATGCTAAAGAGATTGCCACTCGCTTCC
GCTGGTGGCAGCCAAGACATGACGGCCTGGATCAGAACGACTGGGCCATT
GACAATGTCCTCATCTCAGGCTCTGCTagcactcgcaaacaaaattacatgatgaatttttcac
gacaacatgggctcagacgtttctacaacagaagacgaaggtcacttaggcgatacccat
225Z pFUSEN-6xHISCR-50-R5-6-CtermH3447R (amino acid sequence-
translation of the sequence by domains)
IL2SS-LINKER-6Xhis-tag(SEQ ID NO: 62)-cr50 of reln-LINKER-R5-6 OF RELN-c-
terminus reln H3447R
(SEQ ID NO: 19)
MYRMQLLSCIALSLALVTNSGGHhhhhhGGeqcgaimhgnavtfcepygprelittglntttasv
lqfsigsgscrfsysdpsiivlyaknnsadwiqlekirapsnvstiihilylpedakgenvqfqwkqenlrvgevyeacwa
lgsrggQTNATRFRLWQPYNNGKKEEIWIVDDFIIDGNNVNNPVMLLDTFDFGP
REDNWFFYPGGNIGLYCPYSSKGAPEEDSAMVFVSNEVGEHSITTRDLNVNE
NTIIQFEINVGCSTDSSSADPVRLEFSRDFGATWHLLLPLCYHSSSHVSSLCSTE
HHPSSTYYAGTMQGWRREVVHFGKLHLCGSVRFRWYQGFYPAGSQPVTWA
IDNVYIGPQCEEMCNGQGSCINGTKCICDPGYSGPTCKISTKNPDFLKDDFEG
QLESDRFLLMSGGKPSRKCGILSSGNNLFFNEDGLRMLMTRDLDLSHARFVQ
FFMRLGCGKGVPDPRSQPVLLQYSLNGGLSWSLLQEFLFSNSSNVGRYIALEI
PLKARSGSTRLRWWQPSENGHFYSPWVIDQILIGGNISGNMVLEDDFTTLDSR
KWLLHPGGTKMPVCGSTGDALVFIEKASTRYVVSTDVAVNEDSFLQIDFAVS
CSVTDSCYAIELEYSVDLGLSWHPLVRDCLPTNVECSRYHLQRILVSDTFNK
WTRITLPLPPYTRSQATRFRWHQPAPFDKQQTWAIDNVYIGDGCIDMCSGHG
RCIQGNCVCDEQWGGLYCDDPETSLPTQLKDNFNRAPSSQNWLTVNGGKLS
TVCGAVASGMALHFSGGCSRLLVTVDLNLTNAEFIQFYFMYGCLITPNNRNQ
GVLLEYSVNGGITWNLLMEIFYDQYSKPGFVNILLPPDAKEIATRFRWWQPR
HDGLDQNDWAIDNVLISGSAstrkqnymmnfsrqhglrrfynrrrrslrryp
225SU pFUSEN-IL2signalpeptide-6XHistag-hReelin(AALeu1220-Ile2660) C-
term-REELIN H3447R (nucleotide sequence)
IL2SS-LINKER-6Xhis-tag(SEQ ID NO: 62)-LINKER-l1120-Ile2660 hreln-LINKER-
c-terminus reln H3447R
(SEQ ID NO: 20)
ATGTACAGGATGCAACTCCTGTCTTGCATTGCACTAAGTCTTGCACTT
GTCACGAATTCGGGTGGTcatcatcatcaccatcacGGAGGTTCTAGActgtccgagaagca
gaagcagatcatcccagttatcaatccaactttacctcagaacttttatgagaagccagcttttgattaccctatgaatcag
atgagtgtgtggttgatgttggctaatgaaggaatggttaaaaatgaaaccttctgtgctgccacaccatcagcaatgat
atttggaaaatcagatggagatcgatttgcagtaactcgagatttgaccctgaaacctggatatgtgctacagttcaagct
aaacataggttgtgccaatcaattcagcagtactgctccagttcttcttcagtactctcatgatgctggtatgtcctggtttct
ggtgaaagaaggctgttacccggcttctgcaggcaaaggatgcgaaggaaactccagagaactaagtgagcccacc
atgtatcacacaggggactttgaagaatggacaagaatcaccattgttattccaaggtctcttgcatccagcaagacca
gattccgatggatccaggagagcagctcacagaaaaacgtgcctccatttggtttagatggagtgtacatatccgagcc
ttgtcccagttactgcagtggccatggggactgcatttcaggagtgtgtttctgtgacctgggatatactgctgcacaagg
aacctgtgtgtcaaatgtccccaatcacaatgagatgttcgataggtttgaggggaagctcagccctctgtggtacaaga
taacaggtgcccaggttggaactggctgtggaacacttaacgatggcaaatctctctacttcaatggccctgggaaaag
ggaagcccgtacggtccctctggacaccaggaatatcagacttgttcaattttatatacaaattggaagcaaaacttcag
gcattacctgcatcaaaccaagaactagaaatgaagggcttattgttcagtattcaaatgacaatgggatactctggcat
ttgcttcgagagttggacttcatgtccttcctggaaccacagatcatttccattgacctgccacaggacgcgaagacacct
gcaacggcatttcgatggtggcaaccgcaacatgggaagcattcagcccagtgggctttggatgatgttcttataggaa
tgaatgacagctctcaaactggatttcaagacaaatttgatggctctatagatttgcaagccaactggtatcgaatccaa
ggaggtcaagttgatattgactgtctctctatggatactgctctgatattcactgaaaacataggaaaacctcgttatgctg
agacctgggattttcatgtgtcagcatctacctttttgcagtttgaaatgagcatgggctgtagcaagcccttcagcaactc
ccacagtgtacagctccagtattctctgaacaatggcaaggactggcatcttgtcaccgaagagtgtgttcctccaacca
ttggctgtctgcattacacggaaagttcaatttacacctcggaaagattccagaattggaagcggatcactgtctaccttc
cactctccaccatttctcccaggacccggttcagatggattcaggccaactacactgtgggggctgattcctgggcgatt
gataatgttgtactggcctcagggtgcccttggatgtgctcaggacgagggatttgtgatgctggacgctgtgtgtgtgac
cggggctttggtggaccctattgtgttcctgttgttcctctgccctcgattcttaaagacgatttcaatgggaatttacatcctg
acctttggcctgaagtgtatggtgcagagagggggaatctgaatggtgaaaccatcaaatctggaacatctctaattttt
aaaggggaaggactaaggatgcttatttcaagagatctagattgtacaaatacaatgtatgtccagttttcacttagattt
atagcaaaaagtaccccagagagatctcactctattctgttacaattctccatcagtggaggaatcacttggcacctgat
ggatgaattttactttcctcaaacaacgaatatacttttcatcaatgttcccttgccatacactgcccadaccaatgctaca
agattcagactctggcaaccttataataacggtaagaaagaagaaatctggattgttgatgacttcattatcgatggaaa
taatgtaaacaaccctgtgatgctcttggatacatttgattttgggcccagagaagacaattggtttttctatcctggtggta
acatcggtctttattgtccatattcttcaaagggggcacctgaagaagattcagctatggtgtttgtttcaaatgaagttggt
gagcattccattaccacccgtgacctaaatgtgaatgagaacaccatcatacaatttgagatcaacgttggctgttcgac
tgatagctcatccgcggatccagtgagactggaattttcaagggacttcggggcgacctggcaccttctgctgcccctct
gctaccacagcagcagccacgtcagctctttatgctccaccgagcaccaccccagcagcacctactacgcaggaacc
atgcagggctggaggagggaggtcgtgcactttgggaagctgcacctttgtggatctgtccgtttcagatggtaccagg
gattttaccctgccggctctcagccagtgacatgggccattgataatgtctacatcggtccccagtgtgaggagatgtgta
atggacaggggagctgtatcaatggaaccaaatgtatatgtgaccctggctactcaggtccaacctgtaaaataagca
ccaaaaatcctgattttctcaaagatgatttcgaaggtcagctagaatctgatagattcttattaatgagtggtgggaaac
catctcgaaagtgtggaatcctttctagtggaaacaacctctttttcaatgaagatggcttgcgcatgttgatgacacgag
acctggatttatcacatgctagatttgtgcagttcttcatgagactgggatgtggtaaaggcgttcctgaccccaggagtc
aacccgtgctcctacagtattctctcaacggtggcctctcgtggagtcttcttcaggagttccttttcagcaattccagcaat
gtgggcaggtacattgccctggagatacccttgaaagcccgttctggttctactcgccttcgctggtggcaaccgtctgag
aatgggcacttctacagcccctgggttatcgatcagattcttattggaggaaatatttctggtaatatggtcttggaagatg
atttcacaacccttgatagtaggaaatggctgcttcacccaggaggcaccaagatgcccgtgtgtggctctactggtgat
gccctggtcttcattgaaaaggccagcacccgttacgtggtcagcacagacgtcgccgtgaatgaggattccttcctac
agatagacttcgctgtctcctgctcagtcacagactcttgttatgcgattgaattggaatactcagtagatcttggattgtcat
ggcacccattggtaagggactgtctgcctaccaatgtggaatgcagtcgctatcatctgcaacggatcctggtgtcagac
actttcaacaagtggactagaatcactctgcctctccctccttataccaggtcccaagccactcgtttccgttggcatcaac
cagctccttttgacaagcagcagacatgggcaatagataatgtctatatcggggatggctgcatagacatgtgcagtgg
ccatgggagatgcatccagggaaactgcgtctgtgatgaacagtggggggcctgtactgtgatgaccccgagacctc
tcttccaacccaactcaaagacaacttcaatcgagctccatccagtcagaactggctgactgtgaacggagggaaatt
gagtacagtgtgtggagccgtggcgtcgggaatggctctccatttcagtgggggttgtagtcgattattagtcactgtgga
tctaaacctcactaatgctgagttcatccaattttacttcatgtatgggtgcctgattacaccaaacaaccgtaaccaaggt
gttctcttggaatattctgtcaatggaggcattacctggaacctgctcatggagattttctatgaccagtacagtaagcccg
ggtttgtgaatatccttctccctcctgatgctaaagagattgccactcgcttccgctggtggcagccaagacatgacggcc
tggatcagaacgactgggccattgacaatgtcctcatcACCGGTGGATCTGGAGGGagcactcgcaaa
caaaattacatgatgaatttttcacgacaacatgggctcagacgtttctacaacagaagacgaaggtcacttaggcg
atacccat
225SU pFUSEN-IL2signalpeptide-6XHistag(SEQ ID NO: 62)-
hReelin(AALeu1220-Ile2660) C-term-REELIN H3447R (amino acid sequence-
translation of the sequence by domains)
IL2SS-LINKER-6Xhis-tag(SEQ ID NO: 62)-LINKER-l1120-Ile2660 hreln-LINKER-
c-terminus reln H3447R
(SEQ ID NO: 21)
MYRMQLLSCIALSLALVTNSGGhhhhhhGGSRlsekqkqiipvinptlpqnfyekpafdypmn
qmsvwlmlanegmvknetfcaatpsamifgksdgdrfavtrdltlkpgyvlqfklnigcanqfsstapvllqyshdagm
swflvkegcypasagkgcegnsrelseptmyhtgdfeewtritiviprslassktrfrwiqesssqknvppfgldgvyise
pcpsycsghgdcisgvcfcdlgytaaqgtcvsnvpnhnemfdrfegklsplwykitgaqvgtgcgtlndgkslyfngpg
kreartvpldtrnirlvqfyiqigsktsgitcikprtrneglivqysndngilwhllreldfmsflepqiisidlpqdaktpataf
rwwqpqhgkhsaqwalddvligmndssqtgfqdkfdgsidlqanwyriqggqvdidclsmdtaliftenigkpryae
twdfhvsastflqfemsmgcskpfsnshsvqlqyslnngkdwhlvteecvpptigclhytessiytserfqnwkritvylpl
stisprtrfrwiqanytvgadswaidnvvlasgcpwmcsgrgicdagrcvcdrgfggpycvpvvplpsilkddfngnl
hpdlwpevygaergnlngetiksgtslifkgeglrmlisrdldctntmyvqfslrfiakstpershsillqfsisggitwhlm
defyfpqttnilfinvplpytaqtnatrfrlwqpynngkkeeiwivddfiidgnnvnnpvmlldtfdfgprednwffypgg
niglycpysskgapeedsamvfvsnevgehsittrdlnvnentiiqfeinvgcstdsssadpvrlefsrdfgatwhlllplc
yhssshvsslcstehhpsstyyagtmqgwrrevvhfgklhlcgsvrfrwyqgfypagsqpvtwaidnvyigpqceemc
ngqgscingtkcicdpgysgptckistknpdflkddfegqlesdrfllmsggkpsrkcgilssgnnlffnedglrmlmtrdl
dlsharfvqffmrlgcgkgvpdprsqpvllqyslngglswsllqeflfsnssnvgryialeiplkarsgstrlrwwqpsen
ghfyspwvidqiliggnisgnmvleddfttldsrkwllhpggtkmpvcgstgdalvfiekastryvvstdvavnedsflqi
dfavscsvtdscyaieleysvdlglswhplvrdclptnvecsryhlqrilvsdtfnkwtritlplppytrsqatrfrwhqpap
fdkqqtwaidnvyigdgcidmcsghgrciqgncvcdeqwgglycddpetslptqlkdnfnrapssqnwltvnggklst
vcgavasgmalhfsggcsrllvtvdlnltnaefiqfyfmygclitpnnrnqgvlleysvnggitwnllmeifydqyskpgf
vnillppdakeiatrfrwwqprhdgldqndwaidnvliTGGSGGstrkqnymmnfsrqhglrrfynrrrrslr
ryp
225SV pFUSEN-IL2signalpeptide-6XHistag(SEQ ID NO: 62)-
hReelin(AALeu1220-Ile2660) Cterm-REELINWT (nucleotide sequence)
IL2SS-LINKER-6Xhis-tag(SEQ ID NO: 62)-LINKER-l1120-Ile2660 hreln-LINKER-
c-terminus reln wt
(SEQ ID NO: 22)
AtgtacaggatgcaactcctgtcttgcattgcactaagtcttgcacttgtcacgaattcgGGTGGTcatcatcatc
accatcacGGAGGTTCTAGActgtccgagaagcagaagcagatcatcccagttatcaatccaactttacctca
gaacttttatgagaagccagcttttgattaccctatgaatcagatgagtgtgtggttgatgttggctaatgaaggaatggtt
aaaaatgaaaccttctgtgctgccacaccatcagcaatgatatttggaaaatcagatggagatcgatttgcagtaactc
gagatttgaccctgaaacctggatatgtgctacagttcaagctaaacataggttgtgccaatcaattcagcagtactgctc
cagttcttcttcagtactctcatgatgctggtatgtcctggtttctggtgaaagaaggctgttacccggcttctgcaggcaaa
ggatgcgaaggaaactccagagaactaagtgagcccaccatgtatcacacaggggactttgaagaatggacaaga
atcaccattgttattccaaggtctcttgcatccagcaagaccagattccgatggatccaggagagcagctcacagaaaa
acgtgcctccatttggtttagatggagtgtacatatccgagccttgtcccagttactgcagtggccatggggactgcatttc
aggagtgtgtttctgtgacctgggatatactgctgcacaaggaacctgtgtgtcaaatgtccccaatcacaatgagatgtt
cgataggtttgaggggaagctcagccctctgtggtacaagataacaggtgcccaggttggaactggctgtggaacactt
aacgatggcaaatctctctacttcaatggccctgggaaaagggaagcccgtacggtccctctggacaccaggaatatc
agacttgttcaattttatatacaaattggaagcaaaacttcaggcattacctgcatcaaaccaagaactagaaatgaag
ggcttattgttcagtattcaaatgacaatgggatactctggcatttgcttcgagagttggacttcatgtccttcctggaacca
cagatcatttccattgacctgccacaggacgcgaagacacctgcaacggcatttcgatggtggcaaccgcaacatggg
aagcattcagcccagtgggctttggatgatgttcttataggaatgaatgacagctctcaaactggatttcaagacaaattt
gatggctctatagatttgcaagccaactggtatcgaatccaaggaggtcaagttgatattgactgtctctctatggatactg
ctctgatattcactgaaaacataggaaaacctcgttatgctgagacctgggattttcatgtgtcagcatctacctttttgcag
tttgaaatgagcatgggctgtagcaagcccttcagcaactcccacagtgtacagctccagtattctctgaacaatggcaa
ggactggcatcttgtcaccgaagagtgtgttcctccaaccattggctgtctgcattacacggaaagttcaatttacacctc
ggaaagattccagaattggaagcggatcactgtctaccttccactctccaccatttctcccaggacccggttcagatgga
ttcaggccaactacactgtgggggctgattcctgggcgattgataatgttgtactggcctcagggtgcccttggatgtgctc
aggacgagggatttgtgatgctggacgctgtgtgtgtgaccggggctttggtggaccctattgtgttcctgttgttcctctgc
cctcgattcttaaagacgatttcaatgggaatttacatcctgacctttggcctgaagtgtatggtgcagagagggggaatc
tgaatggtgaaaccatcaaatctggaacatctctaatttttaaaggggaaggactaaggatgcttatttcaagagatcta
gattgtacaaatacaatgtatgtccagttttcacttagatttatagcaaaaagtaccccagagagatctcactctattctgtt
acaattctccatcagtggaggaatcacttggcacctgatggatgaattttactttcctcaaacaacgaatatacttttcatc
aatgttcccttgccatacactgcccaaaccaatgctacaagattcagactctggcaaccttataataacggtaagaaag
aagaaatctggattgttgatgacttcattatcgatggaaataatgtaaacaaccctgtgatgctcttggatacatttgatttt
gggcccagagaagacaattggtttttctatcctggtggtaacatcggtctttattgtccatattcttcaaagggggcacctg
aagaagattcagctatggtgtttgtttcaaatgaagttggtgagcattccattaccacccgtgacctaaatgtgaatgaga
acaccatcatacaatttgagatcaacgttggctgttcgactgatagctcatccgcggatccagtgagactggaattttcaa
gggacttcggggcgacctggcaccttctgctgcccctctgctaccacagcagcagccacgtcagctctttatgctccacc
gagcaccaccccagcagcacctactacgcaggaaccatgcagggctggaggagggaggtcgtgcactttgggaag
ctgcacctttgtggatctgtccgtttcagatggtaccagggattttaccctgccggctctcagccagtgacatgggccattg
ataatgtctacatcggtccccagtgtgaggagatgtgtaatggacaggggagctgtatcaatggaaccaaatgtatatg
tgaccctggctactcaggtccaacctgtaaaataagcaccaaaaatcctgattttctcaaagatgatttcgaaggtcagc
tagaatctgatagattcttattaatgagtggtgggaaaccatctcgaaagtgtggaatcctttctagtggaaacaacctctt
tttcaatgaagatggcttgcgcatgttgatgacacgagacctggatttatcacatgctagatttgtgcagttcttcatgaga
ctgggatgtggtaaaggcgttcctgaccccaggagtcaacccgtgctcctacagtattctctcaacggtggcctctcgtg
gagtcttcttcaggagttccttttcagcaattccagcaatgtgggcaggtacattgccctggagatacccttgaaagcccg
ttctggttctactcgccttcgctggtggcaaccgtctgagaatgggcacttctacagcccctgggttatcgatcagattctta
ttggaggaaatatttctggtaatatggtcttggaagatgatttcacaacccttgatagtaggaaatggctgcttcacccag
gaggcaccaagatgcccgtgtgtggctctactggtgatgccctggtcttcattgaaaaggccagcacccgttacgtggtc
agcacagacgtcgccgtgaatgaggattccttcctacagatagacttcgctgtctcctgctcagtcacagactcttgttatg
cgattgaattggaatactcagtagatcttggattgtcatggcacccattggtaagggactgtctgcctaccaatgtggaat
gcagtcgctatcatctgcaacggatcctggtgtcagacactttcaacaagtggactagaatcactctgcctctccctcctt
ataccaggtcccaagccactcgtttccgttggcatcaaccagctccttttgacaagcagcagacatgggcaatagataa
tgtctatatcggggatggctgcatagacatgtgcagtggccatgggagatgcatccagggaaactgcgtctgtgatgaa
cagtggggtggcctgtactgtgatgaccccgagacctctcttccaacccaactcaaagacaacttcaatcgagctccat
ccagtcagaactggctgactgtgaacggagggaaattgagtacagtgtgtggagccgtggcgtcgggaatggctctcc
atttcagtgggggttgtagtcgattattagtcactgtggatctaaacctcactaatgctgagttcatccaattttacttcatgta
tgggtgcctgattacaccaaacaaccgtaaccaaggtgttctcttggaatattctgtcaatggaggcattacctggaacct
gctcatggagattttctatgaccagtacagtaagcccgggtttgtgaatatccttctccctcctgatgctaaagagattgcc
actcgcttccgctggtggcagccaagacatgacggcctggatcagaacgactgggccattgacaatgtcctcatcAC
CGGTGGATCTGGAGGGagcactcgcaaacaaaattacatgatgaatttttcacgacaacatgggctca
gacatttctacaacagaagacgaaggtcacttaggcgatacccat
225SV pFUSEN-IL2signalpeptide-6XHistag(SEQ ID NO: 62)-
hReelin(AALeu1220-Ile2660) Cterm-REELINWT (amino acid sequence-
translation of the sequence by domains)
IL2SS-LINKER-6Xhis-tag(SEQ ID NO: 62)-LINKER-l1120-Ile2660 hreln-LINKER-
c-terminus reln wt
(SEQ ID NO: 23)
MYRMQLLSClALSLALVTNSGGhhhhhhGGSRlsekqkqiipvinptlpqnfyekpafdypmn
qmsvwlmlanegmvknetfcaatpsamifgksdgdrfavtrdltlkpgyvlqfklnigcanqfsstapvllqyshdagm
swflvkegcypasagkgcegnsrelseptmyhtgdfeewtritiviprslassktrfrwiqesssqknvppfgldgvyise
pcpsycsghgdcisgvcfcdlgytaaqgtcvsnvpnhnemfdrfegklsplwykitgaqvgtgcgtlndgkslyfngpg
kreartvpldtrnirlvqfyigigsktsgitcikprtrneglivqysndngilwhllreldfmsflepqiisidlpqdaktpataf
rwwqpqhgkhsaqwalddvligmndssqtgfqdkfdgsidlqanwyriqggqvdidclsmdtaliftenigkpryae
twdfhvsastflqfemsmgcskpfsnshsvqlqyslnngkdwhlvteecvpptigclhytessiytserfqnwkritvylpl
stisprtrfrwiqanytvgadswaidnvvlasgcpwmcsgrgicdagrcvcdrgfggpycvpvvplpsilkddfngnl
hpdlwpevygaergnlngetiksgtslifkgeglrmlisrdldctntmyvqfslrfiakstpershsillqfsisggitwhlm
defyfpqttnilfinvplpytaqtnatrfrlwqpynngkkeeiwivddfiidgnnvnnpvmlldtfdfgprednwffypgg
niglycpysskgapeedsamvfvsnevgehsittrdlnvnentiiqfeinvgcstdsssadpvrlefsrdfgatwhlllplc
yhssshvsslcstehhpsstyyagtmqgwrrevvhfgklhlcgsvrfrwyqgfypagsqpvtwaidnvyigpqceemc
ngqgscingtkcicdpgysgptckistknpdflkddfegqlesdrfllmsggkpsrkcgilssgnnlffnedglrmlmtrdl
dlsharfvqffmrlgcgkgvpdprsqpvllqyslngglswsllqeflfsnssnvgryialeiplkarsgstrlrwwqpsen
ghfyspwvidqiliggnisgnmvleddfttldsrkwllhpggtkmpvcgstgdalvfiekastryvvstdvavnedsflqi
dfavscsvtdscyaieleysvdlglswhplvrdclptnvecsryhlqrilvsdtfnkwtritlplppytrsqatrfrwhqpap
fdkqqtwaidnvyigdgcidmcsghgrciqgncvcdeqwgglycddpetslptqlkdnfnrapssqnwltvnggklst
vcgavasgmalhfsggcsrllvtvdlnltnaefiqfyfmygclitpnnrnqgvlleysvnggitwnllmeifydqyskpgf
vnillppdakeiatrfrwwqprhdgldqndwaidnvliTGGSGGstrkqnymmnfsrqhglrhfynrrrrslrry
p
225ZZ pFUSEN-Il2ss6xHISMini-RELN-FspCR50-R5-6-CtermH3447R
(nucleotide sequence)
IL2SS-LINKER-6Xhis-tag(SEQ ID NO: 62)-LINKER-fspcr50 of reln-LINKER-R5-6
OF RELN-c-terminus reln h3447r
(SEQ ID NO: 24)
ATGTACAGGATGCAACTCCTGTCTTGCATTGCACTAAGTCTTGCACTT
GTCACGAATTCGGGTGGCcatcatcatcaccatcacACCGGTGGTgcggctggctattaccccc
gcttttcgcccttctttttcctgtgcacccaccacggggagctggaaggggatggggagcagggcgaggtgctcatttcc
ctgcatattgcgggcaaccccacctactacgttccgggacaagaataccatgtgacaatttcaacaagcaccttttttga
cggcttgctggtgacaggactatacacatctacaagtgttcaggcatcacagagcattggaggttccagtgctttcggatt
tgggatcatgtctgaccaccagtttggtaaccagtttatgtgcagtgtggtagcctctcacgtgagtcacctgcccacaac
caacctcagtttcatctggattgctccacctgcgggcacaggctgtgtgaatttcatggctacagcaacacaccggggcc
aggttattttcaaagatgctttagcccagcagttgtgtgaacaaggagctccaacagatgtcactgtgcacccacatcta
gctgaaatacatagtgacagcattatcctgagagatgactttgactcctaccaccaactgcaattaaatccaaatatatg
ggttgaatgtaacaactgtgagactggagaacagtgtggcgcgattatgcatggcaatgccgtcaccttctgtgaacca
tatggcccacgagaactgattaccacaggccttaatacaacaacagcttctgtcctccaattttccattgggtcaggttcat
gtcgctttagttattcagaccccagcatcatcgtgttatatgccaagaataactctgcggactggattcagctagagaaaa
ttagagccccttccaatgtcagcacaatcatccatatcctctaccttcctgaggacgccaaaggggagaatgtccaattt
cagtggaagcaggaaaatcttcgtgtaggtgaagtgtatgaagcctgctgggccttagataacatcttgatcatcaatG
GTTCTAGAGGAGGACAAACCAATGCTACAAGATTCAGACTCTGGCAACCTT
ATAATAACGGTAAGAAAGAAGAAATCTGGATTGTTGATGACTTCATTATCGA
TGGAAATAATGTAAACAACCCTGTGATGCTCTTGGATACATTTGATTTTGGG
CCCAGAGAAGACAATTGGTTTTTCTATCCTGGTGGTAACATCGGTCTTTATT
GTCCATATTCTTCAAAGGGGGCACCTGAAGAAGATTCAGCTATGGTGTTTG
TTTCAAATGAAGTTGGTGAGCATTCCATTACCACCCGTGACCTAAATGTGAA
TGAGAACACCATCATACAATTTGAGATCAACGTTGGCTGTTCGACTGATAG
CTCATCCGCGGATCCAGTGAGACTGGAATTTTCAAGGGACTTCGGGGCGAC
CTGGCACCTTCTGCTGCCCCTCTGCTACCACAGCAGCAGCCACGTCAGCTC
TTTATGCTCCACCGAGCACCACCCCAGCAGCACCTACTACGCAGGAACCAT
GCAGGGCTGGAGGAGGGAGGTCGTGCACTTTGGGAAGCTGCACCTTTGTG
GATCTGTCCGTTTCAGATGGTACCAGGGATTTTACCCTGCCGGCTCTCAGCC
AGTGACATGGGCCATTGATAATGTCTACATCGGTCCCCAGTGTGAGGAGAT
GTGTAATGGACAGGGGAGCTGTATCAATGGAACCAAATGTATATGTGACCC
TGGCTACTCAGGTCCAACCTGTAAAATAAGCACCAAAAATCCTGATTTTCT
CAAAGATGATTTCGAAGGTCAGCTAGAATCTGATAGATTCTTATTAATGAGT
GGTGGGAAACCATCTCGAAAGTGTGGAATCCTTTCTAGTGGAAACAACCTC
TTTTTCAATGAAGATGGCTTGCGCATGTTGATGACACGAGACCTGGATTTAT
CACATGCTAGATTTGTGCAGTTCTTCATGAGACTGGGATGTGGTAAAGGCG
TTCCTGACCCCAGGAGTCAACCCGTGCTCCTACAGTATTCTCTCAACGGTG
GCCTCTCGTGGAGTCTTCTTCAGGAGTTCCTTTTCAGCAATTCCAGCAATGT
GGGCAGGTACATTGCCCTGGAGATACCCTTGAAAGCCCGTTCTGGTTCTAC
TCGCCTTCGCTGGTGGCAACCGTCTGAGAATGGGCACTTCTACAGCCCCTG
GGTTATCGATCAGATTCTTATTGGAGGAAATATTTCTGGTAATATGGTCTTGG
AAGATGATTTCACAACCCTTGATAGTAGGAAATGGCTGCTTCACCCAGGAG
GCACCAAGATGCCCGTGTGTGGCTCTACTGGTGATGCCCTGGTCTTCATTG
AAAAGGCCAGCACCCGTTACGTGGTCAGCACAGACGTCGCCGTGAATGAG
GATTCCTTCCTACAGATAGACTTCGCTGTCTCCTGCTCAGTCACAGACTCTT
GTTATGCGATTGAATTGGAATACTCAGTAGATCTTGGATTGTCATGGCACCC
ATTGGTAAGGGACTGTCTGCCTACCAATGTGGAATGCAGTCGCTATCATCTG
CAACGGATCCTGGTGTCAGACACTTTCAACAAGTGGACTAGAATCACTCTG
CCTCTCCCTCCTTATACCAGGTCCCAAGCCACTCGTTTCCGTTGGCATCAAC
CAGCTCCTTTTGACAAGCAGCAGACATGGGCAATAGATAATGTCTATATCGG
GGATGGCTGCATAGACATGTGCAGTGGCCATGGGAGATGCATCCAGGGAAA
CTGCGTCTGTGATGAACAGTGGGGTGGCCTGTACTGTGATGACCCCGAGAC
CTCTCTTCCAACCCAACTCAAAGACAACTTCAATCGAGCTCCATCCAGTCA
GAACTGGCTGACTGTGAACGGAGGGAAATTGAGTACAGTGTGTGGAGCCG
TGGCGTCGGGAATGGCTCTCCATTTCAGTGGGGGTTGTAGTCGATTATTAGT
CACTGTGGATCTAAACCTCACTAATGCTGAGTTCATCCAATTTTACTTCATGT
ATGGGTGCCTGATTACACCAAACAACCGTAACCAAGGTGTTCTCTTGGAAT
ATTCTGTCAATGGAGGCATTACCTGGAACCTGCTCATGGAGATTTTCTATGA
CCAGTACAGTAAGCCCGGATTTGTGAATATCCTTCTCCCTCCTGATGCTAAA
GAGATTGCCACTCGCTTCCGCTGGTGGCAGCCAAGACATGACGGCCTGGAT
CAGAACGACTGGGCCATTGACAATGTCCTCATCTCAGGCTCTGCTagcactcgc
aaacaaaattacatgatgaatttttcacgacaacatgggctcagacgtttctacaacagaagacgaaggtcactta
ggcgatacccat
225ZZ pFUSEN-Il2ss6xHISMini-RELN-FspCR50-R5-6-CtermH3447R
(amino acid sequence-translation of the sequence by domains)
IL2SS-LINKER-6Xhis-tag(SEQ ID NO: 62)-LINKER-fspcr50 of reln-LINKER-R5-6
OF RELN-c-terminus reln h3447r
(SEQ ID NO: 25)
MYRMQLLSCIALSLALVTNSGGhhhhhhTGGaagyyprfspffflcthhgelegdgeqgevlisl
hiagnptyyvpgqeyhvtiststffdgllvtglytstsvqasqsiggssafgfgimsdhqfgnqfmcsvvashvshlpttnls
fiwiappagtgcvnfmatathrgqvifkdalaqqlceqgaptdvtvhphlaeihsdsiilrddfdsyhqlqlnpniwvec
nncetgeqcgaimhgnavtfcepygprelittglntttasvlqfsigsgscrfsysdpsiivlyaknnsadwiqlekiraps
nvstiihilylpedakgenvqfqwkqenlrvgevyeacwaldniliinGSRGGQTNATRFRLWQPYNN
GKKEEIWIVDDFIIDGNNVNNPVMLLDTFDFGPREDNWFFYPGGNIGLYCPYS
SKGAPEEDSAMVFVSNEVGEHSITTRDLNVNENTIIQFEINVGCSTDSSSADPV
RLEFSRDFGATWHLLLPLCYHSSSHVSSLCSTEHHPSSTYYAGTMQGWRREV
VHFGKLHLCGSVRFRWYQGFYPAGSQPVTWAIDNVYIGPQCEEMCNGQGSC
INGTKCICDPGYSGPTCKISTKNPDFLKDDFEGQLESDRFLLMSGGKPSRKCGI
LSSGNNLFFNEDGLRMLMTRDLDLSHARFVQFFMRLGCGKGVPDPRSQPVLL
QYSLNGGLSWSLLQEFLFSNSSNVGRYIALEIPLKARSGSTRLRWWQPSENGH
FYSPWVIDQILIGGNISGNMVLEDDFTTLDSRKWLLHPGGTKMPVCGSTGDA
LVFIEKASTRYVVSTDVAVNEDSFLQIDFAVSCSVTDSCYAIELEYSVDLGLS
WHPLVRDCLPTNVECSRYHLQRILVSDTFNKWTRITLPLPPYTRSQATRFRWH
QPAPFDKQQTWAIDNVYIGDGCIDMCSGHGRCIQGNCVCDEQWGGLYCDDP
ETSLPTQLKDNFNRAPSSQNWLTVNGGKLSTVCGAVASGMALHFSGGCSRLL
VTVDLNLTNAEFIQFYFMYGCLITPNNRNQGVLLEYSVNGGITWNLLMEIFY
DQYSKPGFVNILLPPDAKEIATRFRWWQPRHDGLDQNDWAIDNVLISGSAstr
kqnymmnfsrqhglrrfynrrrrslrryp
225SW pFUSEN-IL2ss-6xHis-hReelin R3-R6 CT-H3447R (nucleotide sequence)
IL2SS-LINKER-6Xhis-tag(SEQ ID NO: 62)-LINKER-r3 of reln-LINKER-R6 OF
RELN OF RELN-LINKER-c-terminus reln wt
(SEQ ID NO: 26)
ATGTACAGGATGCAACTCCTGTCTTGCATTGCACTAAGTCTTGCACTT
GTCACGAATTCGGGTGGTcatcatcatcaccatcacGGAGGTTCTAGAttcagcagtactgct
ccagttcttcttcagtactctcatgatgctggtatgtcctggtttctggtgaaagaaggctgttacccggcttctgcaggcaa
aggatgcgaaggaaactccagagaactaagtgagcccaccatgtatcacacaggggactttgaagaatggacaag
aatcaccattgttattccaaggtctcttgcatccagcaagaccagattccgatggatccaggagagcagctcacagaaa
aacgtgcctccatttggtttagatggagtgtacatatccgagccttgtcccagttactgcagtggccatggggactgcattt
caggagtgtgtttctgtgacctgggatatactgctgcacaaggaacctgtgtgtcaaatgtccccaatcacaatgagatg
ttcgataggtttgaggggaagctcagccctctgtggtacaagataacaggtgcccaggttggaactggctgtggaacac
ttaacgatggcaaatctctctacttcaatggccctgggaaaagggaagcccgtacggtccctctggacaccaggaatat
cagacttgttcaattttatatacaaattggaagcaaaacttcaggcattacctgcatcaaaccaagaactagaaatgaa
gggcttattgttcagtattcaaatgacaatgggatactctggcatttgcttcgagagttggacttcatgtccttcctggaacc
acagatcatttccattgacctgccacaggacgcgaagacacctgcaacggcatttcgatggtggcaaccgcaacatgg
gaagcattcagcccagtgggctttggatgatgttcttataggaatgaatgacagctctcaaactggatttcaagacaaatt
tgatggctctataGGAGGAGGTACCCTTGATAGTAGGAAATGGCTGCTTCACCCAG
GAGGCACCAAGATGCCCGTGTGTGGCTCTACTGGTGATGCCCTGGTCTTCA
TTGAAAAGGCCAGCACCCGTTACGTGGTCAGCACAGACGTCGCCGTGAAT
GAGGATTCCTTCCTACAGATAGACTTCGCTGTCTCCTGCTCAGTCACAGAC
TCTTGTTATGCGATTGAATTGGAATACTCAGTAGATCTTGGATTGTCATGGC
ACCCATTGGTAAGGGACTGTCTGCCTACCAATGTGGAATGCAGTCGCTATCA
TCTGCAACGGATCCTGGTGTCAGACACTTTCAACAAGTGGACTAGAATCAC
TCTGCCTCTCCCTCCTTATACCAGGTCCCAAGCCACTCGTTTCCGTTGGCAT
CAACCAGCTCCTTTTGACAAGCAGCAGACATGGGCAATAGATAATGTCTATA
TCGGGGATGGCTGCATAGACATGTGCAGTGGCCATGGGAGATGCATCCAGG
GAAACTGCGTCTGTGATGAACAGTGGGGTGGCCTGTACTGTGATGACCCCG
AGACCTCTCTTCCAACCCAACTCAAAGACAACTTCAATCGAGCTCCATCCA
GTCAGAACTGGCTGACTGTGAACGGAGGGAAATTGAGTACAGTGTGTGGA
GCCGTGGCGTCGGGAATGGCTCTCCATTTCAGTGGGGGTTGTAGTCGATTA
TTAGTCACTGTGGATCTAAACCTCACTAATGCTGAGTTCATCCAATTTTACT
TCATGTATGGGTGCCTGATTACACCAAACAACCGTAACCAAGGTGTTCTCTT
GGAATATTCTGTCAATGGAGGCATTACCTGGAACCTGCTCATGGAGATTTTC
TATGACCAGTACAGTACCGGTGGATCTGGAGGGagcactcgcaaacaaaattacatgatg
aatttttcacgacaacatgggctcagacgtttctacaacagaagacgaaggtcacttaggcgatacccatg
225SW pFUSEN-IL2ss-6xHis-hReelin R3-R6 CT-H3447R (amino acid sequence-
translation of the sequence by domains)
IL2SS-LINKER-6Xhis-tag(SEQ ID NO: 62)-LINKER-r3 of reln-LINKER-R6 OF
RELN-LINKER-c-terminus reln wt
(SEQ ID NO: 27)
MYRMQLLSCIALSLALVTNSGGhhhhhhGGSRfsstapvllqyshdagmswflvkegcypasa
gkgcegnsrelseptmyhtgdfeewtritiviprslassktrfrwiqesssqknvppfgldgvyisepcpsycsghgdcis
gvcfcdlgytaaqgtcvsnvpnhnemfdrfegklsplwykitgaqvgtgcgtlndgkslyfngpgkreartvpldtrnirl
vqfyiqigsktsgitcikprtrneglivqysndngilwhllreldfmsflepqiisidlpqdaktpatafrwwqpqhgkhsa
qwalddvligmndssqtgfqdkfdgsiGGGTLDSRKWLLHPGGTKMPVCGSTGDALVFIE
KASTRYVVSTDVAVNEDSFLQIDFAVSCSVTDSCYAIELEYSVDLGLSWHPLV
RDCLPTNVECSRYHLQRILVSDTFNKWTRITLPLPPYTRSQATRFRWHQPAPF
DKQQTWAIDNVYIGDGCIDMCSGHGRCIQGNCVCDEQWGGLYCDDPETSLP
TQLKDNFNRAPSSQNWLTVNGGKLSTVCGAVASGMALHFSGGCSRLLVTVD
LNLTNAEFIQFYFMYGCLITPNNRNQGVLLEYSVNGGITWNLLMEIFYDQYST
GGSGGstrkqnymmnfsrqhglrrfynrrrrslrryp
233A pFUSEN-IL2ss-6xHis-hReelinR3-R6CT-WT (nucleotide sequence)
IL2SS-LINKER-6Xhis-tag(SEQ ID NO: 62)-LINKER-r3 of reln-LINKER-R6 OF
RELN-LINKER-c-terminus reln H3447R
(SEQ ID NO: 28)
ATGTACAGGATGCAACTCCTGTCTTGCATTGCACTAAGTCTTGCACTT
GTCACGAATTCGGGTGGTcatcatcatcaccatcacGGAGGTTCTAGATtcagcagtactgct
ccagttcttcttcagtactctcatgatgctggtatgtcctggtttctggtgaaagaaggctgttacccggcttctgcaggcaa
aggatgcgaaggaaactccagagaactaagtgagcccaccatgtatcacacaggggactttgaagaatggacaag
aatcaccattgttattccaaggtctcttgcatccagcaagaccagattccgatggatccaggagagcagctcacagaaa
aacgtgcctccatttggtttagatggagtgtacatatccgagccttgtcccagttactgcagtggccatggggactgcattt
caggagtgtgtttctgtgacctgggatatactgctgcacaaggaacctgtgtgtcaaatgtccccaatcacaatgagatg
ttcgataggtttgaggggaagctcagccctctgtggtacaagataacaggtgcccaggttggaactggctgtggaacac
ttaacgatggcaaatctctctacttcaatggccctgggaaaagggaagcccgtacggtccctctggacaccaggaatat
cagacttgttcaattttatatacaaattggaagcaaaacttcaggcattacctgcatcaaaccaagaactagaaatgaa
gggcttattgttcagtattcaaatgacaatgggatactctggcatttgcttcgagagttggacttcatgtccttcctggaacc
acagatcatttccattgacctgccacaggacgcgaagacacctgcaacggcatttcgatggtggcaaccgcaacatgg
gaagcattcagcccagtgggctttggatgatgttcttataggaatgaatgacagctctcaaactggatttcaagacaaatt
tgatggctctataGGAGGAGGTACCCTTGATAGTAGGAAATGGCTGCTTCACCCAG
GAGGCACCAAGATGCCCGTGTGTGGCTCTACTGGTGATGCCCTGGTCTTCA
TTGAAAAGGCCAGCACCCGTTACGTGGTCAGCACAGACGTCGCCGTGAAT
GAGGATTCCTTCCTACAGATAGACTTCGCTGTCTCCTGCTCAGTCACAGAC
TCTTGTTATGCGATTGAATTGGAATACTCAGTAGATCTTGGATTGTCATGGC
ACCCATTGGTAAGGGACTGTCTGCCTACCAATGTGGAATGCAGTCGCTATCA
TCTGCAACGGATCCTGGTGTCAGACACTTTCAACAAGTGGACTAGAATCAC
TCTGCCTCTCCCTCCTTATACCAGGTCCCAAGCCACTCGTTTCCGTTGGCAT
CAACCAGCTCCTTTTGACAAGCAGCAGACATGGGCAATAGATAATGTCTATA
TCGGGGATGGCTGCATAGACATGTGCAGTGGCCATGGGAGATGCATCCAGG
GAAACTGCGTCTGTGATGAACAGTGGGGTGGCCTGTACTGTGATGACCCCG
AGACCTCTCTTCCAACCCAACTCAAAGACAACTTCAATCGAGCTCCATCCA
GTCAGAACTGGCTGACTGTGAACGGAGGGAAATTGAGTACAGTGTGTGGA
GCCGTGGCGTCGGGAATGGCTCTCCATTTCAGTGGGGGTTGTAGTCGATTA
TTAGTCACTGTGGATCTAAACCTCACTAATGCTGAGTTCATCCAATTTTACT
TCATGTATGGGTGCCTGATTACACCAAACAACCGTAACCAAGGTGTTCTCTT
GGAATATTCTGTCAATGGAGGCATTACCTGGAACCTGCTCATGGAGATTTTC
TATGACCAGTACAGTACCGGTGGATCTGGAGGGagcactcgcaaacaaaattacatgatg
aatttttcacgacaacatgggctcagacatttctacaacagaagacgaaggtcacttaggcgatacccat
233A pFUSEN-IL2ss-6xHis-hReelinR3-R6CT-WT (amino acid sequence-
translation of the sequence by domains)
IL2SS-LINKER-6Xhis-tag(SEQ ID NO: 62)-LINKER-r3 of reln-LINKER-R6 OF
RELN-LINKER-c-terminus reln H3447R
(SEQ ID NO: 29)
MYRMQLLSCIALSLALVTNSGGhhhhhhGGSRfsstapvllqyshdagmswflvkegcypasa
gkgcegnsrelseptmyhtgdfeewtritiviprslassktrfrwiqesssqknvppfgldgvyisepcpsycsghgdcis
gvcfcdlgytaaqgtcvsnvpnhnemfdrfegklsplwykitgaqvgtgcgtlndgkslyfngpgkreartvpldtrnirl
vqfyiqigsktsgitcikprtrneglivqysndngilwhllreldfmsflepqiisidlpqdaktpatafrwwqpqhgkhsa
qwalddvligmndssqtgfqdkfdgsiGGGtldsrkwllhpggtkmpvcgstgdalvfiekastryvvstdvavned
sflqidfavscsvtdscyaieleysvdlglswhplvrdclptnvecsryhlqrilvsdtfnkwtritlplppytrsqatrfrwh
qpapfdkqqtwaidnvyigdgcidmcsghgrciqgncvcdeqwgglycddpetslptqlkdnfnrapssqnwltvn
ggklstvcgavasgmalhfsggcsrllvtvdlnltnaefiqfyfmygclitpnnrnqgvlleysvnggitwnllmeifyd
qysTGGSGGstrkqnymmnfsrqhglrhfynrrrrslrryp
233C pFUSEN-Il2ss-6xHISR5-6CtermH3447R (nucleotide sequence)
IL2SS-LINKER-6Xhis-tag(SEQ ID NO: 62)-LINKER-r5-6 of reln-c-terminus reln
H3447R
(SEQ ID NO: 30)
ATGTACAGGATGCAACTCCTGTCTTGCATTGCACTAAGTCTTGCACTT
GTCACGAATTCGGGTGGCcaccatcatcaccatcacGGAGGTTCTAGAGGAGGAcaa
accaatgctacaagattcagactctggcaaccttataataacggtaagaaagaagaaatctggattgttgatgacttcat
tatcgatggaaataatgtaaacaaccctgtgatgctcttggatacatttgattttgggcccagagaagacaattggtttttct
atcctggtggtaacatcggtctttattgtccatattcttcaaagggggcacctgaagaagattcagctatggtgtttgtttca
aatgaagttggtgagcattccattaccacccgtgacctaaatgtgaatgagaacaccatcatacaatttgagatcaacgt
tggctgttcgactgatagctcatccgcggatccagtgagactggaattttcaagggacttcggggcgacctggcaccttc
tgctgcccctctgctaccacagcagcagccacgtcagctctttatgctccaccgagcaccaccccagcagcacctacta
cgcaggaaccatgcagggctggaggagggaggtcgtgcactttgggaagctgcacctttgtggatctgtccgtttcaga
tggtaccagggattttaccctgccggctctcagccagtgacatgggccattgataatgtctacatcggtccccagtgtgag
gagatgtgtaatggacaggggagctgtatcaatggaaccaaatgtatatgtgaccctggctactcaggtccaacctgta
aaataagcaccaaaaatcctgattttctcaaagatgatttcgaaggtcagctagaatctgatagattcttattaatgagtg
gtgggaaaccatctcgaaagtgtggaatcctttctagtggaaacaacctctttttcaatgaagatggcttgcgcatgttgat
gacacgagacctggatttatcacatgctagatttgtgcagttcttcatgagactgggatgtggtaaaggcgttcctgaccc
caggagtcaacccgtgctcctacagtattctctcaacggtggcctctcgtggagtcttcttcaggagttccttttcagcaatt
ccagcaatgtgggcaggtacattgccctggagatacccttgaaagcccgttctggttctactcgccttcgctggtggcaa
ccgtctgagaatgggcacttctacagcccctgggttatcgatcagattcttattggaggaaatatttctggtaatatggtctt
ggaagatgatttcacaacccttgatagtaggaaatggctgcttcacccaggaggcaccaagatgcccgtgtgtggctct
actggtgatgccctggtcttcattgaaaaggccagcacccgttacgtggtcagcacagacgtcgccgtgaatgaggatt
ccttcctacagatagacttcgctgtctcctgctcagtcacagactcttgttatgcgattgaattggaatactcagtagatcttg
gattgtcatggcacccattggtaagggactgtctgcctaccaatgtggaatgcagtcgctatcatctgcaacggatcctg
gtgtcagacactttcaacaagtggactagaatcactctgcctctccctccttataccaggtcccaagccactcgtttccgtt
ggcatcaaccagctccttttgacaagcagcagacatgggcaatagataatgtctatatcggggatggctgcatagacat
gtgcagtggccatgggagatgcatccagggaaactgcgtctgtgatgaacagtggggtggcctgtactgtgatgaccc
cgagacctctcttccaacccaactcaaagacaacttcaatcgagctccatccagtcagaactggctgactgtgaacgg
agggaaattgagtacagtgtgtggagccgtggcgtcgggaatggctctccatttcagtgggggttgtagtcgattattagt
cactgtggatctaaacctcactaatgctgagttcatccaattttacttcatgtatgggtgcctgattacaccaaacaaccgt
aaccaaggtgttctcttggaatattctgtcaatggaggcattacctggaacctgctcatggagattttctatgaccagtaca
gtaagcccggatttgtgaatatccttctccctcctgatgctaaagagattgccactcgcttccgctggtggcagccaagac
atgacggcctggatcagaacgactgggccattgacaatgtcctcatctcaggctctgctagcactcgcaaacaaaatt
acatgatgaatttttcacgacaacatgggctcagacgtttctacaacagaagacgaaggtcacttaggcgataccc
atga
233C pFUSEN-Il2ss-6xHISR5-6CtermH3447R (amino acid sequence-
translation of the sequence by domains)
IL2SS-LINKER-6Xhis-tag(SEQ ID NO: 62)-LINKER-r5-6 of reln-c-terminus reln
H3447R
(SEQ ID NO: 31)
MYRMQLLSCIALSLALVTNSGGhhhhhhGGSRGGqtnatrfrlwqpynngkkeeiwivddfi
idgnnvnnpvmlldtfdfgprednwffypggniglycpysskgapeedsamvfvsnevgehsittrdlnvnentiiqfei
nvgcstdsssadpvrlefsrdfgatwhlllplcyhssshvsslcstehhpsstyyagtmqgwrrevvhfgklhlcgsvrfr
wyqgfypagsqpvtwaidnvyigpqceemcngqgscingtkcicdpgysgptckistknpdflkddfegqlesdrfll
msggkpsrkcgilssgnnlffnedglrmlmtrdldlsharfvqffmrlgcgkgvpdprsqpvllqyslngglswsllqefl
fsnssnvgryialeiplkarsgstrlrwwqpsenghfyspwvidqiliggniSgnmvleddfttldsrkwllhpggtkmp
vcgstgdalvfiekastryvvstdvavnedsflqidfavscsvtdscyaieleysvdlglswhplvrdclptnvecsryhlqr
ilvsdtfnkwtritlplppytrsqatrfrwhqpapfdkqqtwaidnvyigdgcidmcsghgrciqgncvcdeqwgglyc
ddpetslptqlkdnfnrapssqnwltvnggklstvcgavasgmalhfsggcsrllvtvdlnltnaefiqfyfmygclitpnn
rnqgvlleysvnggitwnllmeifydqyskpgfvnillppdakeiatrfrwwqprhdgldqndwaidnvlisgsastrk
qnymmnfsrqhglrrfynrrrrslrryp
233D pFUSEN-IL2ss-6xHis-hReelinR6-R6CT-H3447R (nucleotide sequence)
IL2SS-LINKER-6Xhis-tag(SEQ ID NO: 62)-LINKER-r6 of reln-LINKER-r6 of reln-
LINKER-c-terminus reln H3447R
(SEQ ID NO: 32)
ATGTACAGGATGCAACTCCTGTCTTGCATTGCACTAAGTCTTGCACTT
GTCACGAATTCGGGTGGCcaccatcatcaccatcacGGAGGTTCTAGAacccttgatagtag
gaaatggctgcttcacccaggaggcaccaagatgcccgtgtgtggctctactggtgatgccctggtcttcattgaaaag
gccagcacccgttacgtggtcagcacagacgtcgccgtgaatgaggattccttcctacagatagacttcgctgtctcctg
ctcagtcacagactcttgttatgcgattgaattggaatactcagtagatcttggattgtcatggcacccattggtaagggac
tgtctgcctaccaatgtggaatgcagtcgctatcatctgcaacggatcctggtgtcagacactttcaacaagtggactag
aatcactctgcctctccctccttataccaggtcccaagccactcgtttccgttggcatcaaccagctccttttgacaagcag
cagacatgggcaatagataatgtctatatcggggatggctgcatagacatgtgcagtggccatgggagatgcatccag
ggaaactgcgtctgtgatgaacagtggggtggcctgtactgtgatgaccccgagacctctcttccaacccaactcaaag
acaacttcaatcgagctccatccagtcagaactggctgactgtgaacggagggaaattgagtacagtgtgtggagccg
tggcgtcgggaatggctctccatttcagtgggggttgtagtcgattattagtcactgtggatctaaacctcactaatgctga
gttcatccaattttacttcatgtatgggtgcctgattacaccaaacaaccgtaaccaaggtgttctcttggaatattctgtca
atggaggcattacctggaacctgctcatggagattttctatgaccagtacagtAGCGGAGGAGGTAcccttga
tagtaggaaatggctgcttcacccaggaggcaccaagatgcccgtgtgtggctctactggtgatgccctggtcttcattg
aaaaggccagcacccgttacgtggtcagcacagacgtcgccgtgaatgaggattccttcctacagatagacttcgctgt
ctcctgctcagtcacagactcttgttatgcgattgaattggaatactcagtagatcttggattgtcatggcacccattggtaa
gggactgtctgcctaccaatgtggaatgcagtcgctatcatctgcaacggatcctggtgtcagacactttcaacaagtgg
actagaatcactctgcctctccctccttataccaggtcccaagccactcgtttccgttggcatcaaccagctccttttgaca
agcagcagacatgggcaatagataatgtctatatcggggatggctgcatagacatgtgcagtggccatgggagatgc
atccagggaaactgcgtctgtgatgaacagtggggtggcctgtactgtgatgaccccgagacctctcttccaacccaac
tcaaagacaacttcaatcgagctccatccagtcagaactggctgactgtgaacggagggaaattgagtacagtgtgtg
gagccgtggcgtcgggaatggctctccatttcagtgggggttgtagtcgattattagtcactgtggatctaaacctcacta
atgctgagttcatccaattttacttcatgtatgggtgcctgattacaccaaacaaccgtaaccaaggtgttctcttggaatat
tctgtcaatggaggcattacctggaacctgctcatggagattttctatgaccagtacagtACCGGTGGATCTG
GAGGGagcactcgcaaacaaaattacatgatgaatttttcacgacaacatgggctcagacgtttctacaacaga
agacgaaggtcacttaggcgatacccat
233D pFUSEN-IL2ss-6xHis-hReelinR6-R6CT-H3447R (amino acid sequence-
translation of the sequence by domains)
IL2SS-LINKER-6Xhis-tag(SEQ ID NO: 62)-LINKER-r6 of reln-LINKER-r6 of reln-
LINKER-c-terminus reln H3447R
(SEQ ID NO: 33)
MYRMQLLSCIALSLALVTNSGGhhhhhhGGSRtldsrkwllhpggtkmpvcgstgdalvfieka
stryvvstdvavnedsflqidfavscsvtdscyaieleysvdlglswhplvrdclptnvecsryhlqrilvsdtfnkwtritlpl
ppytrsqatrfrwhqpapfdkqqtwaidnvyigdgcidmcsghgrciqgncvcdeqwgglycddpetslptqlkdnf
nrapssqnwltvnggklstvcgavasgmalhfsggcsrllvtvdlnltnaefiqfyfmygclitpnnrnqgvlleysvngg
itwnllmeifydqysSGGGtldsrkwllhpggtkmpvcgstgdalvfiekastryvvstdvavnedsflqidfavscsvt
dscyaieleysvdlglswhplvrdclptnvecsryhlqrilvsdtfnkwtritlplppytrsqatrfrwhqpapfdkqqtwa
idnvyigdgcidmcsghgrciqgncvcdeqwgglycddpetslptqlkdnfnrapssqnwltvnggklstvcgavasg
malhfsggcsrllvtvdlnltnaefiqfyfmygclitpnnrnqgvlleysvnggitwnllmeifydqysTGGSGGstrk
qnymmnfsrqhglrrfynrrrrslrryp
233E-pFUSEN-Il2ss-6xHIS-R5-6-FspCR50-CtermH3447R (nucleotide
sequence)
IL2SS-LINKER-6Xhis-tag(SEQ ID NO: 62)-LINKER-r5-6 of reln-LINKER-fspcr50
of reln-LINKER-c-terminus reln H3447R
(SEQ ID NO: 34)
ATGTACAGGATGCAACTCCTGTCTTGCATTGCACTAAGTCTTGCACTT
GTCACGAATTCGGGTGGCcaccatcatcaccatcacGGAGGTTCTAGAGGAGGAcaa
accaatgctacaagattcagactctggcaaccttataataacggtaagaaagaagaaatctggattgttgatgacttcat
tatcgatggaaataatgtaaacaaccctgtgatgctcttggatacatttgattttgggcccagagaagacaattggtttttct
atcctggtggtaacatcggtctttattgtccatattcttcaaagggggcacctgaagaagattcagctatggtgtttgtttca
aatgaagttggtgagcattccattaccacccgtgacctaaatgtgaatgagaacaccatcatacaatttgagatcaacgt
tggctgttcgactgatagctcatccgcggatccagtgagactggaattttcaagggacttcggggcgacctggcaccttc
tgctgcccctctgctaccacagcagcagccacgtcagctctttatgctccaccgagcaccaccccagcagcacctacta
cgcaggaaccatgcagggctggaggagggaggtcgtgcactttgggaagctgcacctttgtggatctgtccgtttcaga
tggtaccagggattttaccctgccggctctcagccagtgacatgggccattgataatgtctacatcggtccccagtgtgag
gagatgtgtaatggacaggggagctgtatcaatggaaccaaatgtatatgtgaccctggctactcaggtccaacctgta
aaataagcaccaaaaatcctgattttctcaaagatgatttcgaaggtcagctagaatctgatagattcttattaatgagtg
gtgggaaaccatctcgaaagtgtggaatcctttctagtggaaacaacctctttttcaatgaagatggcttgcgcatgttgat
gacacgagacctggatttatcacatgctagatttgtgcagttcttcatgagactgggatgtggtaaaggcgttcctgaccc
caggagtcaacccgtgctcctacagtattctctcaacggtggcctctcgtggagtcttcttcaggagttccttttcagcaatt
ccagcaatgtgggcaggtacattgccctggagatacccttgaaagcccgttctggttctactcgccttcgctggtggcaa
ccgtctgagaatgggcacttctacagcccctgggttatcgatcagattcttattggaggaaatatttctggtaatatggtctt
ggaagatgatttcacaacccttgatagtaggaaatggctgcttcacccaggaggcaccaagatgcccgtgtgtggctct
actggtgatgccctggtcttcattgaaaaggccagcacccgttacgtggtcagcacagacgtcgccgtgaatgaggatt
ccttcctacagatagacttcgctgtctcctgctcagtcacagactcttgttatgcgattgaattggaatactcagtagatcttg
gattgtcatggcacccattggtaagggactgtctgcctaccaatgtggaatgcagtcgctatcatctgcaacggatcctg
gtgtcagacactttcaacaagtggactagaatcactctgcctctccctccttataccaggtcccaagccactcgtttccgtt
ggcatcaaccagctccttttgacaagcagcagacatgggcaatagataatgtctatatcggggatggctgcatagacat
gtgcagtggccatgggagatgcatccagggaaactgcgtctgtgatgaacagtggggtggcctgtactgtgatgaccc
cgagacctctcttccaacccaactcaaagacaacttcaatcgagctccatccagtcagaactggctgactgtgaacgg
agggaaattgagtacagtgtgtggagccgtggcgtcgggaatggctctccatttcagtgggggttgtagtcgattattagt
cactgtggatctaaacctcactaatgctgagttcatccaattttacttcatgtatgggtgcctgattacaccaaacaaccgt
aaccaaggtgttctcttggaatattctgtcaatggaggcattacctggaacctgctcatggagattttctatgaccagtaca
gtaagcccggatttgtgaatatccttctccctcctgatgctaaagagattgccactcgcttccgctggtggcagccaagac
atgacggcctggatcagaacgactgggccattgacaatgtcctcatctcaggctctgctGGAGGTGGAACGC
Gtgcggctggctattacccccgcttttcgcccttctttttcctgtgcacccaccacggggagctggaaggggatggggagc
agggcgaggtgctcatttccctgcatattgcgggcaaccccacctactacgttccgggacaagaataccatgtgacaatttc
aacaagcaccttttttgacggcttgctggtgacaggactatacacatctacaagtgttcaggcatcacagagcattggaggtt
ccagtgctttcggatttgggatcatgtctgaccaccagtttggtaaccagtttatgtgcagtgtggtagcctctcacgtgagtca
cctgcccacaaccaacctcagtttcatctggattgctccacctgcgggcacaggctgtgtgaatttcatggctacagcaaca
caccggggccaggttattttcaaagatgctttagcccagcagttgtgtgaacaaggagctccaacagatgtcactgtgcacc
cacatctagctgaaatacatagtgacagcattatcctgagagatgactttgactcctaccaccaactgcaattaaatccaaata
tatgggttgaatgtaacaactgtgagactggagaacagtgtggcgcgattatgcatggcaatgccgtcaccttctgtgaacc
atatggcccacgagaactgattaccacaggccttaatacaacaacagcttctgtcctccaattttccattgggtcaggttcatgt
cgctttagttattcagaccccagcatcatcgtgttatatgccaagaataactctgcggactggattcagctagagaaaattaga
gccccttccaatgtcagcacaatcatccatatcctctaccttcctgaggacgccaaaggggagaatgtccaatttcagtggaa
gcaggaaaatcttcgtgtaggtgaagtgtatgaagcctgctgggccttagataacatcttgatcatcaatACCGGTGG
ATCTGGAGGGagcactcgcaaacaaaattacatgatgaatttttcacgacaacatgggctcagacgtttcta
caacagaagacgaaggtcacttaggcgatacccat
233E pFUSEN-Il2ss-6xHIS-R5-6-FspCR50-CtermH3447R (amino acid sequence-
translation of the sequence by domains)
IL2SS-LINKER-6Xhis-tag(SEQ ID NO: 62)-LINKER-r5-6 of reln-LINKER-fspcr50
of reln-LINKER-c-terminus reln H3447R
(SEQ ID NO: 35)
MYRMQLLSCIALSLALVTNSGGhhhhhhGGSRGGqtnatrfrlwqpynngkkeeiwivddfii
dgnnvnnpvmlldtfdfgprednwffypggniglycpysskgapeedsamvfvsnevgehsittrdlnvnentiiqfein
vgcstdsssadpvrlefsrdfgatwhlllplcyhssshvsslcstehhpsstyyagtmqgwrrevvhfgklhlcgsvrfrw
yqgfypagsqpvtwaidnvyigpqceemcngqgscingtkcicdpgysgptckistknpdflkddfegqlesdrfllms
ggkpsrkcgilssgnnlffnedglrmlmtrdldlsharfvqffmrlgcgkgvpdprsqpvllqyslngglswsllqeflfsn
ssnvgryialeiplkarsgstrlrwwqpsenghfyspwvidqiliggnisgnmvleddfttldsrkwllhpggtkmpvcg
stgdalvfiekastryvvstdvavnedsflqidfavscsvtdscyaieleysvdlglswhplvrdclptnvecsryhlqrilvs
dtfnkwtritlplppytrsqatrfrwhqpapfdkqqtwaidnvyigdgcidmcsghgrciqgncvcdeqwgglycddp
etslptqlkdnfnrapssqnwltvnggklstvcgavasgmalhfsggcsrllvtvdlnltnaefiqfyfmygclitpnnrnq
gvlleysvnggitwnllmeifydqyskpgfvnillppdakeiatrfrwwqprhdgldqndwaidnvlisgsaGGGT
Raagyyprfspffflcthhgelegdgeqgevlislhiagnptvvvpgqeyhytiststffdgllvtglvtstsvqasqsigg
ssafgfgimsdhqfgnqfmcsvvashvshlpttnlsfiwiappagtgcvnfmatathrgqvifkdalaqqlceqgaptd
vtvhphlaeihsdsiilrddfdsyhqlqlnpniwvecnncetgeqcgaimhgnavtfcepygprelittglntttasvlqf
sigsgscrfsysdpsiivlyaknnsadwiqlekirapsnvstiihilylpedakgenvqfqwkqenlrvgevyeacwal
dniliinTGGSGGstrkqnymmnfsrqhglrrfynrrrrslrryp
233F pFUSEN-IL2ss-6xHis-FspCR50-R6-R6 Cterm RELN-H3447R (nucleotide
sequence)
IL2SS-LINKER-6Xhis-tag(SEQ ID NO: 62)-LINKER-fspcr50 of reln-LINKER-r6 of
reln-LINKER-r6 of reln-LINKER-c-terminus reln H3447R
(SEQ ID NO: 36)
ATGTACAGGATGCAACTCCTGTCTTGCATTGCACTAAGTCTTGCACTT
GTCACGAATTCGGGTGGCcatcatcatcaccatcacGGAGGTGGGGcggctggctattacccc
cgcttttcgcccttctttttcctgtgcacccaccacggggagctggaaggggatggggagcagggcgaggtgctcatttccc
tgcatattgcgggcaaccccacctactacgttccgggacaagaataccatgtgacaatttcaacaagcaccttttttgacggc
ttgctggtgacaggactatacacatctacaagtgttcaggcatcacagagcattggaggttccagtgctttcggatttgggatc
atgtctgaccaccagtttggtaaccagtttatgtgcagtgtggtagcctctcacgtgagtcacctgcccacaaccaacctcag
tttcatctggattgctccacctgcgggcacaggctgtgtgaatttcatggctacagcaacacaccggggccaggttattttca
aagatgctttagcccagcagttgtgtgaacaaggagctccaacagatgtcactgtgcacccacatctagctgaaatacatag
tgacagcattatcctgagagatgactttgactcctaccaccaactgcaattaaatccaaatatatgggttgaatgtaacaactgt
gagactggagaacagtgtggcgcgattatgcatggcaatgccgtcaccttctgtgaaccatatggcccacgagaactgatt
accacaggccttaatacaacaacagcttctgtcctccaattttccattgggtcaggttcatgtcgctttagttattcagacccca
gcatcatcgtgttatatgccaagaataactctgcggactggattcagctagagaaaattagagccccttccaatgtcagcaca
atcatccatatcctctaccttcctgaggacgccaaaggggagaatgtccaatttcagtggaagcaggaaaatcttcgtgtag
gtgaagtgtatgaagcctgctgggccttagataacatcttgatcatcaatGGAGGTTCTAGAacccttgatagtag
gaaatggctgcttcacccaggaggcaccaagatgcccgtgtgtggctctactggtgatgccctggtcttcattgaaaag
gccagcacccgttacgtggtcagcacagacgtcgccgtgaatgaggattccttcctacagatagacttcgctgtctcctg
ctcagtcacagactcttgttatgcgattgaattggaatactcagtagatcttggattgtcatggcacccattggtaagggac
tgtctgcctaccaatgtggaatgcagtcgctatcatctgcaacggatcctggtgtcagacactttcaacaagtggactag
aatcactctgcctctccctccttataccaggtcccaagccactcgtttccgttggcatcaaccagctccttttgacaagcag
cagacatgggcaatagataatgtctatatcggggatggctgcatagacatgtgcagtggccatgggagatgcatccag
ggaaactgcgtctgtgatgaacagtggggtggcctgtactgtgatgaccccgagacctctcttccaacccaactcaaag
acaacttcaatcgagctccatccagtcagaactggctgactgtgaacggagggaaattgagtacagtgtgtggagccg
tggcgtcgggaatggctctccatttcagtgggggttgtagtcgattattagtcactgtggatctaaacctcactaatgctga
gttcatccaattttacttcatgtatgggtgcctgattacaccaaacaaccgtaaccaaggtgttctcttggaatattctgtca
atggaggcattacctggaacctgctcatggagattttctatgaccagtacagtAGCGGAGGAGGTAcccttga
tagtaggaaatggctgcttcacccaggaggcaccaagatgcccgtgtgtggctctactggtgatgccctggtcttcattg
aaaaggccagcacccgttacgtggtcagcacagacgtcgccgtgaatgaggattccttcctacagatagacttcgctgt
ctcctgctcagtcacagactcttgttatgcgattgaattggaatactcagtagatcttggattgtcatggcacccattggtaa
gggactgtctgcctaccaatgtggaatgcagtcgctatcatctgcaacggatcctggtgtcagacactttcaacaagtgg
actagaatcactctgcctctccctccttataccaggtcccaagccactcgtttccgttggcatcaaccagctccttttgaca
agcagcagacatgggcaatagataatgtctatatcggggatggctgcatagacatgtgcagtggccatgggagatgc
atccagggaaactgcgtctgtgatgaacagtggggtggcctgtactgtgatgaccccgagacctctcttccaacccaac
tcaaagacaacttcaatcgagctccatccagtcagaactggctgactgtgaacggagggaaattgagtacagtgtgtg
gagccgtggcgtcgggaatggctctccatttcagtgggggttgtagtcgattattagtcactgtggatctaaacctcacta
atgctgagttcatccaattttacttcatgtatgggtgcctgattacaccaaacaaccgtaaccaaggtgttctcttggaatat
tctgtcaatggaggcattacctggaacctgctcatggagattttctatgaccagtacagtACCGGTGGATCTG
GAGGGagcactcgcaaacaaaattacatgatgaatttttcacgacaacatgggctcagacgtttctacaacaga
agacgaaggtcacttaggcgatacccat
233F pFUSEN-IL2ss-6xHis-FspCR50-R6-R6 Cterm RELN-H3447R (amino acid
sequence-translation of the sequence by domains)
IL2SS-LINKER-6Xhis-tag(SEQ ID NO: 62)-LINKER-fspcr50 of reln-LINKER-r6 of
reln-LINKER-r6 of reln-LINKER-c-terminus reln H3447R
(SEQ ID NO: 37)
MYRMQLLSCIALSLALVTNSGGhhhhhhGGGaagyyprfspffflcthhgelegdgeqgevlisl
hiagnptyyvpgqeyhvtiststffdgllvtglytstsvqasqsiggssafgfgimsdhqfgnqfmcsvvashvshlpttn
lsfiwiappagtgcvnfmatathrgqvifkdalaqqlceqgaptdvtvhphlaeihsdsiilrddfdsyhqlqlnpniwv
ecnncetgeqcgaimhgnavtfcepygprelittglntttasvlqfsigsgscrfsysdpsiivlyaknnsadwiqlekira
psnvstiihilylpedakgenvqfqwkqenlrvgevyeacwaldniliinGGSRtldsrkwllhpggtkmpvcgstg
dalvfiekastryvvstdvavnedsflqidfavscsvtdscyaieleysvdlglswhplvrdclptnvecsryhlqrilvsdtf
nkwtritlplppytrsqatrfrwhqpapfdkqqtwaidnvyigdgcidmcsghgrciqgncvcdeqwgglycddpets
lptqlkdnfnrapssqnwltvnggklstvcgavasgmalhfsggcsrllvtvdlnltnaefiqfyfmygclitpnnrnqgvl
leysvnggitwnllmeifydqysSGGGtldsrkwllhpggtkmpvcgstgdalvfiekastryvvstdvavnedsflqi
dfavscsvtdscyaieleysvdlglswhplvrdclptnvecsryhlqrilvsdtfnkwtritlplppytrsqatrfrwhqpap
fdkqqtwaidnvyigdgcidmcsghgrciqgncvcdeqwgglycddpetslptqlkdnfnrapssqnwltvnggklst
vcgavasgmalhfsggcsrllvtvdlnltnaefiqfyfmygclitpnnrnqgvlleysvnggitwnllmeifydqysTGG
SGGstrkqnymmnfsrqhglrrfynrrrrslrryp
225RR pFUSEN-Il2ss-flagMini-RELNFspCR50R5-6CtermH3447R (nucleotide
sequence)
IL2SS-LINKER-flag tag-LINKER-fspcr50 of reln-LINKER-R5-6 OF RELN-c-
terminus reln H3447R
(SEQ ID NO: 38)
ATGTACAGGATGCAACTCCTGTCTTGCATTGCACTAAGTCTTGCACTT
GTCACGAATTCGGGTGGCgactacaaagacgatgacgacaagACCGGTGGTgcggctggcta
ttacccccgcttttcgcccttctttttcctgtgcacccaccacggggagctggaaggggatggggagcagggcgaggtg
ctcatttccctgcatattgcgggcaaccccacctactacgttccgggacaagaataccatgtgacaatttcaacaagcac
cttttttgacggcttgctggtgacaggactatacacatctacaagtgttcaggcatcacagagcattggaggttccagtgc
tttcggatttgggatcatgtctgaccaccagtttggtaaccagtttatgtgcagtgtggtagcctctcacgtgagtcacctgc
ccacaaccaacctcagtttcatctggattgctccacctgcgggcacaggctgtgtgaatttcatggctacagcaacacac
cggggccaggttattttcaaagatgctttagcccagcagttgtgtgaacaaggagctccaacagatgtcactgtgcacc
cacatctagctgaaatacatagtgacagcattatcctgagagatgactttgactcctaccaccaactgcaattaaatcca
aatatatgggttgaatgtaacaactgtgagactggagaacagtgtggcgcgattatgcatggcaatgccgtcaccttctg
tgaaccatatggcccacgagaactgattaccacaggccttaatacaacaacagcttctgtcctccaattttccattgggtc
aggttcatgtcgctttagttattcagaccccagcatcatcgtgttatatgccaagaataactctgcggactggattcagcta
gagaaaattagagccccttccaatgtcagcacaatcatccatatcctctaccttcctgaggacgccaaaggggagaat
gtccaatttcagtggaagcaggaaaatcttcgtgtaggtgaagtgtatgaagcctgctgggccttagataacatcttgatc
atcaatGGTTCTAGAGGAGGACAAACCAATGCTACAAGATTCAGACTCTGGCA
ACCTTATAATAACGGTAAGAAAGAAGAAATCTGGATTGTTGATGACTTCATT
ATCGATGGAAATAATGTAAACAACCCTGTGATGCTCTTGGATACATTTGATT
TTGGGCCCAGAGAAGACAATTGGTTTTTCTATCCTGGTGGTAACATCGGTC
TTTATTGTCCATATTCTTCAAAGGGGGCACCTGAAGAAGATTCAGCTATGGT
GTTTGTTTCAAATGAAGTTGGTGAGCATTCCATTACCACCCGTGACCTAAAT
GTGAATGAGAACACCATCATACAATTTGAGATCAACGTTGGCTGTTCGACT
GATAGCTCATCCGCGGATCCAGTGAGACTGGAATTTTCAAGGGACTTCGGG
GCGACCTGGCACCTTCTGCTGCCCCTCTGCTACCACAGCAGCAGCCACGTC
AGCTCTTTATGCTCCACCGAGCACCACCCCAGCAGCACCTACTACGCAGGA
ACCATGCAGGGCTGGAGGAGGGAGGTCGTGCACTTTGGGAAGCTGCACCT
TTGTGGATCTGTCCGTTTCAGATGGTACCAGGGATTTTACCCTGCCGGCTCT
CAGCCAGTGACATGGGCCATTGATAATGTCTACATCGGTCCCCAGTGTGAG
GAGATGTGTAATGGACAGGGGAGCTGTATCAATGGAACCAAATGTATATGT
GACCCTGGCTACTCAGGTCCAACCTGTAAAATAAGCACCAAAAATCCTGAT
TTTCTCAAAGATGATTTCGAAGGTCAGCTAGAATCTGATAGATTCTTATTAAT
GAGTGGTGGGAAACCATCTCGAAAGTGTGGAATCCTTTCTAGTGGAAACA
ACCTCTTTTTCAATGAAGATGGCTTGCGCATGTTGATGACACGAGACCTGG
ATTTATCACATGCTAGATTTGTGCAGTTCTTCATGAGACTGGGATGTGGTAA
AGGCGTTCCTGACCCCAGGAGTCAACCCGTGCTCCTACAGTATTCTCTCAA
CGGTGGCCTCTCGTGGAGTCTTCTTCAGGAGTTCCTTTTCAGCAATTCCAG
CAATGTGGGCAGGTACATTGCCCTGGAGATACCCTTGAAAGCCCGTTCTGG
TTCTACTCGCCTTCGCTGGTGGCAACCGTCTGAGAATGGGCACTTCTACAG
CCCCTGGGTTATCGATCAGATTCTTATTGGAGGAAATATTTCTGGTAATATGG
TCTTGGAAGATGATTTCACAACCCTTGATAGTAGGAAATGGCTGCTTCACC
CAGGAGGCACCAAGATGCCCGTGTGTGGCTCTACTGGTGATGCCCTGGTCT
TCATTGAAAAGGCCAGCACCCGTTACGTGGTCAGCACAGACGTCGCCGTG
AATGAGGATTCCTTCCTACAGATAGACTTCGCTGTCTCCTGCTCAGTCACAG
ACTCTTGTTATGCGATTGAATTGGAATACTCAGTAGATCTTGGATTGTCATG
GCACCCATTGGTAAGGGACTGTCTGCCTACCAATGTGGAATGCAGTCGCTA
TCATCTGCAACGGATCCTGGTGTCAGACACTTTCAACAAGTGGACTAGAAT
CACTCTGCCTCTCCCTCCTTATACCAGGTCCCAAGCCACTCGTTTCCGTTGG
CATCAACCAGCTCCTTTTGACAAGCAGCAGACATGGGCAATAGATAATGTC
TATATCGGGGATGGCTGCATAGACATGTGCAGTGGCCATGGGAGATGCATCC
AGGGAAACTGCGTCTGTGATGAACAGTGGGGTGGCCTGTACTGTGATGAC
CCCGAGACCTCTCTTCCAACCCAACTCAAAGACAACTTCAATCGAGCTCCA
TCCAGTCAGAACTGGCTGACTGTGAACGGAGGGAAATTGAGTACAGTGTG
TGGAGCCGTGGCGTCGGGAATGGCTCTCCATTTCAGTGGGGGTTGTAGTCG
ATTATTAGTCACTGTGGATCTAAACCTCACTAATGCTGAGTTCATCCAATTTT
ACTTCATGTATGGGTGCCTGATTACACCAAACAACCGTAACCAAGGTGTTC
TCTTGGAATATTCTGTCAATGGAGGCATTACCTGGAACCTGCTCATGGAGAT
TTTCTATGACCAGTACAGTAAGCCCGGATTTGTGAATATCCTTCTCCCTCCT
GATGCTAAAGAGATTGCCACTCGCTTCCGCTGGTGGCAGCCAAGACATGAC
GGCCTGGATCAGAACGACTGGGCCATTGACAATGTCCTCATCTCAGGCTCT
GCTagcactcgcaaacaaaattacatgatgaatttttcacgacaacatgggctcagacgtttctacaacagaaga
cgaaggtcacttaggcgatacccat
225RR pFUSEN-Il2ss-flagMini-RELNFspCR50R5-6CtermH3447R (amino acid
sequence-translation of the sequence by domains)
IL2SS-LINKER-flag tag-LINKER-fspcr50 of reln-LINKER-R5-6 OF RELN-c-
terminus reln H3447R
(SEQ ID NO: 39)
MYRMQLLSCIALSLALVTNSGGdykddddkTGGaagyyprfspffflcthhgelegdgeqgevli
slhiagnptyyvpgqeyhvtiststffdgllvtglytstsvqasqsiggssafgfgimsdhqfgnqfmcsvvashvshlpttn
lsfiwiappagtgcvnfmatathrgqvifkdalaqqlceqgaptdvtvhphlaeihsdsiilrddfdsyhqlqlnpniwve
cnncetgeqcgaimhgnavtfcepygprelittglntttasvlqfsigsgscrfsysdpsiivlyaknnsadwiqlekirap
snvstiihilylpedakgenvqfqwkqenlrvgevyeacwaldniliinGSRGGQTNATRFRLWQPYNN
GKKEEIWIVDDFIIDGNNVNNPVMLLDTFDFGPREDNWFFYPGGNIGLYCPYS
SKGAPEEDSAMVFVSNEVGEHSITTRDLNVNENTIIQFEINVGCSTDSSSADPV
RLEFSRDFGATWHLLLPLCYHSSSHVSSLCSTEHHPSSTYYAGTMQGWRREV
VHFGKLHLCGSVRFRWYQGFYPAGSQPVTWAIDNVYIGPQCEEMCNGQGSCI
NGTKCICDPGYSGPTCKISTKNPDFLKDDFEGQLESDRFLLMSGGKPSRKCGIL
SSGNNLFFNEDGLRMLMTRDLDLSHARFVQFFMRLGCGKGVPDPRSQPVLL
QYSLNGGLSWSLLQEFLFSNSSNVGRYIALEIPLKARSGSTRLRWWQPSENGH
FYSPWVIDQILIGGNISGNMVLEDDFTTLDSRKWLLHPGGTKMPVCGSTGDAL
VFIEKASTRYVVSTDVAVNEDSFLQIDFAVSCSVTDSCYAIELEYSVDLGLSWH
PLVRDCLPTNVECSRYHLQRILVSDTFNKWTRITLPLPPYTRSQATRFRWHQPA
PFDKQQTWAIDNVYIGDGCIDMCSGHGRCIQGNCVCDEQWGGLYCDDPETSL
PTQLKDNFNRAPSSQNWLTVNGGKLSTVCGAVASGMALHFSGGCSRLLVTVD
LNLTNAEFIQFYFMYGCLITPNNRNQGVLLEYSVNGGITWNLLMEIFYDQYSK
PGFVNILLPPDAKEIATRFRWWQPRHDGLDQNDWAIDNVLISGSAstrkqnymm
nfsrqhglrrfynrrrrslrryp

In some embodiments, the proteins and nucleic acids used herein are at least 80%, 85%, 90%, 95%, 97%, 98%, or 99% identical to a sequence provided herein, so long as they retain desired functionality of the parental sequence. In some embodiments, the proteins can include a sequence provided herein with exactly, at least, or up to one, two, three, four, five, six, seven, eight, nine, or ten altered amino acids.

Residues that can be changed without destroying function can be identified, e.g., by aligning similar sequences and making conservative substitutions in non-conserved regions (see, e.g., the alignments provided herein). To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In some embodiments, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

Increased RELN signaling can also be achieved by increasing activity or expression levels of DAB1 using different approaches including increasing its phosphorylation, decreasing dephosphorylation or increasing dimerization or oligomerization. An exemplary sequence of DAB1 is included here. DAB1 expression may be achieved via mRNA administration. Dimerization of DAB1 may be achieved by expressing a DAB1 fusion to FKBP, and the activity of the protein fusion can be controlled by administration of rapamycin. An exemplary Dab1 dimerising construct protein sequence (Dab1 in bold followed by FKBP) is as follows:

(SEQ ID NO: 40)
MASRSTETELQVAVKTSAKKDSRKKGQDRSEATLIKRFKGEGVRYKAKLIGIDEVSA
ARGDKLCQDSMMKLKGVVAGARSKGEHKOKIFLTISFGGIKIFDEKTGALQHHHAVH
EISYIAKDITDHRAFGYVCGKEGNHRFVAIKTAQAAEPVILDLRDLFQLIYELKORE
ELEKKAQKDKQCEQAVYQTILEEDVEDPVYQYIVFEAGHEPIRDPETEENIYQVPTS
QKKEGVYDVPKSQPVSNGYSFEDFEERFAAATPNRNLPTDFDEIFEATKAVTQLELF
GDMSTPPDITSPPTPATPGDAFIPSSSQTLPASADVFSSVPFGTAAVPSGYVAMGAV
LPSFWGQQPLVQQQMVMGAQPPVAQVMPGAQPIAWGQPGLFPATQQPWPTVAGQFPP
AAFMPTQTVMPLPAAMFQGPLTPLATVPGTSDSTRSSPQTDKPRQKMGKETFKDFQM
AQPPPVPSRKPDQPSLTCTSEAFSSYFNKVGVAQDTDDCDDFDISQLNLTPVTSTTP
STNSPPTPAPROSSPSKSSASHASDPTTDDIFEEGFESPSKSEEQEAPDGSQASSNS
DPFGEPSGEPSGDNISPQAGSSRGVQVETISPGDGRTFPKRGQTCVVHYTGMLEDGK
KVDSSRDRNKPFKFMLGKQEVIRGWEEGVAQMSVGQRAKLTISPDYAYGATGHPGII
PPHATLVEDVELLKLE

An exemplary sequence encoding the Dab1 dimerising construct is provided below in the Examples.

RAP clustering can also be used increase reelin signaling; see, e.g., Strasser et al., Mol Cell Biol. 2004 February; 24(3):1378-86. An exemplary sequence of a protein fusion of RAP to IgG2 follows (RAP in bold capital case, linker in capital case italics, igG2 in lower case, linker in capital case, RELN CTR with the H3447R variant in lower case bold) (this corresponds to 225T exemplary sequence):

(SEQ ID NO: 41)
MAPRRVRSFLRGLPALLLLLLFLGPWPAASHGGKYSREKNQPKPSPKRE
SGEEFRMEKLNQLWEKAQRLHLPPVRLAELHADLKIQERDELAWKKLK
LDGLDEDGEKEARLIRNLNVILAKYGLDGKKDARQVTSNSLSGTQEDGL
DDPRLEKLWHKAKTSGKFSGEELDKLWREFLHHKEKVHEYNVLLETLS
RTEEIHENVISPSDLSDIKGSVLHSRHTELKEKLRSINQGLDRLRRVSHQG
YSTEAEFEEPRVIDLWDLAQSANLTDKELEAFREELKHFEAKIEKHNHYQ
KQLEIAHEKLRHAESVGDGERVSRSREKHALLEGRTKELGYTVKKHLQ
DLSGRISRARHNELTGGSGGNSaplerkssvecppcpappvagpsvflfppkpkdtlmisrtpevtc
vvvdvshedpevqfnwyvdgvevhnaktkpreeqfnstfrvvsvltvvhqdwlngkeykckvsnkglpapiektis
ktkgqprepqvytlppsreemtknqvsltclvkgfypsdiavewesngqpennykttppmldsdgsfflyskltvdks
rwqqgnvfscsvmhealhnhytqkslslspgaRTGGGGSGGGGSstrkqnymmnfsrqhglrrfynrr
rrslrryp

The HNEL (SEQ ID NO:61) sequence may be removed to enhance secretion.

Also provided herein are nucleic acids encoding the proteins described herein (e.g., mini-reelin and variants described in table A), as well as cells expressing the proteins, and nucleic acids encoding the variants, including vectors such as viral vectors.

### Gene Editing

CRISPR-based methods can also be used to increase levels or activity of reelin. For example, CRISPR-Cas9 can be used to introduce a H3447R or H3447K mutation and/or a R3454A mutation, optionally H3447R or H3447K in combination with an R3454A mutation, into a reelin gene of a subject who has one or more wild-type reelin alleles. For example, prime editing (see, e.g., Anzalone et al., Nature volume 576, pages149-157 (2019)) can be used to introduce the alteration, optionally using an ssODN comprising the sequence: TTGATTCTTCATGGGTATCGCCTAAGTGACCTTCGTCTTCTGTTGTAGAAAc GTCTGAGTCCATGTTGTCGTGAAAAATTCATCATGTAATTTTGTTTGCGA GTGCTGTTAAAATCAAA(SEQ ID NO:42), and a single guide RNA (sgRNA), or a tracrRNA and crNA, comprising a spacer region having the sequence:

(SEQ ID NO: 43)
TGAATTTTTCACGACAACAT.

In some embodiments, the ssODN includes two mismatches with the wild type RELN gene; the first mismatch restore the mutation H3447R (CAT to CGT), a second, optional mismatch destroys the PAM (NGG) from GGG to GGA, as shown below.

| | | |
|---|---|---|
| SSODN seq | TTGATTCTTCATGGGTATCGCCTAAGTGACCTTCGTCTTCTGTTGTAGAAACGTCTGAGT | 60 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\| | |
| REIN 70880 | TTGATTCTTCATGGGTATCGCCTAAGTGACCTTCGTCTTCIGITGTAGAAATGTCTGAGC | 70939 |
| SSODN seq | CCATGTTGTCGTGAAAAATTCATCATGTAATTTIGTTTGCGAGIGCTGTTAAAATCAAA | 119 |
| (SEQ ID NO: 42) | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| RELN 70940 | CCATGTTGTCGTGAAAAATTCATCATGTAATTTIGTTTGCGAGTGCTGTTAAAATCAAA | 70998 |
| (SEQ ID NO: 63) | | |

Similarly, the ssODN can be designed to restore H3447R or H3447K mutation and/or a R3454A mutation, optionally H3447R or H3447K in combination with an R3454A mutation.

The Cas9 nuclease from S. pyogenes can be guided via simple base pair complementarity between 17-20 nucleotides of an engineered guide RNA (gRNA), e.g., a single guide RNA or crRNA/tracrRNA pair, and the complementary strand of a target genomic DNA sequence of interest that lies next to a protospacer adjacent motif (PAM), e.g., a PAM matching the sequence NGG or NAG (Shen et al., Cell Res (2013); Dicarlo et al., Nucleic Acids Res (2013); Jiang et al., Nat Biotechnol 31, 233-239 (2013); Jinek et al., Elife 2, e00471 (2013); Hwang et al., Nat Biotechnol 31, 227-229 (2013); Cong et al., Science 339, 819-823 (2013); Mali et al., Science 339, 823-826 (2013c); Cho et al., Nat Biotechnol 31, 230-232 (2013); Jinek et al., Science 337, 816-821 (2012)). The engineered CRISPR from Prevotella and Francisella 1 (Cpf1, also known as Cas12a) nuclease can also be used, e.g., as described in Zetsche et al., Cell 163, 759-771 (2015); Schunder et al., Int J Med Microbiol 303, 51-60 (2013); Makarova et al., Nat Rev Microbiol 13, 722-736 (2015); Fagerlund et al., Genome Biol 16, 251 (2015). Unlike SpCas9, Cpf1/Cas12a requires only a single 42-nt crRNA, which has 23 nt at its 3′ end that are complementary to the protospacer of the target DNA sequence (Zetsche et al., 2015). Furthermore, whereas SpCas9 recognizes an NGG PAM sequence that is 3′ of the protospacer, AsCpf1 and LbCp1 recognize TTTN PAMs that are found 5′ of the protospacer (Id.).

In some embodiments, the present system utilizes a wild type or variant Cas9 protein from S. pyogenes or Staphylococcus aureus, or a wild type or variant Cpf1 protein from Acidaminococcus sp. BV3L6 or Lachnospiraceae bacterium ND2006 either as encoded in bacteria or codon-optimized for expression in mammalian cells and/or modified in its PAM recognition specificity and/or its genome-wide specificity. A number of variants have been described; see, e.g., WO 2016/141224, PCT/US2016/049147, Kleinstiver et al., Nat Biotechnol. 2016 August; 34(8):869-74; Tsai and Joung, Nat Rev Genet. 2016 May; 17(5):300-12; Kleinstiver et al., Nature. 2016 Jan. 28; 529(7587):490-5; Shmakov et al., Mol Cell. 2015 Nov. 5; 60(3):385-97; Kleinstiver et al., Nat Biotechnol. 2015 December; 33(12):1293-1298; Dahlman et al., Nat Biotechnol. 2015 November; 33(11):1159-61; Kleinstiver et al., Nature. 2015 Jul. 23; 523(7561):481-5; Wyvekens et al., Hum Gene Ther. 2015 July; 26(7):425-31; Hwang et al., Methods Mol Biol. 2015; 1311:317-34; Osborn et al., Hum Gene Ther. 2015 February; 26(2):114-26; Konermann et al., Nature. 2015 Jan. 29; 517(7536):583-8; Fu et al., Methods Enzymol. 2014; 546:21-45; and Tsai et al., Nat Biotechnol. 2014 June; 32(6):569-76, inter alia.

In some embodiments, TrueCut Cas9 Protein v2 is used. Cas9 and analogs are shown in Table B, and engineered protospacer-adjacent motif (PAM) or high-fidelity variants are shown in Table C.

TABLE B
List of Exemplary Cas9 or Cas12a Orthologs

| | UniProt or GenBank Accession |
|---|---|
| Ortholog | Number |
| S. pyogenes Cas9 (SpCas9) | Q99ZW2.1 |
| S. aureus Cas9 (SaCas9) | J7RUA5.1 |
| S. thermophilus Cas9 (St1Cas9) | G3ECR1.2 |
| S. pasteurianus Cas9 (SpaCas9) | BAK30384.1 |
| C. jejuni Cas9 (CjCas9) | Q0P897.1 |
| F. novicida Cas9 (FnCas9) | A0Q5Y3.1 |
| P. lavamentivorans Cas9 (PlCas9) | A7HP89.1 |
| C. lari Cas9 (ClCas9) | G1UFN3.1 |
| Pasteurella multocida Cas9 | Q9CLT2.1 |
| F. novicida Cpf1 (FnCpf1) | A0Q7Q2.1 |
| M. bovoculi Cpf1 (MbCpf1) | WP_052585281.1 |
| A. sp. BV3L6 Cpf1 (AsCpf1) | U2UMQ6.1 |
| L. bacterium N2006 (LbCpf1) | A0A182DWE3.1 |

TABLE C
List of Exemplary High Fidelity and/or PAM-relaxed RGN Orthologs

| Published | | |
|---|---|---|
| HF/PAM-RGN | | |
| variants | PMID/USSN | Mutations* |
| S. pyogenes Cas9 | 26628643 | K810A/K1003A/R1060A (1.0); |
| (SpCas9) | | K848A/K1003A/R1060A(1.1) |
| eSpCas9 | | |
| S. pyogenes Cas9 | 29431739 | M495V/Y515N/K526E/R661Q; |
| (SpCas9) | | (M495V/Y515N/K526E/R661S; |
| evoCas9 | | M495V/Y515N/K526E/R661L) |
| S. pyogenes Cas9 | 26735016 | N497A/R661A/Q695A/Q926A |
| (SpCas9) HF1 | | |
| S. pyogenes Cas9 | 30082871 | R691A |
| (SpCas9) HiFi | | |
| Cas9 | | |
| S. pyogenes Cas9 | 28931002 | N692A, M694A, Q695A, H698A |
| (SpCas9) | | |
| HypaCas9 | | |
| S. pyogenes Cas9 | 30082838 | F539S, M763I, K890N |
| (SpCas9) Sniper- | | |
| Cas9 | | |
| S. pyogenes Cas9 | 29512652 | A262T, R324L, S409I, E480K, E543D, M694I, |
| (SpCas9) xCas9 | | E1219V |
| S. pyogenes Cas9 | 30166441 | R1335V, L1111R, D1135V, G1218R, |
| (SpCas9) | | E1219F, A1322R, T1337R |
| SpCas9-NG | | |
| S. pyogenes Cas9 | 26098369 | D1135V, R1335Q, T1337R; |
| (SpCas9) | | D1135V/G1218R/R1335E/T1337R |
| VQR/VRER | | |
| S. aureus Cas9 | 26524662 | E782K/N968K/R1015H |
| (SaCas9)-KKH | | |
| enAsCas12a | U.S. Ser. No. | One or more of: E174R, S170R, S542R, K548R, |
| | 15/960,271 | K548V, N551R, N552R, K607R, K607H, e.g., |
| | | E174R/S542R/K548R, E174R/S542R/K607R, |
| | | E174R/S542R/K548V/N552R, |
| | | S170R/S542R/K548R, S170R/E174R, |
| | | E174R/S542R, S170R/S542R, |
| | | E174R/S542R/K548R/N551R, |
| | | E174R/S542R/K607H, S170R/S542R/K607R, or |
| | | S170R/S542R/K548V/N552R |
| enAsCas12a-HF | U.S. Ser. No. | One or more of: E174R, S542R, K548R, e.g., |
| | 15/960,271 | E174R/S542R/K548R, E174R/S542R/K607R, |
| | | E174R/S542R/K548V/N552R, |
| | | S170R/S542R/K548R, S170R/E174R, |
| | | E174R/S542R, S170R/S542R, |
| | | E174R/S542R/K548R/N551R, |
| | | E174R/S542R/K607H, S170R/S542R/K607R, or |
| | | S170R/S542R/K548V/N552R, with the addition of |
| | | one or more of: N282A, T315A, N515A and K949A |
| enLbCas12a(HF) | U.S. Ser. No. | One or more of T152R, T152K, D156R, D156K, |
| | 15/960,271 | Q529K, G532R, G532K, G532Q, K538R, K538V, |
| | | D541R, Y542R, M592A, K595R, K595H, K595S or |
| | | K595Q, e.g., D156R/G532R/K538R, |
| | | D156R/G532R/K595R, |
| | | D156R/G532R/K538V/Y542R, |
| | | T152R/G532R/K538R, T152R/D156R, |
| | | D156R/G532R, T152R/G532R, |
| | | D156R/G532R/K538R/D541R, |
| | | D156R/G532R/K595H, T152R/G532R/K595R, |
| | | T152R/G532R/K538V/Y542R, optionally with the |
| | | addition of one or more of: N260A, N256A, K514A, |
| | | D505A, K881A, S286A, K272A, K897A |
| enFnCas12a(HF) | U.S. Ser. No. | One or more of T177A, K180R, K180K, E184R, |
| | 15/960,271 | E184K, T604K, N607R, N607K, N607Q, K613R, |
| | | K613V, D616R, N617R, M668A, K671R, K671H, |
| | | K671S, or K671Q, e.g., E184R/N607R/K613R, |
| | | E184R/N607R/K671R, |
| | | E184R/N607R/K613V/N617R, |
| | | K180R/N607R/K613R, K180R/E184R, |
| | | E184R/N607R, K180R/N607R, |
| | | E184R/N607R/K613R/D616R, |
| | | E184R/N607R/K671H, K180R/N607R/K671R, |
| | | K180R/N607R/K613V/N617R, optionally with the |
| | | addition of one or more of: N305A, N301A, K589A, |
| | | N580A, K962A, S334A, K320A, K978A |

*predicted based on UniRule annotation on the UniProt database.

In some embodiments an RGN sequence is modified to include a nuclear localization sequences (NLSs), e.g., at the C- and/or N-terminus of the RGN protein, and a mini-polyadenylation signal (or Poly-A sequence). Exemplary NLSs include SV40 large T antigen NLS (PKKKRRV)(SEQ ID NO:44); PKiKKRKV(SEQ ID NO:45); KRTADGSEFES)PKKKRKV(SEQ ID NO:46); and nucleoplasmin NLS (KRPAATKIKAGQAKKKK)(SEQ ID NO:47). Other NLSs are known in the art; see, e.g., Cokol et al., EMBO Rep. 2000 Nov. 15; 1(5):411-415; Freitas and Cunha, Curr Genomics. 2009 December; 10(8): 550-557. An exemplary polyadenylation signal is

(SEQ ID NO: 48)
TAGCAATAAAGGATCGTTTATTTTCATTGGAAGCGTGTG
TTGGTTTTTTGATCAGGCGCG.

Guide RNAs appropriate for the RGN should be used; in some embodiments, the gRNAs used in the present disclosure can be unimolecular or modular, as known in the art.

In some embodiments, ribonucleoprotein complexes (RNPs), or gene therapy vectors or mRNA encoding CRISPR/Cas9 constructs, ssODN, and sgRNA, e.g., the above ssODN and sgRNA, are administered to introduce the H3447R or H3447K mutation and/or a R3454A mutation, optionally H3447R or H3447K in combination with an R3454A mutation in RELN into the whole brain or more specifically into the entorhinal cortex, e.g., using stereotactic injection as described herein. In some embodiments, where the Cas9 protein is administered in a viral vector, the sequence can be split across two vectors (see, e.g., Truong et al., Nucleic Acids Res. 2015 Jul. 27; 43(13):6450-8).

### Demethylation of the RELN Promoter

Methylation of the RELN promoter reduces expression of reelin protein (Chen et al., Nucleic Acids Res. 2002 Jul. 1; 30(13):2930-9. Thus the present methods can include inducing demethylation of the RELN promoter. As one example, CRISPR/Cas-based demethylases, in which a demethylase (e.g., Tet1 catalytic domain (Tet-CD)) is linked to a catalytically inactive (dead) Cas protein (Xu et al., Cell Discovery (2016) 2, 16009), can be used to trigger demethylation of the promoter sequence of RELN to de-repress and therefore increase expression of reelin (57). The promoter sequence of the RELN gene is listed below, with proposed gRNAs for use with dCas9 targeting to regulate methylation.

### RELN Promoter Sequence Features:

346-365 misc_feature: sgRNA1—ggccaaaggggctggccttc (SEQ ID NO: 49)
383-384 misc_feature: −458 Lintas et al. (56)
430-431 misc_feature: −411 Lintas et al. (56)
439-440 misc_feature: −402 Lintas et al. (56)
394-413 misc_feature: sgRNA2—ctctgcggggctttgacgtc (SEQ ID NO: 50)
441-460 misc_feature: sgRNA3—gtcctcgacagcgtccccgt (SEQ ID NO: 51)
1001-1003 misc_feature: orf start

RELN Promoter Sequence:
(SEQ ID NO: 52)
acaaatcaccatttccaggcaccactcgagtctgccaattccactttcgc
ttaatctcattttaaaatcctctacagatgaggctctgtcgctgccgcga
ggggccgggcggccgcggcgggagggacagggggcctgggtgggaaggga
gcgcgagggcccctagcggttcgcggcctcgggcggattcccagaacgag
tgcgggccgcccggactggcagactgcgagccccgaggggcgcgggacgc
agccggctcagacaaagaaaccggcgccagcggccgtccctgccgcccct
ctccttccctcacgcatcctcccaggaaaaacagggcacactgacggcca
aaggggctggccttccccttcagacagctggaCGggaggtgccctctgcg
gggctttgacgtccctcgcagaagagtcgCGggctcagCGgtcctcgaca
gcgtccccgtcccgctccccggcgggcgcccctccctgtcctcccgggtg
cgaaccgggcgctggccggggactccggggacgcgtgcgcccctcgccgc
gcgaggtgccgccgagccagcccgagagggcggggggcgggcggggcggc
gcgcgggggcgggggagcggccgggacacgtgtggcggcggcggggggga
cgcggcgcccggggctttaagaaggtgtggagcggggcgggcgctttccc
aggcctggccgaggggcgtcgcgcagaggcggcggcggcgcacggaggcg
gcagacgacgcgctctcggcgcccgcagccccggtcccgcgctcccgcgg
cccaaagtaactttgggagccgccgtctcccgcggaacttctcggcgggg
gcccgctcccaggcccgctcccgagcccgttccgctcccgtccgccttct
tctcgccttctctccgcgtggctcctccgtcccggcgtctccaaaactga
atgagcgagcggcgcgtagggcgcgcggcggcggcggcggcggcggcggc
ATGGAGCGCAGTGGCTGGGCCCGGCAGACTTTCCTCCTAGCGCTGTTGCT
GGGGGCGACGCTGAGGGCGCGCGCGGCGGCTGGCTATTACCCCCGCTTTT
CGCCCTTCTTTTTCCTGTGCACCCACCACGGGGAGCTGGAAGGGGATGGG
GAGCAGGGCGAGGTGCTCATTTCCCTGCATATTGCGGGCAACCCCACCTA
CTACGTTCCGGGACAAGAATACCATGGTAGGTACCGCAGCACCTCCGCGC
TCGCCGCCGCCGGGTGCGCCCGCCAGCGCACCTTTCTCATCCCTTTCCTT
CTTCCAAGGGGCCTTGGAAACAAGAACCATGACAACCCTGGCCCCAGCGA
TGCGCTTGGTCCAGGGAGCGAGAATAAAGTTAGTGGAGAATGGAGTCACA
AGTTGGGATTCATTCTCCATGTCCCCACTCCCCGAACGCATCTCCTGGTA
ATGAGCTGGTGCTTGTGCTAAACGCCTGAGAATTCTTGGAGGCCGGTGTC
TTTCCCCCTCACTCCCCGTACTTACCGCAGGCACTTTAAATTGAACCTGC
GCATTTTGTTTCTTTACTCCTAGGACTTGGATTTCAGACCTTTGTCCTGC
TCCCGGTCCCCCCAGATGGGTCCCCGGGGGGGCGTTGGGGGCTGTCGCTG
GGCTTTGCGGGCAGGCTGGTGACAGATTGAAGCGCCCAGGTCTGTGGCTG
CCCCGCGCCGCTCGACCGCAAAGTTCCCGCTGCTCACCAGCCAGGAGTCC
CTGGCATTTCATTCAGTGTCATACAGCCCTCCTGAAAGGGAGCGGCTGCT
TCCAGCTCCCTATTCTTTTGAAAGTGTGATTTAATATGTCAGTAATTGAT
GATTTTTCTGATTCCTGCCTTACTTTTACATGGTGCTTCTTGTTAGAATT
ATCTTTGTGATGTTTATTAATGACTTTGTATTTTATCTGCACAGCTTTAA
TTTCCAGGTAGAGGCTGGAATCCCTTATAATTTAGAGGTATCTTAAAAAT

In some embodiments, ribonucleoprotein complexes (RNPs), gene therapy, or mRNA encoding CRISPR/Cas9 constructs and the above ssODN and sgRNA are administered to introduce the H3447R or H3447K mutation and/or a R3454A mutation, optionally H3447R or H3447K in combination with an R3454A mutation in RELN into the whole brain or more specifically into the entorhinal cortex, e.g., using stereotactic injection as described herein.

Other compounds can also be used to reduce methylation of the RELN promoter and thus increase expression of the reelin gene, including administration of hsa_circRNA_102049, which acts as a sponge for hsa-miR-214-3p (Wang et al., Bioengineered. 2022 February; 13(2):2272-2284).

### Delivery Vectors

Nucleic acids encoding a Reelin or CRISPR/cas polypeptide (e.g., wild type, variant, peptide, or fragment thereof) can be incorporated into a gene construct to be used as a part of a gene therapy protocol. For example, described herein are targeted expression vectors for in vivo delivery and expression of a polynucleotide that encodes a Reelin polypeptide or active fragment thereof in particular cell types, especially cerebral cortical neuronal cells. Expression constructs of such components can be administered in any effective carrier, e.g., any formulation or composition capable of effectively delivering the component gene to cells in vivo. Approaches include insertion of the gene in viral vectors, preferably adeno-associated virus. Viral vectors typically transduce cells directly.

Viral vectors capable of highly efficient transduction of CNS neurons may be employed, including any serotypes of rAAV (e.g., AAV1-AAV12) vectors, recombinant or chimeric AAV vectors, as well as lentivirus or other suitable viral vectors. In some embodiments, a polynucleotide encoding Reelin is operably linked to promoter suitable for expression in the CNS. For example, a neuron subtype-specific specific promoter, such as the alpha-calcium/calmodulin kinase 2A promoter may be used to target excitatory neurons. Alternatively, a pan neuronal promoter, such as the synapsin I promoter, may be used to drive Reelin expression. Other exemplary promoters include, but are not limited to, a cytomegalovirus (CMV) early enhancer/promoter; a hybrid CMV enhance/chicken β-actin (CBA) promoter; a promoter comprising the CMV early enhancer element, the first exon and first intron of the chicken β-actin gene, and the splice acceptor of the rabbit β-globin gene (commonly call the “CAG promoter”); or a 1.6-kb hybrid promoter composed of a CMV immediate-early enhancer and CBA intron 1/exon 1 (commonly called the CAGGS promoter; Niwa et al. Gene, 108:193-199 (1991)). The CAGGS promoter (Niwa et al., 1991) has been shown to provide ubiquitous and long-term expression in the brain (Klein et al., Exp. Neurol. 176:66-74 (2002)).A typical approach for in vivo introduction of nucleic acid into a cell is by use of a viral vector containing nucleic acid, e.g., a cDNA encoding a Reelin. Among other things, infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

A viral vector system particularly useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro and Immunol. 158:97-129 (1992)). AAV vectors efficiently transduce various cell types and can produce long-term expression of transgenes in vivo. Although AAV vector genomes can persist within cells as episomes, vector integration has been observed (see for example Deyle and Russell, Curr Opin Mol Ther. 2009 August; 11(4): 442-447; Asokan et al., Mol Ther. 2012 April; 20(4): 699-708; Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989)). AAV vectors, such as AAV2, have been extensively used for gene augmentation or replacement and have shown therapeutic efficacy in a range of animal models as well as in the clinic; see, e.g., Mingozzi and High, Nature Reviews Genetics 12, 341-355 (2011); Deyle and Russell, Curr Opin Mol Ther. 2009 August; 11(4): 442-447; Asokan et al., Mol Ther. 2012 April; 20(4): 699-708. AAV vectors containing as little as 300 base pairs of AAV can be packaged and can produce recombinant protein expression. Protocols for producing recombinant retroviruses and for infecting cells in vitro or in vivo with such viruses are known in the art, e.g., can be found in Ausubel, et al., eds., Current Protocols in Molecular Biology, Greene Publishing Associates, (1989), Sections 9.10-9.14, and other standard laboratory manuals. The use of AAV vectors to deliver constructs for expression in the brain has been described, e.g., in Iwata et al., Sci Rep. 2013; 3:1472; Hester et al., Curr Gene Ther. 2009 October; 9(5):428-33; Doll et al., Gene Therapy 1996, 3(5):437-447; and Foley et al., J Control Release. 2014 Dec. 28; 196:71-8.

Thus, in some embodiments, the Reelin encoding nucleic acid is present in a vector for gene therapy, such as an AAV vector. In some instances, the AAV vector is selected from the group consisting of AAV1, AAV2, AAV3, AAV4, AAV5, AAV6, AAV7, AAV8, AAV9, AAVrh10, AAV11, and AAV12.

A vector as described herein can be a pseudotyped vector. Pseudotyping provides a mechanism for modulating a vector's target cell population. For instance, pseudotyped AAV vectors can be utilized in various methods described herein. Pseudotyped vectors are those that contain the genome of one vector, e.g., the genome of one AAV serotype, in the capsid of a second vector, e.g., a second AAV serotype. Methods of pseudotyping are well known in the art. For instance, a vector may be pseudotyped with envelope glycoproteins derived from Rhabdovirus vesicular stomatitis virus (VSV) serotypes (Indiana and Chandipura strains), rabies virus (e.g., various Evelyn-Rokitnicki-Abelseth ERA strains and challenge virus standard (CVS)), Lyssavirus Mokola virus, a rabies-related virus, vesicular stomatitis virus (VSV), Mokola virus (MV), lymphocytic choriomeningitis virus (LCMV), rabies virus glycoprotein (RV-G), glycoprotein B type (FuG-B), a variant of FuG-B (FuG-B2) or Moloney murine leukemia virus (MuLV). A virus may be pseudotyped for transduction of one or more neurons or groups of cells. In addition, the capsid can be altered to include one or more peptides that increase expression in the CNS, see, e.g., Yao et al., Nat Biomed Eng. 2022 Oct. 10; Chatterjee et al., Gene Ther. 2022 June; 29(6):390-397; Meng et al., Mol Ther Methods Clin Dev. 2021 Feb. 27; 21:28-41; Zhang et al., Biomaterials. 2022 February; 281:121340; Gray, Cell Gene Ther. Insights 5, 1361-1368 (2019); Nonnenmacher et al., Mol. Ther. Methods Clin. Dev. 20, 366-378 (2021).

Without limitation, illustrative examples of pseudotyped vectors include recombinant AAV2/1, AAV2/2, AAV2/5, AAV2/6, AAV2/7, AAV2/8, AAV9, AAVrh10, AAV11, and AAV12 serotype vectors. It is known in the art that such vectors may be engineered to include a transgene encoding a human protein or other protein. In particular instances, the present disclosures can include a pseudotyped AAV9 or AAVrh10 viral vector including a nucleic acid as disclosed herein. See Viral Vectors for Gene Therapy: Methods and Protocols, ed. Machida, Humana Press, 2003.

In some instances, a particular AAV serotype vector may be selected based upon the intended use, e.g., based upon the intended route of administration.

Various methods for application of AAV vector constructs in gene therapy are known in the art, including methods of modification, purification, and preparation for administration to human subjects (see, e.g., Viral Vectors for Gene Therapy: Methods and Protocols, ed. Machida, Humana Press, 2003). In addition, AAV based gene therapy targeted to cells of the CNS has been described (see, e.g., U.S. Pat. Nos. 6,180,613 and 6,503,888). High titer AAV preparations can be produced using techniques known in the art, e.g., as described in U.S. Pat. No. 5,658,776

A vector construct refers to a polynucleotide molecule including all or a portion of a viral genome and a transgene. In some instances, gene transfer can be mediated by a DNA viral vector, such as an adenovirus (Ad) or adeno-associated virus (AAV). Other vectors useful in methods of gene therapy are known in the art. For example, a construct as disclosed herein can include an alphavirus, herpesvirus, retrovirus, lentivirus, or vaccinia virus.

Adenoviruses are a relatively well characterized group of viruses, including over 50 serotypes (see, e.g., WO 95/27071, which is herein incorporated by reference). Adenoviruses are tractable through the application of techniques of molecular biology and may not require integration into the host cell genome. Recombinant Ad-derived vectors, including vectors that reduce the potential for recombination and generation of wild-type virus, have been constructed (see, e.g., international patent publications WO 95/00655 and WO 95/11984, which are herein incorporated by reference). Wild-type AAV has high infectivity and is capable of integrating into a host genome with a high degree of specificity (see, e.g., Hermonat and Muzyczka 1984 Proc. Natl. Acad. Sci., USA 81:6466-6470 and Lebkowski et al. 1988 Mol. Cell. Biol. 8:3988-3996).

Non-native regulatory sequences, gene control sequences, promoters, non-coding sequences, introns, or coding sequences can be included in a nucleic acid as disclosed herein. The inclusion of nucleic acid tags or signaling sequences, or nucleic acids encoding protein tags or protein signaling sequences, is further contemplated herein. Typically, the coding region is operably linked with one or more regulatory nucleic acid components.

A promoter included in a nucleic acid as disclosed herein can be a tissue- or cell type-specific promoter, a promoter specific to multiple tissues or cell types, an organ-specific promoter, a promoter specific to multiple organs, a systemic or ubiquitous promoter, or a nearly systemic or ubiquitous promoter. Promoters having stochastic expression, inducible expression, conditional expression, or otherwise discontinuous, inconstant, or unpredictable expression are also included within the scope of the present disclosure. A promoter can include any of the above characteristics or other promoter characteristics known in the art.

In clinical settings, the gene delivery systems for the therapeutic gene can be introduced into a subject by any of a number of methods, each of which is familiar in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by catheter (see U.S. Pat. No. 5,328,470) or by stereotactic injection, e.g., optionally into the cisterna magna, cerebral ventricles, lumbar intrathecal space, direct injection into hippocampus (e.g., Chen et al., PNAS USA 91: 3054-3057 (1994)) and/or the entorhinal cortex. In some embodiments, delivery methods of reelin-expressing virus include intravenous, intrathecal, intracerebroventricular, intracisternal, and stereotactic intraparenchymal administration.

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

### Reelin Formulations and Pharmaceutical Compositions

In some embodiments, the reelin polynucleotides as disclosed herein for delivery to a target tissue in vivo are encapsulated or associated with in a nanoparticle. Methods for nanoparticle packaging are well known in the art, and are described, for example, in Bose S, et al (Role of Nucleolin in Human Parainfluenza Virus Type 3 Infection of Human Lung Epithelial Cells. J. Virol. 78:8146. 2004); Dong Y et al. Poly(d,l-lactide-co-glycolide)/montmorillonite nanoparticles for oral delivery of anticancer drugs. Biomaterials 26:6068. 2005); Lobenberg R. et al (Improved body distribution of 14C-labelled AZT bound to nanoparticles in rats determined by radioluminography. J Drug Target 5:171.1998); Sakuma S R et al (Mucoadhesion of polystyrene nanoparticles having surface hydrophilic polymeric chains in the gastrointestinal tract. Int J Pharm 177:161. 1999); Virovic L et al. Novel delivery methods for treatment of viral hepatitis: an update. Expert Opin Drug Deliv 2:707.2005); and Zimmermann E et al, Electrolyte- and pH-stabilities of aqueous solid lipid nanoparticle (SLN) dispersions in artificial gastrointestinal media. Eur J Pharm Biopharm 52:203. 2001). In some embodiments, one or more polynucleotides is delivered to a target tissue in vivo in a vesicle, e.g. a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid). In some embodiments, lipid-based nanoparticles (LNP) are used; see, e.g., Robinson et al., Mol Ther. 2018 Aug. 1; 26(8):2034-2046; U.S. Pat. No. 9,956,271B2.

The present methods and compositions can include microvesicles or a preparation thereof that contains one or more therapeutic molecules, e.g., polynucleotides or RNA, as described herein. “Microvesicles”, as the term is used herein, refers to membrane-derived microvesicles, which includes a range of extracellular vesicles, including exosomes, microparticles and shed microvesicles secreted by many cell types under both normal physiological and pathological conditions. See, e.g., EP2010663B1. The methods and compositions described herein can be applied to microvesicles of all sizes. In some embodiments, 30 to 200 nm, In some embodiments, 30 to 800 nm, In some embodiments, up to 2 μm. The methods and compositions described herein can also be more broadly applied to all extracellular vesicles, a term which encompasses exosomes, shed microvesicles, oncosomes, ectosomes, and retroviral-like particles. Such a microvesicle or preparation is produced by the herein described methods. As the term is used herein, a microvesicle preparation refers to a population of microvesicles obtained/prepared from the same cellular source. Such a preparation is generated, for example, in vitro, by culturing cells expressing the nucleic acid molecule of the instant invention and isolating microvesicles produced by the cells. Methods of isolating such microvesicles are known in the art (Thery et al., Isolation and characterization of exosomes from cell culture supernatants and biological fluids, in Current Protocols Cell Biology, Chapter 3, 322, (John Wiley, 2006); Palmisano et al., (Mol Cell Proteomics. 2012 August; 11(8):230-43) and Waldenstrom et al., ((2012) PLoS ONE 7(4): e34653)), some examples of which are described herein. Such techniques for isolating microvesicles from cells in culture include, without limitation, sucrose gradient purification/separation and differential centrifugation, and can be adapted for use in a method or composition described herein. See, e.g., EP2010663B1.

In some embodiments, the microvesicles are isolated by gentle centrifugation (e.g., at about 300 g) of the culture medium of the donor cells for a period of time adequate to separate cells from the medium (e.g., about 15 minutes). This leaves the microvesicles in the supernatant, to thereby yield the microvesicle preparation. In some embodiments, the culture medium or the supernatant from the gentle centrifugation, is more strongly centrifuged (e.g., at about 16,000 g) for a period of time adequate to precipitate cellular debris (e.g., about 30 minutes). This leaves the microvesicles in the supernatant, to thereby yield the microvesicle preparation. In some embodiments, the culture medium, the gentle centrifuged preparation, or the strongly centrifuged preparation is subjected to filtration (e.g., through a 0.22 um filter or a 0.8 um filter, whereby the microvesicles pass through the filter. In some embodiments, the filtrate is subjected to a final ultracentrifugation (e.g. at about 110,000 g) for a period of time that will adequately precipitate the microvesicles (e.g. for about 80 minutes). The resulting pellet contains the microvesicles and can be resuspended in a volume of buffer that yields a useful concentration for further use, to thereby yield the microvesicle preparation. In some embodiments, the microvesicle preparation is produced by sucrose density gradient purification. In some embodiments, the microvesicles are further treated with DNAse (e.g., DNAse I) and/or RNAse and/or proteinase to eliminate any contaminating DNA, RNA, or protein, respectively, from the exterior. In some embodiments, the microvesicle preparation contains one or more RNAse inhibitors.

The molecules contained within the microvesicle preparation will comprise the therapeutic molecule. Typically the microvesicles in a preparation will be a heterogeneous population, and each microvesicle will contain a complement of molecule that may or may not differ from that of other microvesicles in the preparation. The content of the therapeutic molecules in a microvesicle preparation can be expressed either quantitatively or qualitatively. One such method is to express the content as the percentage of total molecules within the microvesicle preparation. By way of example, if the therapeutic molecule is an mRNA, the content can be expressed as the percentage of total RNA content, or alternatively as the percentage of total mRNA content, of the microvesicle preparation. Similarly, if the therapeutic molecule is a protein, the content can be expressed as the percentage of total protein within the microvesicles. In some embodiments, therapeutic microvesicles, or a preparation thereof, produced by the method described herein contain a detectable, statistically significantly increased amount of the therapeutic molecule as compared to microvesicles obtained from control cells (cells obtained from the same source which have not undergone scientific manipulation to increase expression of the therapeutic molecule). In some embodiments, the therapeutic molecule is present in an amount that is at least about 10%, 20%, 30% 40%, 50%, 60%, 70% 80% or 90%, more than in microvesicles obtained from control cells. Higher levels of enrichment may also be achieved. In some embodiments, the therapeutic molecule is present in the microvesicle or preparation thereof, at least 2 fold more than control cell microvesicles. Higher fold enrichment may also be obtained (e.g., 3, 4, 5, 6, 7, 8, 9 or 10 fold).

In some embodiments, a relatively high percentage of the microvesicle content is the therapeutic molecule (e.g., achieved through overexpression or specific targeting of the molecule to microvesicles). In some embodiments, the microvesicle content of the therapeutic molecule is at least about 10%, 20%, 30% 40%, 50%, 60%, 70% 80% or 90%, of the total (like) molecule content (e.g., the therapeutic molecule is an mRNA and is about 10% of the total mRNA content of the microvesicle). Higher levels of enrichment may also be achieved. In some embodiments, the therapeutic molecule is present in the microvesicle or preparation thereof, at least 2 fold more than all other such (like) molecules. Higher fold enrichment may also be obtained (e.g., 3, 4, 5, 6, 7, 8, 9 or 10 fold).

### Reelin CTR

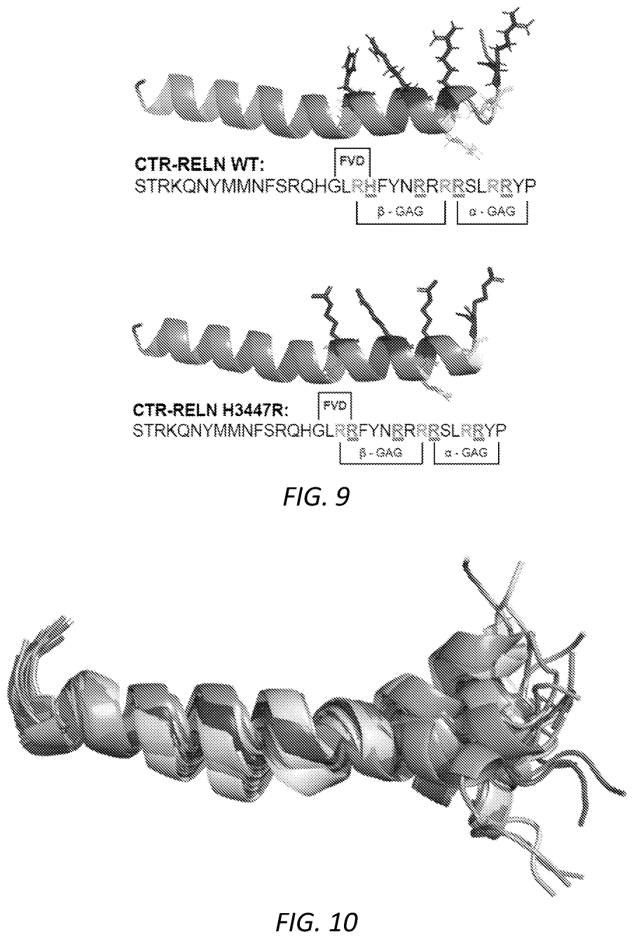

Initial assessment of the Reelin CTR sequence indicated that the sequence is highly conserved across mammalian species, suggesting that previously uncharacterized Reelin CTR may play an important neurobiological role (11). We show that AA in position 3447 is aligned on the same side as other arginines in a known heparin-binding motif Therefore, a basic AA in this position has the potential to contribute to HSPG interactions (FIG. 9). Building on this initial result, we used NMR to elucidate the previously unknown structure of the Reelin CTR (FIG. 10). We find that the Reelin CTR is primarily an alpha-helical structure with the last 16 AAs having increased flexibility. Here, increased flexibility in the secondary structure may indicate a potential binding site.

Additionally, a basic AA in position 3447 favored heparin interaction as observed by HPLC. H3447R Reelin has a significantly later peak retention time in comparison to R3446H or WT peptides across two different experimental designs. We considered significant differences in peak retention times to be at least greater than 30 seconds. An acidic AA (H3447D) had significantly less interaction in comparison to neutral (WT) or basic AAs (H3447R, H3447K). In sum, the Reelin CTR has a charge-based interaction with heparin, and the 3447 position plays a role in these interactions.

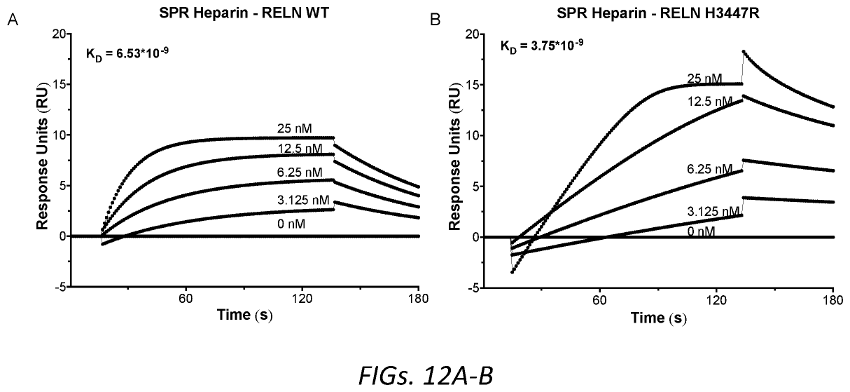

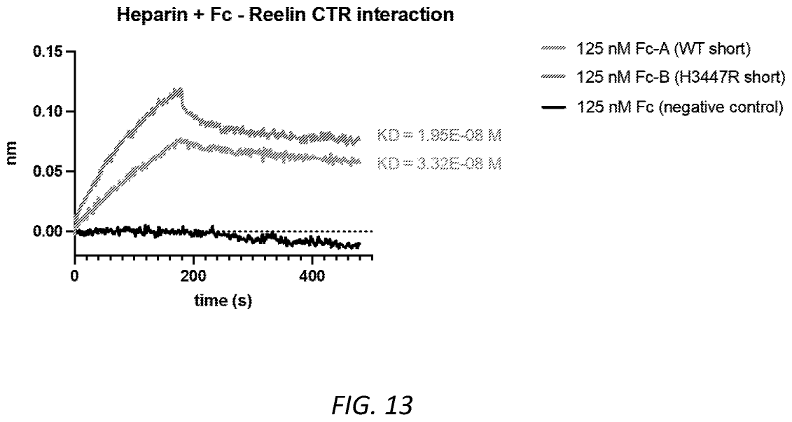

To gain further insights into the kinetics of this interaction, heparin—Reelin CTR peptide interaction was assessed using SPR. H3447R Reelin had ˜2-fold more interaction with heparin as compared to Reelin WT peptide (FIGS. 12A-B); to verify this small difference, we additionally assessed this interaction using BLI and found the same difference with fc-fusion peptides (FIG. 13). In this case, the fc-fusion peptides are likely to be short due to the method of production. Therefore, using two different methods, we find that H3447R has 2-fold more interaction in comparison to WT Reelin.

We then used ITC to further understand the thermodynamic properties of this interaction and found that H3447R is favored to interact, especially in vivo. Nonetheless, we also found that with long peptides, the Kₐ, ΔH, and ΔG, and ΔS were similar. This may partially be explained because this method assess binding in solution whereas the kinetic and HPLC methods assess binding with heparin attached to a surface; therefore, the configuration of the Reelin peptide and potential binding site exposure may be affected. Significantly, the short peptides showed more positive entropy and more negative Gibbs free energy in comparison to the Reelin WT short variant. Since the Reelin CTR is normally cleaved by furin, the short peptide is the most representative in vivo variant. Therefore, H3447R Reelin—HSPG interactions may be thermodynamically favored in comparison to WT Reelin in vivo. Additionally, the negative controls for ITC showed that basic amino acids in the 3446-3447 position play a significant role in heparin interaction, and there is ˜-100× less affinity without at least 1 basic amino acid in the binding site.

We also found that H3447R has 10-fold more interaction with NRP1 in comparison to WT fc-fusion peptides. This experiment suggests the Reelin CTR may have additional interactions at the cell surface.

Overall, these data indicate that H3447R has increased interaction with heparin in comparison to WT. Without wishing to be bound by theory, Reelin first binds to HSPGs and subsequently LDL receptors or other receptors, where the Reelin CTR interacts with HSPGs and the Reelin intermediate domain interacts with LDL receptors. These cell surface interactions may eventually modulate downstream NFT formation since it has previously been shown that dysregulation of Reelin may lead to tau hyperphosphorylation (43, 44).

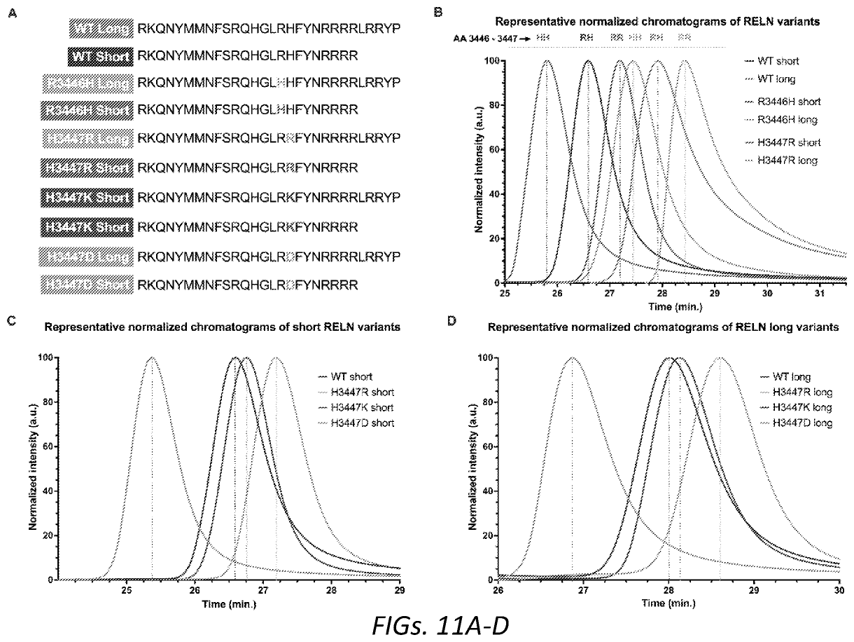

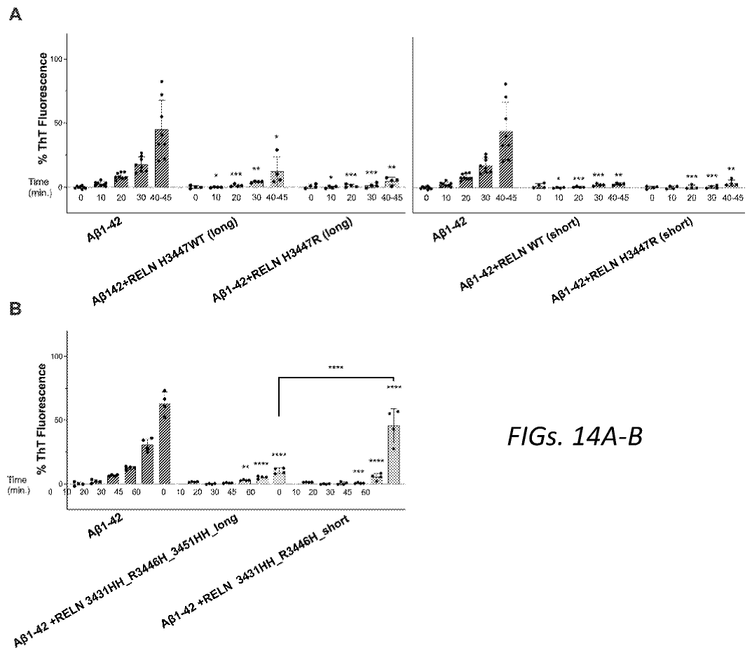

Significantly, RELN CTR decreased amyloid aggregation, suggesting a role in the progression of AD pathology (FIG. 14A. However, mutations in position 3447 including the Reelin-COLBOS variant H3447R did not significantly affect Aβ aggregation compared to WT, though there was a trend towards higher reduction. The patient described herein who was resilient to ADAD had high levels of amyloid plaque burden. These results indicate that the flexibility of the N-terminal region may play a role in the seeding of Aβ, since H is less flexible than R or K amino acids (45). Furthermore, the relative flexibility of the alpha—GAG binding site may help against propagation of Aβ plaques (FIGS. 14A-B). In contrast, our results show the beta—GAG site interacts with GAGs and NRP1, and the H3447R mutation optimizes these interactions (FIGS. 11A-D, 12A-B, 13).

The RELN CTR may have multiple interaction partners including GAGs, NRP1, amyloid, and potentially others. The RELN H3447R mutation found in the AD-protected case is clearly not neutral and may contribute to the AD-protection phenotype via multiple mechanisms. We show that the RELN CTR-GAG interaction is reproducible across various methods, and the H3447R mutation enhances a binding site that may impact interactions with other multiple molecules relevant to neurodegenerative diseases.

Provided herein are compositions comprising RELN CTR peptides. These peptides can comprise or consisting of the RELN-CTR wild type (WT) or RELN-CTR H3447R/K variant peptide (preferably H3447R: RKQNYMMNFSRQHGLRRFYNRRRRSLRRYP)(SEQ ID NO:53). In some embodiments, the CTR peptides are variants that can include one or more alternative or additional mutations as described herein, e.g., as shown in Table 1, and/or a mutation at G3444, e.g., G3444H or G3444P.

TABLE 1
Exemplary Reelin CTR peptide properties

| | | | | | | | SEQ |
|---|---|---|---|---|---|---|---|
| | | | | | | | ID |
| Sample | Sequence | Purity | λ_{exc1} | λₑₘ₁ | λ_{exc2} | λₑₘ₁ | NO: |
| WT short | RKQNYMMNFSRQHGLRHFYNRRRR | >95% | 224 | 302 | 276 | 302 | 59 |
| H3447R short | RKQNYMMNFSRQHGLRRFYNRRRR | >95% | 228 | 299 | 274 | 302 | 64 |
| H3447D short | RKQNYMMNFSRQHGLRDFYNRRRR | >95% | 222 | 302 | 274 | 302 | 76 |
| H3447K short | RKQNYMMNFSRQHGLRKFYNRRRR | >95% | 225 | 302 | 275 | 302 | 74 |
| R3446H short | RKQNYMMNFSRQHGLHHFYNRRRR | >95% | 225 | 303 | 275 | 303 | 71 |
| WT long | RKQNYMMNFSRQHGLRHFYNRRRRSLRRYP | >95% | 224 | 302 | 276 | 302 | 56 |
| H3447R long | RKQNYMMNFSRQHGLRRFYNRRRRSLRRYP | >95% | 224 | 302 | 274 | 302 | 53 |
| H3447D long | RKQNYMMNFSRQHGLRDFYNRRRRSLRRYP | >95% | 224 | 301 | 274 | 302 | 57 |
| H3447K long | RKQNYMMNFSRQHGLRKFYNRRRRSLRRYP | >95% | 224 | 301 | 274 | 303 | 58 |
| R3446H long | RKQNYMMNFSRQHGLHHFYNRRRRSLRRYP | >95% | 225 | 303 | 275 | 303 | 77 |

In some embodiments, the Reelin CTR peptides are at least 80%, 85%, 90%, 95%, 97%, 98%, or 99% identical to a sequence provided herein, so long as they retain desired functionality of the parental sequence.

In some embodiments, the peptides can include a sequence provided herein with exactly, at least, or up to one, two, three, four, five, six, seven, eight, nine, or ten altered amino acids.

Also provided are compositions comprising the CTR peptides, optionally in a pharmaceutically acceptable carrier, and optionally mixed with a non-reelin protein or nucleic acid. The nucleic acid can be, e.g., an mRNA encoding a therapeutic or prophylactic agent such as an antigen for a vaccine. The CTR peptides can also be in a fusion protein with a non-reelin sequence, e.g., wherein the CTR peptide sequence is at the N terminus, C terminus, or inserted internally at a position that doesn't affect function of the non-reelin sequence.

### Pharmaceutical Compositions and Methods of Administration

The methods described herein include the use of pharmaceutical compositions comprising or consisting of a therapeutic agent described herein, e.g., a Reelin protein or fragment thereof, e.g., a mini-Reelin; or a nucleic acid encoding the same, as described herein, as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., a mRNA encoding a therapeutic or diagnostic protein.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration. In some embodiments, the composition is delivered to the brain, e.g., by administration into the cisterna magna, cerebral ventricles, lumbar intrathecal space, direct injection into hippocampus (e.g., Chen et al., PNAS USA 91: 3054-3057 (1994)). In some embodiments, delivery methods of reelin-expressing virus include intravenous, intrathecal, intracerebroventricular, intracistemal, and stereotactic intraparenchymal administration. In some embodiments, the compositions are administered in or around the entorhinal cortex of the brain.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

Ocular administration can be achieved via intravitreal or subretinal injection of biologics including protein, nucleic acids or vectors including viruses or via electroporation of DNA plasmids in the ciliary body.

### Methods of Determining Risk of Developing AD

Included herein are methods for determining risk of identifying AD in a subject, e.g., a human subject. The methods rely on detection of the H3447R variant of the RELN gene in the subject's DNA. The methods include obtaining a sample comprising genomic DNA from a subject and evaluating the presence of the H3447R variant in the sample.

As used herein the term “sample”, when referring to the material to be tested for the presence of the H3447R variant, includes inter alia tissue (including buccal cells from a swab) or blood. Various methods are well known within the art for the identification and/or isolation and/or purification of genomic DNA from a sample. For example, nucleic acids contained in the sample can be first isolated according to standard methods, for example using lytic enzymes, chemical solutions, or isolated by nucleic acid-binding resins following the manufacturer's instructions.

The presence and/or level of a H3447R variant nucleic acid can be evaluated using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559); RNAse protection assay; Northern blot; various types of nucleic acid sequencing (Sanger, pyrosequencing, NextGeneration Sequencing); or gene array/chips) (Lehninger Biochemistry (Worth Publishers, Inc., current addition; Sambrook, et al, Molecular Cloning: A Laboratory Manual (3. Sup.rd Edition, 2001); Bernard (2002) Clin Chem 48(8): 1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence of the H3447R variant. In some embodiments a technique suitable for the detection of alterations in the structure or sequence of nucleic acids, such as the presence of deletions, amplifications, or substitutions, can be used for the detection of the H3447R variant.

Gene arrays can be prepared by selecting probes that comprise the H3447R variant polynucleotide sequence, and then immobilizing such probes to a solid support or surface. For example, the probes may comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g., by PCR), or non-enzymatically in vitro.

A subject who has the H3447R variant in their genome can be identified as having a lower risk of developing AD as compared to a subject who does not have the H3447R variant. In some embodiments, the subject has, or is also identified as having, a APOE4 variant allele.

### EXAMPLES

The invention is further described in the following examples, which do not limit the scope of the invention described in the claims.

### Example 1. Resilience to Autosomal Dominant Alzheimer's Disease in a Reelin-COLBOS (H3447R) Variant Carrier

### Methods

The following methods and materials were used in this Example 1.

### Clinical Assessments

The patient was evaluated after providing informed written consent approved by the institutional review board of the University of Antioquia and Massachusetts General Hospital (MGH).

### Brain Imaging

We used Pittsburgh Compound B (PiB) and Flortaucipir (FTP) positron emission tomography (PET) to image cerebral Aβ and tau burden, respectively, in vivo. Structural magnetic resonance imaging (MRI) and both PET scans were conducted at MGH. The ¹⁸F-fludeoxyglucose PET was performed at the University of Antioquia and the procedures and data analyses were performed as previously described (2).

### Genetic and Molecular Studies

We conducted whole exome, whole genome sequencing (WGS), and Genomizer analysis (v10.10) of the subject to obtain a ranking of AD-related potential risk factors as previously shown (2) and as described in detail below.

### Mouse Model

We generated a knock-in mouse model carrying the RELN-COLBOS variant via homologous recombination as fee-for-service (Cyagen). Additional details about mouse model and in vivo analyses are described in the methods section of the supplementary appendix.

### Neuropathology

Postmortem interval was 210 minutes after death, brain weight 745.4 grams. Following five days of fixation in 4% paraformaldehyde and sample preparation in paraffin, three μm thick sections from specified brain regions were cut, deparaffinized and stained with hematoxylin and eosin or processed for immunohistochemical staining using antibodies as described in the additional neuropathological characterization section.

### Additional Plasma Nfl Assay Details

Plasma neurofilament light (NfL) analysis was conducted by the Clinical Neurochemistry Laboratory at Sahlgrenska University Hospital (Mölndal, Sweden) as previously reported (1). The experimenter was masked to clinical or genetic data of the proband, to avoid any bias.

### Additional Genome Sequencing Details

Whole genome sequencing analysis called 44 million variants from the case's genome. The variants went through multiple filters using Genomiser: population allele frequency <=2%, variant effect filter that exclude intergenic, untranslated regions (UTR), and non-coding intronic regions, regulatory feature filter removes all non-regulatory, non-coding variants over 20 Kb from a known gene, and hiPhive prioritizer phenotype gene priority score >=0.401. 6,779 variants passed filtering and were ranked by the composite Exomiser scores from gene-phenotype scores and variant pathogenicity scores, and the compatibility with the mode of inheritance. The top three ranked variants were manually reviewed for pathogenicity. We also manually searched a list of 23 genes previously associated to Alzheimer's including AAGAB, ABCC8, AKT2, APOE, APP, BEAN1, GATA1, GCK, HMGA1, HNF1B, HNF4A, LDB3, PAX4, PSEN1, PSEN2, ABCA7, SORL1 CACNA1G, HFE, MPO, NOS3, and PLAU. The gene variants from WGS were extracted and annotated in the Alissa Interpret platform for independent pathogenicity interpretation (Agilent, Santa Clara, CA). We calculated “C scores” using Combined Annotation Dependent Depletion CADD (2, 3), to contrast pathogenicity of human-derived vs. simulated variants. The regulatory Mendelian mutation (ReMM) framework uses machine learning techniques to train a classifier to predict the potential of an arbitrary position in the non-coding genome to cause a Mendelian disease if mutated (4).

### Additional Single Cell RNA Sequencing Details

We used a Ficoll gradient to isolate peripheral blood mononuclear cells and these cells were analyzed by scRNA sequencing using a protocol previously published (1).

Additional RELN genotyping by Sanger DNA sequencing details

The DNA was first extracted using Gentra Puregene Kit (Qiagen). 50 μL of the obtained samples (both RELN H3447R and RELN H3447 carriers) were amplified via polymerase chain reaction (PCR) using one L of ten M primers (forward:5′-GTCCCAGCCTTTAGTTCCT-3′(SEQ ID NO:54); reverse: 3′-CAACTTTCACGGACACATCAA-5′) (SEQ ID NO:55)pre-mixed in PCR Master Mix 2× (K0171, Thermo Fisher) using the following protocol: three minutes at 94° C. for initial denaturation, 33 cycles, 30 seconds at 94° C., 35 seconds at 62° C. for annealing, 35 seconds at 72° C. for the elongation, five minutes at 72° C. for final extension. Horizontal electrophoresis was done at 100 V using 1.5% Agarose gel in TAE buffer (Tris-acetate EDTA, T8280-1L, Sigma Aldrich) and mixed with GelRed (41003-T, Biotium). Fluorescence of positive bands was detected via Bio-RAD Molecular Imager Gel Doc XR+ and acquired via Image Lab software (ver. 6.0.1, Biorad). Purification of the amplified DNA was done using QIAquick Gel Extraction kit (Qiagen) and sequenced by MGH CCIB DNA core using the 3730xl sequencer (Applied Biosystems) as previously published(1).

### Additional Cell Culture Details

Plasmid encoding for full-length murine recombinant RELN was a gift from Dr. Tom Curran via Addgene (plasmid #122444(5)). The plasmid was subsequently mutagenized to obtain the H3448R mutation homologous of human RELN H3447R as fee-for-service by CustomDNAConstructs (New York, USA). We produced WT and RELN-COLBOS in Flp-In T-Rex 293 mammalian cells (R78007, Thermo Fisher Scientific) via transient transfection and used them for receptor binding assays via ELISA. Plasmids encoding for the CTR-RELN-Fc fused peptides were obtained from CustomDNAConstructs as fee-for-service. All constructs were overexpressed in Flp-In T-Rex 293 mammalian cells (R78007, Thermo Fisher Scientific) via transient transfection using lipofectamine 2000 according to manufacturer (11668030, Thermo Fisher). Five hours post-transfection, Opti-MEM was used to collect conditioned media. Cells were incubated for 24 hours, and supernatant was collected and cleared from cell debris by centrifugation for three minutes, 1,800 rcf at room temperature (R.T.). Primary CD1 brain cortex mouse neurons (M-CX-400, Lonza) were cultured in neurobasal media (Gibco) supplemented with B-27 (Thermo Fisher), glutamax (Gibco) and normocin (Invivogen). Cells were plated on poly-L-lysine (Sigma) coated wells and treated at day six post-liquid nitrogen recovery. Treatments with recombinant RELN (RELN WT or RELN H3448R, four μg/mL) were incubated for either five minutes or one hour at 37° C., 5% CO₂ in the presence of ten μM Mg-132 (ab141003, Abcam). Cells were washed in ice cold dPBS (Gibco) and lysed in RIPA (9806, Cell Signaling) supplemented with ten μM Mg-132, Triton-X100 (Sigma Aldrich), proteases inhibitor cocktail (4693159001, Millipore) and phosphatases inhibitors (4906837001, Sigma Aldrich, and P0044, Millipore). Protein concentration was determined by Pierce Bicinchonic acid (BCA) protein assay kit (23227, Thermo Fisher) according to manufacturer's instructions. Samples were prepared containing ten μL Laemmli buffer (Boston Bioproducts) and four μL of one M DTT (Sigma Aldrich) and diluted to a final volume of 40 μL with water and denatured five min at 90° C.

### Additional Western Blotting Details

Twenty μg of total cell lysates were prepared in four μL one M 1,4-dithiothreitol (DTT; Sigma Aldrich) and ten μL Laemmli buffer (Boston Bioproducts) to a final volume of 40 μL and denatured by heat for five min. at 90° C. Samples were separated electrophoretically for one h at 90 V using 4-20% pre-cast gradient gels (Mini-PROTEAN TGX, Bio-Rad) and SDS-Tris-Glycine buffer (Bio-Rad). Proteins were transferred to 0.45 μm nitrocellulose membranes for one h at 90 V in ice-cold 20% Methanol Tris-Glycine buffer (Bio-Rad). To detect pDAB1 levels, proteins were transferred on PVDF membranes using the iBlot2 dry transfer system (IB210025, Thermo Fisher). Total protein levels were detected using Licor Membranes were blocked for one h with Odyssey Blocking Buffer (LI-COR Biosciences) or for two h with five % dry milk (M17200-100.0, RPI), and both proteases and phosphatases inhibitor cocktails when blocked for anti pDAB1 western blotting. β-Tubulin (ms; 1:2,000; 86298S, Cell Signaling), anti-phospho-Dab1 (Rb; 1:7,500; MBS8511213, MyBiorsorce), total tau (ms, 1:1,000, ab80579, Abcam), phosphorylated tau (Ser396, rb, 1:1,000, 44-752G, Thermo Fisher) and anti RELN antibody (ms, 1:1,000, clone CR-50, D223-3, MBL) were used as primary antibodies and incubated in blocking buffer for either two h at room temperature or 18 h at four ° C. After washing the blots for three times with TBS-T buffer (Pierce, Thermo Fisher), secondary antibodies were incubated either one h or 45 minutes at R.T. (IRDye 800CW donkey anti-mouse, 925-32212, or IRDye 680CW donkey anti-rb; 1:10,000, 925-68073, Li-COR). Immunoreactive bands were detected using the Odyssey Infrared Imaging System and visualized on the Image Studio software (version 2.1, LI-COR Biosciences). Detection of Dab1 was obtained with anti-rb-HRP conjugated antibody (HAF008, R&D Systems) followed by five minutes incubation with West pico Super Signal™ West Pico PLUS Chemiluminescent Substrate and acquisition on SyngeneG:Box Digital ECL detection system.

### Additional Heparin-Sepharose Affinity Chromatography Details

We tested changes in binding to heparin of RELN variants chromatographically using an optimized version of a protocol previously published by our laboratory (1). Briefly, after equilibration of the heparin column (BioVision 6554-1) at R.T., columns were washed with five volumes of degassed 20 mM TRIS-HCl buffer (pH 7.5). Recombinant C-terminus RELN peptides were produced and purified by Innovagen (Sweden): (WT) RKQNYMMNFSRQHGLRHFYNRRRRSLRRYP(SEQ ID NO:56), and (H3447R) RKQNYMMNFSRQHGLRRFYNRRRRSLRRYP(SEQ ID NO:53). One mL of 50 μg/mL peptide (H3447 or WT, and H3447R) was recycled through the column five times and the last flow through collected for further analysis. The column was washed five times with the same buffer and the protein eluted using a 0.05 M step gradient of NaCl in 20 mM Tris-HCl (0-1M, one mL per fraction). To ensure the complete release of the protein, the column was washed with five M NaCl 20 mM Tris-HCl. Three independent experiments were conducted for C-terminus RELN WT and H3447R. All eluted fractions were analyzed spectroscopically by reading the absorbance at 280 nm using Nanodrop 2000 Spectrophotometer. Blank corrected fractions were subsequently analyzed using GraphPad Prism 8.

### Additional ELISA Details

Enzyme-linked immunosorbent assay (ELISA) was used to quantify changes in binding of RELN variants (media-derived full-length or CTR-RELN) to either VLDLr or ApoEr2 following an optimized version of a previously published protocol (6). Briefly, ELISA strips (DY008, R&D Systems) were coated with one ng/μL (100 μL/well) of VLDLr (8444-VL, R&D) or ApoEr2 (TP320903, OnGene) receptors diluted in 25 mM Tris HCl, 140 mM NaCl, 27 mM KCl, two mM CaCl₂), pH 7.4 (TBS-C buffer). After 18 h at four ° C. incubation, plates were blocked one h with three % BSA (22070008-6, Bioworld), 0.05% Tween-20 (Sigma Aldrich) TBS-C buffer. We assessed binding via incubation with 100 μL/well of serial dilutions of recombinant RELN protein (Ser1221-Gln2666, 8546-MR-050, R&D) or RELN variants in TBS-C buffer for one hour at room temperature. For detection, we used goat anti-mouse RELN primary antibody (LS-C793521-100, LS-Bio, 1:2,000, 100 μL/well) as primary antibody for one h at R.T. and (1:10,000; 100 μL/well, donkey anti-goat IgG H&L, HRP, ab6885, Abcam) as secondary antibody for 30 min. at R.T. Between each of the previous steps, plates were washed four times with TBS-C buffer (200 μL/well). Plates were washed three times prior to initiating the colorimetric reaction (DY008, R&D Systems). Stop solution (50 μL/well, DY008, R&D Systems) was added upon five min. incubation and the absorbance of the samples was measured at 450 nm using the Synergy 2 microplate reader (BioTek Instruments). Processing and analysis of the data was done using Gen5 1.11 software and GraphPad Prism, respectively.

### Additional SPR Assay Details

Surface plasmon resonance assay (SPR) was used for performing binding kinetics using a Biacore 3000 Instrument (GE Healthcare) at 25° C. following previously published protocols (7 24) as fee-for-service by Precision Antibody (Maryland, USA). Biotin-labeled heparin (B9806, Sigma Aldrich) was covalently linked to streptavidin-coated chip and unoccupied sites were blocked with biocytin. Antigen was flowed over the chip using a range of single analyte concentrations prepared in DPBS buffer and using a flow rate of 30 μL/min. Binding of antigen to the ligand was monitored in real time to obtain on (ka) and off (kd) rates. The equilibrium constant (K_{D}) was calculated from the observed ka and kd. Accuracy of the SPR analysis was determined via Chi square (χ2) analysis as described in the statistical analysis section. We included in the analyses the WT and H3447R peptides and peptides with the hypothetical H3447D change (RKQNYMMNFSRQHGLRDFYNRRRRSLRRYP)(SEQ ID NO:57) and the H3447K change (RKQNYMMNFSRQHGLRKFYNRRRRSLRRYP)(SEQ ID NO:58), also produced and purified by Innovagen (Sweden).

### Additional Mouse Model and In Vivo Analyses Details

We generated the RELNH3448R-Tg knock in (KI) mouse model at TACONIC by introducing the H3448R (CAC to CGT) mutation into exon 64 in 3′ homology arm of the RELN gene. Gene targeting was obtained using C57BL/6 ES cells. KI mice were generated upon injecting targeted ES cells into the blastocysts that were introduced into the foster mothers for generating the mouse crossings. Mice were euthanized using chambers saturated with CO₂. Cerebella were dissected and stored at −80° C. upon cervical dislocation and ensuring a postmortem interval less than three minutes. All procedures were conducted using protocols approved by institutional animal care and Mass Eye and Ear committee. Brain homogenates from dissected cerebella were obtained in modified RIPA buffer (Cell signaling) supplemented with protease (Roche) and phosphatase inhibitors (Sigma) and using a tissue homogenizer (two times 15″ pulses). Homogenized tissue was then vortexed 20″ every 10 min. for one hour and centrifuged ten min. 10,000 rpm four ° C. Soluble protein fraction was then analyzed using BCA assay (Pierce

We measured via western blotting levels of RELN (clone CR-50, D223-3, MBL), Dab1 (clone G-5, sc-271136, Santa Cruz), phospho-Dab1 (Tyr232, MBS8511213, My Biosource), in the cerebellum of adult male and female mice (six to 12 months, n=3-4 per genotype) either wild type, heterozygous or homozygous for the RELN H3448R mutation.

### Additional Neuropathological Characterization Details

Postmortem interval of brain tissue was 210 minutes after death. The brain presented frontal lobe predominant atrophy; brain and associated structures weight was 745-4 g and the interuncal distance was 2.3 cm. Following five days of fixation in four % paraformaldehyde and sample preparation, three m thick sections from medial frontal gyrus (MFG), superior temporal gyrus (STG), medial temporal gyrus (MTG), inferior temporal gyrus (ITG), hippocampus/collateral sulcus (HP-C), hippocampus/uncus (HP-Uncus), amygdala (Amy), insula (Ins), inferior parietal lobe (IPL), occipital lobe (OL), gyrus cinguli (GC), lentiform nucleus (LN), caudate nucleus (CN), thalamus/hypothalamus (T-H), cerebellum (CB), midbrain/pons (MP) and medulla oblongata (MO) were cut, deparaffinized and stained with hematoxylin and eosin (HE) or processed for immunohistochemistry (IHC) staining for amyloid beta (As, 1:100; BAM-10, Mob410; DBS Emergo Europe, The Hague, The Netherlands), hyperphosphorylated tau (ptau, 1:100; AT8, MN1020, Thermo Fisher, Dreieich, DE), ionized calcium binding adaptor molecule 1 (Iba1, 1:500; 019-19741; Wako, Neuss, Germany), glial fibrillary acidic protein (GFAP, 1:200; M0761, DAKO GmbH, Jena, DE), C-terminal Reelin (RELN-CT, 1:200; E-5, sc-25346, Santa Cruz Biotechnology Inc., Heidelberg, DE) and Apolipoprotein E (ApoE, 1:100; Goat polyclonal, AB947, Merck Millipore, Darmstadt, DE), and specific secondary antibodies anti-mouse and anti-rabbit (P0260 and P0447, respectively, DAKO GmbH, Jena, DE). neuronal nuclei (NeuN; 1:100; MAB377; Merck/Millipore, Darmstadt, Germany), Visualization was achieved with 3,3′-Diaminobenzidine (DAB, Ventana, Roche ΔG, Basel, Switzerland) and the Ultraview Universal Detection Kit (Roche ΔG, Basel, Switzerland) according to manufacturer instructions. Automatic immunostaining was performed with a Ventana Benchmark XT system (Roche ΔG, Basel, Switzerland) according to manufacturer instructions. Selected brain areas were also stained with luxol fast blue (LFB) for myelin staining and Kluver-Barrera (KV) staining's. Cresyl violet staining was used for neuronal perikarya. Neuropathological workup was performed diagnosis by experienced morphologists masked to the origin of the sample (M.G. and D.S-F.). Sections were scanned using a Hamamatsu NanoZoomer automatic digital slide scanner (Hamamatsu Photonics, Hamamatsu, Japan) and obtained images and regions of interest (cortex for cortical areas, whole stained sections for non-cortical areas) at a resolution of at least one pixel per m. Signal intensity, together with particles and total area was assessed, after performing color deconvolution and thresholding, in the brown (DAB) color channel by using ImageJ Software (version 1.52p, NIH, Bethesda, MA, USA,) (8). Neuronal counting was performed manually and normalized by area in selected regions of interest in the hippocampal and parahippocampal structures. Information on the statistical analysis is reported in the dedicated section.

### Additional Immunoprecipitation Details

Mouse frontal cortex tissue was homogenized by two 15″ pulses in ice cold M-PER protein extraction reagent (78503, Thermo Fisher) supplemented with phosphatases and proteases inhibitors using a homogenizer as described in the previous paragraph. 100 μg of total proteins was pulled down using either anti-phosphotyrosine magnetic beads (clone 4G10, 16-282, Millipore), anti-total Dab1 agarose beads (clone G-5, sc-271136 AC, Santa Cruz) and anti-normal mouse IgG isotype control agarose beads (SC-2343, Santa Cruz). Prior immunoprecipitating the sample over night at four ° C., ten % was removed and analyzed as input. Beads were washed in PBS upon collecting the unbound fraction and subsequently boiled in 4× reducing sample buffer for six minutes to allow the release of the immune precipitated proteins.

### Additional Sequencing Analysis Via Mass Spectrometry

Immunoprecipitated fractions obtained using the anti-Dab1 conjugated beads were electrophoretically separated on a ten % acrylamide precast gel (Biorad) and stained using blue Coomassie (Thermo Fisher). Excised gel bands were analyzed as a fee-for-service at the Taplin Biological Mass Spectrometry Facility (Harvard Medical School, Boston, MA). Gel bands were subsequently dehydrated in acetonitrile followed to speed-vac. Rehydration of the bands were obtained in a solution of 50 mM ammonium bicarbonate supplemented with 12.5 ng/μL modified sequencing-grade trypsin (Promega, Madison, WI) at four ° C. Samples was washed with 50 mM ammonium bicarbonate solution. Upon overnight incubation a 37° C., protein extraction was obtained by removing the ammonium bicarbonate solution. Proteins were washed in a of 50% acetonitrile and one % formic acid (9). Reconstituted samples sequenced using nano-scale reverse-phase HPLC capillary column upon gradient elution using acetonitrile and formic acid via an electrospray ionization-LTQ Orbitrap Velos Pro ion-trap mass spectrometer (Thermo Fisher Scientific, Waltham, MA). Protein-specific fragment ions sequence was analyzed using the know peptide sequences using Sequest(10) (Thermo Fisher Scientific, Waltham, MA).

### Additional Statistical Analysis Details

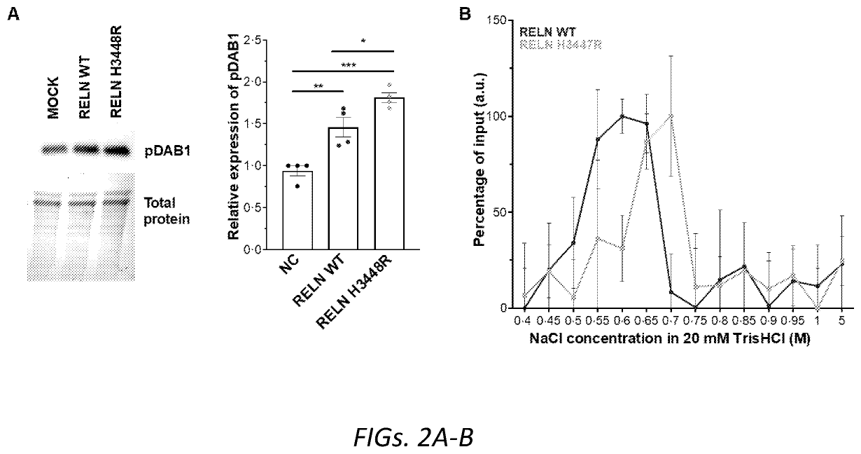

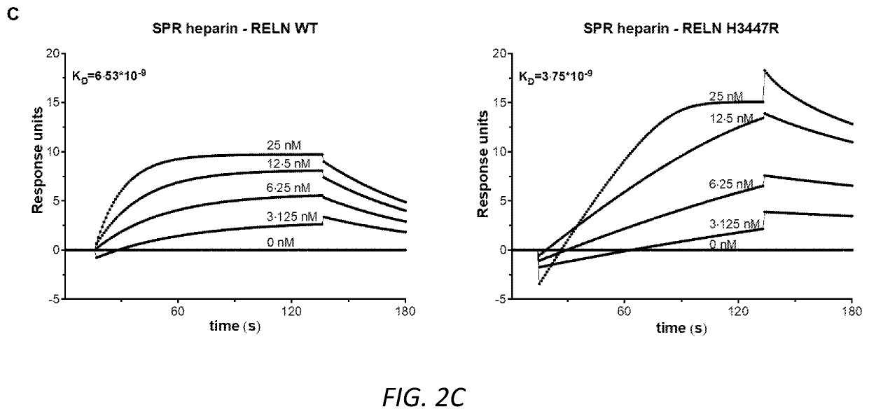

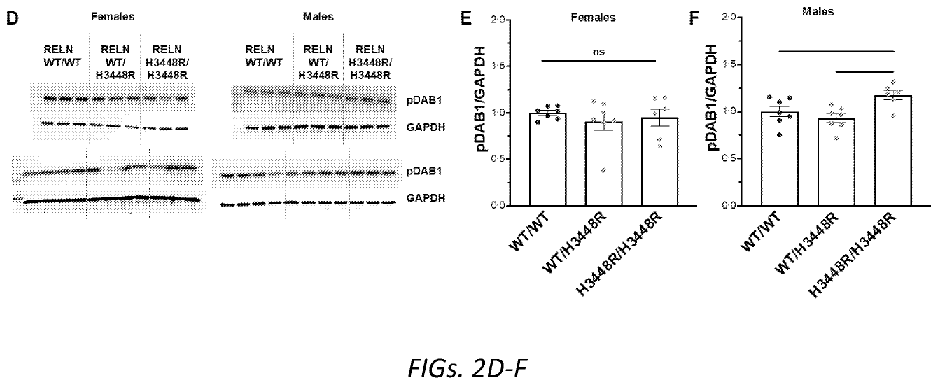

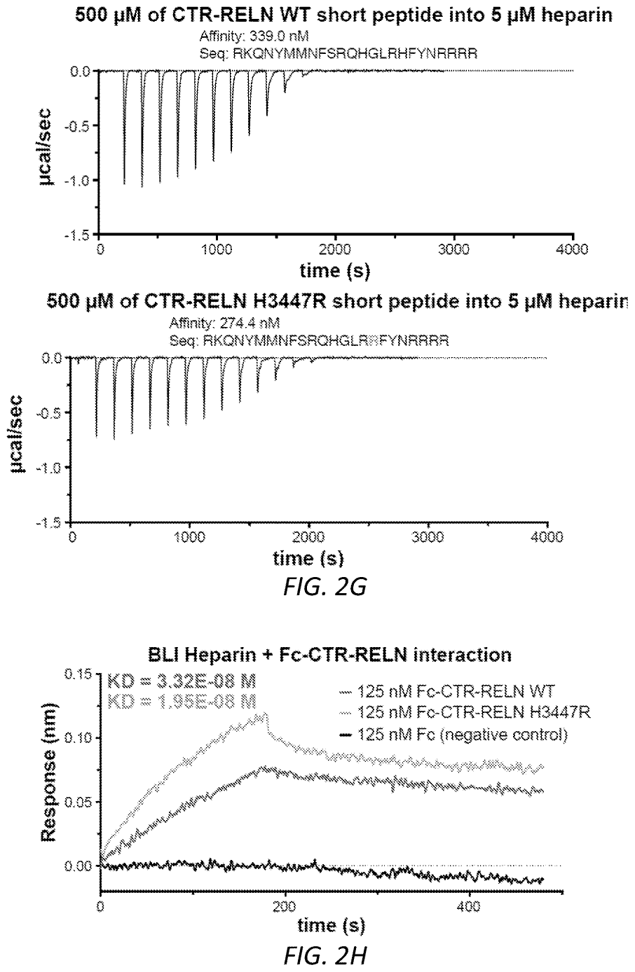

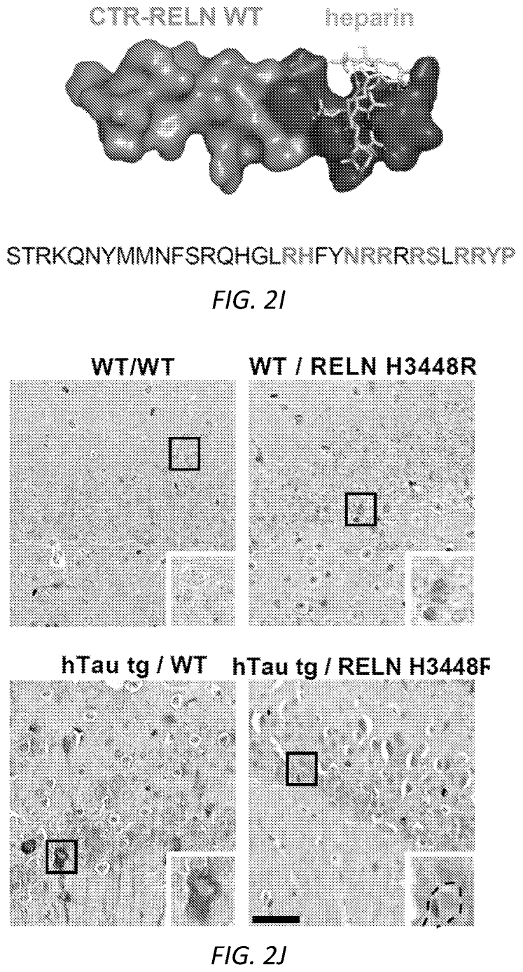

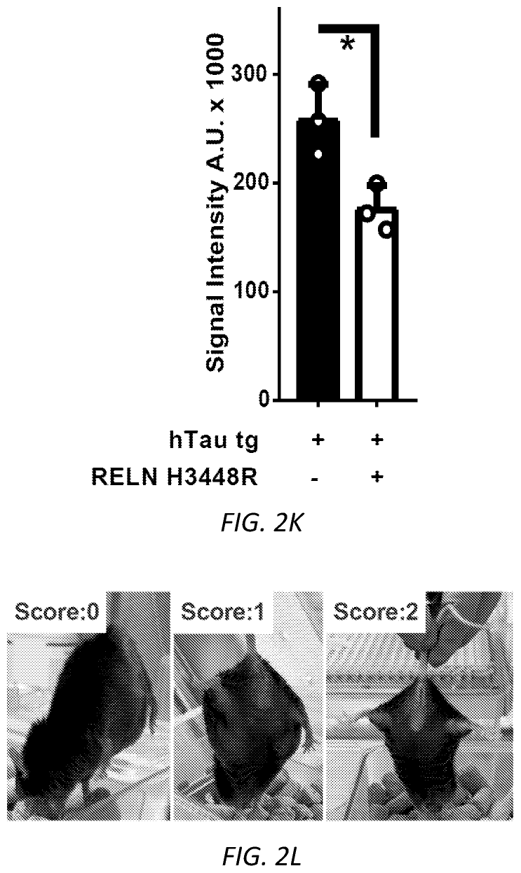

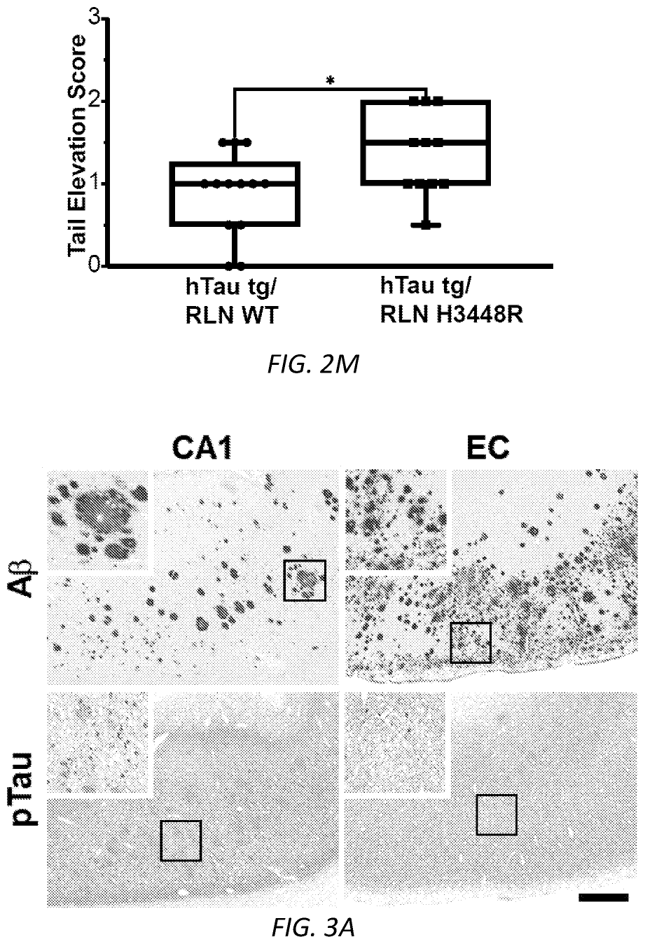

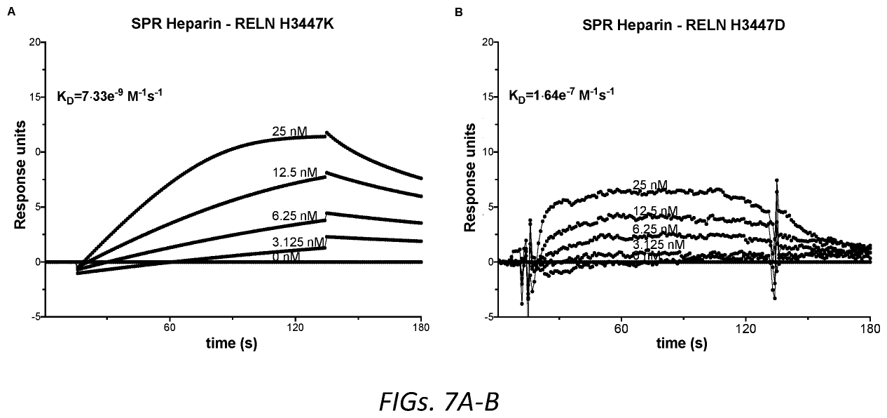

All data presented is expressed as averaged values and error expressed either as standard error of the mean (s. e. m.) or standard deviation (SD). Statistical analyses were performed using GraphPad Prism 8 (GraphPad Software, La Jolla California USA, graphpad.com). P values less than 0.05 and a α of 0.05 were considered as statistically significant. We used Kruskal-Wallis (Dunn's post-hoc analysis for multiple comparisons of four independent experiments to compare changes between primary cortical neurons treated with either MOCK, RELN WT or RELN H3448R and presented data as mean±s. e. m. For the SPR data (FIG. 2C & FIGS. 7A-B), we verified the accuracy of the results via Chi square (χ2) analysis and compared the sensorgrams (colored line) obtained experimentally with the sensorgrams generated mathematically by the BIAnalysis software (black line). Values ranging from one to two were interpreted as significant (accurate), and those below one as highly significant (highly accurate). Western blotting analyses presented in FIGS. 2E-F were done using one-way ANOVA followed by Fisher's LSD test for multiple comparisons by using GraphPad Prism 9. Neuropathological data (FIG. 3) was analyzed, and graphs generated by using GraphPad Prism 6 (GraphPad Software, Inc., La Jolla, CA, USA) and R statistical software (R Foundation for Statistical Computing, Vienna, Austria; R-project.org.). Analyses included distribution analysis and correlation analysis were performed using Spearman's Rho test. Brain color maps were created using the cerebroViz package for R. Statistical significance of all analyses was determined with * p≤0-05, **p≤0.01 and ***p≤0.001.

### Example 1.1—Case Report

We identified a male carrier of the PSEN1 E280A mutation who remained cognitively intact until age 67. He completed five years of formal education in his home country (Colombia), and worked until he retired at age 64. He was married and had two children. First assessment at age 67 revealed limited verbal learning skills and language difficulties in the context of functional independence. The patient was diagnosed with MCI, characterized by short-term memory and verbal fluency declines at age 70.

At age 72, his language had deteriorated further. He progressed to mild dementia at age 72 (Table 2). Cognitive decline was preceded by a urinary tract infection-related episode of septic shock. At age 73, he required assistance with basic and instrumental activities of daily living meeting criteria for moderate dementia. He died at the age of 74 years from aspiration pneumonia and his relatives agreed to a brain donation for neuropathology.

The subject's sister carried the PSEN1 E280A mutation, had severe dementia when she was first evaluated at age 64, and progressed to end-stage at age 72 (see pedigree in FIG. 5A). According to family, she had depression, hypothyroidism, hypertension and cognitive decline at age 58 and developed dementia at age 61. Dementia was preceded by ocular trauma and tibia fracture after a fall, which required surgery under general anesthesia. She died at age 73 of sepsis of pulmonary origin.

TABLE 2
Test Scores and Percentiles for Age & Education. The subject's age and education-
adjusted neuropsychological test scores reveal a pattern of significant global
and progressive cognitive decline during a 5-year assessment period.

| | Initial | | 3-year | | 4-year | | 5-year | | | |
|---|---|---|---|---|---|---|---|---|---|---|
| | visit | | visit | | visit | | visit | | | |
| | (67 yo) | | (70 yo) | | (72 yo) | | (73 yo) | | | |
| Cognitive Tests | Raw Scores (Percentiles*) | | | | | | | | Mean (SD)* | |
| MMSE/30 | 29 | (61st) | 23 | (1st) | 16 | (<1st) | 7 | (<1st) | 28.33 | (2.24) |
| Naming/15 | 14 | (86th) | 15 | (95th) | 13 | (70th) | 11 | (30th) | 12 | (1.89) |
| CERAD Word List | 7 | (1st) | 5 | (<1st) | 5 | (<1st) | 1 | (<1st) | 15.58 | (3.62) |
| Learning/30 | | | | | | | | | | |
| CERAD Word List Delayed | 3 | (13th) | 2 | (5th) | 2 | (5th) | 0 | (1st) | 5.46 | (2.13) |
| Recall/10 | | | | | | | | | | |
| CERAD Word List | 3 | (<1st) | 6 | (<1st) | 9 | (39th) | 2 | (<1st) | 9.37 | (1.3) |
| Recognition/10 | | | | | | | | | | |
| CERAD Constructional | 10 | (66th) | 4 | (<1st) | 5 | (1st) | 4 | (<1st) | 9.29 | (1.78) |
| Praxis Copy/11 | | | | | | | | | | |
| CERAD Constructional | 9 | (73rd) | 5 | (27th) | 0 | (2nd) | 0 | (2nd) | 6.92 | (3.36) |
| Praxis Recall/11 | | | | | | | | | | |
| Semantic Fluency | 8 | (2nd) | 6 | (<1st) | 4 | (<1st) | 3 | (<1st) | 16.79 | (4.17) |
| (Animals) | | | | | | | | | | |

| | | | | | | | | | |
|---|---|---|---|---|---|---|---|---|---|
| Phonemic Fluency (FAS) | 26 | (68th) | 7 | (13th) | 14 | (30th) | . . . | 20.39 | (12) |
| Raven's Matrices Test/12 | 9 | (73rd) | 7 | (37th) | 5 | (12th) | . . . | 7.71 | (2.23) |

| | | | | | |
|---|---|---|---|---|---|
| GDS/15 | 5 | 4 | 2 | . . . | . . . |
| EDG/7 | 1 | 3 | 5 | 6 | . . . |

Note. MMSE, Mini Mental Status Examination; CERAD, Consortium to Establish a Registry for Alzheimer's Disease Test Battery; GDS, Geriatric Depression Scale; EDG, Global Deterioration Scale. Percentiles were calculated using age and education-based norms from a Colombian population at age 65 and above and expressed with mean and standard deviation (SD); see Torres, Vila-Castelar et al. 2019 (11) for full description of the neuropsychological measures and normative data. The following guidelines can be used for a qualitative interpretation of his performance: 90-75 percentiles=above average; 74-25 percentiles=average; 24-9 percentiles=below average; 8-2 percentiles=borderline; 1=extremely low.***

### Example 1.2—Identification of RELN-COLBOS Mutation

The patient was enrolled in the Colombia-Boston biomarker research study (COLBOS) and underwent neuroimaging examinations at MGH when he was 73 years old. Amyloid PET, measured by cortical-to-cerebellar Pittsburgh compound B (PiB), revealed that the subject's levels of cortical Aβ plaque burden were higher (distribution volume ratio, DVR=1.77) compared to that of younger MCI carriers from this kindred with a typical age of onset (DVRs 1.49-1.60, FIG. 1). Tau tangle burden in the inferior temporal lobe, measured by flortaucipir (FTP), was similar to that seen in younger PSEN1 E280A MCI carriers with typical age of onset (1.78 SUVR). However, he had relatively limited tau pathology in the entorhinal cortex (EC=1·34 SUVR, FIG. 1A, C) and in other neocortical regions, such as the posterior cingulate cortex and precuneus (PCC=1-51; Precuneus=1·49 SUVR, FIG. 1A), which usually show greater levels of tau pathology in PSEN1 E280A carriers who develop MCI and dementia at typical age (3) (FIG. 1). Sparing of the entorhinal cortex from tau pathology is a salient feature in the RELN-COLBOS case that could be critical for the protection phenotype.

Measurements of metabolic rate for glucose in the precuneus-to-whole brain region using fluorodeoxyglucose PET showed relatively preserved metabolism (FIG. 1). He had brain atrophy, measured by MRI-based hippocampal-to-whole brain volume similar to typical MCI carriers. These imaging findings suggest that in this patient as well as in the APOE3 Christchurch homozygote case,(2) protection against ADAD dementia may be mediated through a mechanism limiting regional tau pathology even in the face of high amyloid burden and substantial tau pathology in brain regions other than the medial temporal lobe (FIG. 1).

Our genetic analyses confirmed that the subject was a heterozygote carrier of the PSEN1 E280A mutation (confirmed by single cell RNA sequencing), ruled out the presence of the Christchurch mutation (subject was APOE3APOE3, and had normal blood lipid profile), and identified a heterozygous variant in RELN (H3447R, FIG. 5B), which we named “RELN-COLBOS”, as the most promising missense variant potentially contributing to the phenotype in the protected subject. The RELN-COLBOS variant was only found in the subject and his sister (also APOE3 APOE3), who also had late onset of cognitive decline. In brief, we focused on the RELN-COLBOS variant because it ranked in the top three candidate genes in the Genomizer priority score analysis and because RELN is functionally closely related to APOE, the gene mutated in the other case with extreme protection against ADAD (2).

### Example 1.3—Molecular Analysis of RELN-COLBOS Variant

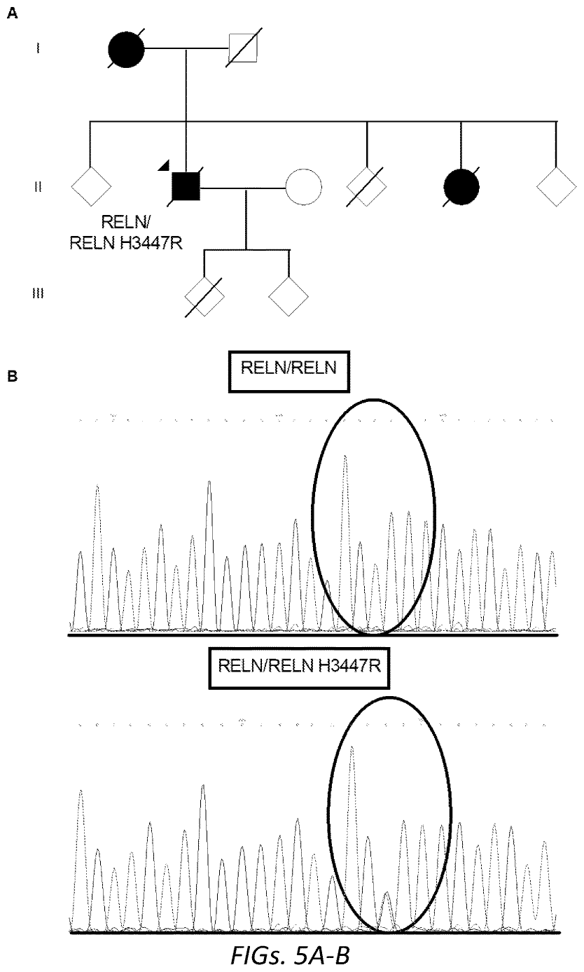

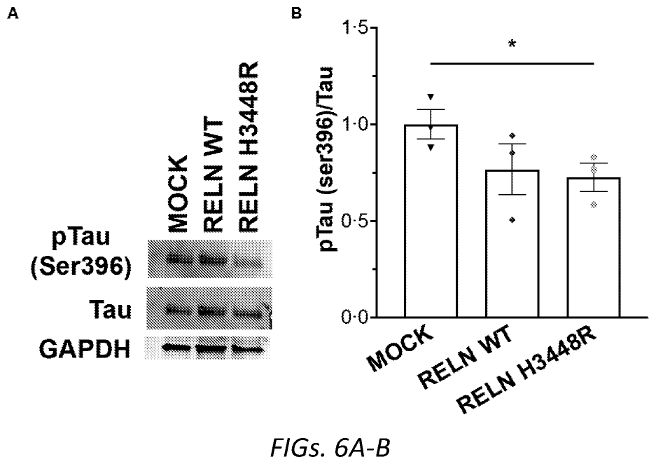

RELN binding triggers clustering and activation of VLDLr and APOER2, leading to a signaling cascade regulating disabled 1 (Dab1) activity, thus resulting in diminished tau phosphorylation.(4-7) RELN-COLBOS was about twice as effective at triggering Dab1 phosphorylation compared to wild type (WT) RELN (FIG. 2A, p=0.0246) and reduced tau phosphorylation at position 396, an early marker of tauopathy (8-10), in primary culture mouse cortical neurons (FIGS. 6A-B). RELN COLBOS did not directly impact binding of RELN to VLDLr or APOER2 in cell-free binding assays.

The C-terminal region of RELN (CTR-RELN), where the H3447R variant is located, modulates signaling indirectly via interactions with a previously unidentified co-receptor on cell membranes.(11) CTR-RELN has many basic amino acids that are extremely well conserved across species (11), which we hypothesized could mediate interactions with glycosaminoglycans (GAGs). Interactions with GAGs are a rate-limiting step in the interaction of ApoE with some receptors (12) whereas GAGs' role in RELN activity has not been fully resolved (11, 13). We used affinity chromatography to examine heparin (a type of GAG) binding of recombinant CTR RELN peptides. CTR RELN WT and CTR RELN H3447R bound to heparin. CTR RELN H3447R required higher NaCl salt concentrations to be released from the heparin column suggesting increased binding affinity (FIG. 2B). Surface plasmon resonance (SPR) measures of kinetic constants showed that the affinity of CTR RELN H3447R is about twice that observed in the WT (FIG. 2C).

Substitution of histidine at position 3447 with aspartic acid, a highly acidic amino acid (H3447D), reduced heparin interactions whereas substitution for lysine (H3447K), a basic amino acid, had minimal effects, further supporting a critical role for position 3447 in GAG binding. Surface plasmon resonance (SPR) measures of kinetic constants showed that the affinity of CTR RELN H3447R is about twice that observed in the WT: H3447R>WT>H3447K>H3447D (FIG. 2C, & FIG. 7A, B). RELN H3447D could not be produced in sufficient quantities for signaling analysis. This limitation is consistent with previous reports of specific mutations of the C-terminus that limit secretion of RELN (52).

We used ELISA to evaluate direct binding of recombinant RELN H3448R to canonical RELN receptors VLDLr and APOER2 (53) to further examine the molecular mechanism leading to the observed gain-of-function. We used experimental conditions with RELN H3448R, RELN WT, or with an equimolar mixture of WT and variant proteins because RELN oligomerization is a critical property modulating binding to receptors (53). In all conditions, full-length RELN H3448R and RELN WT did not differ in their ability to bind to VLDLr or APOER2. We further determined that the C-terminal region of RELN (CTR-RELN) WT or CTR-RELN H3447R recombinant peptides were unable to bind directly to VLDLr or APOER2. This finding confirms previous reports demonstrating that the fifth and sixth reelin repeats of RELN (R5-6) are necessary to mediate binding to the receptors (53). We concluded that the gain-of-function of the RELN H3447R variant was not likely explained by direct changes in binding affinity for VLDLr or APOER2 receptors.

### Example 1.4—Disease-Modifying Effects of RELN-COLBOS Mutation

We generated a knock-in mouse model carrying the equivalent of the RELN-COLBOS variant (H3448R or mRELN-H3448R (11)) to further support the genetic imputation of causality, a common practice to study rare variants. This mouse model is viable, fertile, and lacks overt structural and phenotypic brain abnormalities of RELN loss-of-function variants (e.g. cortex lamination defects, abnormal neuronal migration, and cerebellar hypotrophy) (14, 15). Analyses of cerebellum from mice with the mRELN-COLBOS confirmed the observation of a gain-of-function for RELN-H3448R as determined by enhanced phosphorylation of Dab1 in males (FIG. 2D, F, p=0·0284) and revealed a propensity for the formation of higher molecular protein oligomers for RELN-COLBOS, a feature that may be critical for enhanced activity (16). The cerebellum is a target of RELN phenotypes in mice and humans (14, 15). Morphological analysis revealed a mild, yet statistically significant increase in the number of cerebellar neurons in mice with the RELN-COLBOS variant. This supports the hypothesis of a gain-of-function mechanism, although the neuronal density phenotype was not observed in other brain regions.

This mouse model allowed us to examine sexually dimorphic effects of the RELN-COLBOS variant, a feature that has been described for conditions linked to genetic variations in RELN including schizophrenia, bipolar disease, autism, and Alzheimer's disease (17-22). Increased Dab1 phosphorylation and enhanced oligomerization of RELN were observed only in male mice (FIG. 2D). This finding was consistent with our observation of optimal association of RELN-COLBOS with protection against ADAD in a male vs. a female case. Homozygosity was required to detect changes in Dab1 activity and in GSK3β activity (another downstream target of RELN signaling) associated with the RELN-COLBOS variant. Altogether, these data indicate that RELN H3447R is a gain-of-function (hypermorph) variant.

To attempt to correlate the phenotypes of RELN-COLBOS in mice and human, we employed a crossbreeding strategy using our knock-in mouse model and a tauopathy mouse model, specifically the STOCK Tg(Pmp-MAPT*P301L)JNPL3Hlmc mouse from Dr. Hutton's lab, distributed by Taconic. This mouse model expresses a mutation in the tau gene, leading to accumulation of tau tangles and neuronal loss in specific brain regions, commonly used to study tauopathies²³. The decision to use this mouse model was based on the known effects of RELN signaling on tau phosphorylation²¹, as well as our clinical observations of a relative reduction of tauopathy in certain brain regions from post-mortem human brain samples of the protected case. Our study found that male P301L mice expressing the RELN-COLBOS allele had a substantial reduction of human tau phosphorylation (ptau205) in the hippocampus (FIG. 2J-K) and medulla oblongata as compared to controls (FIG. 2J). We also observed that the abnormal limb clasping response, which is a common consequence of tauopathy in mice, was significantly rescued in RELN-COLBOS mice with the tau transgene (FIG. 2L-M). Although additional studies of this model are necessary, our findings strongly support our hypothesis of RELN-COLBOS is a gain-of-function mutation and it is likely genetically implicated in the resilience to tauopathy. This analysis also shows direct evidence of a rescue in a mouse model with a MAPT mutation linked to frontotemporal dementia.

Postmortem examination of the case indicated neuropathological evidence of severe AD (classified as CERAD C, Braak VI stage and Thal phase 5) with extensive amyloid and tau pathology (FIG. 3A). Recently, we reported the neuropathological profile of the PSEN1 E280A carrier homozygous for the APOE Christchurch mutation. This case showed a unique pathological phenotype among PSEN1 E280A cases with remarkably low ptau pathology in most brain regions except in the visual primary cortex (23). In contrast, side-by-side comparisons showed that the RELN-COLBOS case had more ptau pathology relative to the APOEch case except in specific regions. Both cases showed extensive Aβ pathology in all evaluated areas, albeit with some individual differences.

We focused our analysis on hippocampus and associated cortices because these structures are known to be affected early in AD (24). Neurons within layer II of the entorhinal cortex and entorhinal cortex neurons in general are particularly vulnerable to aging and AD (25). We measured neuronal density in hippocampal and parahippocampal areas of the RELN-COLBOS case, the AD resistant APOEch case, typical PSEN1 E280A cases and typical sporadic AD cases (FIG. 3B). We found that lower AD pathology was associated with high neuronal density in entorhinal cortex of the RELN-COLBOS case compared to the APOEch case or the FAD and sporadic AD controls (FIG. 3C). This association was not apparent in other subregions such as CA1 (FIG. 3C; S17 & S18). RELN-COLBOS and APOEch cases showed distinctively lower intraneuronal ApoE signal compared with FAD and sporadic AD controls (FIG. 3D) whereas RELN-COLBOS showed higher Reelin intracellular signal in the white matter (FIG. 3D).

Neuropathological findings are consistent with our in vivo neuroimaging observations and confirm a potential role for the integrity of the entorhinal cortex as a target of RELN-mediated mechanisms critical for the resilience to ADAD.

### Example 2. In Silico Modeling of Reelin

In order to understand the potential importance of the Reelin CTR, computational analysis was used to show that the RELN CTR, including basic AA likely to form a charge-based binding site, was highly conserved across mammalian species. We also show that the Reelin CTR is likely to be intrinsically disordered. Intrinsically disordered regions (IDRs) within proteins lack defined tertiary structure and may be important to several biological functions, including protein interaction. Additionally, IDRs may play a role in interactions within plaque deposits in individuals with neurodegenerative diseases including AD. Previous work has shown that the Reelin CTR is highly conserved across vertebrate species (11). This work also showed that Reelin was not required for secretion but may play a role in downstream signaling.

### Methods

A phylogenetic tree was created using BEAST, which uses Bayesian Markov Chain Monte Carlo (MCMC) to create the tree based on the Reelin CTR sequences. PONDR® was used to assess intrinsically disordered regions of RELN. Specifically, VL-XT (Variously Long disordered regions and X-ray characterized Terminal disordered regions) is a combination of three feedforward neural networks. The individual neural networks were trained on long regions of disorder (greater than 39 AAs), n-terminal disorder, and c-terminal disorder.

### Results

### The Reelin CTR is Highly Conserved

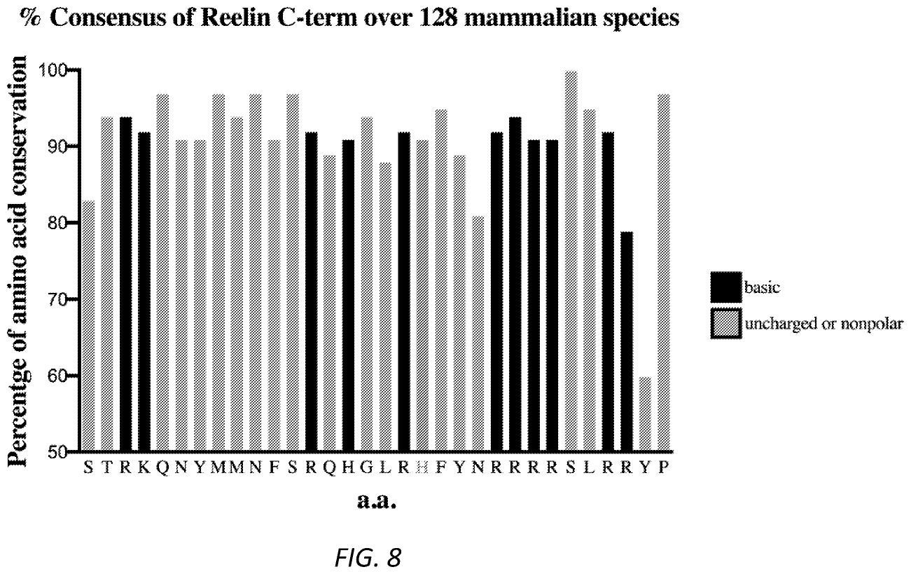

The Reelin CTR is also highly conserved across mammalian species (FIG. 8). It is therefore hypothesized that the C-terminal region of Reelin is important for Reelin binding to HSPG and regulation of subsequent downstream pathways. Among species that were available in the National Center for Biotechnology Information (NCBI) database, there is a high level of conservation at position 3447 across most mammalian species except for in horses (Equus caballus, Equus asinus, Equus przewalskii).

### The Reelin CTR Contains a GAG-Binding Motif

Initial structural data suggests that there may be two binding sites at the c-terminal region (FIG. 9). The key AA positions that interact with GAGs can be predicted based on the orientation of arginines in the known heparin-binding motif ((B)Bxx(x/B)BxxB(B)), where B represents positively charged AAs and (x) represents non-consensus AAs (29). Basic AA interactions with acidic GAGs are largely driven by electrostatic forces (30). Therefore, it is hypothesized that these basic amino acids may be especially important for GAG interaction, and the c-terminal region may contain a binding site that interacts with GAGs.

### H3447 is Oriented in the Same Direction as Other Basic Amino Acids

Bioinformatic analyses showed that the RELN CTR is highly conserved across mammalian species, including the H3447R position (purple) found to be mutated in the AD resilient case. Many of the conserved amino acids are highly basic residues suggesting the possibility of binding to GAGs. The basic AAs shown in magenta, including position 3447, are oriented on the same side which may indicate a primary role in interactions with heparin. Here, the orientation of basic amino acids may contribute to creating a binding site.

### Conclusion

The Reelin CTR is highly conserved across a wide variety of species. The basic AAs that are hypothesized to contribute to GAG interaction have >90% conservation. The only species with a different AA in position 3447 are in the horse family (Q3447), which notably is uncharged and should not make a significant difference compared to weakly basic histidine. Furthermore, these basic AAs, including position H3447R, are oriented on the same side of the alpha-helix, which has previously been shown to be an important aspect of GAG interaction.

### Example 3: Kinetics of Reelin-Heparin Interactions

In order to further understand the kinetics of RELN CTR—Heparin interaction, Surface plasmon resonance (SPR) adds additional support to the proposed neuroprotective mechanism of the H3447R variant. The equilibrium dissociation constant (K_{D}) shows that H3447R has ˜2× the binding affinity of H3447. Furthermore, bio-layer interferometry was used to further corroborate these results.

### Methods

### Surface Plasmon Resonance

Surface plasmon resonance assay (SPR) was used for performing binding kinetics using a Biacore 3000 Instrument (GE Healthcare) at 25° C. following previously published protocols 34 24 fee-for-service by Precision Antibody (Maryland, USA). Heparin was covalently linked to streptavidin coated chip at 13 response units (RU) concentration and unoccupied sites were blocked with biocytin. Antigen was flowed over the chip using a range of single analyte concentrations prepared in DPBS buffer and using a flow rate of 30 μL/min. Binding of antigen to the ligand was monitored in real time to obtain on (ka) and off (kd) rates. The equilibrium constant (KD) was calculated from the observed ka and kd. Accuracy of the SPR analysis was determined via Chi square (2) analysis as described in the statistical analysis section.

### Bio-Layer Interferometry (BLI)

We used Fc-fusion Reelin CTR peptides since peptides alone would be smaller than the limit of detection in this experimental design. To assess heparin—Fc-Reelin interaction, the octet system (bio-layer interferometry) was used to assess heparin-protein kinetics. 50 μg/mL biotinylated heparin was immobilized on the biosensor tip surface for 300 sec on preconditioned biosensors. This was followed by quenching with biocytin at 50 μg/ml, baseline buffer diluent for 120 sec, 200 nM of analyte (Fc-fusion proteins) for 120 sec, and disassociation in assay buffer for 120 sec.

Bio-layer interferometry (BLI) was additionally used to assess NRP1-protein kinetics at 30° C. and 1000 rpm agitation. 1 mg/mL NRP1 (R&D 3870-N1-025) was biotinylated at a 1:2 molar ratio, desalted, and immobilized on the SA biosensor tip (Pall ForteBio) surface. This was followed by 1) 180 seconds baseline buffer diluent, 2) loading of the ligand (NRP1), 3) 180 seconds baseline buffer diluent, 4) 240 seconds association (analyte), and 5) 300 seconds disassociation in assay buffer. Assay buffer: SD Buffer (pH7.4 PBS, 0.05% tween20, 0.01% BSA).

The experimental data were fit with the 1:1 binding model and analyzed with global fitting using Octet Data Analysis software to calculate K_{D}.

### Results

We recently showed that the RELN CTR H3447R peptide had 2-fold higher interaction with heparin relative to RELN WT with surface plasmon resonance (SPR; submitted to NEJM). These kinetics were assessed with a synthetic peptide (AA3431 to AA3460). We designed Fc-fused RELN CTR proteins to confirm this finding in a protein produced in mammalian cells. The Fc-fusion RELN WT and H3447R interactions with heparin were assessed using bio-layer interferometry (BLI; FIG. 1C). These proteins are cleaved by furin during production in mammalian cells, resulting in short RELN CTR variants lacking the last six amino acids. In this configuration, RELN H3447R also has about 2-fold higher affinity to heparin compared to WT. We found that that the Kₐ is the main contributor to the difference in K_{D} values, which indicates that either the variant needs less energy to interact with heparin or that this variant saturates the heparin substrate twice as fast in comparison to WT. Therefore, the CTR sequence upstream of Furin cleavage may contribute to CTR-heparin affinity in the presence of the H3447R mutation.

### Conclusion

Kinetic data shows that RELN H3447R has ˜2-fold increased interaction with heparin compared to RELN WT. Because the difference is relatively small, it is relevant that this difference was seen across two different experimental systems with both long and short RELN variants. These data indicate that RELN H3447R may increase association of RELN and GAGs and may therefore have a competitive advantage compared to other molecular or protein interactions with GAGs in vivo.

### Example 4: Affinity Data of Reelin-Heparin Interactions

High performance liquid chromatography (HPLC) was used to assess the interaction between HSPGs and the reelin CTR. Delayed peak interactions in isotonic PBS supplemented with 1M KCl indicate increased peptide-heparin interaction. Our data indicated delayed peak retention time between 0-2 arginines; this difference in peak retention time was observed in both long and short peptides. Therefore, the basic amino acids in the 3446-3447 positions are important to heparin interaction. Since all short peptides have earlier peak retention times, there may be a second binding site in the last 6 AAs of the long peptide sequence. We also show a basic mutation at the 3447 position has a later peak retention time in comparison to neutral (H) or acidic (D) AAs.

### Methods

### Heparin Column Chromatography

To assess Reelin-heparin binding affinity, Reelin variants were run on a heparin column with steps of 0.05M in a range of 0.05-1M NaCl over 20 1 mL column volumes in 20 mM Tris HCl, as described in Arboleda et. al 2019 (2). Sample fractions were eluted at an increasing NaCl step size to determine the ionic strength needed to disrupt the binding between Reelin and heparin. Therefore, the stronger the interaction, the higher concentration of salt would be needed to disrupt the bond.

### High Performance Liquid Chromatography (HPLC)

HPLC gives a more accurate 50 μl of 0.3 μg/μL uncleaved or cleaved WT Reelin peptide was used at 0.3 ml/min in 0.15MKCl, 10 mM PBS. 0-13.5 min: 0.15M KCl to load sample, 13.5-14.5 min: ramp to 0.5M KCl, 14.5-24.5 min: 0.5-1M KCl gradient (ramp), 24.5-45 min: 1M KCl (isocratic elution), 45-55 min: 1M KCl at 0.6 ml/min (wash), 55.0-56.0 min: ramp to 0.15M KCl at 0.3 ml/min, 56.0-59.0 min: 0.15M KCl (reset column). Fluorescence Intensities were measured at an excitation wavelength of 260 nm and emission wavelength of 290 nm, based on fluorescence properties of aromatic amino acids. To assess fluorescence properties of peptides, 15 ul of ˜0.6 ug/mL of peptide was diluted in 500 μL of 0.15MKCl, 10 mM PBS, and peak excitation and emission of peptides was calculated from the 2D graphical data.

### Method Development for HPLC

The column chromatography method was adapted from previous research from the laboratory (2). Based on the column chromatography data, the initial protocol used a NaCl gradient from 0-1 M and a 5M salt wash to reset the HPLC column. However, the high salt gradient was corrosive to the HPLC machine parts. Therefore, KCl was considered because the salt has a higher ionic strength compared to NaCl. A lower molar concentration of a buffer with KCl could be used to disrupt heparin—Reelin CTR interactions compared to NaCl to help keep the HPLC pumps free of residual salts and potential corrosion. Since a lower concentration of salt was used, the amount of time needed at an isotonic hold of 1M KCl was extended until the peptide signal could not be detected before the column was equilibrated for the next sample.

### Results

Heparin Column Chromatography Shows RELNH3447R Enables Increased Interaction with Heparin

Eluted peptides from heparin column chromatography were quantified at each 0.05 M NaCl step were assessed with both nanodrop and ELISA. Overall, nanodrop measurements had sharper, more defined peaks compared to ELISA perhaps because ELISA is dependent on antibody affinity to the RELN CTR while nanodrop measurements directly quantify the amount of peptide. The peak elution step is determined as the maximum ratio to input per sample.

For all RELN variants long (uncleaved) peptides had later peak retention times compared to short peptides, suggesting that the last 6 AAs may include a heparin binding site. Furthermore, basic AAs in position 3447 (R, K) have a peak elution at a higher salt concentration compared to WT or H3447D. The charge of AAs at position 3447 may therefore be an important factor in RELN CTR—GAG interaction.

While heparin column chromatography was able to quantify RELN CTR—heparin interaction to an extent, HPLC can give more accurate quantification of this interaction using a gradient. Representative normalized peaks show that RELN H3447R has delayed peak times compared to RELN WT for both long and short peptides. Significant differences are characterized as average peaks with greater than 30 seconds between retention times.

### Conclusion

Chromatography data showed that the RELN H3447R mutation increased heparin interaction as a model for overall GAG interaction. The difference in retention time of RELN peptide variants is due to the AA mutation rather than a change in fluorescence properties or a change in detectability of the peptide since the peak excitation and emission spectra remain similar across peptide variants. Additional mutations can include substitutions in other key arginine residues (R3451, R3454, and R3458) that are oriented with H3447R to contribute to RELN CTR—GAG interaction.

### Example 5: Other Potential Reelin CTR Interactions

Recent work shows that the reelin CTR also interacts with neuropilin (NRP1) (46). This study found that the last 6 AAs after the furin cleavage site were critical for NRP1 interaction. The uncleaved “long” CTR-RELN interacts with NRP1 and the cleaved “short” CTR-RELN does not (46). BLI was therefore used to further assess whether the H3447R mutation affects NRP1-RELN CTR interactions. All known NRP1 binding domains have been shown to have a C-terminal arginine with a motif called CendR (R/KXXR/K) (47). Specifically, the C-terminal arginine is relevant to binding to the NRP1 b1-b2 domain (48). Therefore, by sequence alone, the RELN CTR may have additional interactions beyond GAGs. In particular, RELN H3447R may contribute to optimizing the NRP1 binding motif especially after furin cleavage.

### Methods

### Docking

NRP1 and RELN CTR structures were uploaded to ClusPro 2.0 (cluspro.org/), to model these protein interactions. In brief, the top 1000 rotatamers with the lowest scores out of 70,000 rotations are chosen. The algorithm clusters these 1000 rotations, which entails ranking positions with the most neighbors within a 9 Å RMSD radius. The top position then becomes the center of the first cluster, and this process is repeated to rank up to 30 clusters based on size (i.e. number of neighbors). Next, energy minimization removes steric overlaps. Therefore, ClusPro considers the lowest energy structures of the largest clusters rather than just energy minimization alone (49, 50).

The output of the ClusPro algorithm is a list of most probable conformations. One model for each ApoE peptide was taken as preliminary data for further analysis with PyMol Version 2.3.3 (pymol.org/). In the future, the top three lowest-energy models ranked by ClusPro will be assessed. Polar contacts between peptides were defined by the PyMol software as contacts within 3 Å through the ‘Measurement Mode’ function.

### Bio-Layer Interferometry (BLI)

Bio-layer interferometry (BLI) was used to assess NRP1-protein kinetics at 30° C. and 1000 rpm agitation using previously published methods. 1 mg/mL NRP1 (R&D 3870-N1-025) was biotinylated at a 1:2 molar ratio, desalted, and immobilized on the SA biosensor tip (Pall ForteBio) surface. This was followed by 1) 180 seconds baseline buffer diluent, 2) loading of the ligand (NRP1), 3) 180 seconds baseline buffer diluent, 4) 240 seconds association (analyte), and 5) 300 seconds disassociation in assay buffer. Assay buffer: SD Buffer (pH7.4 PBS, 0.05% tween20, 0.01% BSA).

The experimental data were fit with the 1:1 binding model and analyzed with global fitting using Octet Data Analysis software to calculate K_{D}.

### Results

Molecular docking suggests that the alpha- and beta-GAG binding sites overlap with the NRP1 binding site. However, n-terminal AAs of the RELN CTR may also contribute to NRP1 interactions.

Vascular endothelial growth factor (VEGF) is known to interact with NRP1 and was therefore used as a positive control (31). As previously mentioned, our Fc-fusion proteins were made in mammalian cells thus were expected to be cleaved by Furin. Previously published data concluded that WT CTR did not bind NRP1 after removal of the last six amino acids by Furin.

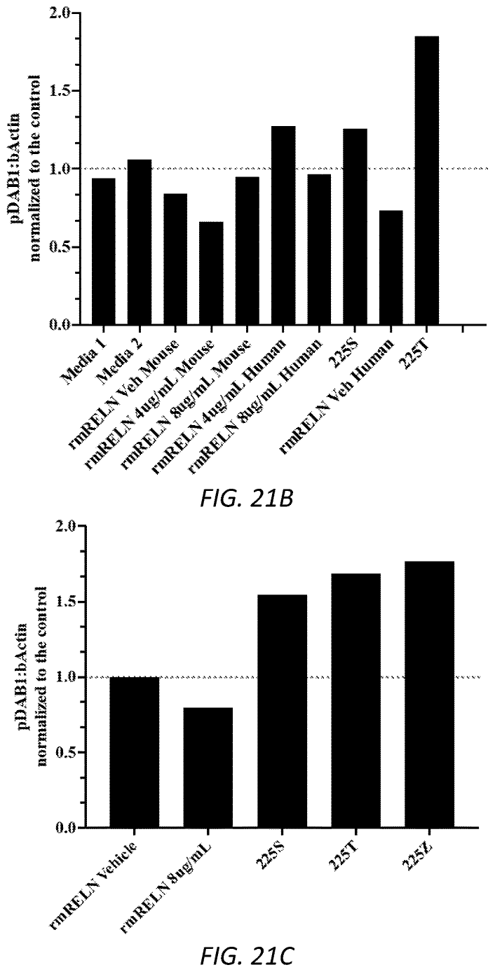

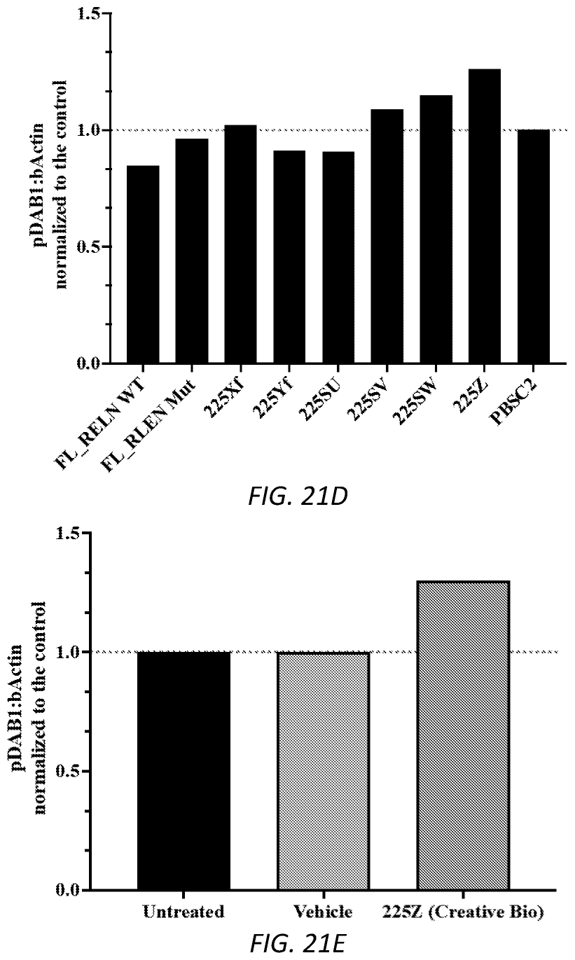

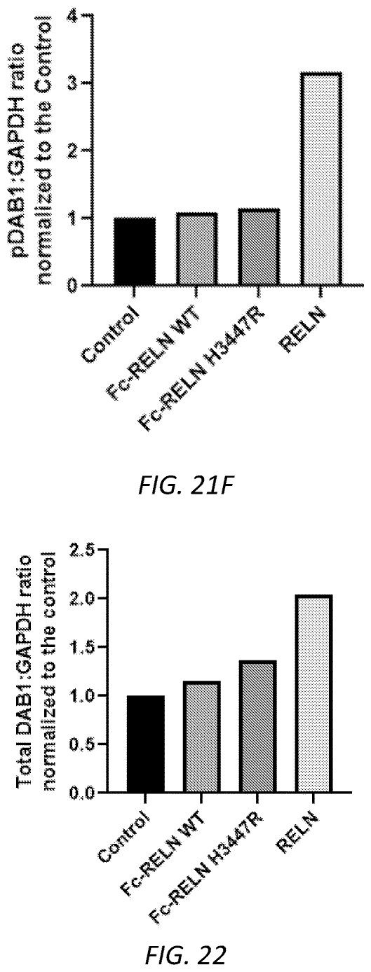

We confirmed enhanced interactions of CTR-RELN H3447R with heparin through the use of isothermal titration calorimetry (ITC, FIG. 2G) and biolayer interferometry (BLI, FIG. 2H). The Kₐ was found to be the main contributor to the difference in Kₐ values, and the mutant CTR-RELN was found to have a more negative Gibbs free energy in comparison to WT, suggesting that the COLBOS mutation enables spontaneous CTR-RELN reactions with heparin. Our nuclear magnetic resonance (NMR, FIG. 21, FIG. 9) study revealed that the CTR-RELN may have an alpha-helix structure including a flexible region with a domain we named “Flexibility Vertex” in the presence of trifluoroethanol, though it may be unstructured under native conditions as revealed by circular dicroism (Table 3).

TABLE 3
Secondary structure analysis of circular dichroism spectroscopy
data of CTR-RELN WT alone or with 50% TFE

| | | 50% 2,2,2 - |
|---|---|---|
| Secondary structure | Peptide only | Trifluoroethanol (TFE) |
| Helix (regular) | 0.0% | 22.0% |
| Helix (structural) | 0.6% | 11.9% |
| Antiparallel β-sheet | 14.6% | |
| 33.4% | | |
| Parallel β-sheet | 0.0% | 0.0% |
| β-turn | 15.5% | 10.4% |
| Other | 50.5% | 41.0% |
| RMSD | 0.1573 | 0.1008 |

Heparin binding analyses of a library of mutant CTR-RELN peptides uncovered two binding sites for GAGs which we named as “alpha-GAG binding site” and “beta-GAG binding site” (FIG. 9). The alpha-GAG binding site is located in the last six amino acids and overlaps with a previously identified binding site for Neurophilin 1 (NRP1), which is released by Furin. The beta-GAG binding site is located upstream of the Furin cleavage site and spans amino acids 3446 through 3451. Our research also found that CTR-RELN COLBOS has a 10-fold higher affinity for NRP-1 compared to the wild-type version of CTR-RELN (Table 4), due to the optimization of the beta-GAG binding site. We conducted extensive studies of mutant CTR-RELN interactions with heparin by HPLC (FIGS. 11-D), BLI (FIG. 2H), and NMR structure (FIG. 21, 9) to support these assertions.

TABLE 4
Analysis of binding parameter of CTR-RELN-NRP-1 interactions

| Analyte | KD | ka | kd |
|---|---|---|---|
| Fc | / | / | / |
| Fc-WT | 6.10E−07 M | 9.40E3 M⁻¹ · s⁻¹ | 5.73E−3 s⁻¹ |
| Fc-H3447R | 7.93E−08 M | 1.29E4 M⁻¹ · s⁻¹ | 1.02E−3 s⁻¹ |
| VEGF | 5.53E−09 M | 2.72E5 M⁻¹ · s⁻¹ | 1.50E−3 s⁻¹ |

Our data showed the RELN CTR peptide without the alpha-GAG binding site located in the last six amino acids of RELN CTR had 100-fold less affinity for NRP1 than that of VEGF, consistent with the previously published data. However, with the H3447R mutation, the affinity was 10-fold higher in comparison to WT, suggesting that H3447R forms a new binding site or significantly increase affinity for NRP1 via the beta-GAG site. In sum, RELN H3447R peptide has ˜10-fold increased affinity to NRP1 in comparison to WT RELN peptide, much closer to the affinity range of VEGF, thus it is gain-of-function.

### Conclusion

Previously published data showed that NRP1 interaction was lost in WT short RELN peptides. Data here adds more to the story, where WT short has significantly less interaction with NRP1, especially compared to VEGF. However, these data support the hypothesis that RELN H3447R adds a new beta-GAG binding site or substantially optimizes it, considering the mutation rescues RELN CTR—NRP1 interaction by ˜10-fold. Accordingly, RELN gain-of-function may be achieved by introducing the H3447R variant and/or reducing furin cleavage of the RELN-CTR by introducing the R3454A.

### Example 6. Structural Analysis of Reelin

Circular dichroism (CD) was used to further understand the unstructured nature of the Reelin CTR. The modelling results showed that the Reelin CTR may have higher fluctuations in the most disordered area of the Reelin protein. Furthermore, recently published data showed a high resolution structure of the CR8 within the C-terminus of Reelin, but were unable to characterize the Reelin CTR, potentially due to the flexibility of the domain (32). NMR helped get a high resolution structure conditions by using circular dichroism (CD) and found that 50% TFE (2, 2, 2-Trifluoroethanol) was needed to stabilize the WT Reelin peptide structure. Both approaches give insight into the unstructured region of the RELN CTR. In silico approaches add additional support, showing that the flexible region can be predicted through a combination of the RELN CTR sequence as well as known disordered regions of other proteins.

### Methods

### Normal Mode Analysis (NMA)

NMA is a principle component analysis (PCA) based method that considers the harmonic potential of the peptide, where the force field is simplified to a ball- and spring elastic network model. The model considers the harmonic potential of the Ca atom of each AA as a node connected by springs. Here, the energy function is minimized by diagonalizing the Hessian matrix consisting of the second derivative of potential energies (33).

### Predictor of Natural Disordered Regions (PONDR) VL-XT Disorder Analysis

This algorithm uses feed-forward neural networks to predict regions of disorder based on the AA sequence. VL-XT combines three feedforward neural networks trained on: 1) variously long regions (VL), 2) X-Ray characterized N-terminus, and 3) X-ray characterized C-terminus (XT). Compared to NMA, VL-XT is able to use known disordered regions of other proteins to predict regions of disorder.

### Circular Dichroism

The CD signal was recorded at the wavelengths ranging from 190 to 260 nm at 0.1 nm intervals using a 1 mm pathlength quartz cuvette. The samples were scanned at 50 nm/min with a 1 nm bandwidth and a 2 second integration time. Data was plotted as the average of four spectra. Data was deconvoluted using the online deconvolution software BeStSel (bestsel.elte.hu/).

### 2D Nuclear Magnetic Resonance (NMR) Structures

The NMR structure was completed as a fee-for service. The final NMR sample consisted of 50% (V/V) H₂O, 50% (V/V) of 98% pure 2,2,2-Trifluoroethanol-d₂ (CF₃CD₂OH), 0.25 mM DSS (4,4-dimethyl-4-silapentane-1-sulfonic acid, dissolved in H₂O) as the internal standard, and ˜3.3 mM reelin peptide. ¹H TOCSY (80 ms spin lock time), NOESY (200 ms mixing time), ¹H-¹⁵N HSQC, ¹H-¹³C HSQC experiments were performed at 25° C. on an Agilent 600 MHz NMR Spectrometer (DD2). Data was analyzed with NMRPipe and CCPNmr 2.4.2; structures were calculated with Cns 1.2.1 and aria 2.3.2. In total 200 structures were calculated, and the 20 lowest energy structures were selected.

### Results

Structural Characterization of the RELN CTR Reveals an Alpha Helix with a Flexible Arm

We determined RELN CTR was highly unstructured under native conditions using circular dichroism (CD). Addition of 50% TFE (2, 2, 2-Trifluoroethanol) stabilized the unstructured CTR and enabled characterization of the domain. With 50% TFE, the peptide secondary structure is more closely an alpha-helix, with characteristic negative minima at 208 nm and 222 nm. We cannot exclude the presence of unstructured regions within the RELN CTR even in the presence of TFE. The structure of disordered peptides may account for the “antiparallel β-sheet” or “other categories” found as a larger percentage structure in the peptide alone compared to the peptide with 50% TFE (34,35).

The CD results informed experimental conditions for NMR to resolve the Reelin CTR at atomic resolution. NMR is advantageous for smaller proteins, since it can capture dynamics and forgoes the need for crystallization (51). The 20 lowest energy structures were used to create the final NMR structure of WT RELN CTR with an approximate resolution of 2.8 Å. With this technique, we confirmed that the C-terminal domain of RELN in the presence of TFE forms an alpha helix with a highly flexible arm towards the C-terminal region. The vertex of the flexible region spans from the glycine residue at position 3444 to the histidine 3447 mutated in the protected case. We have named this region the flexibility vertex domain (FVD). Consensus motifs for GAG binding include basic amino acids on the same side of the alpha helix and on contiguous turns separated by ˜3.5 amino acids to allow for strong bonds (36). Additionally, the 20 lowest energy structures show that the helix containing position 3447 is unwinded, potentially exposing H3447R for GAG interactions.

### Reelin CTR—GAG Interactions Depend on Basic AAs Near H3447R

Our structural data in conjunction with our functional analysis indicate that the alpha-GAG binding site is likely to depend upon R3454, R3457, and R3458. As we concluded earlier from our functional studies, R3446 also mediates GAG interactions probably in conjunction with R3451 comprising the beta-GAG binding side. It is clear from the structure that the arginine introduced by the mutation at position 3447 found in the protected case is properly orientated for GAG interactions. Importantly, the new beta-GAG binding site is not cleaved by Furin, becoming a constitutively active site for RELN interaction with GAGs, NRP1 and potentially other receptors. Polar contacts between the WT RELN CTR and heparin suggest that this interaction is likely due to basic amino acids in the alpha- and beta-GAG binding sites (FIG. 9).

Reelin C-Terminal Peptides have More Atomic Fluctuation in the Alpha- and Beta-GAG Binding Sites

The glycine residue is marked to show the structured region found in NMR spectra towards n-terminus region of the peptide. Data suggests the last 6 AAs of H3447R long have more fluctuation in comparison to WT Reelin long. WT and H3447R Reelin short peptides were comparable; overall, the short peptides had decreased fluctuation in comparison to long peptides. Atomic fluctuation was measured as the root mean square deviation across all normal modes.

Modelling Shows that the Reelin CTR has Increased Disorder

To assess the flexibility of the region, the harmonic potential of each amino acid was first assessed using Normal Mode Analysis (NMA) (37). NMA shows that long peptides have increased flexibility, with RELN H3447R long having increased flexibility in comparison to WT long. Reelin short peptides show similar flexibility. NMA overall shows that long peptides have increased flexibility compared to short peptides; however, since all other peptides besides WT long were modelled peptides, these results may not be fully accurate. In contrast, Predictor of Natural Disordered Region (PONDR) analysis uses only the AA sequence to score the relative disorder of RELN variants. WT Long has a score of 0.9032 in AAs 3444-3460 compared to H3447R long which has a score of 0.9225 in AAs 3440-3460. Therefore, the H3447R mutation increases disorder in the RELN CTR and may contribute to increased ability to bind GAGs or inhibit AB plaque formation. In contrast, R3446H decreases the disordered region to 3447-3460 with an average score of 0.8583. Arginines in the beta-GAG binding site may therefore contribute to increased disorder in the Reelin CTR. VL-XT, a neural network-based approach, more closely predicted the disordered region of the RELN CTR found in experimental data. Here, the predicted disordered region at the RELN C-terminus started at G3444, which was also shown in the 20 lowest energy NMR structures. Because RELN CTR is extremely well conserved across species and because it is also highly disordered it stands to reason that its function require a highly disordered nature.

### Conclusion

The RELN CTR has an unstructured region beginning with the FVD that also contains the alpha- and beta-GAG binding site. The RELN H3447R mutation creates a new beta-GAG binding site at a position with an orientation that aligns with the other basic amino acids within the GAG binding motif to contribute to GAG interaction. Furthermore, computational analysis confirms that the region from G3444—P3460 is highly disordered. Disordered regions may contribute to increased cellular interactions with GAGs or other proteins. NMA and VL-XT, a machine learning based approach using known disordered regions, were used to estimate potential flexibility within the RELN CTR.

### Example 7. The Reelin CTR Modulates Aβ Aggregation

While the previous experiments suggest a mechanism of protection based on RELN—GAG interaction, it is important to assess if RELN H3447R directly modulates known hallmarks of Alzheimer's disease. Previous studies suggest that disordered molecular regions may modulate Aβ aggregation (38); it has also been reported that purified RELN reduces amyloid 3 (AD) aggregation in vitro (39). Therefore, we examined whether this effect was attributed to the CTR-RELN.

### Methods

Thioflavin T (ThT) binds to amyloids and has also been shown to linearly correlate to amyloid concentration. Therefore, the ThT assay can be used to quantify AB aggregation using previously developed methods (40).

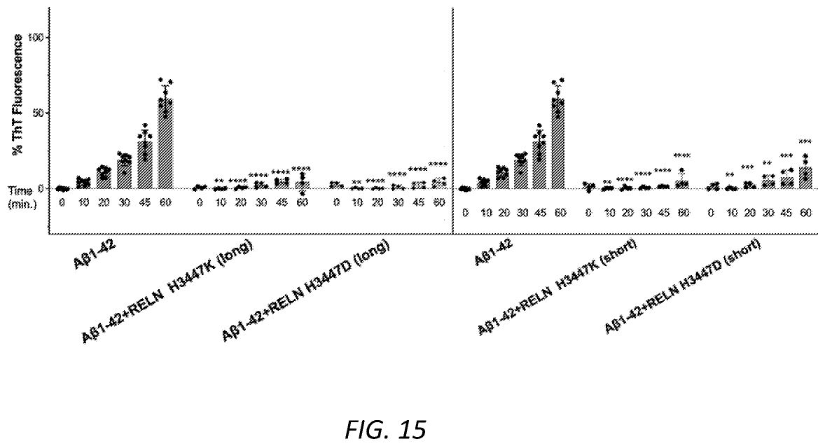

We incubated CTR-RELN with Aβ and monitored aggregation using a ThioflavinT fluorescence assay normalized to the maximum ThT emission of Aβ only. We also assayed Aβ in the presence of morin, a known inhibitor (41), as a negative control. Long and short WT and H3447R RELN CTR peptides were included to better understand the potential role of the H3447R mutation in amyloid pathology (FIGS. 14 and 15. Negative controls included long and short RELN CTR peptides with neutrally charged AAs (histidines) in place of basic AAs in the alpha- and beta-GAG binding sites.

### Results

The RELN peptide alone does not trigger ThT fluorescence (FIG. 15). We found that RELN CTR significantly reduced amyloid aggregation and that the long peptides were as effective as the short peptides (FIGS. 14A-B, 15). RELN H3447R variant peptides were similarly effective at inhibiting ThT aggregation as WT for both long and short variants (FIG. 14A-B). Interestingly, the antiaggregating effect of CTR-RELN was significantly reduced when R3431, K3432, and R3446 were replaced with histidines in the short peptide variant, thus suggesting that N-terminal amino acids may play a primary role in interacting with the amyloid seed (FIG. 14B). H3447D long and H3447K long similarly decrease aggregation, but H3447D short had ˜3-fold higher aggregation compared to H3447K short. Therefore, the charge of the beta-GAG binding site may not be the only contributing factor affecting interaction with Aβ plaques. This hypothesis is further supported by controls (FIG. 14B) where several arginines in the RELN CTR were substituted with histidines, including in the alpha-GAG binding site. In this case, there was significantly higher Aβ aggregation in the sample with short peptides compared to long peptides. Negative controls show minimal ThT fluorescence with the peptide alone without AD.

### Conclusion

The RELN CTR may play a role in AB aggregation, but the mechanism may rely on the overall flexibility of the peptide rather than the charge of the interaction, considering amyloidogenicity of AB is enhanced by sequence enrichment of hydrophobic AA residues (42). It has previously been shown that intrinsically disordered protein (IDPs), such as certain cleaved APP variants, can form seeds for amyloid aggregation. Proteins that have IDPs can potentially interact with amyloid aggregates in a sequence independent manner (Ikeda et al., Sci Rep. 2020 Jul. 23; 10(1):12334). However, in general, net charge and hydrophobicity may increase the likelihood of disordered regions. Therefore, substituting arginines for histidines may have some contribution towards increased aggregation.

RELN peptides with more flexible AAs (H) in key positions at the N-terminus, alpha-, and beta-GAG sites decreased ThT fluorescence to a similar level as WT Long. However, AB aggregation increased ˜4.5-fold with a similar short peptide with the beta—GAG site removed.

Additional peptides can include substituting the most flexible AA in the RELN CTR (G3444) to a less flexible AA such as histidine with intermediate flexibility or proline which would significantly decrease flexibility.

### Example 8. The Reelin CTR Enhances Cell Membrane Penetration

The highly basic structure of the C-Terminal domain of RELN and its ability to interact with membranes might make this domain suitable to induce cell membrane penetration of other proteins (as in protein fusion or as mixture) or nucleic acids (like mRNA).

To evaluate this, HMRECs were cultured in a 24 well plate until 80% confluency complete endothelial growth media 2 (EGM-2, Lonza, Switzerland). For the treatments, 5 μg of mRNA were added to all the formulations. We prepared mRNA/peptide conjugates by mixing each amount of peptide (RKQNYMMNFSRQHGLRHFYNRRRR) (SEQ ID NO:59) and mRNA prior to dilution in EGM-2. HMRECs were cultured with treatment media for 24 hours followed by fluorescence measurements in a microplate reader Synergy H1 (Biotek, VT) upon exciting at 482 nm and recording emission at 520 nm.

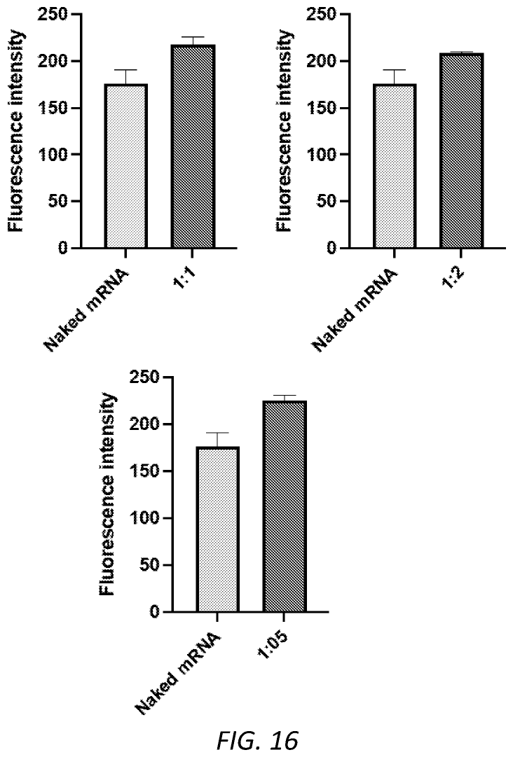

The results, shown in FIG. 16, showed an increase in expression when the mRNA was mixed with the CTR.

### Example 9. Exemplary Sequence Encoding Dab1 Dimerizing Construct

| | |
|---|---|
| LOCUS | pHom_1_hDAB1_1_ 7138 bp ds-DNA circular 26-OCT- |
| | 2022 |
| DEFINITION | Mammalian vector encoding an FKBP homodimerizer domain, for |
| | creating soluble fusion proteins that can be dimerized with a |
| | drug. Also known as pC4-FV1E. |
| ACCESSION | . |
| VERSION | . |
| KEYWORDS | pHom-1 |
| SOURCE | synthetic DNA construct |
| ORGANISM | synthetic DNA construct |
| REFERENCE | 1 (bases 1 to 5371) |
| AUTHORS | Clontech (TaKaRa) |
| TITLE | Direct Submission |
| JOURNAL | Exported Tuesday, Oct. 25, 2022 from SnapGene Viewer 4.3.10 |
| | https://www.snapgene.com |
| COMMENT | Clone into the XbaI site to fuse DmrB to the C-terminus of the |
| | partner protein, or into the Spel site to fuse DmrB to the |
| | N-terminus of the partner protein. |
| COMMENT | ApEinfo: methylated: 1 |
| FEATURES | Location/Qualifiers |
| source | join (1..687, 2455..7138) |
| | /organism = ″synthetic DNA construct″ |
| | /lab_host = ″Mammalian Cells″ |
| | /mol_type = ″other DNA″ |
| | /locus_tag = ″source: synthetic DNA construct″ |
| | /label = ″source: synthetic DNA construct″ |
| | /ApEinfo_label = ″source: synthetic DNA construct″ |
| | /ApEinfo_fwdcolor = ″#00DF87″ |
| | /ApEinfo_revcolor = ″#00DF87″ |
| | /ApEinfo_graphicformat = ″arrow data {{0 0.5 0 1 2 0 0 -1 0 |
| | -0.5} {} 0} width 5 offset 0″ |
| | /ApEinfo_hidden |
| enhancer | 19..322 |
| | /note = ″human cytomegalovirus immediate early enhancer″ |
| | /locus_tag = ″CMV enhancer″ |
| | /label = ″CMV enhancer″ |
| | /ApEinfo_label = ″CMV enhancer″ |
| | /ApEinfo_fwdcolor = ″#00D95A″ |
| | /ApEinfo_revcolor = ″#00D95A″ |
| | /ApEinfo_graphicformat = ″arrow data {{0 0.5 0 1 2 0 0 -1 |
| | 0 -0.5} {} 0} width 5 offset 0″ |
| promoter | 323..526 |
| | /note = ″human cytomegalovirus (CMV) immediate early |
| | promoter″ |
| | /locus_tag = ″CMV promoter″ |
| | /label = ″CMV promoter″ |
| | /ApEinfo_label = ″CMV promoter″ |
| | /ApEinfo fwdcolor = ″#008065″ |
| | /ApEinfo revcolor = ″#008065″ |
| | /ApEinfo graphicformat = ″arrow data { {0 0.5 0 1 2 0 0 -1 |
| | 0 -0.5} {} 0} width 5 offset 0″ |
| 5′UTR | 616..655 |
| | /note = ″5′ untranslated region from the herpes simplex |
| | virus thymidine kinase gene″ |
| | /locus_tag = ″HSV TK 5′-UTR″ |
| | /label = ″HSV TK 5′-UTR″ |
| | /ApEinfo_label = ″HSV TK 5′-UTR″ |
| | /ApEinfo fwdcolor = ″#13AAFE″ |
| | /ApEinfo revcolor = ″#13AAFE″ |
| | /ApEinfo graphicformat = ″arrow data {{0 0.5 0 1 2 0 0 -1 |
| | 0 -0.5} {} 0} width 5 offset 0″ |
| CDS | 676..678 |
| | /codon_start = ″1″ |
| | /product = ″start codon″ |
| | /translation = ″M″ |
| | /locus_tag = ″ATG″ |
| | /label = ″ATG″ |
| | /ApEinfo_label = ″ATG″ |
| | /ApEinfo_fwdcolor = ″#3A93FE″ |
| | /ApEinfo_revcolor = ″#3A93FE″ |
| | /ApEinfo_graphicformat = ″arrow data {{0 0.5 0 1 2 0 0 -1 |
| | 0 -0.5} {} 0} width 5 offset 0″ |
| CDS | 2455..2775 |
| | /codon_start = ″1″ |
| | /product = ″F36V mutant of FK506-binding protein FKBP12″ |
| | /note = ″binds synthetic ligands that do not bind wild- |
| | type |
| | FKBP″ |

/translation = ″GVQVETISPGDGRTFPKRGQTCVVHYTGMLEDGKKVDSSRDRNK
PFKFMLGKQEVIRGWEEGVAQMSVGQRAKLTISPDYAYGATGHPGIIPPHATLVEDVE
LLKLE″ (SEQ ID NO: 80)

| | |
|---|---|
| | /locus_tag = ″DmrB″ |
| | /label = ″DmrB″ |
| | /ApEinfo_label = ″DmrB″ |
| | /ApEinfo_fwdcolor = ″#2D64FE″ |
| | /ApEinfo_revcolor = ″#2D64FE″ |
| | /ApEinfo_graphicformat = ″arrow data {{0 0.5 0 1 2 0 0 -1 |
| | 0 -0.5} {} 0} width 5 offset 0″ |
| CDS | 2782..2808 |
| tag″ | /codon_start = ″1″ |
| | /product = ″HA (human influenza hemagglutinin) epitope |
| | /translation = ″YPYDVPDYA″ (SEQ ID NO: 79) |
| | /locus_tag = ″HA″ |
| | /label = ″HA″ |
| | /ApEinfo_label = ″HA″ |
| | /ApEinfo_fwdcolor = ″#00891F″ |
| | /ApEinfo_revcolor = ″#00891F″ |
| | /ApEinfo_graphicformat = ″arrow data {{0 0.5 0 1 2 0 0 -1 |
| | 0 -0.5} {} 0} width 5 offset 0″ |
| intron | 2840..3412 |
| | /note = ″intron from rabbit beta-globin gene″ |
| | /locus_tag = ″beta-globin intron″ |
| | /label = ″beta-globin intron″ |
| | /ApEinfo_label = ″beta-globin intron″ |
| | /ApEinfo_fwdcolor = ″#00C6C3″ |
| | /ApEinfo_revcolor = ″#00C6C3″ |
| | /ApEinfo_graphicformat = ″arrow data {{0 0.5 0 1 2 0 0 -1 |
| | 0 -0.5} {} 0} width 5 offset 0″ |
| polyA site | 3609..3664 |
| | /note = ″rabbit beta-globin polyadenylation signal″ |
| | /locus_tag = ″beta-globin poly (A) signal″ |
| | /label = ″beta-globin poly (A) signal″ |
| | /ApEinfo_label = ″beta-globin poly (A) signal″ |
| | /ApEinfo_fwdcolor = ″#00DOFA″ |
| | /ApEinfo_revcolor = ″#00DOFA″ |
| | /ApEinfo graphicformat = ″arrow data {{0 0.5 0 1 2 0 0 -1 |
| | 0 -0.5} {} 0} width 5 offset 0″ |
| promoter | complement (3997..4192) |
| | /locus_tag = ″SV40 promoter″ |
| | /label = ″SV40 promoter″ |
| | /ApEinfo_label = ″SV40 promoter″ |
| | /ApEinfo_fwdcolor = ″#00D308″ |
| | /ApEinfo_revcolor = ″#00D308″ |
| | /ApEinfo_graphicformat = ″arrow data {{0 0.5 0 1 2 0 0 -1 |
| | 0 -0.5} {} 0} width 5 offset 0″ |
| rep_origin | 4017..4152 |
| | /note = ″SV40 origin of replication″ |
| | /locus_tag = ″SV40 ori″ |
| | /label = ″SV40 ori″ |
| | /ApEinfo_label = ″SV40 ori″ |
| | /ApEinfo_fwdcolor = ″#2D64FE″ |
| | /ApEinfo_revcolor = ″#2D64FE″ |
| | /ApEinfo_graphicformat = ″arrow data {{0 0.5 0 1 2 0 0 -1 |
| | 0 -0.5} {} 0} width 5 offset 0″ |
| rep origin | complement (4429..5017) |
| | /direction = ″LEFT″ |
| | /note = ″high-copy-number ColE1/pMB1/pBR322/pUC origin of |
| | replication″ |
| | /locus_tag = ″ori″ |
| | /label = ″ori″ |
| | /ApEinfo_label = ″ori″ |
| | /ApEinfo_fwdcolor = ″#EE92FE″ |
| | /ApEinfo_revcolor = ″#EE92FE″ |
| | /ApEinfo_graphicformat = ″arrow data {{0 0.5 0 1 2 0 0 -1 |
| | 0 -0.5} {} 0} width 5 offset 0″ |
| CDS | complement (5188..6060) |
| | /codon_start = ″1″ |
| | /product = ″beta-lactamase″ |
| | /note = ″confers resistance to ampicillin, carbenicillin, |
| | and related antibiotics″ |

/translation = ″MSIQHFRVALIPFFAAFCLPVFAHPETLVKVKDAEDQLGARVGY
IELDLNSGKILESFRPEERFQVEVAMMSTFKVLLCGAVLSRIDAGQEQLGRRIHYSQN
DLVEYSPVTEKHLTDGMTVRELCSAAITMSDNTAANLLLTTIGGPKELTAFLHNMGDH
VTRLDRWEPELNEAIPNDERDTTMPVAMATTLRKLLTGELLTLASRQQLIDWMEADKV
AGPLLRSALPAGWFIADKSGAGERGSRGIIAALGPDGKPSRIVVIYTTGSQATMDERN
RQIAEIGASLIKHW″ (SEQ ID NO: 81)

| | |
|---|---|
| | /locus_tag = ″AmpR″ |
| | /label = ″AmpR″ |
| | /ApEinfo_label = ″AmpR″ |
| | /ApEinfo_fwdcolor = ″#A600FA″ |
| | /ApEinfo_revcolor = ″#A600FA″ |
| | /ApEinfo_graphicformat = ″arrow data {{0 0.5 0 1 2 0 0 -1 |
| | 0 -0.5} {} 0} width 5 offset 0″ |
| promoter | complement (6061..6165) |
| | /gene = ″bla″ |
| | /locus_tag = ″AmpR promoter″ |
| | /label = ″AmpR promoter″ |
| | /ApEinfo_label = ″AmpR promoter″ |
| | /ApEinfo_fwdcolor = ″#4856FE″ |
| | /ApEinfo_revcolor = ″#4856FE″ |
| | /ApEinfo_graphicformat = ″arrow data {{0 0.5 0 1 2 0 0 -1 |
| | 0 -0.5} {} 0} width 5 offset 0″ |
| rep_origin | complement (6497..6952) |
| | /direction = ″LEFT″ |
| | /note = ″f1 bacteriophage origin of replication; arrow |
| | indicates direction of (+) strand synthesis″ |
| | /locus_tag = ″f1 ori″ |
| | /label = ″f1 ori″ |
| | /ApEinfo_label = ″f1 ori″ |
| | /ApEinfo_fwdcolor = ″#FE09C9″ |
| | /ApEinfo_revcolor = ″#FE09C9″ |
| | /ApEinfo_graphicformat = ″arrow data {{0 0.5 0 1 2 0 0 -1 |
| | 0 -0.5} {} 0} width 5 offset 0″ |
| misc_feature | 688..2448 |
| | /locus_tag = ″hDAB1″ |
| | /label = ″hDAB1″ |
| | /ApEinfo_label = ″hDAB1″ |
| | /ApEinfo_fwdcolor = ″#ffff00″ |
| | /ApEinfo_revcolor = ″green″ |
| | /ApEinfo_graphicformat = ″arrow data { {0 0.5 0 1 2 0 0 -1 |
| | 0 -0.5} {} 0} width 5 offset 0″ |

ORIGIN

| | |
|---|---|
| 1 | acgcgttcga gctcgccccg ttacataact tacggtaaat ggcccgcctg gctgaccgcc |
| 61 | caacgacccc cgcccattga cgtcaataat gacgtatgtt cccatagtaa cgccaatagg |
| 121 | gactttccat tgacgtcaat gggtggagta tttacggtaa actgcccact tggcagtaca |
| 181 | tcaagtgtat catatgccaa gtacgccccc tattgacgtc aatgacggta aatggcccgc |
| 241 | ctggcattat gcccagtaca tgaccttatg ggactttcct acttggcagt acatctacgt |
| 301 | attagtcatc gctattacca tggtgatgcg gttttggcag tacatcaatg ggcgtggata |
| 361 | gcggtttgac tcacggggat ttccaagtct ccaccccatt gacgtcaatg ggagtttgtt |
| 421 | ttggcaccaa aatcaacggg actttccaaa atgtcgtaac aactccgccc cattgacgca |
| 481 | aatgggcggt aggcgtgtac ggtgggaggt ctatataagc agagctcgtt tagtgaaccg |
| 541 | tcagatcgcc tggagacgcc atccacgctg ttttgacctc catagaagac accgggaccg |
| 601 | atccagcctc cgggggatct tggtggcgtg aaactcccgc acctcttcgg ccagcgaatt |
| 661 | ccagaagccg ccaccatggc ttctagatca actgagacag aacttcaagt agctgtgaaa |
| 721 | accagcgcca agaaagactc cagaaagaaa ggtcaggatc gcagtgaagc cactttgata |
| 781 | aagaggttta aaggtgaagg ggtccggtac aaagccaaat tgatcgggat tgatgaagtt |
| 841 | tccgcagctc ggggagacaa gttatgtcaa gattccatga tgaaactcaa gggcgttgtt |
| 901 | gctggcgctc gttccaaagg agaacacaaa cagaaaatct ttttaaccat ctcctttgga |
| 961 | ggaatcaaaa tctttgatga gaagacaggg gcccttcagc atcatcatgc tgttcatgaa |
| 1021 | atatcctaca ttgcaaagga cattacagat caccgggcct ttggatatgt ttgtgggaag |
| 1081 | gaagggaatc acagatttgt ggccataaaa acagcccagg cggctgaacc tgttattctg |
| 1141 | gacttgagag atctctttca actcatttat gaattgaagc aaagagaaga attagaaaaa |
| 1201 | aaggcacaaa aggataagca gtgtgaacaa gctgtgtacc agacaatatt ggaagaggat |
| 1261 | gttgaagatc ctgtgtacca gtacattgtg tttgaggctg gacacgagcc aatccgtgat |
| 1321 | cccgaaacgg aagaaaacat ttatcaggtt cccaccagcc aaaagaagga aggtgtttat |
| 1381 | gatgtgccaa aaagtcaacc tgtaagtaat ggctattcgt ttgaggattt tgaagaacgg |
| 1441 | tttgctgcag ccaccccgaa cagaaacctg cccacagact ttgatgagat ttttgaggca |
| 1501 | acgaaggctg tgacccaatt agaacttttt ggggacatgt ccacaccccc tgatataacc |
| 1561 | tctcccccca ctcctgcaac tccaggtgat gcctttatcc catcttcatc tcagaccctt |
| 1621 | ccagcgagtg cagatgtgtt tagttctgta cctttcggca ctgctgctgt accctcaggt |
| 1681 | tacgttgcaa tgggcgctgt cctcccgtcc ttctggggtc agcagcccct cgtccaacag |
| 1741 | cagatggtca tgggtgccca gccaccagtc gctcaggtga tgccgggggc tcagcccatc |
| 1801 | gcatggggcc agccgggtct ctttcctgcc actcagcagc cctggccaac tgtggccggg |
| 1861 | cagtttccgc cagccgcctt catgcccaca caaactgtta tgcctttgcc agctgccatg |
| 1921 | ttccaaggtc ccctcacccc ccttgccacc gtcccaggca cgagtgactc caccaggtca |
| 1981 | agtccacaga ccgacaagcc caggcagaaa atgggcaaag aaacgtttaa ggatttccag |
| 2041 | atggcccagc ctccgcccgt gccctcccgc aaacccgacc agccctccct cacctgtacc |
| 2101 | tcagaggcct tctccagtta cttcaacaaa gtcggggtgg cacaggatac agacgactgt |
| 2161 | gatgactttg acatctccca gttgaatttg acccctgtga cttctaccac accatcgacc |
| 2221 | aactcacctc caaccccagc ccctagacag agctctccat ccaaatcatc tgcatcccat |
| 2281 | gccagtgatc ctaccacaga tgacatcttt gaagagggct ttgaaagtcc cagcaaaagc |
| 2341 | gaagagcaag aagctcctga tggatcacag gcctcatcca acagtgatcc atttggtgag |
| 2401 | cccagtgggg agcccagtgg tgataatata agtccacagg ccggtagctc tagaggagtg |
| 2461 | caggtggaga ctatctcccc aggagacggg cgcaccttcc ccaagcgcgg ccagacctgc |
| 2521 | gtggtgcact acaccgggat gcttgaagat ggaaagaaag ttgattcctc ccgggacaga |
| 2581 | aacaagccct ttaagtttat gctaggcaag caggaggtga tccgaggctg ggaagaaggg |
| 2641 | gttgcccaga tgagtgtggg tcagagagcc aaactgacta tatctccaga ttatgcctat |
| 2701 | ggtgccactg ggcacccagg catcatccca ccacatgcca ctctcgtctt cgatgtggag |
| 2761 | cttctaaaac tggaaactag ttatccgtac gacgtaccag actacgcata agaaaagtga |
| 2821 | ggatcctgag aacttcaggg tgagtttggg gacccttgat tgttctttct ttttcgctat |
| 2881 | tgtaaaattc atgttatatg gagggggcaa agttttcagg gtgttgttta gaatgggaag |
| 2941 | atgtcccttg tatcaccatg gaccctcatg ataattttgt ttctttcact ttctactctg |
| 3001 | ttgacaacca ttgtctcctc ttattttctt ttcattttct gtaacttttt cgttaaactt |
| 3061 | tagcttgcat ttgtaacgaa tttttaaatt cacttttgtt tatttgtcag attgtaagta |
| 3121 | ctttctctaa tcactttttt ttcaaggcaa tcagggtata ttatattgta cttcagcaca |
| 3181 | gttttagaga acaattgtta taattaaatg ataaggtaga atatttctgc atataaattc |
| 3241 | tggctggcgt ggaaatattc ttattggtag aaacaactac atcctggtca tcatcctgcc |
| 3301 | tttctcttta tggttacaat gatatacact gtttgagatg aggataaaat actctgagtc |
| 3361 | caaaccgggc ccctctgcta accatgttca tgccttcttc tttttcctac agctcctggg |
| 3421 | caacgtgctg gttgttgtgc tgtctcatca ttttggcaaa ggattcactc ctcaggtgca |
| 3481 | ggctgcctat cagaaggtgg tggctggtgt ggccaatgcc ctggctcaca aataccactg |
| 3541 | agatcttttt ccctctgcca aaaattatgg ggacatcatg aagccccttg agcatctgac |
| 3601 | ttctggctaa taaaggaaat ttattttcat tgcaatagtg tgttggaatt ttttgtgtct |
| 3661 | ctcactcgga aggacatatg ggagggcaaa tcatttaaaa catcagaatg agtatttggt |
| 3721 | ttagagtttg gcaacatatg ccatatgctg gctgccatga acaaaggtgg ctataaagag |
| 3781 | gtcatcagta tatgaaacag ccccctgctg tccattcctt attccataga aaagccttga |
| 3841 | cttgaggtta gatttttttt atattttgtt ttgtgttatt tttttcttta acatccctaa |
| 3901 | aattttcctt acatgtttta ctagccagat ttttcctcct ctcctgacta ctcccagtca |
| 3961 | tagctgtccc tcttctctta tgaagatccc tcgaggagct ttttgcaaaa gccctaggcc |
| 4021 | tccaaaaaag cctcttcact acttctggaa tagctcagag gccgaggcgg cctcggcctc |
| 4081 | tgcataaata aaaaaaatta gtcagccatg gggcggagaa tgggcggaac tgggcggagt |
| 4141 | taggggcggg atgggcggag ttaggggcgg gactatggtt gctgactaat tgctgcatta |
| 4201 | atgaatcggc caacgcgcgg ggagaggcgg tttgcgtatt gggcgctctt ccgcttcctc |
| 4261 | gctcactgac tcgctgcgct cggtcgttcg gctgcggcga gcggtatcag ctcactcaaa |
| 4321 | ggcggtaata cggttatcca cagaatcagg ggataacgca ggaaagaaca tgtgagcaaa |
| 4381 | aggccagcaa aaggccagga accgtaaaaa ggccgcgttg ctggcgtttt tccataggct |
| 4441 | ccgcccccct gacgagcatc acaaaaatcg acgctcaagt cagaggtggc gaaacccgac |
| 4501 | aggactataa agataccagg cgtttccccc tggaagctcc ctcgtgcgct ctcctgttcc |
| 4561 | gaccctgccg cttaccggat acctgtccgc ctttctccct tcgggaagcg tggcgctttc |
| 4621 | tcatagctca cgctgtaggt atctcagttc ggtgtaggtc gttcgctcca agctgggctg |
| 4681 | tgtgcacgaa ccccccgttc agcccgaccg ctgcgcctta tccggtaact atcgtcttga |
| 4741 | gtccaacccg gtaagacacg acttatcgcc actggcagca gccactggta acaggattag |
| 4801 | cagagcgagg tatgtaggcg gtgctacaga gttcttgaag tggtggccta actacggcta |
| 4861 | cactagaagg acagtatttg gtatctgcgc tctgctgaag ccagttacct tcggaaaaag |
| 4921 | agttggtagc tcttgatccg gcaaacaaac caccgctggt agcggtggtt tttttgtttg |
| 4981 | caagcagcag attacgcgca gaaaaaaagg atctcaagaa gatcctttga tcttttctac |
| 5041 | ggggtctgac gctcagtgga acgaaaactc acgttaaggg attttggtca tgagattatc |
| 5101 | aaaaaggatc ttcacctaga tccttttaaa ttaaaaatga agttttaaat caatctaaag |
| 5161 | tatatatgag taaacttggt ctgacagtta ccaatgctta atcagtgagg cacctatctc |
| 5221 | agcgatctgt ctatttcgtt catccatagt tgcctgactc cccgtcgtgt agataactac |
| 5281 | gatacgggag ggcttaccat ctggccccag tgctgcaatg ataccgcgag acccacgctc |
| 5341 | accggctcca gatttatcag caataaacca gccagccgga agggccgagc gcagaagtgg |
| 5401 | tcctgcaact ttatccgcct ccatccagtc tattaattgt tgccgggaag ctagagtaag |
| 5461 | tagttcgcca gttaatagtt tgcgcaacgt tgttgccatt gctacaggca tcgtggtgtc |
| 5521 | acgctcgtcg tttggtatgg cttcattcag ctccggttcc caacgatcaa ggcgagttac |
| 5581 | atgatccccc atgttgtgca aaaaagcggt tagctccttc ggtcctccga tcgttgtcag |
| 5641 | aagtaagttg gccgcagtgt tatcactcat ggttatggca gcactgcata attctcttac |
| 5701 | tgtcatgcca tccgtaagat gcttttctgt gactggtgag tactcaacca agtcattctg |
| 5761 | agaatagtgt atgcggcgac cgagttgctc ttgcccggcg tcaatacggg ataataccgc |
| 5821 | gccacatagc agaactttaa aagtgctcat cattgccacc tccacctgaa aacgttcttc |
| 5881 | ggggcgaaaa ctctcaagga tcttaccgct gttgagatcc agttcgatgt aacccactcg |
| 5941 | tgcacccaac tgatcttcag catcttttac tttcaccagc gtttctgggt gagcaaaaac |
| 6001 | aggaaggcaa aatgccgcaa aaaagggaat aagggcgaca cggaaatgtt gaatactcat |
| 6061 | actcttcctt tttcaatatt attgaagcat ttatcagggt tattgtctca tgagcggata |
| 6121 | catatttgaa tgtatttaga aaaataaaca aataggggtt ccgcgcacat ttccccgaaa |
| 6181 | agtgccacct gacgtctaag aaaccattat tatcatgaca ttaacctata aaaataggcg |
| 6241 | tatcacgagg ccctttcgtc tcgcgcgttt cggtgatgac ggtgaaaacc tctgacacat |
| 6301 | gcagctcccg gagacggtca cagcttgtct gtaagcggat gccgggagca gacaagcccg |
| 6361 | tcagggcgcg tcagcgggtg ttggcgggtg tcggggctgg cttaactatg cggcatcaga |
| 6421 | gcagattgta ctgagagtgc accatatgcg gtgtgaaata ccgcacagat gcgtaaggag |
| 6481 | aaaataccgc atcaggaaat tgtaaacgtt aatattttgt taaaattcgc gttaaatttt |
| 6541 | tgttaaatca gctcattttt taaccaatag gccgaaatcg gcaaaatccc ttataaatca |
| 6601 | aaagaataga ccgagatagg gttgagtgtt gttccagttt ggaacaagag tccactatta |
| 6661 | aagaacgtgg actccaacgt caaagggcga aaaaccgtct atcagggcga tggcccacta |
| 6721 | cgtgaaccat caccctaatc aagttttttg gggtcgaggt gccgtaaagc actaaatcgg |
| 6781 | aaccctaaag ggagcccccg atttagagct tgacggggaa agccggcgaa cgtggcgaga |
| 6841 | aaggaaggga agaaagcgaa aggagcgggc gctagggcgc tggcaagtgt agcggtcacg |
| 6901 | ctgcgcgtaa ccaccacacc cgccgcgctt aatgcgccgc tacagggcgc gtcgcgccat |
| 6961 | tcgccattca ggctgcgcaa ctgttgggaa gggcgatcgg tgcgggcctc ttcgctatta |
| 7021 | cgccagctgg cgaaaggggg atgtgctgca aggcgattaa gttgggtaac gccagggttt |
| 7081 | tcccagtcac gacgttgtaa aacgacggcc agtgaattgt aatacgactc actatagg |

(SEQ ID NO: 78)
//

The following is a codon-optimized sequence encoding the Dab1—FKBP fusion protein:

(SEQ ID NO: 60)
ATG GCC AGT AGG TCA ACT GAA ACG GAG CTC CAA GTG GCA GTT AAA
ACC AGC GCG AAG AAG GAT AGT CGC AAG AAG GGA CAG GAC AGA TCA
GAA GCT ACT CTC ATC AAA AGG TTC AAG GGC GAG GGG GTG AGG TAT
AAA GCG AAA TTG ATT GGT ATT GAC GAA GTA TCA GCT GCA CGA GGA
GAC AAG CTT TGT CAA GAC TCT ATG ATG AAA CTT AAA GGT GTG GTA
GCC GGT GCA CGG TCA AAG GGA GAA CAC AAA CAG AAG ATA TTC TTG
ACA ATT TCC TTT GGG GGA ATA AAG ATT TTT GAC GAA AAG ACT GGT
GCC CTT CAA CAT CAT CAT GCG GTC CAC GAA ATC TCT TAT ATA GCA
AAG GAC ATA ACT GAT CAC AGG GCC TTC GGG TAT GTT TGC GGT AAG
GAA GGT AAC CAT CGC TTC GTG GCC ATT AAA ACT GCT CAG GCG GCA
GAA CCC GTT ATA CTC GAT CTT CGC GAC TTG TTT CAA CTC ATC TAC
GAG TTG AAG CAA AGA GAG GAG CTT GAA AAG AAA GCC CAG AAA GAT
AAA CAG TGC GAA CAA GCT GTA TAC CAG ACA ATA CTC GAG GAA GAC
GTT GAA GAC CCT GTA TAT CAG TAC ATA GTT TTT GAG GCA GGG CAT
GAA CCG ATA AGA GAT CCA GAG ACT GAG GAA AAC ATT TAT CAG GTC
CCG ACA AGC CAA AAA AAG GAG GGT GTG TAC GAT GTG CCA AAG TCC
CAG CCC GTC AGT AAT GGC TAC AGT TTC GAG GAC TTC GAA GAG CGG
TTC GCC GCA GCT ACG CCC AAC CGC AAC CTC CCA ACC GAT TTC GAC
GAG ATA TTT GAG GCA ACC AAG GCC GTT ACA CAG CTC GAA CTC TTC
GGC GAT ATG TCT ACT CCA CCT GAC ATC ACG AGC CCT CCT ACA CCC
GCG ACT CCG GGC GAC GCA TTC ATT CCA TCC TCC AGT CAA ACC CTC
CCG GCT TCC GCG GAC GTG TTT TCC TCT GTC CCG TTC GGA ACC GCA
GCG GTT CCG TCT GGA TAT GTT GCG ATG GGG GCA GTG TTG CCC TCT
TTT TGG GGC CAA CAG CCA CTG GTT CAA CAG CAG ATG GTC ATG GGC
GCC CAG CCC CCG GTT GCT CAG GTC ATG CCA GGA GCT CAA CCC ATA
GCG TGG GGC CAG CCA GGA CTG TTC CCC GCG ACT CAG CAA CCC TGG
CCA ACC GTG GCT GGG CAA TTT CCA CCA GCG GCA TTC ATG CCT ACG
CAG ACC GTC ATG CCT CTG CCC GCT GCT ATG TTT CAA GGC CCC CTT
ACA CCT CTG GCA ACG GTG CCT GGA ACT TCC GAC TCC ACA AGA AGT
TCC CCG CAG ACC GAT AAG CCG CGG CAA AAA ATG GGT AAA GAA ACT
TTT AAA GAT TTT CAA ATG GCG CAA CCT CCG CCG GTA CCG AGC AGA
AAA CCG GAC CAG CCA AGC CTC ACC TGC ACA TCT GAA GCC TTT TCT
AGC TAT TTT AAT AAG GTC GGT GTA GCT CAA GAC ACG GAT GAC TGT
GAT GAT TTT GAT ATC TCA CAA CTC AAC TTG ACA CCA GTC ACC AGC
ACA ACT CCC AGT ACT AAC AGT CCG CCA ACT CCC GCG CCG CGC CAG
TCC TCA CCG TCT AAA TCT TCC GCT TCA CAT GCA TCT GAT CCT ACG
ACG GAC GAT ATC TTC GAA GAA GGT TTT GAA TCA CCG AGT AAG AGC
GAA GAA CAA GAA GCT CCA GAT GGT TCT CAA GCG TCC TCC AAT AGC
GAT CCT TTT GGG GAA CCA AGC GGG GAG CCA TCT GGT GAC AAT ATA
TCA CCA CAG GCA GGC AGT AGT CGC GGC GTC CAA GTT GAG ACG ATT
TCC CCT GGT GAT GGA CGG ACA TTC CCC AAG CGG GGC CAG ACC TGT
GTT GTT CAC TAC ACG GGA ATG TTG GAA GAC GGT AAA AAA GTC GAC
AGT AGT CGC GAT CGG AAC AAG CCA TTC AAA TTC ATG CTG GGC AAA
CAA GAA GTT ATC CGG GGC TGG GAA GAA GGC GTC GCA CAA ATG TCT
GTA GGG CAA AGA GCA AAA CTC ACA ATA AGT CCA GAC TAC GCG TAT
GGC GCA ACT GGC CAC CCT GGT ATT ATA CCG CCA CAT GCG ACG CTG
GTC TTC GAC GTA GAA TTG TTG AAG TTG GAA

### Example 10. Analysis of miniRELN Constructs in HREC Cells

Both in vitro overexpression and recombinant proteins were used to evaluate mini-RELN constructs in human retinal endothelial cells (HREC). The constructs used in Examples 10-12 were as shown in Table 5.

TABLE 5
Exemplary constructs

| Name | Construct |
|---|---|
| 225Q | hG2Fc-R5-6-C-termH3447R |
| 225R | flag-CR-50-R5-6-CtermH3447R |
| 225S | FcIgG-RAP-CTRH3447R |
| 225T | RAP-FcIgG-CTRH3447R |
| 225Xf | IL2ss-FcigG-APOE-R154S-CTRH3447R |
| 225Yf | IL2ss-FcigG-APOE-CTRH3447R |
| 225Z | 6xHISCR-50-R5-6-CtermH3447R |
| 225SU | IL2signalpeptide-6XHistag(SEQ ID NO: 62)- |
| | hReelin(AALeu1220-Ile2660) C-term-REELIN H3447R |
| 225SV | IL2signalpeptide-6XHistag(SEQ ID NO: 62)- |
| | hReelin(AALeu1220-Ile2660) Cterm-REELINWT |
| 225ZZ | Il2ss6xHISMini-RELNFspCR50R5-6CtermH3447R |
| 225SW | IL2ss-6xHis-hReelin R3-R6 CT-H3447R |
| 233A | IL2ss-6xHis-hReelinR3-R6CT-WT |
| 233C | Il2ss-6xHISR5-6CtermH3447R |
| 233D | IL2ss-6xHis-hReelinR6-R6CT-H3447R |
| 233E | Il2ss-6xHIS-R5-6-FspCR50-CtermH3447R |
| 233F | IL2ss-6xHis-FspCR50-R6-R6 Cterm RELN-H3447R |
| 225RR | Il2ss-flagMini-RELNFspCR50R5-6CtermH3447R |

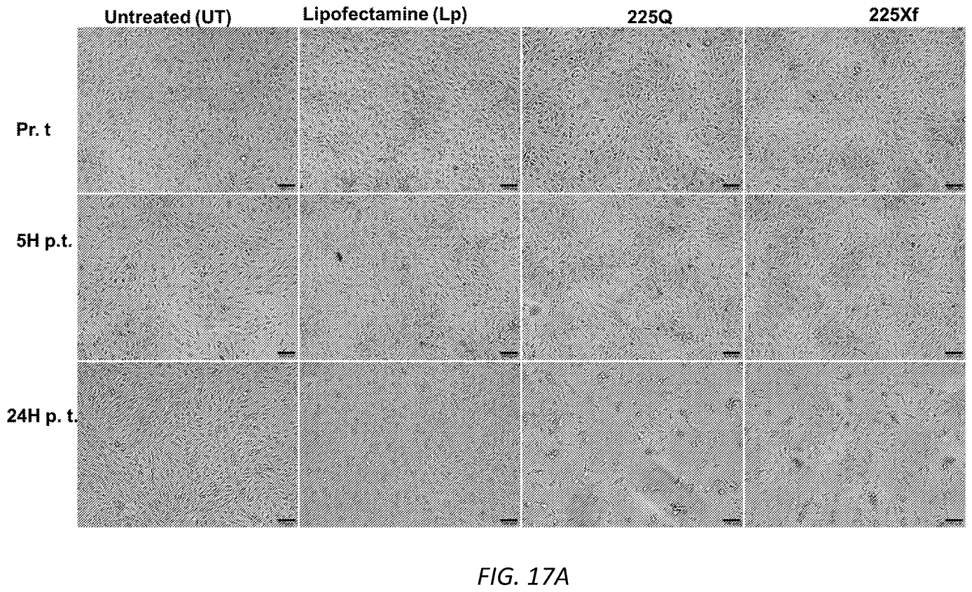

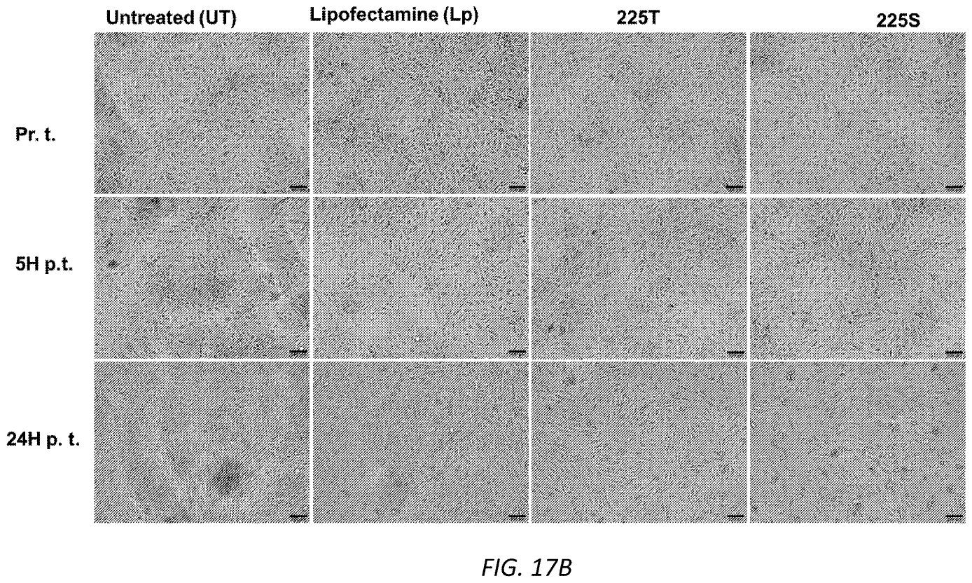

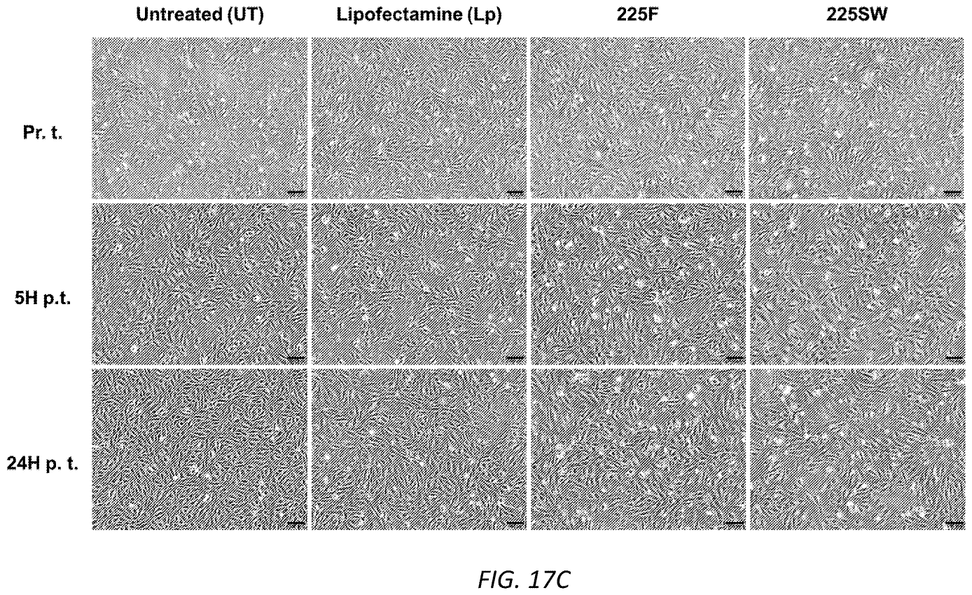

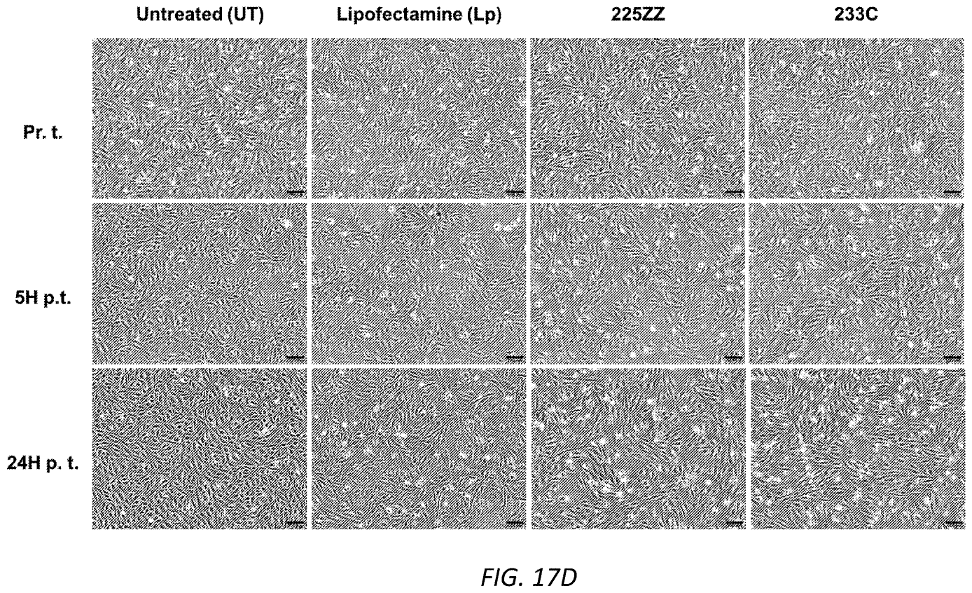

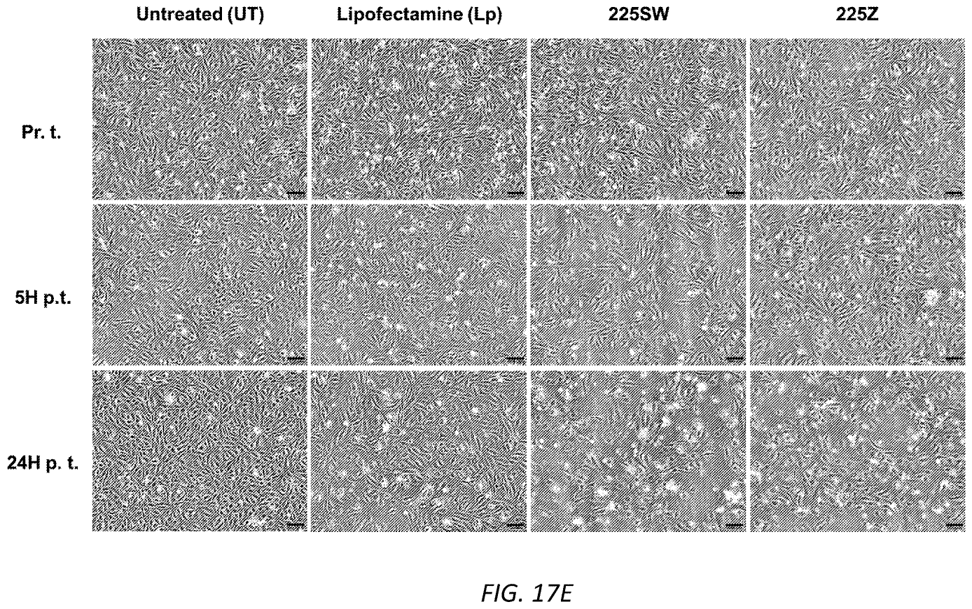

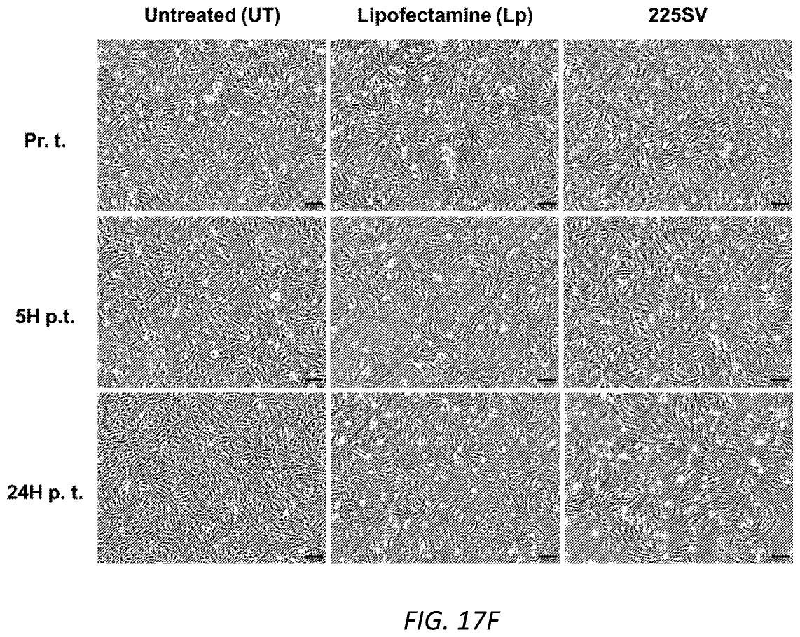

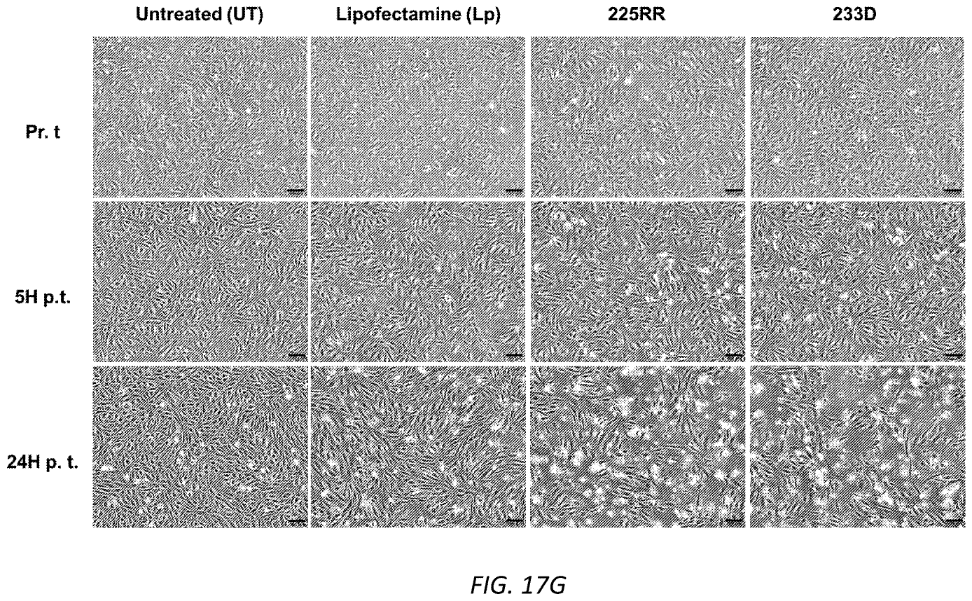

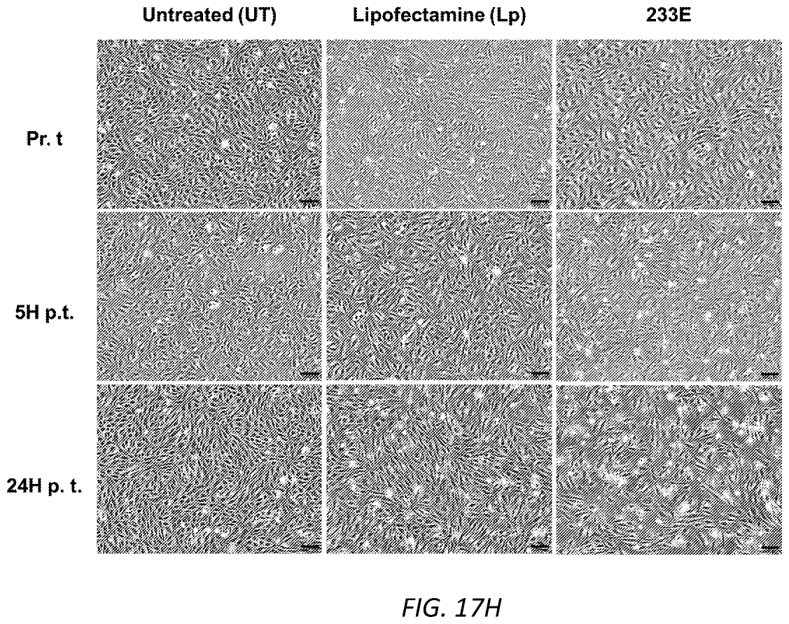

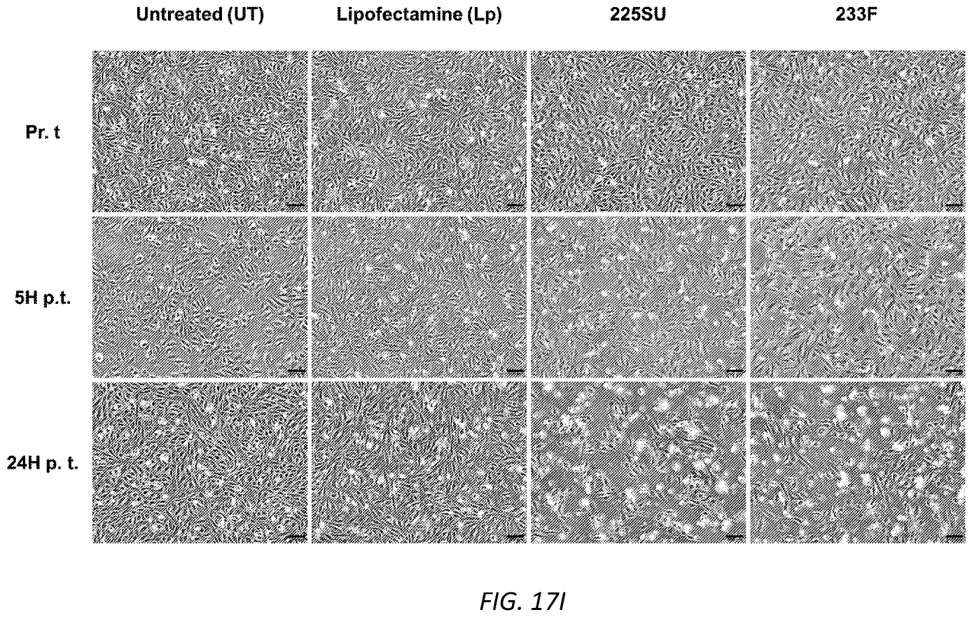

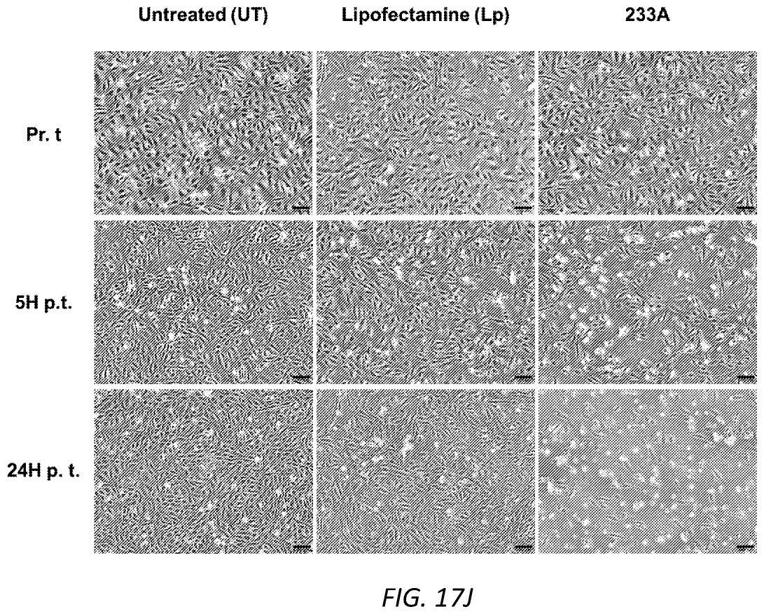

For in vitro overexpression, the HRECs were cultured in EBM-2 media enriched with EGM-2 Single Quots (Lonza) at 37° C. and 5% CO2 and experimentally tested at passage 7. 24 h prior experiment, cells were plated on a 6-well plate at 500,000 cells/mL. The cell transfection of plasmids was done with Lipofectamine 2000 (5 μL, Life technologies) according to the manufacturer. In detail, one day prior to transfection, cells were seeded to be 70% confluent at moment of transfection. The day of transfection lipofectamine was diluted and incubated for 10 minutes at room temperature, to allow the formation of the lipophilic complex. Subsequently, each plasmid (1 μg, Invivogen) was added to the lipid complex of Lipofectamine and incubated for 10 minutes. We used OPTIMEM media (Gibco) to block the transfection after 5 hours, to allow cells to recover and facilitate replication. Prior 5 h and 24 h post-transfection, cells were imaged using 10× magnification light microscope (Olympus CKX53). The results, shown in FIGS. 17A-J, demonstrated integrity of mammalian cells expressing all mini-RELN constructs.

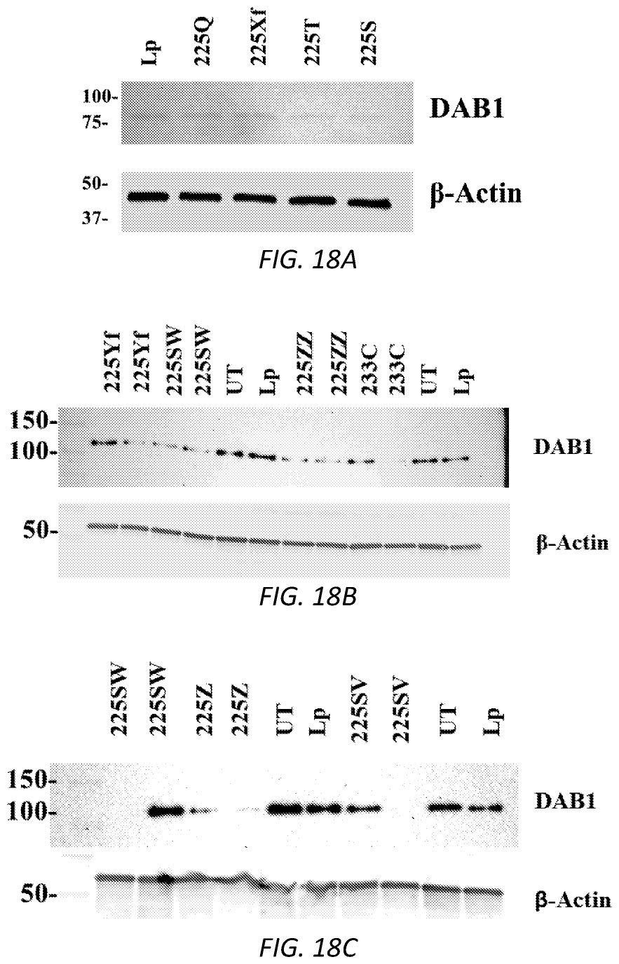

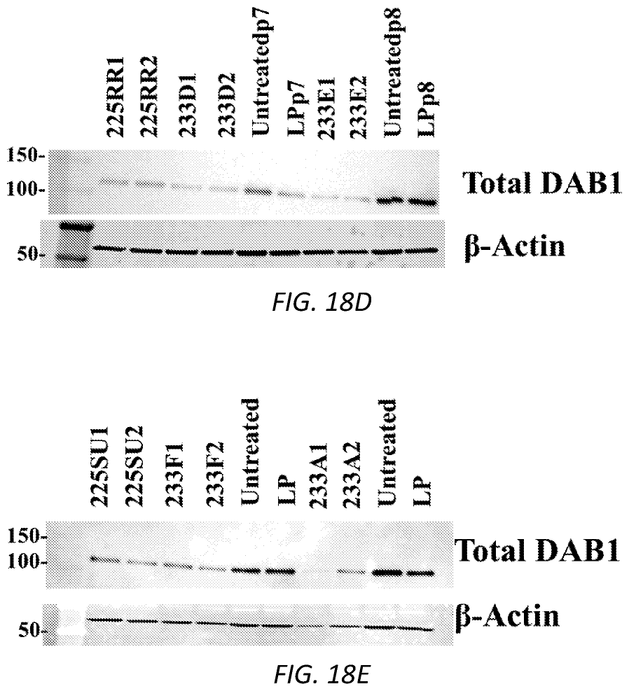

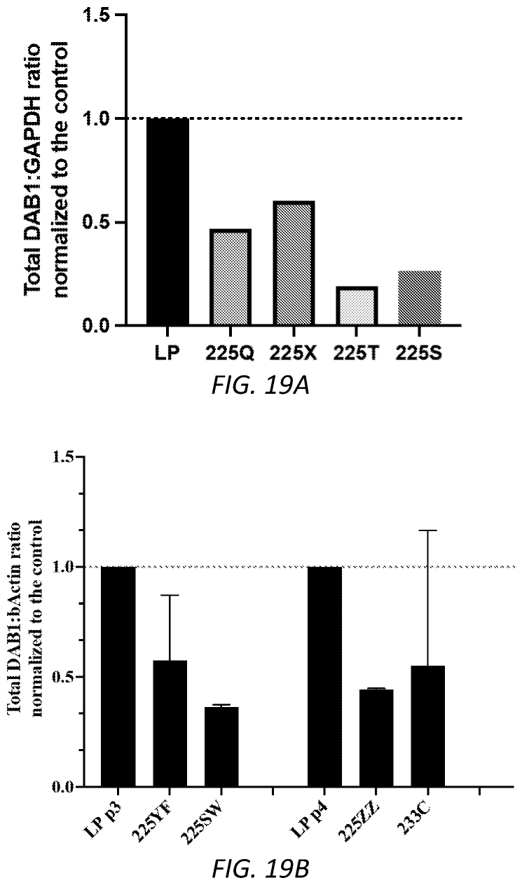

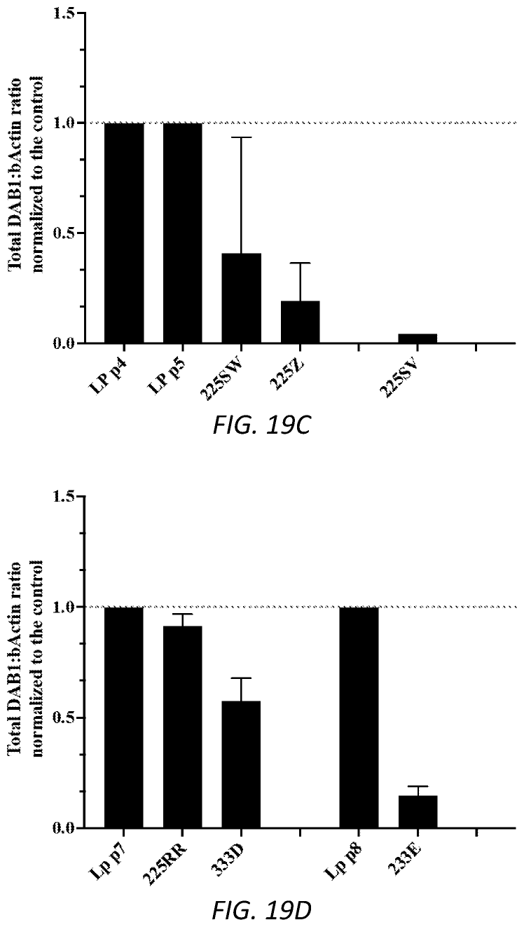

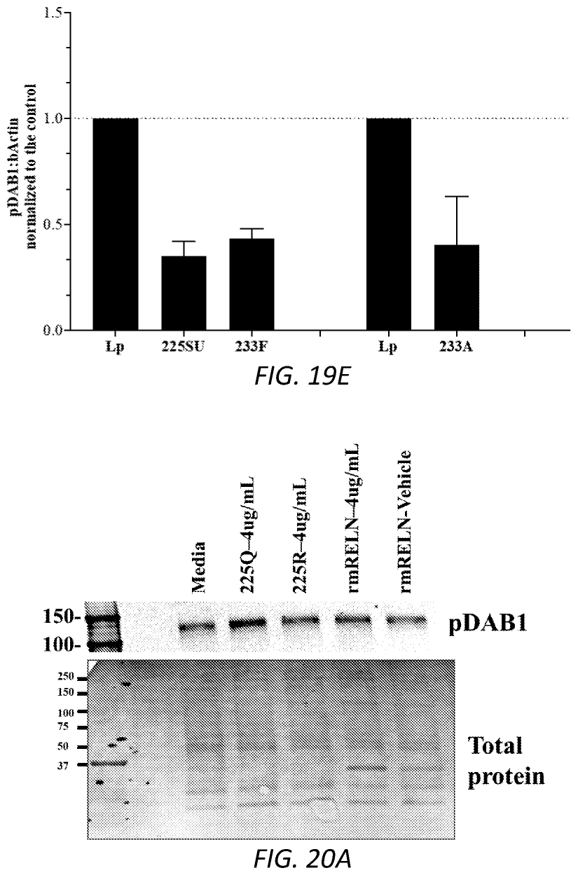

24 h post-transfection, cells were harvested for western blotting analysis. Total protein levels were quantified via BCA assay. For Western Blotting, 7 μg protein homogenates were prepared under reducing and denaturing conditions using Laemmli buffer enriched with 10 mM DTT and 5 min boiling. Samples were electrophoretically separated in a 4-20% Gel at 90V and protein transferred to a PVDF membrane using iBlot™ 2 Dry Blotting System. The membrane was blocked using Intercept® (TBS) Blocking Buffer for 2 hours. We detected β-actin (1:5000, mouse, #66009-1-Ig, Proteintech), and total DAB1(1:100, SC-red tube; sc-271136 Dab1 (G-5), Santa Cruz). Antibodies were diluted using Intercept® (TBS) Blocking Buffer and incubated overnight at 4C under gentle shaking. Prior to secondary antibody incubation, membranes were washed 2 times using TBS-T for 10 minutes. As secondary antibodies, we used IR Dye 800n CW Goat-anti-Rabbit (925-32211, Licor), IR Dye 680 CW Goat-anti-Mouse (925-68070, Licor) both diluted 1:10000 in blocking buffer and HRP (m-IgG1 BP-HRP-SC-525408, Santa Cruz) diluted 1:1000 also in blocking buffer. for 1 h at room temperature under gentle shaking. Membranes were washed three times with TBS-T prior IR detection using Odyssey imager. To quantify the results, each band was quantified using ImageJ and data expressed as DAB1:β-actin ratio normalized to the control. FIGS. 18A-E, showed a reduction in total Dab1 presumably as a result of increased signaling activity of Dab1 triggered by mini-RELN constructs. The reduction of total DAB1 was quantified as shown in FIGS. 19A-E. These experiments showed that the mini-RELN constructs could function directly in cells that express them. The results, shown in FIGS. 18A-E, demonstrate that expression of the mini-RELN constructs triggers sustained activation of RELN signaling detected as significant turnover and reduction of total DAB1. Data is showing reduced levels of DAB1 in the presence of the mini-RELN constructs as compared to lipofectamine (Lp) for all constructs. These findings allowed us to conclude that all mini RELN constructs tested were active.

All the tested constructs triggered significant activation, with 225Z being the most active. 225Z include the CR50 domain of RELN to induce oligomerization, R5-6 to bind the receptors, and the RELN C-term with the COLBOS variant to bind HSPGs.

Our analyses showed that the modular design described herein was effective at activating RELN signaling also when including RAP to bind the receptor, Fc to induce oligomers, and RELN C-term with the COLBOS variant to bind HSPGs. In this design Fc replaced CR50 of RELN and RAP replaced the receptor binding domain. In this design miniRELN constructs are shown to be efficacious when produced by a cell like HREC and signal in the same cell in an autocrine fashion.

The experimental evidence showing that we can replace modules within the mini-RELN domain according to their function (e.g., Fc vs. CR-50 and RAP vs. R5-6) without compromising efficacy demonstrates that our chosen modules are sufficient to support RELN signaling.

For in vitro screening of constructs using recombinant mini-RELN on HRECs, the cells were cultured in EBM-2 media enriched with EGM-2 Single Quots (Lonza) at 37° C. and 5% CO2 and experimentally tested at passage 7. 24 h prior experiment, cells were plated on a 6-well plate at 500,000 cells/mL. Two hours before the experiment, the cells were washed with PBS to remove residual growth media and then incubated with a starving media including EBM-2, 1% GlutaMax+0.2% Normicin. Treatments was conducted using recombinant mini RELN constructs obtained from Innovagen and Recombinant Mouse Reelin Protein from R&D systems (3820-MR-025/CF). Each treatment was incubated for 5 minutes at 4 ug/mL concentration and cells were subsequently harvested in lysis buffer was applied constituted of RIPA (Cell signaling), proteases inhibitors (cOmplete™, Mini, EDTA-free Protease Inhibitor Cocktail, Roche), phosphatases inhibitors (PhosSTOP, Roche), 1 μL of proteasome inhibitor MG-132 10 mM, 1% Triton X 100. The lysates were scraped and centrifugated at 15000 rpm for 10 minutes at 4° C. Total protein levels were quantified via BCA assay. For Western Blotting, 3.5 ug protein homogenates were prepared under reducing and denaturing conditions using Laemmli buffer enriched with 10 mM DTT and 5 min boiling. Samples were electrophoretically separated in a 4-20% Gel at 90V and protein transferred to a PVDF membrane using iBlot™ 2 Dry Blotting System. The membrane was blocked using Intercept® (TBS) Blocking Buffer for 2 hours. We detected pDAB1 (Tyr232; 1:1000, rabbit, #3325 Cell signaling), and total protein staining (Licor). Antibodies were diluted using Intercept® (TBS) Blocking Buffer and incubated overnight at 4C under gentle shaking. Prior secondary antibody incubation, membranes were washed 2 times using TBS-T for 10 minutes. As secondary antibodies we used IR Dye 800n CW Goat-anti-Rabbit (925-32211, Licor) diluted 1:10000 in blocking buffer for 1 h at room temperature under gentle shaking. Membranes were washed three times with TBST-T prior IR detection using Odyssey imager. To quantify the results, each band was quantified using ImageJ and data expressed as pDABL:total protein ratio normalized to the control.

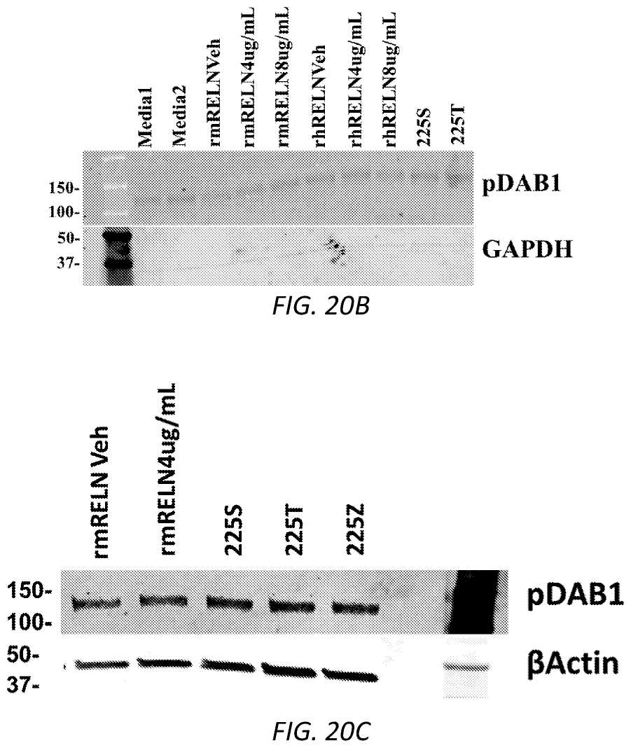

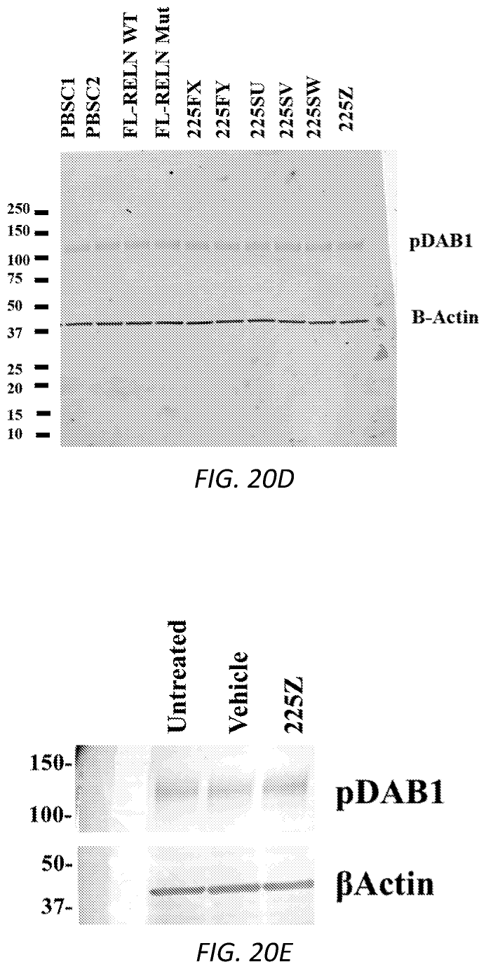

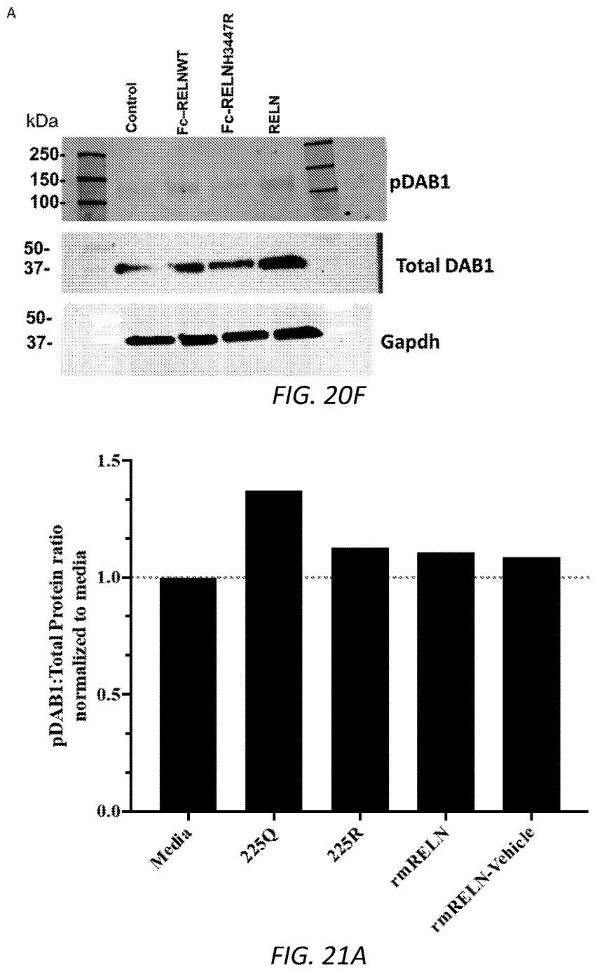

RELN triggered phosphorylation and proteosomal degradation of Dab1. FIGS. 20A-F showed increased levels of phosphorylated DAB1 (pDAB1) after treatment with the mini-RELN purified constructs. pDAB1 levels were quantified in FIGS. 21A-E. These experiments show that the mini-RELN constructs also work in a non-cell autonomous manner.

As seen in FIG. 21A, increased pDAB1 levels upon 225Q treatment. rmRELN lacking the C-term of RELN (rmRELN) was inferior in efficacy compared to 225Q, which contained C-term RELN with the COLBOS variant. This finding suggests that a module containing the C-term of RELN or a GAG binding domain is useful for optimal RELN signaling. Signaling by miniRELN was measured by directly detecting phosphorylated Dab1 (pDab1) after short term treatment. In this design the miniRELN peptides are shown to work when produced by some cells like HEK and signal in other cells like HREC. FIG. 21B shows increased pDAB1 levels upon treatment with 225S and 225T mini-RELN constructs. 225T containing Fc for oligomerization, RAP for binding to receptors, and the C-term domain of RELN with the COLBOS variant was more effective than controls that were larger and lacked the C-term RELN domain with the COLBOS variant (rmRELN mouse and human at much higher concentrations including R3-6 of RELN). This confirms the conclusion of the superiority of constructs containing a GAG binding domain like the C-term domain of RELN with the COLBOS variant. FIG. 21C shows increased levels of pDAB1 in the presence of the mini-RELN constructs of various configurations as compared vehicle; 225Z including CR50, R5-6 of RELN and C-Term of RELN with COLBOS variant was more effective than commercially available positive control lacking a GAG binding domain and lacking an oligomerization domain. Data in FIG. 21D shows increased pDAB1 levels upon treatment with 225SV, 225SW and 225Z. This experiment showed that a miniRELN construct like 225Z including only CR50, R5-6 and the C-term of RELN with the COLBOS variant was more effective at triggering signaling compared to full-length RELN WT and full-length RELN COLBOS. This data demonstrates the mini-RELN constructs are more effective than full-length RELN even when the full-length RELN has the COLBOS variant. FIG. 21E, data showing increased pDAB1 levels upon treatment with 225Z. As shown in FIG. 21F, as expected for constructs that only contain Fc for oligomerization and the C-term of RELN but lack the receptor binding domain, there was no detectable increase in RELN signaling. Therefore, the presence of the receptor binding domain is necessary to activate RELN signaling.

In sum, these results provide evidence that the functional modules that we described in Table A are both necessary and sufficient to stimulate optimal RELN signaling with a therapeutic intent.

Depending on the indication for instance if more or less RELN signaling supplementation is required or depending on the particular route of administration a particular mini-RELN may be more appropriate. For instance, smaller mini-RELN constructs may be preferred for the transnasal delivery.

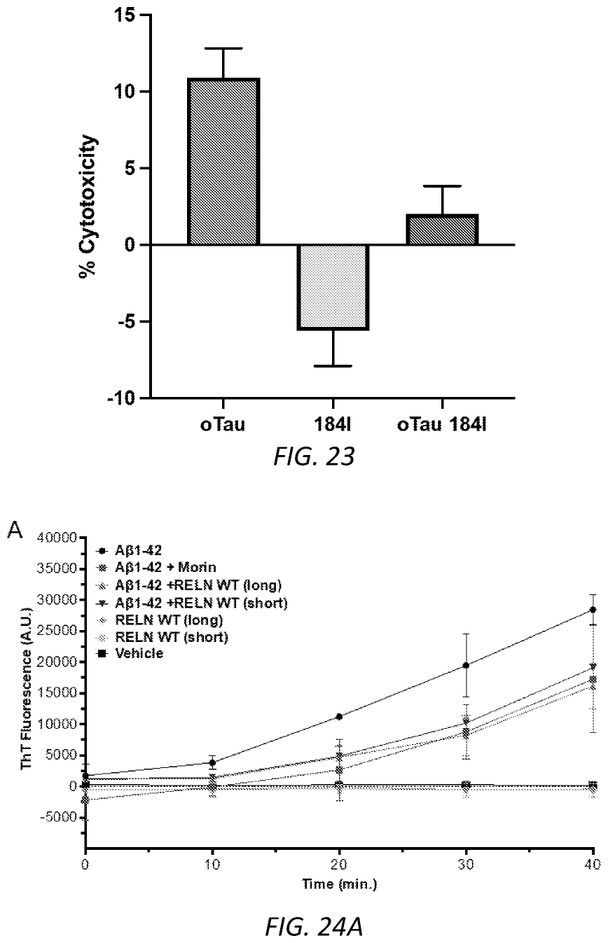

A lactate dehydrogenase assay was performed in SH-SY5Y cells treated 24 h with pre-formed oligomeric Tau (oTau) either alone or in the presence of C-term RELN WT (184I). The lactate dehydrogenase assay was performed as follows. 100 k neuroblastoma (SH-SY5Y, ATCC) cells/well were plated in 96-well TC treated plates in growth media (DMEM/F12, Gibco, 10% heat-inactivated FBS, R&D, 200 ug normocin) and allowed to attach and grow overnight. Next day, test compounds were allowed to thaw on wet ice. Phenol-free and serum free DMEM/F12 no serum no antibiotic media was warmed and a 50 mL aliquot was sterile filtered (0.22 um) as test media. In TC cabinet, thawed test compounds were mixed via pipetting then added to final concentration for each test condition in filtered test media and mixed through pipetting. Test conditions were allowed to pre-incubate for 15 mins at RT in TC cabinet. Wells were gently aspirated and gently washed with 200 uL prewarmed Test media, then gently aspirated and 100 μL of test conditions were added and allowed to incubate in TC incubator at 5% CO2 for 21 hrs. LDH performed using Roche kit according to manufacturer's protocol. The results, seen in FIG. 23, showed a reduction of oTau-derived cytotoxicity in the presence of 184I, thus suggesting a direct interaction with neurotoxic oligomeric tau aggregates. 184I did not show any cytotoxicity. These results suggest a mechanism for direct protection against tau toxicity mediated by a miniRELN construct. Without wishing to be bound by theory, it is proposed that this mechanism does not involve RELN signaling, but is a direct effect mediated via binding of Cterm RELN and tau.

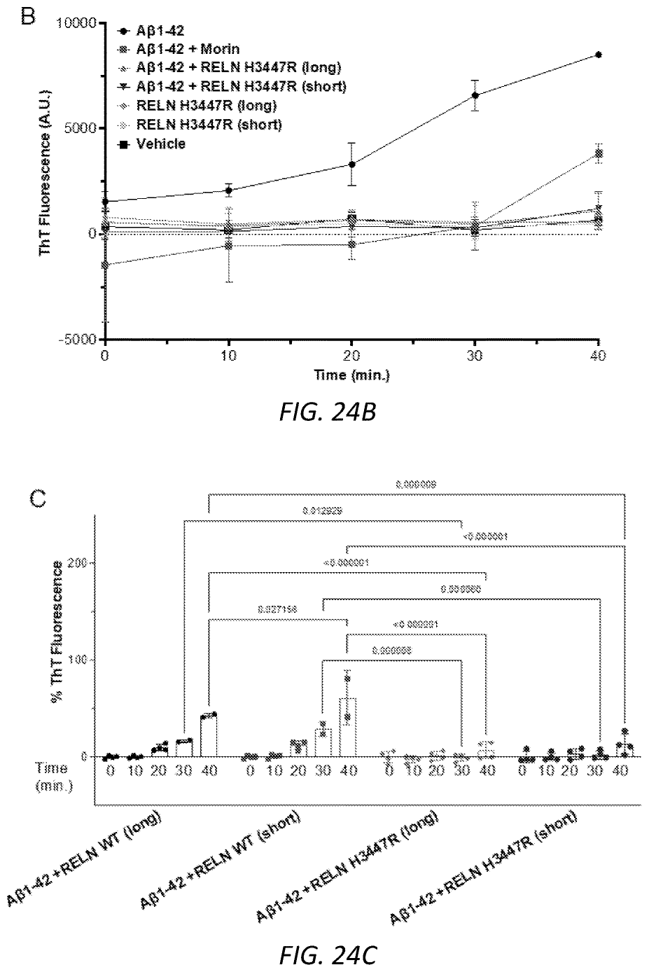

A Thioflavin T (ThT) assay was also performed using synthetic Reelin peptides to assess the differential effects of these proteins on Aβ42 aggregation in vitro using Thioflavin T (SensoLyte ThT O-Amyloid (1-42) Aggregation kit, catalog no. ΔS-72214). Aβ42 was added to a final concentration of 55 μM in solutions of 10 μM of Reelin peptide variants in a transparent, no-binding 96-well plate. Samples were then mixed with 2 mM Thioflavin T dye and fluorescence was read at excitation wavelength/emission wavelength (Ex/Em)=440/484 nm at intermittent time intervals over 2 h. The plate was kept at 37° C. with 15 s shaking between reads. The results, shown in FIGS. 24A-C, showed that the Cterm domain of RELN with and without the COLBOS variant reduced amyloid aggregation. The presence of the COLBOS variant enhanced this effect. In general, it appears that the RELN Cterm domain is highly unstructured. Interactions with tau and/or amyloid and/or HSPGs may allow RELN to assume more stable configurations leading to anti-aggregation properties. These data suggest that so as long as a mini-RELN construct contains the C-term domain of RELN, preferably with the COLBOS variant, it could have protective effects. If the mini-RELN includes the C-term domain only without a receptor binding domain then the protection will not involve signaling, but will still involve direct interactions with tau. If the mini-RELN includes the C-term domain and the receptor binding domain, it will have at least two protective mechanisms, namely RELN signaling and direct interactions with tau.

HEK-derived constructs were tested for overexpression whenever antibodies were available using Western blotting as follows. 15 μg protein homogenates were prepared using Laemmli. Samples were electrophoretically separated in a 4-20% Gel at 90V and protein transferred to a nitrocellulose membrane using 20% Methanol tris-glycine transfer buffer. The membrane was blocked using Intercept® (TBS) Blocking Buffer for 2 hours. We detected anti Fc-taf (1:1,000, goat, Sigma Aldrich), GAPDH (1:5000, mouse, Abcam). Antibodies were diluted using Intercept® (TBS) Blocking Buffer and incubated overnight at 4C under gentle shaking. Prior to secondary antibody incubation, membranes were washed 2 times using TBS-T for 10 minutes. As secondary antibodies, we used IR Dye 800n CW Goat-anti-Rabbit (925-32211, Licor), IR Dye 680 CW Goat-anti-Mouse (925-68070, Licor) both diluted 1:10000 in blocking buffer and HRP (; m-IgG1 BP-HRP-SC-525408, Santa Cruz) diluted 1:1000 also in blocking buffer. for 1 h at room temperature under gentle shaking. Membranes were washed three times with TBS-T prior IR detection using Odyssey imager. To quantify the results, each band was quantified using ImageJ and data expressed as pDAB1:β-actin ratio normalized to the control.

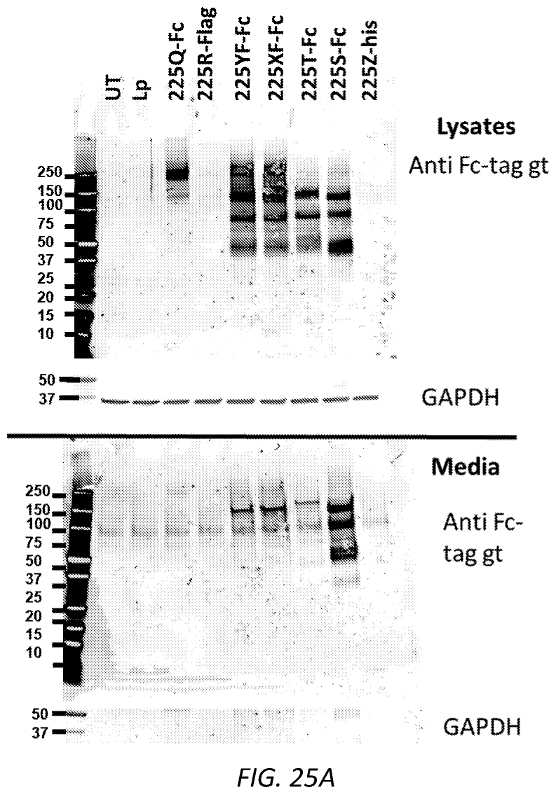

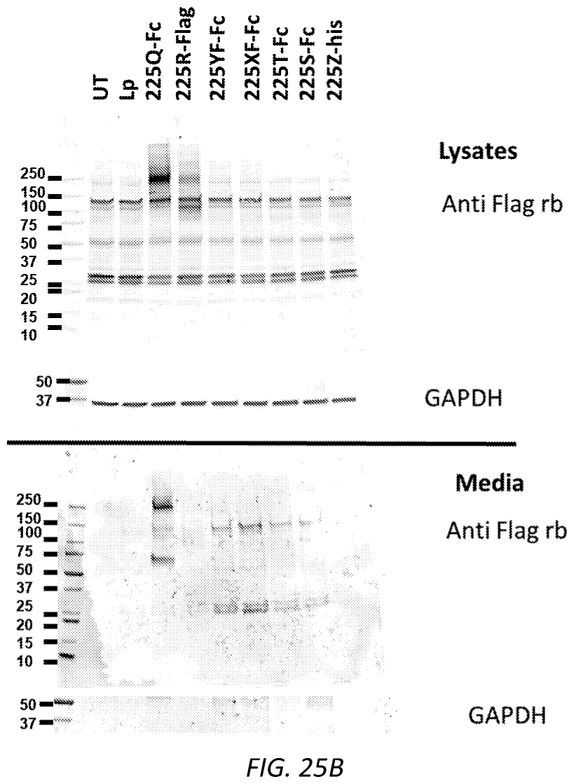

The results, presented in FIGS. 25A-B, provide evidence of protein expression. For 225R, which includes the CR50 domain of RELN, higher molecular weight bands representing miniRELN oligomers were detected (25A-B). Mini-RELN constructs including Fc from IgGs are expected to form oligomers whereas constructs including CR50 and fspCR50 are expected to form higher molecular weight multimers.

The effect of in vitro treatment with RELN or miniRELN protein was also evaluated. HRECs were cultured in EBM-2 media enriched with EGM-2 Single Quots (Lonza) at 37° C. and 5% CO₂ and experimentally tested at passage 7. 24 h prior experiment, cells were plated on a 6-well plate at 500,000 cells/mL. Two hours before the experiment, the cells were washed with PBS to remove residual growth media and then incubated with a starving media including EBM-2, 1% GlutaMax+0,2% Normicin. Treatments was conducted using media derived from HEK 293T cells transiently transfected for 24 h with different mini-RELN constructs in the presence of Lipofectamine 2000. HEK-derived Lipofectamine2000-media was used as negative control. HEK 293T-derived media was tested at different ratio 1:1, 1:4, and 1:14 (Transfected media:Starving media). Each treatment was incubated for 5 minutes and cells were subsequently harvested in lysis buffer was applied constituted of RIPA (Cell signaling), proteases inhibitors (cOmplete™, Mini, EDTA-free Protease Inhibitor Cocktail, Roche), phosphatases inhibitors (PhosSTOP, Roche), 1 μL of proteasome inhibitor MG-132 10 mM, 1% Triton X 100. The lysates were scraped and centrifugated at 15000 rpm for 10 minutes at 4° C. Total protein levels were quantified via BCA assay. For Western Blotting, 7 ug protein homogenates were prepared under reducing and denaturing conditions using Laemmli buffer enriched with 10 mM DTT and 5 min boiling. Samples were electrophoretically separated in a 4-20% Gel at 90V and protein transferred to a PVDF membrane using iBlot™ 2 Dry Blotting System. The membrane was blocked using Intercept® (TBS) Blocking Buffer for 2 hours. We detected pDAB1 (Tyr232; 1:1000, rabbit, #3325 Cell signaling), and Beta actin (1:5000, mouse, #66009-1-Ig, Proteintech. Antibodies were diluted using Intercept® (TBS) Blocking Buffer and incubated overnight at 4C under gentle shaking. Prior secondary antibody incubation, membranes were washes 2 times using TBS-T for 10 minutes. As secondary antibodies we used IR Dye 800n CW Goat-anti-Rabbit (925-32211, Licor) and IR Dye 680 CW Goat-anti-Mouse (925-68070, Licor) both diluted 1:10000 in blocking buffer for 1 h at room temperature under gentle shaking. Membranes were washed three times with TBS-T prior IR detection using Odyssey imager. To quantify the results, each band was quantified using ImageJ and data expressed as pDAB1:βActin ratio normalized to the control.

Transient overexpression of mini-RELN constructs in HEK 293T was obtained as follows. HEK T293Cells (ATCC) were cultured in DMEM media supplemented with 10% fetal bovine serum (FBS, R&D) and 400 uL/L normocine at 37° C. and 5% CO2 and experimentally used up to 10 passages. 24 h prior experiment, cells were plated on a 6-well plate at 300,000 cells/mL. The cell transfection of plasmids was done with Lipofectamine 2000 (7 mL, Life technologies,) according to the manufacturer. In detail, one day prior to transfection, cells were seeded to be 70% confluent at moment of transfection. The day of transfection lipofectamine was diluted and incubated for 10 minutes at room temperature, to allow the formation of the lipophilic complex. Subsequently, each plasmid (3 mg, Invivogen) was added to the lipid complex of Lipofectamine and incubated for 10 minutes. We used OPTIMEM media (Gibco) to block the transfection after 5 Hours, to allow cells to recover and facilitate replication. 24 H post-transfection, cells were harvested, and media cleared from cells by centrifugation and stored under aseptic conditions at −80 until use. Prior, 5 h and 24 h post-transfection, cells were imaged using 10× magnification light microscope (Olympus CKX53).

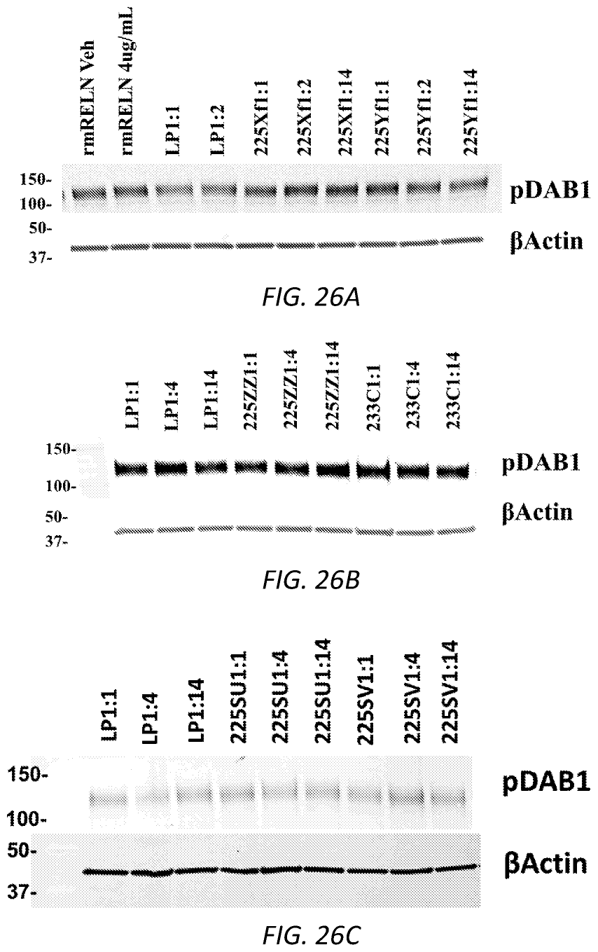

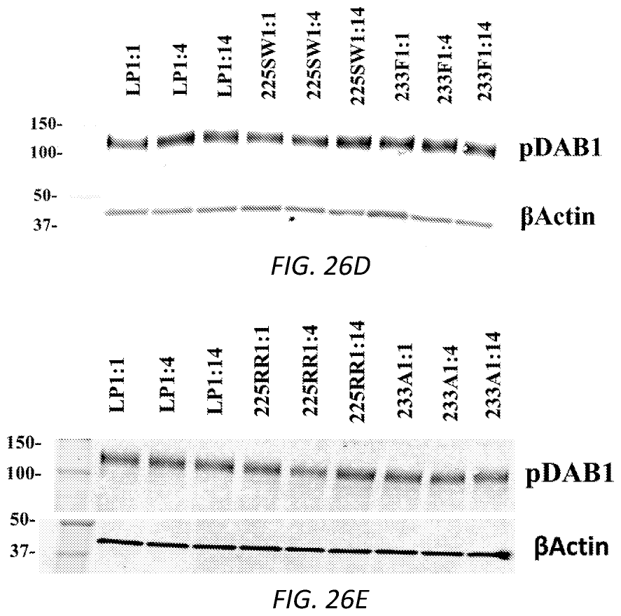

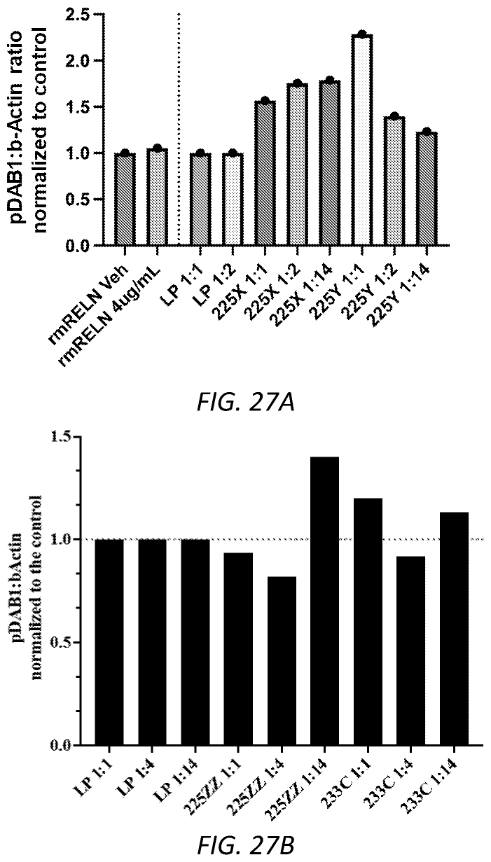

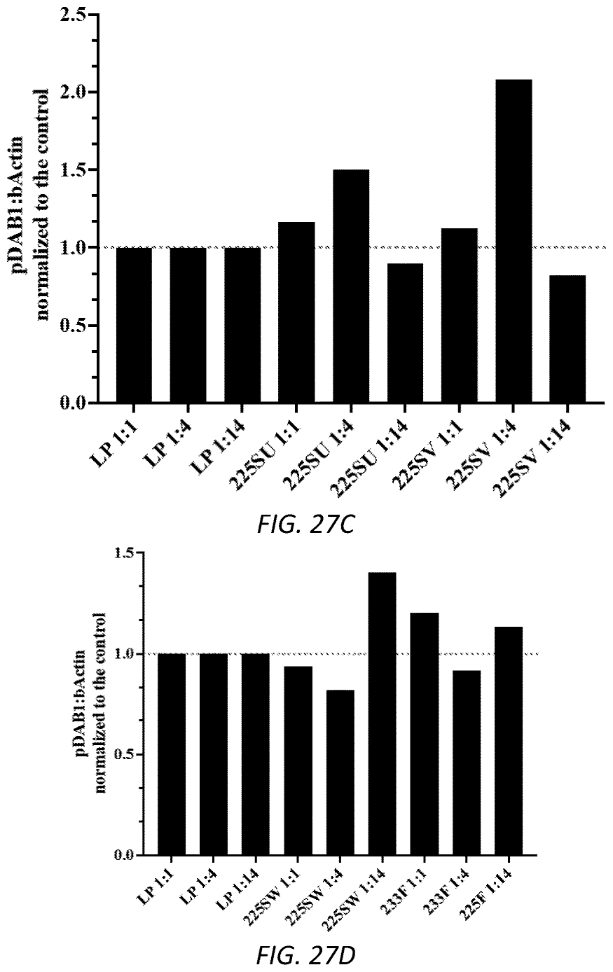

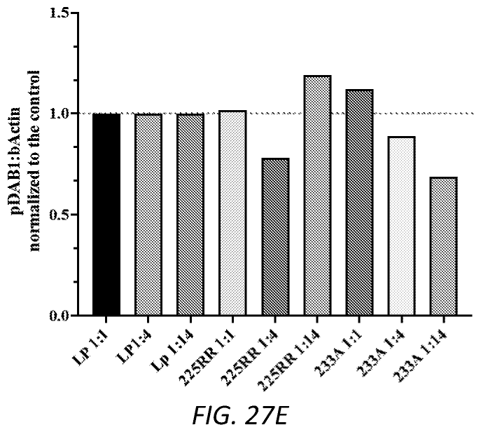

FIGS. 26A and 27A show that miniRELN constructs containing Fc to induce oligomerization, APOE to bind the receptors, and C-TermRELN to bind HSPGs were capable of activating RELN signaling better than a commercially available construct containing only RELN R5-6. As expected 225Yf which includes WT APOE, expected to bind the receptors more efficiently, is more efficacious and clearly dose dependent. 225Xf including APOE with the Christchurch mutation is also effective at activating signaling. The results shown in FIGS. 26B and 27B upon treatment with 225ZZ might be related to the expected property of this construct to oligomerize. FIGS. 26C and 27C show efficacy for 225SU and 225SV miniRELN constructs. FIGS. 26D and 27D show efficacy for miniRELN constructs 225SW and 233F; 233F includes two RELN R6 domains in tandem, a construct that is not naturally occurring. FIGS. 26E and 27E show Dab1 signaling activation with 225RR construct including RELN Cterm with the COLBOS variant, which was more robust compared to controls and compared to 233A, which includes RELN Cterm WT.

### Example 11. In Vivo Validation of Purified Recombinant Mini-RELN Constructs

We used a MIND procure (trans nasal delivery using Minimally Invasive Nasal Depot (MIND), Padmakumar et al., J Control Release. 2021 Mar. 10; 331: 176-186) or intraperitoneal injection to administer miniRELN constructs into mice. MIND leverages the olfactory nerve to bypass the blood brain barrier. Our clinical studies showed the importance of the entorhinal cortex in extreme protection against Alzheimer's. Using the MIND methodology provides one method of reaching the entorhinal cortex with minimal systemic exposure. We showed it is possible to deliver miniRELN to the hippocampal formation, including the entorhinal cortex, which we showed is a region of the brain critical for extreme protection against Alzheimer's. C57Bl/3 (Jackson Laboratory) were housed under regular light/night cycle. All procedures were conducted under approved IACUC animal protocols. Prior to incision, the nasal region was shaved and cleaned using betadine and 70% alcohol and mouse subcutaneously injected with buprenorphine HCl and meloxicam (5 mg/kg). The nasal cavity was then opened using a scalped and nasal mucosa exposed using a microdrill. Once the mucosa was exposed, gel dispersion of either PBS or mini-RELN constructs (Innovagen) in 20% Pluronic (Sigma Aldrich) was subcutaneously delivered in the surgically-generated pocket. Mice were injected with meloxicam for 3 days prior euthanasia at day 4 to collect the brain tissue used for post-mortem analysis.

Western blotting was performed as follows. 20 ug protein homogenates were prepared under reducing and denaturing conditions using Laemmli buffer enriched with 10 mM DTT and 5 min boiling. Samples were electrophoretically separated in a 4-20% Gel at 90V and protein transferred to a PVDF membrane using iBlot™ 2 Dry Blotting System. The membrane was blocked using Intercept® (TBS) Blocking Buffer for 2 hours. We detected pDAB1 (Tyr232; 1:1000, rabbit, #3325 Cell signaling), and Beta actin (1:5000, mouse, #66009-1-Ig, Proteintech). Antibodies were diluted using Intercept® (TBS) Blocking Buffer and incubated overnight at 4C under gentle shaking. Prior secondary antibody incubation, membranes were washes 2 times using TBS-T for 10 minutes. As secondary antibodies we used IR Dye 800n CW Goat-anti-Rabbit (925-32211, Licor) and IR Dye 680 CW Goat-anti-Mouse (925-68070, Licor) both diluted 1:10000 in blocking buffer for 1 h at room temperature under gentle shaking. Membranes were washed three times with TBS-T prior IR detection using Odyssey imager. To quantify the results, each band was quantified using ImageJ and data expressed as pDAB1:βActin ratio normalized to the control.

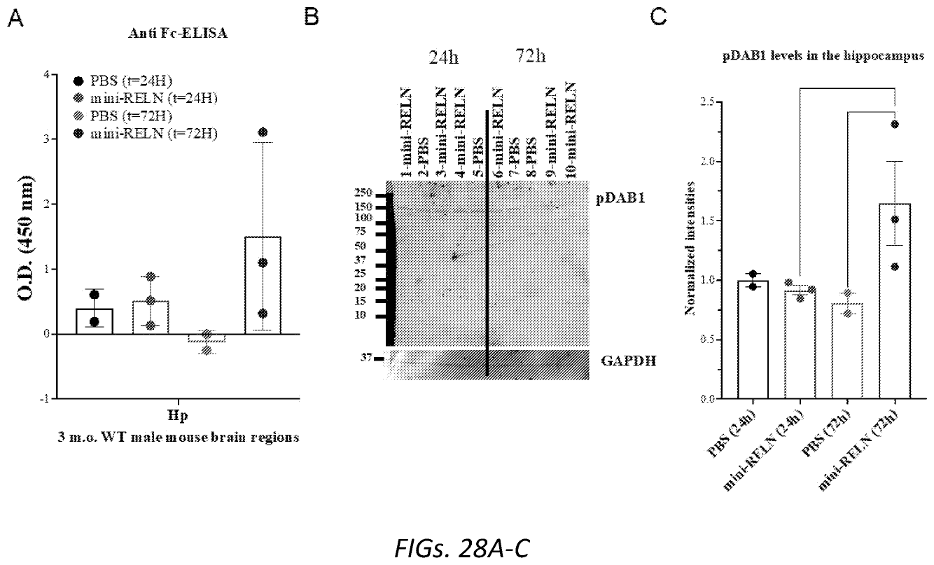

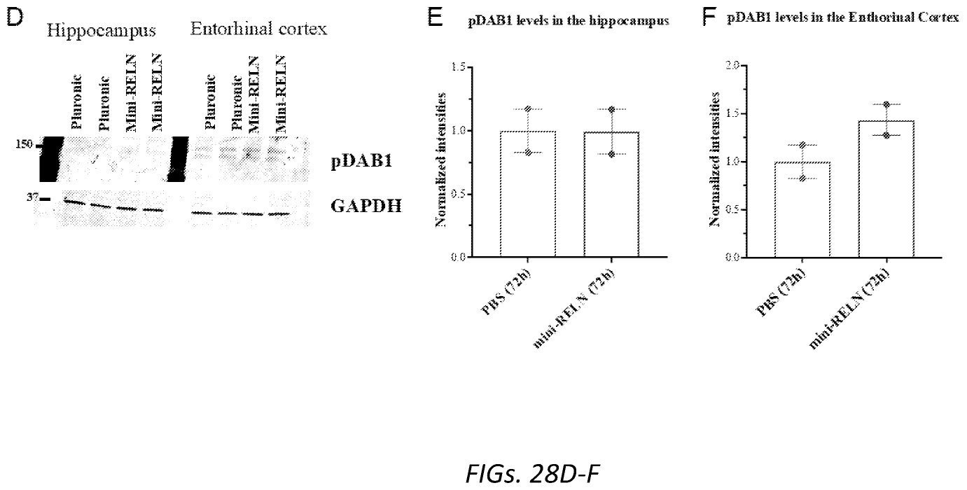

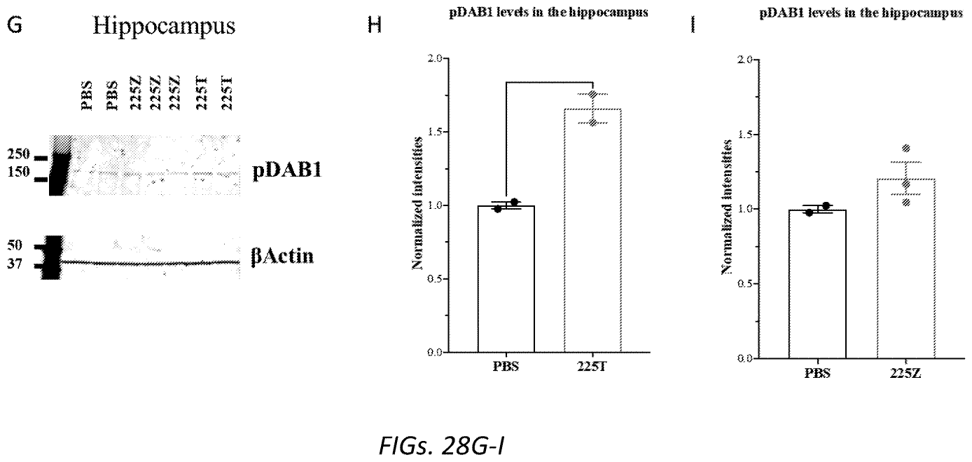

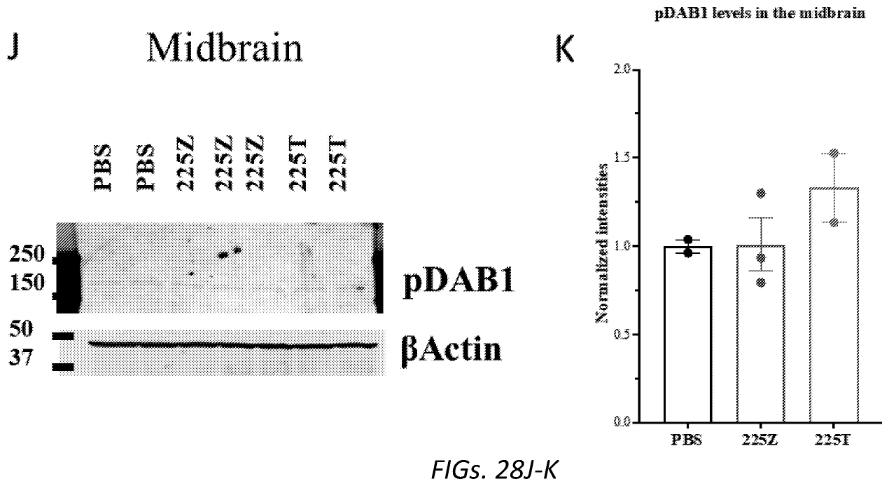

The data, shown in FIGS. 28A-K, demonstrated effective brain delivery of a miniRELN peptide to both the hippocampus and the entorhinal cortex and to the midbrain, leading to increased levels of downstream RELN pathway as confirmed by increased pDAB1 levels. Mice treated with the miniRELN construct have more RELN signaling in the hippocampus and the entorhinal cortex as determined by increased pDab1. The efficacy of the miniRELN treatment was confirmed in WT mice and in mice with a tau mutation leading to tauopathy. 225Z includes CR50 of RELN to induce oligomerization, R5-6 of RELN to mediate receptor binding, and Cterm RELN with the COLBOS mutation to achieve HSPG binding. This shows that these RELN domains were sufficient to drive protective signaling.

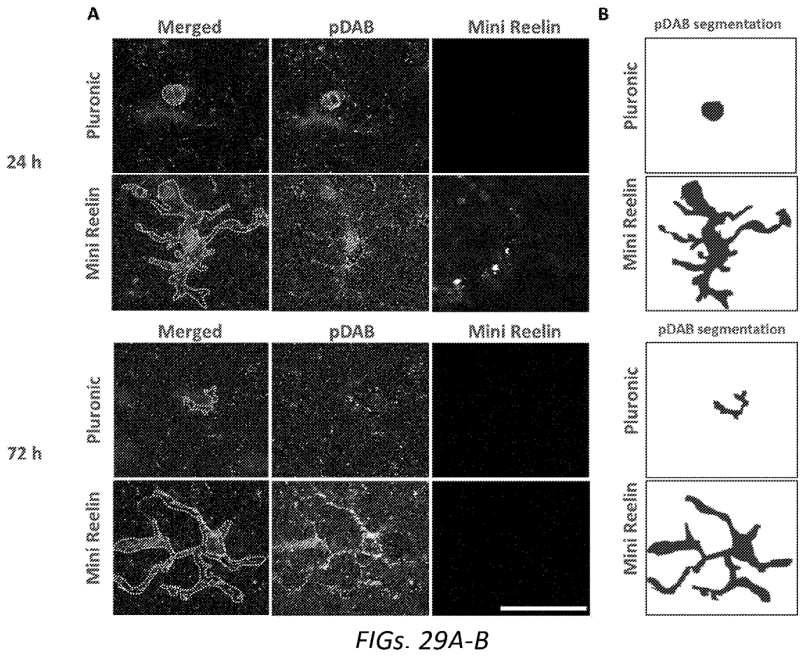

Immunofluorescence staining of murine brains was also used to demonstrate delivery of miniRELN using MIND. 24 or 72 hours after Mini Reelin (225S) or vehicle administration, mice were intracardially perfused with 4% PFA in PBS and brains were harvested and incubated in 4% PFA at 4° C. for 24 hours. Sagittal sections were obtained at 1 mm thickness using a stainless-steel brain matrix. The entorhinal-hippocampal section of each brain was chosen for Clarity using Binaree® Tissue Clearing™ Kit (HRTC-012) following the kit guidelines. Sections were incubated with IgG-Fc tag and pDAB1 primary antibodies for 72 h followed by secondary antibody: Alexa 488 and Alexa 647, and DAPI for 1 h. Subsequently, sections were imaged using confocal SP8 microscope for quantification. The results (exemplary results for construct 225S shown in FIGS. 29A-B) demonstrated the presence of the miniRELN peptide in the brain that was colocalized with brain cells with increased levels of pDab1. This is proof of effective drug delivery, target engagement, and targeted RELN signaling activation, in the hippocampal formation.

### Example 12. Systemic Delivery of Mini-RELN Reduced Tau Pathology in P301S Tau Mice

The effects of systemic delivery of mini-RELN peptides was evaluated in vivo. 6 months old female MAPT P301S Tau Tg mice were injected intraperitoneally on day 1 with 500 μL and on day 2-4 with 250 μL solutions of either vehicle (PBS) or mini-RELN peptides. On Day 5, mice were euthanized with saturating concentration CO2 gas and intracardially perfused with 4% PFA. Brains were subsequently harvested and used for histological analyses.

Immunofluorescence staining of murine brains was performed as follows. 24 hours post fixation with 4% PFA at 4° C., sagittal brain sections were obtained at 1 mm thickness using a stainless-steel brain matrix. The entorhinal-hippocampal section of each brain was chosen for Clarity using Binaree® Tissue Clearing™ Kit (HRTC-012) following the kit guidelines. Sections were incubated with pTauS396 primary antibody for 72 h followed by secondary antibody: Alexa 647, and DAPI for 1 h. Subsequently, sections were imaged using confocal SP8 microscope for quantification. Fourteen images were taken at 63× magnification per group. Fluorescence intensity of pTau was quantified automatically using Matlab (2021a). Otsu's thresholding method was used to get a mask of the image signal and then the mean intensity of the signal was computed.

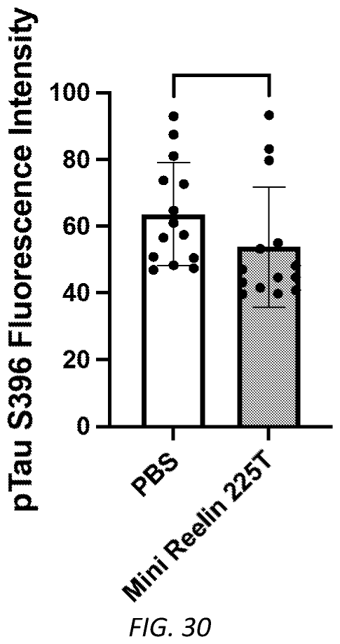

Quantification of pTau S396 fluorescence intensity was used as a hallmark of tau pathology. The results, shown in FIG. 30, showed that pTau S396 levels were significantly reduced when mice were treated with the mini-RELN peptide. This data shows that administration of the miniRELN construct is efficacious when administered systemically, and that the beneficial effect results in significant reduction of tau phosphorylation, a pathological feature of tauopathies like frontotemporal dementia and Alzheimer's disease. This was shown in a mouse model of tauopathy (MAPT P301S).

### REFERENCES

1. Acosta-Baena N, Sepulveda-Falla D, Lopera-Gomez CM, Jaramillo-Elorza M C, Moreno S, Aguirre-Acevedo D C, et al. Pre-dementia clinical stages in presenilin 1 E280A familial early-onset Alzheimer's disease: a retrospective cohort study. Lancet Neurol. 2011; 10(3):213-20.
2. Arboleda-Velasquez J F, Lopera F, O'Hare M, Delgado-Tirado S, Marino C, Chmielewska N, et al. Resistance to autosomal dominant Alzheimer's disease in an APOE3 Christchurch homozygote: a case report. Nat Med. 2019; 25(11):1680-3.
3. Quiroz Y T, Sperling R A, Norton D J, Baena A, Arboleda-Velasquez J F, Cosio D, et al. Association Between Amyloid and Tau Accumulation in Young Adults With Autosomal Dominant Alzheimer Disease. JAMA neurology. 2018; 75(5):548-56.
4. D′Arcangelo G, Homayouni R, Keshvara L, Rice D S, Sheldon M, Curran T. Reelin is a ligand for lipoprotein receptors. Neuron. 1999; 24(2):471-9.
5. Hiesberger T, Trommsdorff M, Howell B W, Goffinet A, Mumby M C, Cooper J A, et al. Direct binding of Reelin to VLDL receptor and ApoE receptor 2 induces tyrosine phosphorylation of disabled-1 and modulates tau phosphorylation.

### Neuron. 1999; 24(2):481-9.

6. Lane-Donovan C, Herz J. ApoE, ApoE Receptors, and the Synapse in Alzheimer's Disease. Trends Endocrinol Metab. 2017; 28(4):273-84.
7. Sheldon M, Rice D S, D′Arcangelo G, Yoneshima H, Nakajima K, Mikoshiba K, et al. Scrambler and yotari disrupt the disabled gene and produce a reeler-like phenotype in mice. Nature. 1997; 389(6652):730-3.
8. Mondragón-Rodriguez S, Perry G, Luna-Muñoz J, Acevedo-Aquino M C, Williams S. Phosphorylation of tau protein at sites Ser(396-404) is one of the earliest events in Alzheimer's disease and Down syndrome. Neuropathol Appl Neurobiol. 2014; 40(2):121-35.
9. Bramblett G T, Goedert M, Jakes R, Merrick S E, Trojanowski J Q, Lee V M. Abnormal tau phosphorylation at Ser396 in Alzheimer's disease recapitulates development and contributes to reduced microtubule binding. Neuron. 1993; 10(6):1089-99.
10. Kanno T, Tsuchiya A, Nishizaki T. Hyperphosphorylation of Tau at Ser396 occurs in the much earlier stage than appearance of learning and memory disorders in 5×FAD mice. Behav Brain Res. 2014; 274:302-6.
11. Nakano Y, Kohno T, Hibi T, Kohno S, Baba A, Mikoshiba K, et al. The extremely conserved C-terminal region of Reelin is not necessary for secretion but is required for efficient activation of downstream signaling. J Biol Chem. 2007; 282(28):20544-52.
12. Mahley R W, and Ji Z. S. Remnant lipoprotein metabolism: key pathways involving cell-surface heparan sulfate proteoglycans and apolipoprotein E. Journal of Lipid Research. 1999; 40(1):1-16.
13. Di Donato V, De Santis F, Albadri S, Auer T O, Duroure K, Charpentier M, et al. An Attractive Reelin Gradient Establishes Synaptic Lamination in the Vertebrate Visual System. Neuron. 2018; 97(5):1049-62 e6.
14. D′Arcangelo G, Miao G G, Chen S C, Soares H D, Morgan J I, Curran T. A protein related to extracellular matrix proteins deleted in the mouse mutant reeler. Nature. 1995; 374(6524):719-23.
15. Wasser C R, Herz J. Reelin: Neurodevelopmental Architect and Homeostatic Regulator of Excitatory Synapses. J Biol Chem. 2017; 292(4):1330-8.
16. Utsunomiya-Tate N, Kubo K, Tate S, Kainosho M, Katayama E, Nakajima K, et al. Reelin molecules assemble together to form a large protein complex, which is inhibited by the function-blocking CR-50 antibody. Proc Natl Acad Sci USA. 2000; 97(17):9729-34.
17. Shifman S, Johannesson M, Bronstein M, Chen S X, Collier D A, Craddock N J, et al. Genome-wide association identifies a common variant in the reelin gene that increases the risk of schizophrenia only in women. PLoS Genet. 2008; 4(2):e28.
18. Ober C, Loisel D A, Gilad Y. Sex-specific genetic architecture of human disease. Nat Rev Genet. 2008; 9(12):911-22.
19. Goes F S, Willour V L, Zandi P P, Belmonte P L, MacKinnon DF, Mondimore F M, et al. Sex-specific association of the Reelin gene with bipolar disorder. Am J Med Genet B Neuropsychiatr Genet. 2010; 153b(2):549-53.
20. Seripa D, Matera M G, Franceschi M, Daniele A, Bizzarro A, Rinaldi M, et al. The RELN locus in Alzheimer's disease. J Alzheimers Dis. 2008; 14(3):335-44.
21. Feher Á, Juhisz A, Pákáski M, Kálmin J, Janka Z. Genetic analysis of the RELN gene: Gender specific association with Alzheimer's disease. Psychiatry Res. 2015; 230(2):716-8.
22. Kelemenova S, Ostatnikova D. Neuroendocrine pathways altered in autism. Special role of reelin. Neuro Endocrinol Lett. 2009; 30(4):429-36.
23. Sepulveda-Falla D, Sanchez J S, Almeida M C, Boassa D, Acosta-Uribe J, Vila-Castelar C, et al. Distinct tau neuropathology and cellular profiles of an APOE3 Christchurch homozygote protected against autosomal dominant Alzheimer's dementia. Acta Neuropathol. 2022; 144(3):589-601.
24. Braak H, Braak E. Evolution of the neuropathology of Alzheimer's disease. Acta neurologica Scandinavica. 1996; 165:3-12.
25. Gomez-Isla T, Price J L, McKeel D W, Jr., Morris J C, Growdon J H, Hyman B T. Profound loss of layer II entorhinal cortex neurons occurs in very mild Alzheimer's disease. J Neurosci. 1996; 16(14):4491-500.
26. Kramer P L, Xu H, Woltjer R L, Westaway S K, Clark D, Erten-Lyons D, et al. Alzheimer disease pathology in cognitively healthy elderly: a genome-wide study. Neurobiol Aging. 2011; 32(12):2113-22.
27. Bracher-Smith M, Leonenko G, Baker E, Crawford K, Graham A C, Salih D A, et al. Whole genome analysis in APOE4 homozygotes identifies the DAB1-RELN pathway in Alzheimer's disease pathogenesis. medRxiv. 2022:2022.04.28.22274418.
28. Lalazar A, Weisgraber K H, Rall S C J, Giladi H, Innerarity T L, Levanon A Z, et al. Site-specific Mutagenesis of Human Apolipoprotein E. J Biochem. 1988; 263(8):3542-5.
29. Martinez-Burgo, B., et al., AC-terminal CXCL 8 peptide based on chemokine-glycosaminoglycan interactions reduces neutrophil adhesion and migration during inflammation. Immunology, 2019. 157(2): p. 173-184.
30. Cardin, A. D. and H. Weintraub, Molecular modeling of protein-glycosaminoglycan interactions. Arteriosclerosis: An Official Journal of the American Heart Association, Inc., 1989. 9(1): p. 21-32.
31. Neufeld, G., O. Kessler, and Y. Herzog, The interaction of Neuropilin-1 and Neuropilin-2 with tyrosine-kinase receptors for VEGF. Neuropilin: From Nervous System to Vascular and Tumor Biology, 2002: p. 81-90.
32. Turk, L. S., et al., Structure of Reelin repeat 8 and the adjacent C-terminal region. Biophysical Journal, 2022.
33. Tirion, M. M., Large amplitude elastic motions in proteins from a single-parameter, atomic analysis. Physical review letters, 1996. 77(9): p. 1905.
34. Bentahir, M., et al., Presenilin clinical mutations can affect γ-secretase activity by different mechanisms. Journal of neurochemistry, 2006. 96(3): p. 732-742.
35. Scheuner, D., et al., Secreted amyloid 0-protein similar to that in the senile plaques of Alzheimer's disease is increased in vivo by the presenilin 1 and 2 and APP mutations linked to familial Alzheimer's disease. Nature medicine, 1996. 2(8): p. 864-870.
36. Lortat-Jacob, H., A. Grosdidier, and A. Imberty, Structural diversity of heparan sulfate binding domains in chemokines. Proceedings of the National Academy of Sciences, 2002. 99(3): p. 1229-1234.
37. Rueda, M., P. Chacón, and M. Orozco, Thorough validation of protein normal mode analysis: a comparative study with essential dynamics. Structure, 2007. 15(5): p. 565-575.
38. Mangione, M. R., et al., Hsp60, amateur chaperone in amyloid-beta fibrillogenesis. Biochimica et Biophysica Acta (BBA)-General Subjects, 2016. 1860(11): p. 2474-2483.
39. Pujadas, L., et al., Reelin delays amyloid-beta fibril formation and rescues cognitive deficits in a model of Alzheimer's disease. Nat Commun, 2014. 5: p. 3443.
40. Xue, C., et al., Thioflavin T as an amyloid dye: fibril quantification, optimal concentration and effect on aggregation. Royal Society open science, 2017. 4(1): p. 160696.
41. Lemkul, J. A. and D. R. Bevan, Morin inhibits the early stages of amyloid beta-peptide aggregation by altering tertiary and quaternary interactions to produce “off-pathway” structures. Biochemistry, 2012. 51(30): p. 5990-6009.
42. Kim, W. and M. H. Hecht, Sequence determinants of enhanced amyloidogenicity of Alzheimer Aβ42 peptide relative to Aβ40. Journal of Biological Chemistry, 2005. 280(41): p. 35069-35076.
43. Deutsch, S. I., R. B. Rosse, and R. M. Lakshman, Dysregulation of tau phosphorylation is a hypothesized point of convergence in the pathogenesis of alzheimer's disease, frontotemporal dementia and schizophrenia with therapeutic implications. Progress in Neuro-Psychopharmacology and Biological Psychiatry, 2006. 30(8): p. 1369-1380.
44. Deutsch, S. I., R. B. Rosse, and L. H. Deutsch, Faulty regulation of tau phosphorylation by the reelin signal transduction pathway is a potential mechanism of pathogenesis and therapeutic target in Alzheimer's disease. European neuropsychopharmacology, 2006. 16(8): p. 547-551.
45. Ruvinsky, A. M. and I. A. Vakser, Sequence composition and environment effects on residue fluctuations in protein structures. The Journal of chemical physics, 2010. 133(15): p. 10B609.
46. Kohno, T., et al., Reelin-Nrp1 interaction regulates neocortical dendrite development in a context-specific manner. Journal of Neuroscience, 2020. 40(43): p. 8248-8261.
47. Teesalu, T., et al., C-end rule peptides mediate neuropilin-1-dependent cell, vascular, and tissue penetration. Proceedings of the National Academy of Sciences, 2009. 106(38): p. 16157-16162.
48. Zanuy, D., et al., Sequence dependence of C-end rule peptides in binding and activation of neuropilin-1 receptor. Journal of structural biology, 2013. 182(2): p. 78-86.
49. Lorenzen, S. and Y. Zhang, Identification of near-native structures by clustering protein docking conformations. Proteins: Structure, Function, and Bioinformatics, 2007. 68(1): p. 187-194.
50. Kozakov, D., et al., The ClusPro web server for protein-protein docking. Nature Protocols, 2017. 12(2): p. 255-278.
51. Kleckner, I. R. and M. P. Foster, An introduction to NMR-based approaches for measuring protein dynamics. Biochimica et Biophysica Acta (BBA)-Proteins and Proteomics, 2011. 1814(8): p. 942-968.
52. de Bergeyck V, Nakajima K, Lambert de Rouvroit C, Naerhuyzen B, Goffinet A M, Miyata T, et al. A truncated Reelin protein is produced but not secreted in the ‘Orleans’ reeler mutation (Reln[rl-Orl]). Brain Res Mol Brain Res. 1997; 50(1-2):85-90.
53. Yasui N, Nogi T, Takagi J. Structural basis for specific recognition of reelin by its receptors. Structure. 2010; 18(3):320-31.
54. Okoro E U, Zhang H, Guo Z, Yang F, Smith Jr C, Yang H. A Subregion of Reelin Suppresses Lipoprotein-Induced Cholesterol Accumulation in Macrophages. PLoS One. 2015 Aug. 28; 10(8):e0136895.
55. Guidotti A, Grayson D R. DNA methylation and demethylation as targets for antipsychotic therapy. Dialogues Clin Neurosci. 2014 September; 16(3):419-29.
56. Lintas C, Persico A M. Neocortical RELN promoter methylation increases significantly after puberty. Neuroreport. 2010 Jan. 27; 21(2):114-8.
57. Kang J G, Park J S, Ko J H, Kim Y S. Regulation of gene expression by altered promoter methylation using a CRISPR/Cas9-mediated epigenetic editing system. Sci Rep. 2019 Aug. 19; 9(1):11960.
58. Abo El Fotoh W M M, Bayomy N R, Kasemy Z A, Barain A M, Shalaby B M, Abd El Naby S A. Genetic Variants and Haplotypes of Tryptophan Hydroxylase 2 and Reelin Genes May Be Linked with Attention Deficit Hyperactivity Disorder in Egyptian Children. ACS Chem Neurosci. 2020 Jul. 15; 11(14):2094-2103.
59. Chen X, Zaro J L, Shen W C. Fusion protein linkers: property, design and functionality. Adv Drug Deliv Rev. 2013 October; 65(10):1357-69.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating or preventing a neurodegenerative disease in a subject, the method comprising administering to the subject an effective amount of a reelin protein or a nucleic acid encoding a reelin protein, preferably wherein the reelin protein comprises a H3447R or H3447K mutation and/or a R3454A mutation, optionally H3447R or H3447K in combination with an R3454A mutation.

2. The method of claim 1, wherein the reelin protein comprises full length reelin, or a mini-reelin comprising: (A) a signal peptide; (B) an oligomerization domain, optionally reelin CR-50 Domain; (C) a receptor binding domain, optionally reelin domains (repeats) 5 and 6 (R5-6); and (D) a GAG binding domain, optionally a C-terminus from reelin (CTR).

3. The method of claim 1, comprising administering a nucleic acid encoding a reelin protein, wherein the nucleic acid is a naked mRNA or DNA encoding the reelin, or is in a viral vector, optionally an AAV vector.

4. A composition comprising a reelin protein or a nucleic acid encoding a reelin protein, preferably wherein the reelin protein comprises a H3447R or H3447K mutation and/or a R3454A mutation, optionally H3447R or H3447K in combination with an R3454A mutation.

5. The composition of claim 4, wherein the reelin protein comprises full length reelin.

6. The composition of claim 4, wherein the reelin protein comprises a mini-reelin comprising A) a signal peptide; (B) an oligomerization domain, optionally reelin CR-50 Domain; (C) a receptor binding domain, optionally reelin domains (repeats) 5 and 6 (R5-6); and (D) a GAG binding domain, optionally a C-terminus from reelin (CTR).

7. The composition of claim 4, comprising a nucleic acid encoding a reelin protein, optionally wherein the nucleic acid is a naked mRNA or DNA encoding the reelin or is in a viral vector, optionally an AAV vector.

8. (canceled)

9. The method of claim 1, wherein the neurodegenerative disease is Alzheimer's disease, frontotemporal dementia, memory loss, cognitive impairment, amyotrophic lateral sclerosis (ALS), cognitive decline associated with aging, age-related macular degeneration, glaucoma, diabetic retinopathy or inherited retinal degeneration, stroke, brain trauma or concussion, retinal trauma, small vessel disease like cerebral autosomal dominant arteriopathy with subcortical infarcts and leukoencephalopathy (CADASIL), and wet-age related macular degeneration.

10. A composition comprising or consisting of a reelin C-terminal region (CTR), and optionally a carrier, preferably wherein the CTR comprises a H3447R or H3447K mutation and/or a R3454A mutation, optionally H3447R or H3447K in combination with an R3454A mutation, optionally comprising or consisting of a sequence as shown in Table 1.

11. The composition of claim 8, further comprising a non-reelin nucleic acid, optionally mRNA, optionally wherein the mRNA encodes a therapeutic peptide.

12. The composition of claim 8, further comprising an isolated non-reelin protein, optionally complexed with or fused to the RELN CTR.

13. A method of delivering a nucleic acid or protein to a cell, the method comprising administering to the cell an effective amount of the composition of claim 10.

14. A method of treating or preventing a neurodegenerative disease in a subject, the method comprising administering to the subject an effective amount of an agent that reduces methylation of the RELN promoter in an amount sufficient to increase RELN expression in the subject, wherein the agent that reduces methylation promoter is:
(i) a fusion protein comprising a catalytically inactive CRISPR/Cas protein fused to a demethylation domain, and a guide RNA directing the fusion protein to demethylate a cytosine in the RELN promoter, optionally administered as a RNP; or
(ii) a nucleic acid encoding a fusion protein comprising a catalytically inactive CRISPR/Cas protein fused to a demethylation domain, and a guide RNA directing the fusion protein to demethylate a cytosine in the RELN promoter, optionally administered as mRNA or in one or more vectors, optionally viral vectors, optionally adeno associated viral AAV vectors.

15. A method of treating or preventing a neurodegenerative disease in a subject, the method comprising administering to the subject an effective amount of:
(i) a CRISPR/Cas protein, a guide RNA directing the Cas protein to the region of a RELN allele comprising H3447 or R3454, and an ssODN comprising a sequence comprising a H3447R or H3447K mutation and/or a R3454A mutation, optionally one or more ssODNs comprising a sequence comprising a H3447R or H3447K in combination with an R3454A mutation, for insertion into the RELN allele, optionally administered as a RNP; or
(ii) a nucleic acid encoding a CRISPR/Cas protein, a guide RNA directing the Cas protein to the region of a RELN allele comprising a H3447, and an ssODN comprising a sequence comprising a H3447R or H3447K mutation and/or a R3454A mutation, optionally H3447R or H3447K in combination with an R3454A mutation for insertion into the RELN allele, optionally administered as naked DNA or mRNA and/or in one or more vectors, optionally viral vectors, optionally adeno associated viral AAV vectors.

16. The method of claim 14, wherein the administering is to the entorhinal cortex.

17. The method of claim 14, wherein the disease is Alzheimer's disease, frontotemporal dementia, memory loss, cognitive impairment, amyotrophic lateral sclerosis (ALS), cognitive decline associated with aging, age-related macular degeneration, glaucoma, diabetic retinopathy or inherited retinal degeneration, stroke, brain trauma or concussion, retinal trauma, small vessel disease like cerebral autosomal dominant arteriopathy with subcortical infarcts and leukoencephalopathy (CADASIL), and wet-age related macular degeneration

18. A method to assess risk of developing Alzheimer's Disease in a subject, the method comprising determining the presence or absence of an H3447R variant allele in a RELN gene of the subject, wherein the presence of the H3447 variant indicates that the subject has a reduced risk of developing AD as compared to a subject who does not have the H3447R variant.

19. The method of claim 18, wherein the subject has an APOE4 variant sequence.


SEQUENCE LISTING (81 sequences)

SEQ ID NO: 1 - protein, 901 aa, synthetic construct
EQCGAIMHGNAVTFCEPYGPRELITTGLNTTTASVLQFSIGSGSCRFSYSDPSIIVLYAKNNSADWIQLEKIRAPSNVSTIIHILYLPEDAKGENVQFQWKQENLRVGEVYEACWALGSRGGQTNATRFRLWQPYNNGKKEEIWIVDDFIIDGNNVNNPVMLLDTFDFGPREDNWFFYPGGNIGLYCPYSSKGAPEEDSAMVFVSNEVGEHSITTRDLNVNENTIIQFEINVGCSTDSSSADPVRLEFSRDFGATWHLLLPLCYHSSSHVSSLCSTEHHPSSTYYAGTMQGWRREVVHFGKLHLCGSVRFRWYQGFYPAGSQPVTWAIDNVYIGPQCEEMCNGQGSCINGTKCICDPGYSGPTCKISTKNPDFLKDDFEGQLESDRFLLMSGGKPSRKCGILSSGNNLFFNEDGLRMLMTRDLDLSHARFVQFFMRLGCGKGVPDPRSQPVLLQYSLNGGLSWSLLQEFLFSNSSNVGRYIALEIPLKARSGSTRLRWWQPSENGHFYSPWVIDQILIGGNISGNMVLEDDFTTLDSRKWLLHPGGTKMPVCGSTGDALVFIEKASTRYVVSTDVAVNEDSFLQIDFAVSCSVTDSCYAIELEYSVDLGLSWHPLVRDCLPTNVECSRYHLQRILVSDTFNKWTRITLPLPPYTRSQATRFRWHQPAPFDKQQTWAIDNVYIGDGCIDMCSGHGRCIQGNCVCDEQWGGLYCDDPETSLPTQLKDNFNRAPSSQNWLTVNGGKLSTVCGAVASGMALHFSGGCSRLLVTVDLNLTNAEFIQFYFMYGCLITPNNRNQGVLLEYSVNGGITWNLLMEIFYDQYSKPGFVNILLPPDAKEIATRFRWWQPRHDGLDQNDWAIDNVLISGSASTRKQNYMMNFSRQHGLRHFYNRRRRSLRRYP

SEQ ID NO: 2 - DNA, 856 bp, synthetic construct
  source 1..856: mol_type = other DNA
atgtacaggatgcaactcctgtcttgcattgcactaagtcttgcacttgtcacgaattcggcacctctcgagcgcaaatctagtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgcacgtacgagcactcgcaaacaaaattacatgatgaatttttcacgacaacatgggctcagacatttctacaacagaagacgaaggtcacttaggcgatacccat

SEQ ID NO: 3 - protein, 285 aa, synthetic construct
MYRMQLLSCIALSLALVTNSAPLERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGARTSTRKQNYMMNFSRQHGLRHFYNRRRRSLRRYP

SEQ ID NO: 4 - DNA, 856 bp, synthetic construct
  source 1..856: mol_type = other DNA
atgtacaggatgcaactcctgtcttgcattgcactaagtcttgcacttgtcacgaattcggcacctctcgagcgcaaatctagtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgcacgtacgagcactcgcaaacaaaattacatgatgaatttttcacgacaacatgggctcagacgtttctacaacagaagacgaaggtcacttaggcgatacccat

SEQ ID NO: 5 - protein, 285 aa, synthetic construct
MYRMQLLSCIALSLALVTNSAPLERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGARTSTRKQNYMMNFSRQHGLRRFYNRRRRSLRRYP

SEQ ID NO: 6 - DNA, 3881 bp, synthetic construct
  source 1..3881: mol_type = other DNA
atgtacaggatgcaactcctgtcttgcattgcactaagtcttgcacttgtcacgaattcggcacctctcgagcgcaaatctagtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgcacatgtacaggatgcaactcctgtcttgcattgcactaagtcttgcacttgtcacgaattcggcacctctcgagcgcaaatctagtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgcacgtacgggtggtggcggtagcggtggtggcggatcccaaaccaatgctacaagattcagactctggcaaccttataataacggtaagaaagaagaaatctggattgttgatgacttcattatcgatggaaataatgtaaacaaccctgtgatgctcttggatacatttgattttgggcccagagaagacaattggtttttctatcctggtggtaacatcggtctttattgtccatattcttcaaagggggcacctgaagaagattcagctatggtgtttgtttcaaatgaagttggtgagcattccattaccacccgtgacctaaatgtgaatgagaacaccatcatacaatttgagatcaacgttggctgttcgactgatagctcatccgcggatccagtgagactggaattttcaagggacttcggggcgacctggcaccttctgctgcccctctgctaccacagcagcagccacgtcagctctttatgctccaccgagcaccaccccagcagcacctactacgcaggaaccatgcagggctggaggagggaggtcgtgcactttgggaagctgcacctttgtggatctgtccgtttcagatggtaccagggattttaccctgccggctctcagccagtgacatgggccattgataatgtctacatcggtccccagtgtgaggagatgtgtaatggacaggggagctgtatcaatggaaccaaatgtatatgtgaccctggctactcaggtccaacctgtaaaataagcaccaaaaatcctgattttctcaaagatgatttcgaaggtcagctagaatctgatagattcttattaatgagtggtgggaaaccatctcgaaagtgtggaatcctttctagtggaaacaacctctttttcaatgaagatggcttgcgcatgttgatgacacgagacctggatttatcacatgctagatttgtgcagttcttcatgagactgggatgtggtaaaggcgttcctgaccccaggagtcaacccgtgctcctacagtattctctcaacggtggcctctcgtggagtcttcttcaggagttccttttcagcaattccagcaatgtgggcaggtacattgccctggagatacccttgaaagcccgttctggttctactcgccttcgctggtggcaaccgtctgagaatgggcacttctacagcccctgggttatcgatcagattcttattggaggaaatatttctggtaatatggtcttggaagatgatttcacaacccttgatagtaggaaatggctgcttcacccaggaggcaccaagatgcccgtgtgtggctctactggtgatgccctggtcttcattgaaaaggccagcacccgttacgtggtcagcacagacgtcgccgtgaatgaggattccttcctacagatagacttcgctgtctcctgctcagtcacagactcttgttatgcgattgaattggaatactcagtagatcttggattgtcatggcacccattggtaagggactgtctgcctaccaatgtggaatgcagtcgctatcatctgcaacggatcctggtgtcagacactttcaacaagtggactagaatcactctgcctctccctccttataccaggtcccaagccactcgtttccgttggcatcaaccagctccttttgacaagcagcagacatgggcaatagataatgtctatatcggggatggctgcatagacatgtgcagtggccatgggagatgcatccagggaaactgcgtctgtgatgaacagtggggtggcctgtactgtgatgaccccgagacctctcttccaacccaactcaaagacaacttcaatcgagctccatccagtcagaactggctgactgtgaacggagggaaattgagtacagtgtgtggagccgtggcgtcgggaatggctctccatttcagtgggggttgtagtcgattattagtcactgtggatctaaacctcactaatgctgagttcatccaattttacttcatgtatgggtgcctgattacaccaaacaaccgtaaccaaggtgttctcttggaatattctgtcaatggaggcattacctggaacctgctcatggagattttctatgaccagtacagtaagcccggatttgtgaatatccttctccctcctgatgctaaagagattgccactcgcttccgctggtggcagccaagacatgacggcctggatcagaacgactgggccattgacaatgtcctcatctcaggctctgctagcactcgcaaacaaaattacatgatgaatttttcacgacaacatgggctcagacgtttctacaacagaagacgaaggtcacttaggcgatacccat

SEQ ID NO: 7 - protein, 1042 aa, synthetic construct
MYRMQLLSCIALSLALVTNSAPLERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGARTGGGGSGGGGSQTNATRFRLWQPYNNGKKEEIWIVDDFIIDGNNVNNPVMLLDTFDFGPREDNWFFYPGGNIGLYCPYSSKGAPEEDSAMVFVSNEVGEHSITTRDLNVNENTIIQFEINVGCSTDSSSADPVRLEFSRDFGATWHLLLPLCYHSSSHVSSLCSTEHHPSSTYYAGTMQGWRREVVHFGKLHLCGSVRFRWYQGFYPAGSQPVTWAIDNVYIGPQCEEMCNGQGSCINGTKCICDPGYSGPTCKISTKNPDFLKDDFEGQLESDRFLLMSGGKPSRKCGILSSGNNLFFNEDGLRMLMTRDLDLSHARFVQFFMRLGCGKGVPDPRSQPVLLQYSLNGGLSWSLLQEFLFSNSSNVGRYIALEIPLKARSGSTRLRWWQPSENGHFYSPWVIDQILIGGNISGNMVLEDDFTTLDSRKWLLHPGGTKMPVCGSTGDALVFIEKASTRYVVSTDVAVNEDSFLQIDFAVSCSVTDSCYAIELEYSVDLGLSWHPLVRDCLPTNVECSRYHLQRILVSDTFNKWTRITLPLPPYTRSQATRFRWHQPAPFDKQQTWAIDNVYIGDGCIDMCSGHGRCIQGNCVCDEQWGGLYCDDPETSLPTQLKDNFNRAPSSQNWLTVNGGKLSTVCGAVASGMALHFSGGCSRLLVTVDLNLTNAEFIQFYFMYGCLITPNNRNQGVLLEYSVNGGITWNLLMEIFYDQYSKPGFVNILLPPDAKEIATRFRWWQPRHDGLDQNDWAIDNVLISGSASTRKQNYMMNFSRQHGLRRFYNRRRRSLRRYP

SEQ ID NO: 8 - DNA, 2791 bp, synthetic construct
  source 1..2791: mol_type = other DNA
atgtacaggatgcaactcctgtcttgcattgcactaagtcttgcacttgtcacgaattcgggtggtgactacaaagacgatgacgacaagggaggtgaacagtgtggcgcgattatgcatggcaatgccgtcaccttctgtgaaccatatggcccacgagaactgattaccacaggccttaatacaacaacagcttctgtcctccaattttccattgggtcaggttcatgtcgctttagttattcagaccccagcatcatcgtgttatatgccaagaataactctgcggactggattcagctagagaaaattagagccccttccaatgtcagcacaatcatccatatcctctaccttcctgaggacgccaaaggggagaatgtccaatttcagtggaagcaggaaaatcttcgtgtaggtgaagtgtatgaagcctgctgggccttaggaggacaaaccaatgctacaagattcagactctggcaaccttataataacggtaagaaagaagaaatctggattgttgatgacttcattatcgatggaaataatgtaaacaaccctgtgatgctcttggatacatttgattttgggcccagagaagacaattggtttttctatcctggtggtaacatcggtctttattgtccatattcttcaaagggggcacctgaagaagattcagctatggtgtttgtttcaaatgaagttggtgagcattccattaccacccgtgacctaaatgtgaatgagaacaccatcatacaatttgagatcaacgttggctgttcgactgatagctcatccgcggatccagtgagactggaattttcaagggacttcggggcgacctggcaccttctgctgcccctctgctaccacagcagcagccacgtcagctctttatgctccaccgagcaccaccccagcagcacctactacgcaggaaccatgcagggctggaggagggaggtcgtgcactttgggaagctgcacctttgtggatctgtccgtttcagatggtaccagggattttaccctgccggctctcagccagtgacatgggccattgataatgtctacatcggtccccagtgtgaggagatgtgtaatggacaggggagctgtatcaatggaaccaaatgtatatgtgaccctggctactcaggtccaacctgtaaaataagcaccaaaaatcctgattttctcaaagatgatttcgaaggtcagctagaatctgatagattcttattaatgagtggtgggaaaccatctcgaaagtgtggaatcctttctagtggaaacaacctctttttcaatgaagatggcttgcgcatgttgatgacacgagacctggatttatcacatgctagatttgtgcagttcttcatgagactgggatgtggtaaaggcgttcctgaccccaggagtcaacccgtgctcctacagtattctctcaacggtggcctctcgtggagtcttcttcaggagttccttttcagcaattccagcaatgtgggcaggtacattgccctggagatacccttgaaagcccgttctggttctactcgccttcgctggtggcaaccgtctgagaatgggcacttctacagcccctgggttatcgatcagattcttattggaggaaatatttctggtaatatggtcttggaagatgatttcacaacccttgatagtaggaaatggctgcttcacccaggaggcaccaagatgcccgtgtgtggctctactggtgatgccctggtcttcattgaaaaggccagcacccgttacgtggtcagcacagacgtcgccgtgaatgaggattccttcctacagatagacttcgctgtctcctgctcagtcacagactcttgttatgcgattgaattggaatactcagtagatcttggattgtcatggcacccattggtaagggactgtctgcctaccaatgtggaatgcagtcgctatcatctgcaacggatcctggtgtcagacactttcaacaagtggactagaatcactctgcctctccctccttataccaggtcccaagccactcgtttccgttggcatcaaccagctccttttgacaagcagcagacatgggcaatagataatgtctatatcggggatggctgcatagacatgtgcagtggccatgggagatgcatccagggaaactgcgtctgtgatgaacagtggggtggcctgtactgtgatgaccccgagacctctcttccaacccaactcaaagacaacttcaatcgagctccatccagtcagaactggctgactgtgaacggagggaaattgagtacagtgtgtggagccgtggcgtcgggaatggctctccatttcagtgggggttgtagtcgattattagtcactgtggatctaaacctcactaatgctgagttcatccaattttacttcatgtatgggtgcctgattacaccaaacaaccgtaaccaaggtgttctcttggaatattctgtcaatggaggcattacctggaacctgctcatggagattttctatgaccagtacagtaagcccggatttgtgaatatccttctccctcctgatgctaaagagattgccactcgcttccgctggtggcagccaagacatgacggcctggatcagaacgactgggccattgacaatgtcctcatctcaggctctgctagcactcgcaaacaaaattacatgatgaatttttcacgacaacatgggctcagacgtttctacaacagaagacgaaggtcacttaggcgatacccat

SEQ ID NO: 9 - protein, 930 aa, synthetic construct
MYRMQLLSCIALSLALVTNSGGDYKDDDDKGGEQCGAIMHGNAVTFCEPYGPRELITTGLNTTTASVLQFSIGSGSCRFSYSDPSIIVLYAKNNSADWIQLEKIRAPSNVSTIIHILYLPEDAKGENVQFQWKQENLRVGEVYEACWALGGQTNATRFRLWQPYNNGKKEEIWIVDDFIIDGNNVNNPVMLLDTFDFGPREDNWFFYPGGNIGLYCPYSSKGAPEEDSAMVFVSNEVGEHSITTRDLNVNENTIIQFEINVGCSTDSSSADPVRLEFSRDFGATWHLLLPLCYHSSSHVSSLCSTEHHPSSTYYAGTMQGWRREVVHFGKLHLCGSVRFRWYQGFYPAGSQPVTWAIDNVYIGPQCEEMCNGQGSCINGTKCICDPGYSGPTCKISTKNPDFLKDDFEGQLESDRFLLMSGGKPSRKCGILSSGNNLFFNEDGLRMLMTRDLDLSHARFVQFFMRLGCGKGVPDPRSQPVLLQYSLNGGLSWSLLQEFLFSNSSNVGRYIALEIPLKARSGSTRLRWWQPSENGHFYSPWVIDQILIGGNISGNMVLEDDFTTLDSRKWLLHPGGTKMPVCGSTGDALVFIEKASTRYVVSTDVAVNEDSFLQIDFAVSCSVTDSCYAIELEYSVDLGLSWHPLVRDCLPTNVECSRYHLQRILVSDTFNKWTRITLPLPPYTRSQATRFRWHQPAPFDKQQTWAIDNVYIGDGCIDMCSGHGRCIQGNCVCDEQWGGLYCDDPETSLPTQLKDNFNRAPSSQNWLTVNGGKLSTVCGAVASGMALHFSGGCSRLLVTVDLNLTNAEFIQFYFMYGCLITPNNRNQGVLLEYSVNGGITWNLLMEIFYDQYSKPGFVNILLPPDAKEIATRFRWWQPRHDGLDQNDWAIDNVLISGSASTRKQNYMMNFSRQHGLRRFYNRRRRSLRRYP

SEQ ID NO: 10 - DNA, 1975 bp, synthetic construct
  source 1..1975: mol_type = other DNA
atgtacaggatgcaactcctgtcttgcattgcactaagtcttgcacttgtcacgaattcggcacctctcgagcgcaaatctagtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgcacgtacgggtggtggcggtagcggtggtggcggatccatggcgccgcggagggtcaggtcgtttctgcgcgggctcccggcgctgctactgctgctgctcttcctcgggccctggcccgctgcgagccacggcggcaagtactcgcgggagaagaaccagcccaagccgtccccgaaacgcgagtccggagaggagttccgcatggagaagttgaaccagctgtgggagaaggcccagcgactgcatcttcctcccgtgaggctggccgagctccacgctgatctgaagatacaggagagggacgaactcgcctggaagaaactaaagcttgacggcttggacgaagatggggagaaggaagcgagactcatacgcaacctcaatgtcatcttggccaagtatggtctggacggaaagaaggacgctcggcaggtgaccagcaactccctcagtggcacccaggaagacgggctggatgaccccaggctggaaaagctgtggcacaaggcgaagacctctgggaaattctccggcgaagaactggacaagctctggcgggagttcctgcatcacaaagagaaagttcacgagtacaacgtcctgctggagaccctgagcaggaccgaagaaatccacgagaacgtcattagcccctcggacctgagcgacatcaagggcagcgtcctgcacagcaggcacacggagctgaaggagaagctgcgcagcatcaaccagggcctggaccgcctgcgcagggtcagccaccagggctacagcactgaggctgagttcgaggagcccagggtgattgacctgtgggacctggcgcagtccgccaacctcacggacaaggagctggaggcgttccgggaggagctcaagcacttcgaagccaaaatcgagaagcacaaccactaccagaagcagctggagattgcgcacgagaagctgaggcacgcagagagcgtgggcgacggcgagcgtgtgagccgcagccgcgagaagcacgccctgctggaggggcggaccaaggagctgggctacacggtgaagaagcatctgcaggacctgtccggcaggatctccagagctcggcacaacgaactcaccggtggatctggagggagcactcgcaaacaaaattacatgatgaatttttcacgacaacatgggctcagacgtttctacaacagaagacgaaggtcacttaggcgatacccat

SEQ ID NO: 11 - protein, 658 aa, synthetic construct
MYRMQLLSCIALSLALVTNSAPLERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGARTGGGGSGGGGSMAPRRVRSFLRGLPALLLLLLFLGPWPAASHGGKYSREKNQPKPSPKRESGEEFRMEKLNQLWEKAQRLHLPPVRLAELHADLKIQERDELAWKKLKLDGLDEDGEKEARLIRNLNVILAKYGLDGKKDARQVTSNSLSGTQEDGLDDPRLEKLWHKAKTSGKFSGEELDKLWREFLHHKEKVHEYNVLLETLSRTEEIHENVISPSDLSDIKGSVLHSRHTELKEKLRSINQGLDRLRRVSHQGYSTEAEFEEPRVIDLWDLAQSANLTDKELEAFREELKHFEAKIEKHNHYQKQLEIAHEKLRHAESVGDGERVSRSREKHALLEGRTKELGYTVKKHLQDLSGRISRARHNELTGGSGGSTRKQNYMMNFSRQHGLRRFYNRRRRSLRRYP

SEQ ID NO: 12 - DNA, 1981 bp, synthetic construct
  source 1..1981: mol_type = other DNA
atgtacaggatgcaactcctgtcttgcattgcactaagtcttgcacttgtcacgaattccatggcgccgcggagggtcaggtcgtttctgcgcgggctcccggcgctgctactgctgctgctcttcctcgggccctggcccgctgcgagccacggcggcaagtactcgcgggagaagaaccagcccaagccgtccccgaaacgcgagtccggagaggagttccgcatggagaagttgaaccagctgtgggagaaggcccagcgactgcatcttcctcccgtgaggctggccgagctccacgctgatctgaagatacaggagagggacgaactcgcctggaagaaactaaagcttgacggcttggacgaagatggggagaaggaagcgagactcatacgcaacctcaatgtcatcttggccaagtatggtctggacggaaagaaggacgctcggcaggtgaccagcaactccctcagtggcacccaggaagacgggctggatgaccccaggctggaaaagctgtggcacaaggcgaagacctctgggaaattctccggcgaagaactggacaagctctggcgggagttcctgcatcacaaagagaaagttcacgagtacaacgtcctgctggagaccctgagcaggaccgaagaaatccacgagaacgtcattagcccctcggacctgagcgacatcaagggcagcgtcctgcacagcaggcacacggagctgaaggagaagctgcgcagcatcaaccagggcctggaccgcctgcgcagggtcagccaccagggctacagcactgaggctgagttcgaggagcccagggtgattgacctgtgggacctggcgcagtccgccaacctcacggacaaggagctggaggcgttccgggaggagctcaagcacttcgaagccaaaatcgagaagcacaaccactaccagaagcagctggagattgcgcacgagaagctgaggcacgcagagagcgtgggcgacggcgagcgtgtgagccgcagccgcgagaagcacgccctgctggaggggcggaccaaggagctgggctacacggtgaagaagcatctgcaggacctgtccggcaggatctccagagctcggcacaacgaactcaccggtggatctggagggaattcggcacctctcgagcgcaaatctagtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgcacgtacgggtggtggcggtagcggtggtggcggatccagcactcgcaaacaaaattacatgatgaatttttcacgacaacatgggctcagacgtttctacaacagaagacgaaggtcacttaggcgatacccat

SEQ ID NO: 13 - protein, 660 aa, synthetic construct
MYRMQLLSCIALSLALVTNSMAPRRVRSFLRGLPALLLLLLFLGPWPAASHGGKYSREKNQPKPSPKRESGEEFRMEKLNQLWEKAQRLHLPPVRLAELHADLKIQERDELAWKKLKLDGLDEDGEKEARLIRNLNVILAKYGLDGKKDARQVTSNSLSGTQEDGLDDPRLEKLWHKAKTSGKFSGEELDKLWREFLHHKEKVHEYNVLLETLSRTEEIHENVISPSDLSDIKGSVLHSRHTELKEKLRSINQGLDRLRRVSHQGYSTEAEFEEPRVIDLWDLAQSANLTDKELEAFREELKHFEAKIEKHNHYQKQLEIAHEKLRHAESVGDGERVSRSREKHALLEGRTKELGYTVKKHLQDLSGRISRARHNELTGGSGGNSAPLERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGARTGGGGSGGGGSSTRKQNYMMNFSRQHGLRRFYNRRRRSLRRYP

SEQ ID NO: 14 - DNA, 1855 bp, synthetic construct
  source 1..1855: mol_type = other DNA
atgtacaggatgcaactcctgtcttgcattgcactaagtcttgcacttgtcacgaattcggcacctctcgagcgcaaatctagtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgcacgtacgggtggtggcggtagcggtggtggcggatcgatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgagcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcacaccggtggatctggagggagcactcgcaaacaaaattacatgatgaatttttcacgacaacatgggctcagacgtttctacaacagaagacgaaggtcacttaggcgatacccat

SEQ ID NO: 15 - protein, 618 aa, synthetic construct
MYRMQLLSCIALSLALVTNSAPLERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGARTGGGGSGGGGSMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVSLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNHTGGSGGSTRKQNYMMNFSRQHGLRRFYNRRRRSLRRYP

SEQ ID NO: 16 - DNA, 1855 bp, synthetic construct
  source 1..1855: mol_type = other DNA
atgtacaggatgcaactcctgtcttgcattgcactaagtcttgcacttgtcacgaattcggcacctctcgagcgcaaatctagtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtgcacgtacgggtggtggcggtagcggtggtggcggatcgatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcacaccggtggatctggagggagcactcgcaaacaaaattacatgatgaatttttcacgacaacatgggctcagacgtttctacaacagaagacgaaggtcacttaggcgatacccat

SEQ ID NO: 17 - protein, 618 aa, synthetic construct
MYRMQLLSCIALSLALVTNSAPLERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGARTGGGGSGGGGSMKVLWAALLVTFLAGCQAKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNHTGGSGGSTRKQNYMMNFSRQHGLRRFYNRRRRSLRRYP

SEQ ID NO: 18 - DNA, 2794 bp, synthetic construct
  source 1..2794: mol_type = other DNA
atgtacaggatgcaactcctgtcttgcattgcactaagtcttgcacttgtcacgaattcgggtggtcatcatcatcaccatcacggaggtgaacagtgtggcgcgattatgcatggcaatgccgtcaccttctgtgaaccatatggcccacgagaactgattaccacaggccttaatacaacaacagcttctgtcctccaattttccattgggtcaggttcatgtcgctttagttattcagaccccagcatcatcgtgttatatgccaagaataactctgcggactggattcagctagagaaaattagagccccttccaatgtcagcacaatcatccatatcctctaccttcctgaggacgccaaaggggagaatgtccaatttcagtggaagcaggaaaatcttcgtgtaggtgaagtgtatgaagcctgctgggccttaggttctagaggaggacaaaccaatgctacaagattcagactctggcaaccttataataacggtaagaaagaagaaatctggattgttgatgacttcattatcgatggaaataatgtaaacaaccctgtgatgctcttggatacatttgattttgggcccagagaagacaattggtttttctatcctggtggtaacatcggtctttattgtccatattcttcaaagggggcacctgaagaagattcagctatggtgtttgtttcaaatgaagttggtgagcattccattaccacccgtgacctaaatgtgaatgagaacaccatcatacaatttgagatcaacgttggctgttcgactgatagctcatccgcggatccagtgagactggaattttcaagggacttcggggcgacctggcaccttctgctgcccctctgctaccacagcagcagccacgtcagctctttatgctccaccgagcaccaccccagcagcacctactacgcaggaaccatgcagggctggaggagggaggtcgtgcactttgggaagctgcacctttgtggatctgtccgtttcagatggtaccagggattttaccctgccggctctcagccagtgacatgggccattgataatgtctacatcggtccccagtgtgaggagatgtgtaatggacaggggagctgtatcaatggaaccaaatgtatatgtgaccctggctactcaggtccaacctgtaaaataagcaccaaaaatcctgattttctcaaagatgatttcgaaggtcagctagaatctgatagattcttattaatgagtggtgggaaaccatctcgaaagtgtggaatcctttctagtggaaacaacctctttttcaatgaagatggcttgcgcatgttgatgacacgagacctggatttatcacatgctagatttgtgcagttcttcatgagactgggatgtggtaaaggcgttcctgaccccaggagtcaacccgtgctcctacagtattctctcaacggtggcctctcgtggagtcttcttcaggagttccttttcagcaattccagcaatgtgggcaggtacattgccctggagatacccttgaaagcccgttctggttctactcgccttcgctggtggcaaccgtctgagaatgggcacttctacagcccctgggttatcgatcagattcttattggaggaaatatttctggtaatatggtcttggaagatgatttcacaacccttgatagtaggaaatggctgcttcacccaggaggcaccaagatgcccgtgtgtggctctactggtgatgccctggtcttcattgaaaaggccagcacccgttacgtggtcagcacagacgtcgccgtgaatgaggattccttcctacagatagacttcgctgtctcctgctcagtcacagactcttgttatgcgattgaattggaatactcagtagatcttggattgtcatggcacccattggtaagggactgtctgcctaccaatgtggaatgcagtcgctatcatctgcaacggatcctggtgtcagacactttcaacaagtggactagaatcactctgcctctccctccttataccaggtcccaagccactcgtttccgttggcatcaaccagctccttttgacaagcagcagacatgggcaatagataatgtctatatcggggatggctgcatagacatgtgcagtggccatgggagatgcatccagggaaactgcgtctgtgatgaacagtggggtggcctgtactgtgatgaccccgagacctctcttccaacccaactcaaagacaacttcaatcgagctccatccagtcagaactggctgactgtgaacggagggaaattgagtacagtgtgtggagccgtggcgtcgggaatggctctccatttcagtgggggttgtagtcgattattagtcactgtggatctaaacctcactaatgctgagttcatccaattttacttcatgtatgggtgcctgattacaccaaacaaccgtaaccaaggtgttctcttggaatattctgtcaatggaggcattacctggaacctgctcatggagattttctatgaccagtacagtaagcccggatttgtgaatatccttctccctcctgatgctaaagagattgccactcgcttccgctggtggcagccaagacatgacggcctggatcagaacgactgggccattgacaatgtcctcatctcaggctctgctagcactcgcaaacaaaattacatgatgaatttttcacgacaacatgggctcagacgtttctacaacagaagacgaaggtcacttaggcgatacccat

SEQ ID NO: 19 - protein, 931 aa, synthetic construct
MYRMQLLSCIALSLALVTNSGGHHHHHHGGEQCGAIMHGNAVTFCEPYGPRELITTGLNTTTASVLQFSIGSGSCRFSYSDPSIIVLYAKNNSADWIQLEKIRAPSNVSTIIHILYLPEDAKGENVQFQWKQENLRVGEVYEACWALGSRGGQTNATRFRLWQPYNNGKKEEIWIVDDFIIDGNNVNNPVMLLDTFDFGPREDNWFFYPGGNIGLYCPYSSKGAPEEDSAMVFVSNEVGEHSITTRDLNVNENTIIQFEINVGCSTDSSSADPVRLEFSRDFGATWHLLLPLCYHSSSHVSSLCSTEHHPSSTYYAGTMQGWRREVVHFGKLHLCGSVRFRWYQGFYPAGSQPVTWAIDNVYIGPQCEEMCNGQGSCINGTKCICDPGYSGPTCKISTKNPDFLKDDFEGQLESDRFLLMSGGKPSRKCGILSSGNNLFFNEDGLRMLMTRDLDLSHARFVQFFMRLGCGKGVPDPRSQPVLLQYSLNGGLSWSLLQEFLFSNSSNVGRYIALEIPLKARSGSTRLRWWQPSENGHFYSPWVIDQILIGGNISGNMVLEDDFTTLDSRKWLLHPGGTKMPVCGSTGDALVFIEKASTRYVVSTDVAVNEDSFLQIDFAVSCSVTDSCYAIELEYSVDLGLSWHPLVRDCLPTNVECSRYHLQRILVSDTFNKWTRITLPLPPYTRSQATRFRWHQPAPFDKQQTWAIDNVYIGDGCIDMCSGHGRCIQGNCVCDEQWGGLYCDDPETSLPTQLKDNFNRAPSSQNWLTVNGGKLSTVCGAVASGMALHFSGGCSRLLVTVDLNLTNAEFIQFYFMYGCLITPNNRNQGVLLEYSVNGGITWNLLMEIFYDQYSKPGFVNILLPPDAKEIATRFRWWQPRHDGLDQNDWAIDNVLISGSASTRKQNYMMNFSRQHGLRRFYNRRRRSLRRYP

SEQ ID NO: 20 - DNA, 4534 bp, synthetic construct
  source 1..4534: mol_type = other DNA
atgtacaggatgcaactcctgtcttgcattgcactaagtcttgcacttgtcacgaattcgggtggtcatcatcatcaccatcacggaggttctagactgtccgagaagcagaagcagatcatcccagttatcaatccaactttacctcagaacttttatgagaagccagcttttgattaccctatgaatcagatgagtgtgtggttgatgttggctaatgaaggaatggttaaaaatgaaaccttctgtgctgccacaccatcagcaatgatatttggaaaatcagatggagatcgatttgcagtaactcgagatttgaccctgaaacctggatatgtgctacagttcaagctaaacataggttgtgccaatcaattcagcagtactgctccagttcttcttcagtactctcatgatgctggtatgtcctggtttctggtgaaagaaggctgttacccggcttctgcaggcaaaggatgcgaaggaaactccagagaactaagtgagcccaccatgtatcacacaggggactttgaagaatggacaagaatcaccattgttattccaaggtctcttgcatccagcaagaccagattccgatggatccaggagagcagctcacagaaaaacgtgcctccatttggtttagatggagtgtacatatccgagccttgtcccagttactgcagtggccatggggactgcatttcaggagtgtgtttctgtgacctgggatatactgctgcacaaggaacctgtgtgtcaaatgtccccaatcacaatgagatgttcgataggtttgaggggaagctcagccctctgtggtacaagataacaggtgcccaggttggaactggctgtggaacacttaacgatggcaaatctctctacttcaatggccctgggaaaagggaagcccgtacggtccctctggacaccaggaatatcagacttgttcaattttatatacaaattggaagcaaaacttcaggcattacctgcatcaaaccaagaactagaaatgaagggcttattgttcagtattcaaatgacaatgggatactctggcatttgcttcgagagttggacttcatgtccttcctggaaccacagatcatttccattgacctgccacaggacgcgaagacacctgcaacggcatttcgatggtggcaaccgcaacatgggaagcattcagcccagtgggctttggatgatgttcttataggaatgaatgacagctctcaaactggatttcaagacaaatttgatggctctatagatttgcaagccaactggtatcgaatccaaggaggtcaagttgatattgactgtctctctatggatactgctctgatattcactgaaaacataggaaaacctcgttatgctgagacctgggattttcatgtgtcagcatctacctttttgcagtttgaaatgagcatgggctgtagcaagcccttcagcaactcccacagtgtacagctccagtattctctgaacaatggcaaggactggcatcttgtcaccgaagagtgtgttcctccaaccattggctgtctgcattacacggaaagttcaatttacacctcggaaagattccagaattggaagcggatcactgtctaccttccactctccaccatttctcccaggacccggttcagatggattcaggccaactacactgtgggggctgattcctgggcgattgataatgttgtactggcctcagggtgcccttggatgtgctcaggacgagggatttgtgatgctggacgctgtgtgtgtgaccggggctttggtggaccctattgtgttcctgttgttcctctgccctcgattcttaaagacgatttcaatgggaatttacatcctgacctttggcctgaagtgtatggtgcagagagggggaatctgaatggtgaaaccatcaaatctggaacatctctaatttttaaaggggaaggactaaggatgcttatttcaagagatctagattgtacaaatacaatgtatgtccagttttcacttagatttatagcaaaaagtaccccagagagatctcactctattctgttacaattctccatcagtggaggaatcacttggcacctgatggatgaattttactttcctcaaacaacgaatatacttttcatcaatgttcccttgccatacactgcccaaaccaatgctacaagattcagactctggcaaccttataataacggtaagaaagaagaaatctggattgttgatgacttcattatcgatggaaataatgtaaacaaccctgtgatgctcttggatacatttgattttgggcccagagaagacaattggtttttctatcctggtggtaacatcggtctttattgtccatattcttcaaagggggcacctgaagaagattcagctatggtgtttgtttcaaatgaagttggtgagcattccattaccacccgtgacctaaatgtgaatgagaacaccatcatacaatttgagatcaacgttggctgttcgactgatagctcatccgcggatccagtgagactggaattttcaagggacttcggggcgacctggcaccttctgctgcccctctgctaccacagcagcagccacgtcagctctttatgctccaccgagcaccaccccagcagcacctactacgcaggaaccatgcagggctggaggagggaggtcgtgcactttgggaagctgcacctttgtggatctgtccgtttcagatggtaccagggattttaccctgccggctctcagccagtgacatgggccattgataatgtctacatcggtccccagtgtgaggagatgtgtaatggacaggggagctgtatcaatggaaccaaatgtatatgtgaccctggctactcaggtccaacctgtaaaataagcaccaaaaatcctgattttctcaaagatgatttcgaaggtcagctagaatctgatagattcttattaatgagtggtgggaaaccatctcgaaagtgtggaatcctttctagtggaaacaacctctttttcaatgaagatggcttgcgcatgttgatgacacgagacctggatttatcacatgctagatttgtgcagttcttcatgagactgggatgtggtaaaggcgttcctgaccccaggagtcaacccgtgctcctacagtattctctcaacggtggcctctcgtggagtcttcttcaggagttccttttcagcaattccagcaatgtgggcaggtacattgccctggagatacccttgaaagcccgttctggttctactcgccttcgctggtggcaaccgtctgagaatgggcacttctacagcccctgggttatcgatcagattcttattggaggaaatatttctggtaatatggtcttggaagatgatttcacaacccttgatagtaggaaatggctgcttcacccaggaggcaccaagatgcccgtgtgtggctctactggtgatgccctggtcttcattgaaaaggccagcacccgttacgtggtcagcacagacgtcgccgtgaatgaggattccttcctacagatagacttcgctgtctcctgctcagtcacagactcttgttatgcgattgaattggaatactcagtagatcttggattgtcatggcacccattggtaagggactgtctgcctaccaatgtggaatgcagtcgctatcatctgcaacggatcctggtgtcagacactttcaacaagtggactagaatcactctgcctctccctccttataccaggtcccaagccactcgtttccgttggcatcaaccagctccttttgacaagcagcagacatgggcaatagataatgtctatatcggggatggctgcatagacatgtgcagtggccatgggagatgcatccagggaaactgcgtctgtgatgaacagtggggtggcctgtactgtgatgaccccgagacctctcttccaacccaactcaaagacaacttcaatcgagctccatccagtcagaactggctgactgtgaacggagggaaattgagtacagtgtgtggagccgtggcgtcgggaatggctctccatttcagtgggggttgtagtcgattattagtcactgtggatctaaacctcactaatgctgagttcatccaattttacttcatgtatgggtgcctgattacaccaaacaaccgtaaccaaggtgttctcttggaatattctgtcaatggaggcattacctggaacctgctcatggagattttctatgaccagtacagtaagcccgggtttgtgaatatccttctccctcctgatgctaaagagattgccactcgcttccgctggtggcagccaagacatgacggcctggatcagaacgactgggccattgacaatgtcctcatcaccggtggatctggagggagcactcgcaaacaaaattacatgatgaatttttcacgacaacatgggctcagacgtttctacaacagaagacgaaggtcacttaggcgatacccat

SEQ ID NO: 21 - protein, 1511 aa, synthetic construct
MYRMQLLSCIALSLALVTNSGGHHHHHHGGSRLSEKQKQIIPVINPTLPQNFYEKPAFDYPMNQMSVWLMLANEGMVKNETFCAATPSAMIFGKSDGDRFAVTRDLTLKPGYVLQFKLNIGCANQFSSTAPVLLQYSHDAGMSWFLVKEGCYPASAGKGCEGNSRELSEPTMYHTGDFEEWTRITIVIPRSLASSKTRFRWIQESSSQKNVPPFGLDGVYISEPCPSYCSGHGDCISGVCFCDLGYTAAQGTCVSNVPNHNEMFDRFEGKLSPLWYKITGAQVGTGCGTLNDGKSLYFNGPGKREARTVPLDTRNIRLVQFYIQIGSKTSGITCIKPRTRNEGLIVQYSNDNGILWHLLRELDFMSFLEPQIISIDLPQDAKTPATAFRWWQPQHGKHSAQWALDDVLIGMNDSSQTGFQDKFDGSIDLQANWYRIQGGQVDIDCLSMDTALIFTENIGKPRYAETWDFHVSASTFLQFEMSMGCSKPFSNSHSVQLQYSLNNGKDWHLVTEECVPPTIGCLHYTESSIYTSERFQNWKRITVYLPLSTISPRTRFRWIQANYTVGADSWAIDNVVLASGCPWMCSGRGICDAGRCVCDRGFGGPYCVPVVPLPSILKDDFNGNLHPDLWPEVYGAERGNLNGETIKSGTSLIFKGEGLRMLISRDLDCTNTMYVQFSLRFIAKSTPERSHSILLQFSISGGITWHLMDEFYFPQTTNILFINVPLPYTAQTNATRFRLWQPYNNGKKEEIWIVDDFIIDGNNVNNPVMLLDTFDFGPREDNWFFYPGGNIGLYCPYSSKGAPEEDSAMVFVSNEVGEHSITTRDLNVNENTIIQFEINVGCSTDSSSADPVRLEFSRDFGATWHLLLPLCYHSSSHVSSLCSTEHHPSSTYYAGTMQGWRREVVHFGKLHLCGSVRFRWYQGFYPAGSQPVTWAIDNVYIGPQCEEMCNGQGSCINGTKCICDPGYSGPTCKISTKNPDFLKDDFEGQLESDRFLLMSGGKPSRKCGILSSGNNLFFNEDGLRMLMTRDLDLSHARFVQFFMRLGCGKGVPDPRSQPVLLQYSLNGGLSWSLLQEFLFSNSSNVGRYIALEIPLKARSGSTRLRWWQPSENGHFYSPWVIDQILIGGNISGNMVLEDDFTTLDSRKWLLHPGGTKMPVCGSTGDALVFIEKASTRYVVSTDVAVNEDSFLQIDFAVSCSVTDSCYAIELEYSVDLGLSWHPLVRDCLPTNVECSRYHLQRILVSDTFNKWTRITLPLPPYTRSQATRFRWHQPAPFDKQQTWAIDNVYIGDGCIDMCSGHGRCIQGNCVCDEQWGGLYCDDPETSLPTQLKDNFNRAPSSQNWLTVNGGKLSTVCGAVASGMALHFSGGCSRLLVTVDLNLTNAEFIQFYFMYGCLITPNNRNQGVLLEYSVNGGITWNLLMEIFYDQYSKPGFVNILLPPDAKEIATRFRWWQPRHDGLDQNDWAIDNVLITGGSGGSTRKQNYMMNFSRQHGLRRFYNRRRRSLRRYP

SEQ ID NO: 22 - DNA, 4534 bp, synthetic construct
  source 1..4534: mol_type = other DNA
atgtacaggatgcaactcctgtcttgcattgcactaagtcttgcacttgtcacgaattcgggtggtcatcatcatcaccatcacggaggttctagactgtccgagaagcagaagcagatcatcccagttatcaatccaactttacctcagaacttttatgagaagccagcttttgattaccctatgaatcagatgagtgtgtggttgatgttggctaatgaaggaatggttaaaaatgaaaccttctgtgctgccacaccatcagcaatgatatttggaaaatcagatggagatcgatttgcagtaactcgagatttgaccctgaaacctggatatgtgctacagttcaagctaaacataggttgtgccaatcaattcagcagtactgctccagttcttcttcagtactctcatgatgctggtatgtcctggtttctggtgaaagaaggctgttacccggcttctgcaggcaaaggatgcgaaggaaactccagagaactaagtgagcccaccatgtatcacacaggggactttgaagaatggacaagaatcaccattgttattccaaggtctcttgcatccagcaagaccagattccgatggatccaggagagcagctcacagaaaaacgtgcctccatttggtttagatggagtgtacatatccgagccttgtcccagttactgcagtggccatggggactgcatttcaggagtgtgtttctgtgacctgggatatactgctgcacaaggaacctgtgtgtcaaatgtccccaatcacaatgagatgttcgataggtttgaggggaagctcagccctctgtggtacaagataacaggtgcccaggttggaactggctgtggaacacttaacgatggcaaatctctctacttcaatggccctgggaaaagggaagcccgtacggtccctctggacaccaggaatatcagacttgttcaattttatatacaaattggaagcaaaacttcaggcattacctgcatcaaaccaagaactagaaatgaagggcttattgttcagtattcaaatgacaatgggatactctggcatttgcttcgagagttggacttcatgtccttcctggaaccacagatcatttccattgacctgccacaggacgcgaagacacctgcaacggcatttcgatggtggcaaccgcaacatgggaagcattcagcccagtgggctttggatgatgttcttataggaatgaatgacagctctcaaactggatttcaagacaaatttgatggctctatagatttgcaagccaactggtatcgaatccaaggaggtcaagttgatattgactgtctctctatggatactgctctgatattcactgaaaacataggaaaacctcgttatgctgagacctgggattttcatgtgtcagcatctacctttttgcagtttgaaatgagcatgggctgtagcaagcccttcagcaactcccacagtgtacagctccagtattctctgaacaatggcaaggactggcatcttgtcaccgaagagtgtgttcctccaaccattggctgtctgcattacacggaaagttcaatttacacctcggaaagattccagaattggaagcggatcactgtctaccttccactctccaccatttctcccaggacccggttcagatggattcaggccaactacactgtgggggctgattcctgggcgattgataatgttgtactggcctcagggtgcccttggatgtgctcaggacgagggatttgtgatgctggacgctgtgtgtgtgaccggggctttggtggaccctattgtgttcctgttgttcctctgccctcgattcttaaagacgatttcaatgggaatttacatcctgacctttggcctgaagtgtatggtgcagagagggggaatctgaatggtgaaaccatcaaatctggaacatctctaatttttaaaggggaaggactaaggatgcttatttcaagagatctagattgtacaaatacaatgtatgtccagttttcacttagatttatagcaaaaagtaccccagagagatctcactctattctgttacaattctccatcagtggaggaatcacttggcacctgatggatgaattttactttcctcaaacaacgaatatacttttcatcaatgttcccttgccatacactgcccaaaccaatgctacaagattcagactctggcaaccttataataacggtaagaaagaagaaatctggattgttgatgacttcattatcgatggaaataatgtaaacaaccctgtgatgctcttggatacatttgattttgggcccagagaagacaattggtttttctatcctggtggtaacatcggtctttattgtccatattcttcaaagggggcacctgaagaagattcagctatggtgtttgtttcaaatgaagttggtgagcattccattaccacccgtgacctaaatgtgaatgagaacaccatcatacaatttgagatcaacgttggctgttcgactgatagctcatccgcggatccagtgagactggaattttcaagggacttcggggcgacctggcaccttctgctgcccctctgctaccacagcagcagccacgtcagctctttatgctccaccgagcaccaccccagcagcacctactacgcaggaaccatgcagggctggaggagggaggtcgtgcactttgggaagctgcacctttgtggatctgtccgtttcagatggtaccagggattttaccctgccggctctcagccagtgacatgggccattgataatgtctacatcggtccccagtgtgaggagatgtgtaatggacaggggagctgtatcaatggaaccaaatgtatatgtgaccctggctactcaggtccaacctgtaaaataagcaccaaaaatcctgattttctcaaagatgatttcgaaggtcagctagaatctgatagattcttattaatgagtggtgggaaaccatctcgaaagtgtggaatcctttctagtggaaacaacctctttttcaatgaagatggcttgcgcatgttgatgacacgagacctggatttatcacatgctagatttgtgcagttcttcatgagactgggatgtggtaaaggcgttcctgaccccaggagtcaacccgtgctcctacagtattctctcaacggtggcctctcgtggagtcttcttcaggagttccttttcagcaattccagcaatgtgggcaggtacattgccctggagatacccttgaaagcccgttctggttctactcgccttcgctggtggcaaccgtctgagaatgggcacttctacagcccctgggttatcgatcagattcttattggaggaaatatttctggtaatatggtcttggaagatgatttcacaacccttgatagtaggaaatggctgcttcacccaggaggcaccaagatgcccgtgtgtggctctactggtgatgccctggtcttcattgaaaaggccagcacccgttacgtggtcagcacagacgtcgccgtgaatgaggattccttcctacagatagacttcgctgtctcctgctcagtcacagactcttgttatgcgattgaattggaatactcagtagatcttggattgtcatggcacccattggtaagggactgtctgcctaccaatgtggaatgcagtcgctatcatctgcaacggatcctggtgtcagacactttcaacaagtggactagaatcactctgcctctccctccttataccaggtcccaagccactcgtttccgttggcatcaaccagctccttttgacaagcagcagacatgggcaatagataatgtctatatcggggatggctgcatagacatgtgcagtggccatgggagatgcatccagggaaactgcgtctgtgatgaacagtggggtggcctgtactgtgatgaccccgagacctctcttccaacccaactcaaagacaacttcaatcgagctccatccagtcagaactggctgactgtgaacggagggaaattgagtacagtgtgtggagccgtggcgtcgggaatggctctccatttcagtgggggttgtagtcgattattagtcactgtggatctaaacctcactaatgctgagttcatccaattttacttcatgtatgggtgcctgattacaccaaacaaccgtaaccaaggtgttctcttggaatattctgtcaatggaggcattacctggaacctgctcatggagattttctatgaccagtacagtaagcccgggtttgtgaatatccttctccctcctgatgctaaagagattgccactcgcttccgctggtggcagccaagacatgacggcctggatcagaacgactgggccattgacaatgtcctcatcaccggtggatctggagggagcactcgcaaacaaaattacatgatgaatttttcacgacaacatgggctcagacatttctacaacagaagacgaaggtcacttaggcgatacccat

SEQ ID NO: 23 - protein, 1511 aa, synthetic construct
MYRMQLLSCIALSLALVTNSGGHHHHHHGGSRLSEKQKQIIPVINPTLPQNFYEKPAFDYPMNQMSVWLMLANEGMVKNETFCAATPSAMIFGKSDGDRFAVTRDLTLKPGYVLQFKLNIGCANQFSSTAPVLLQYSHDAGMSWFLVKEGCYPASAGKGCEGNSRELSEPTMYHTGDFEEWTRITIVIPRSLASSKTRFRWIQESSSQKNVPPFGLDGVYISEPCPSYCSGHGDCISGVCFCDLGYTAAQGTCVSNVPNHNEMFDRFEGKLSPLWYKITGAQVGTGCGTLNDGKSLYFNGPGKREARTVPLDTRNIRLVQFYIQIGSKTSGITCIKPRTRNEGLIVQYSNDNGILWHLLRELDFMSFLEPQIISIDLPQDAKTPATAFRWWQPQHGKHSAQWALDDVLIGMNDSSQTGFQDKFDGSIDLQANWYRIQGGQVDIDCLSMDTALIFTENIGKPRYAETWDFHVSASTFLQFEMSMGCSKPFSNSHSVQLQYSLNNGKDWHLVTEECVPPTIGCLHYTESSIYTSERFQNWKRITVYLPLSTISPRTRFRWIQANYTVGADSWAIDNVVLASGCPWMCSGRGICDAGRCVCDRGFGGPYCVPVVPLPSILKDDFNGNLHPDLWPEVYGAERGNLNGETIKSGTSLIFKGEGLRMLISRDLDCTNTMYVQFSLRFIAKSTPERSHSILLQFSISGGITWHLMDEFYFPQTTNILFINVPLPYTAQTNATRFRLWQPYNNGKKEEIWIVDDFIIDGNNVNNPVMLLDTFDFGPREDNWFFYPGGNIGLYCPYSSKGAPEEDSAMVFVSNEVGEHSITTRDLNVNENTIIQFEINVGCSTDSSSADPVRLEFSRDFGATWHLLLPLCYHSSSHVSSLCSTEHHPSSTYYAGTMQGWRREVVHFGKLHLCGSVRFRWYQGFYPAGSQPVTWAIDNVYIGPQCEEMCNGQGSCINGTKCICDPGYSGPTCKISTKNPDFLKDDFEGQLESDRFLLMSGGKPSRKCGILSSGNNLFFNEDGLRMLMTRDLDLSHARFVQFFMRLGCGKGVPDPRSQPVLLQYSLNGGLSWSLLQEFLFSNSSNVGRYIALEIPLKARSGSTRLRWWQPSENGHFYSPWVIDQILIGGNISGNMVLEDDFTTLDSRKWLLHPGGTKMPVCGSTGDALVFIEKASTRYVVSTDVAVNEDSFLQIDFAVSCSVTDSCYAIELEYSVDLGLSWHPLVRDCLPTNVECSRYHLQRILVSDTFNKWTRITLPLPPYTRSQATRFRWHQPAPFDKQQTWAIDNVYIGDGCIDMCSGHGRCIQGNCVCDEQWGGLYCDDPETSLPTQLKDNFNRAPSSQNWLTVNGGKLSTVCGAVASGMALHFSGGCSRLLVTVDLNLTNAEFIQFYFMYGCLITPNNRNQGVLLEYSVNGGITWNLLMEIFYDQYSKPGFVNILLPPDAKEIATRFRWWQPRHDGLDQNDWAIDNVLITGGSGGSTRKQNYMMNFSRQHGLRHFYNRRRRSLRRYP

SEQ ID NO: 24 - DNA, 3427 bp, synthetic construct
  source 1..3427: mol_type = other DNA
atgtacaggatgcaactcctgtcttgcattgcactaagtcttgcacttgtcacgaattcgggtggccatcatcatcaccatcacaccggtggtgcggctggctattacccccgcttttcgcccttctttttcctgtgcacccaccacggggagctggaaggggatggggagcagggcgaggtgctcatttccctgcatattgcgggcaaccccacctactacgttccgggacaagaataccatgtgacaatttcaacaagcaccttttttgacggcttgctggtgacaggactatacacatctacaagtgttcaggcatcacagagcattggaggttccagtgctttcggatttgggatcatgtctgaccaccagtttggtaaccagtttatgtgcagtgtggtagcctctcacgtgagtcacctgcccacaaccaacctcagtttcatctggattgctccacctgcgggcacaggctgtgtgaatttcatggctacagcaacacaccggggccaggttattttcaaagatgctttagcccagcagttgtgtgaacaaggagctccaacagatgtcactgtgcacccacatctagctgaaatacatagtgacagcattatcctgagagatgactttgactcctaccaccaactgcaattaaatccaaatatatgggttgaatgtaacaactgtgagactggagaacagtgtggcgcgattatgcatggcaatgccgtcaccttctgtgaaccatatggcccacgagaactgattaccacaggccttaatacaacaacagcttctgtcctccaattttccattgggtcaggttcatgtcgctttagttattcagaccccagcatcatcgtgttatatgccaagaataactctgcggactggattcagctagagaaaattagagccccttccaatgtcagcacaatcatccatatcctctaccttcctgaggacgccaaaggggagaatgtccaatttcagtggaagcaggaaaatcttcgtgtaggtgaagtgtatgaagcctgctgggccttagataacatcttgatcatcaatggttctagaggaggacaaaccaatgctacaagattcagactctggcaaccttataataacggtaagaaagaagaaatctggattgttgatgacttcattatcgatggaaataatgtaaacaaccctgtgatgctcttggatacatttgattttgggcccagagaagacaattggtttttctatcctggtggtaacatcggtctttattgtccatattcttcaaagggggcacctgaagaagattcagctatggtgtttgtttcaaatgaagttggtgagcattccattaccacccgtgacctaaatgtgaatgagaacaccatcatacaatttgagatcaacgttggctgttcgactgatagctcatccgcggatccagtgagactggaattttcaagggacttcggggcgacctggcaccttctgctgcccctctgctaccacagcagcagccacgtcagctctttatgctccaccgagcaccaccccagcagcacctactacgcaggaaccatgcagggctggaggagggaggtcgtgcactttgggaagctgcacctttgtggatctgtccgtttcagatggtaccagggattttaccctgccggctctcagccagtgacatgggccattgataatgtctacatcggtccccagtgtgaggagatgtgtaatggacaggggagctgtatcaatggaaccaaatgtatatgtgaccctggctactcaggtccaacctgtaaaataagcaccaaaaatcctgattttctcaaagatgatttcgaaggtcagctagaatctgatagattcttattaatgagtggtgggaaaccatctcgaaagtgtggaatcctttctagtggaaacaacctctttttcaatgaagatggcttgcgcatgttgatgacacgagacctggatttatcacatgctagatttgtgcagttcttcatgagactgggatgtggtaaaggcgttcctgaccccaggagtcaacccgtgctcctacagtattctctcaacggtggcctctcgtggagtcttcttcaggagttccttttcagcaattccagcaatgtgggcaggtacattgccctggagatacccttgaaagcccgttctggttctactcgccttcgctggtggcaaccgtctgagaatgggcacttctacagcccctgggttatcgatcagattcttattggaggaaatatttctggtaatatggtcttggaagatgatttcacaacccttgatagtaggaaatggctgcttcacccaggaggcaccaagatgcccgtgtgtggctctactggtgatgccctggtcttcattgaaaaggccagcacccgttacgtggtcagcacagacgtcgccgtgaatgaggattccttcctacagatagacttcgctgtctcctgctcagtcacagactcttgttatgcgattgaattggaatactcagtagatcttggattgtcatggcacccattggtaagggactgtctgcctaccaatgtggaatgcagtcgctatcatctgcaacggatcctggtgtcagacactttcaacaagtggactagaatcactctgcctctccctccttataccaggtcccaagccactcgtttccgttggcatcaaccagctccttttgacaagcagcagacatgggcaatagataatgtctatatcggggatggctgcatagacatgtgcagtggccatgggagatgcatccagggaaactgcgtctgtgatgaacagtggggtggcctgtactgtgatgaccccgagacctctcttccaacccaactcaaagacaacttcaatcgagctccatccagtcagaactggctgactgtgaacggagggaaattgagtacagtgtgtggagccgtggcgtcgggaatggctctccatttcagtgggggttgtagtcgattattagtcactgtggatctaaacctcactaatgctgagttcatccaattttacttcatgtatgggtgcctgattacaccaaacaaccgtaaccaaggtgttctcttggaatattctgtcaatggaggcattacctggaacctgctcatggagattttctatgaccagtacagtaagcccggatttgtgaatatccttctccctcctgatgctaaagagattgccactcgcttccgctggtggcagccaagacatgacggcctggatcagaacgactgggccattgacaatgtcctcatctcaggctctgctagcactcgcaaacaaaattacatgatgaatttttcacgacaacatgggctcagacgtttctacaacagaagacgaaggtcacttaggcgatacccat

SEQ ID NO: 25 - protein, 1142 aa, synthetic construct
MYRMQLLSCIALSLALVTNSGGHHHHHHTGGAAGYYPRFSPFFFLCTHHGELEGDGEQGEVLISLHIAGNPTYYVPGQEYHVTISTSTFFDGLLVTGLYTSTSVQASQSIGGSSAFGFGIMSDHQFGNQFMCSVVASHVSHLPTTNLSFIWIAPPAGTGCVNFMATATHRGQVIFKDALAQQLCEQGAPTDVTVHPHLAEIHSDSIILRDDFDSYHQLQLNPNIWVECNNCETGEQCGAIMHGNAVTFCEPYGPRELITTGLNTTTASVLQFSIGSGSCRFSYSDPSIIVLYAKNNSADWIQLEKIRAPSNVSTIIHILYLPEDAKGENVQFQWKQENLRVGEVYEACWALDNILIINGSRGGQTNATRFRLWQPYNNGKKEEIWIVDDFIIDGNNVNNPVMLLDTFDFGPREDNWFFYPGGNIGLYCPYSSKGAPEEDSAMVFVSNEVGEHSITTRDLNVNENTIIQFEINVGCSTDSSSADPVRLEFSRDFGATWHLLLPLCYHSSSHVSSLCSTEHHPSSTYYAGTMQGWRREVVHFGKLHLCGSVRFRWYQGFYPAGSQPVTWAIDNVYIGPQCEEMCNGQGSCINGTKCICDPGYSGPTCKISTKNPDFLKDDFEGQLESDRFLLMSGGKPSRKCGILSSGNNLFFNEDGLRMLMTRDLDLSHARFVQFFMRLGCGKGVPDPRSQPVLLQYSLNGGLSWSLLQEFLFSNSSNVGRYIALEIPLKARSGSTRLRWWQPSENGHFYSPWVIDQILIGGNISGNMVLEDDFTTLDSRKWLLHPGGTKMPVCGSTGDALVFIEKASTRYVVSTDVAVNEDSFLQIDFAVSCSVTDSCYAIELEYSVDLGLSWHPLVRDCLPTNVECSRYHLQRILVSDTFNKWTRITLPLPPYTRSQATRFRWHQPAPFDKQQTWAIDNVYIGDGCIDMCSGHGRCIQGNCVCDEQWGGLYCDDPETSLPTQLKDNFNRAPSSQNWLTVNGGKLSTVCGAVASGMALHFSGGCSRLLVTVDLNLTNAEFIQFYFMYGCLITPNNRNQGVLLEYSVNGGITWNLLMEIFYDQYSKPGFVNILLPPDAKEIATRFRWWQPRHDGLDQNDWAIDNVLISGSASTRKQNYMMNFSRQHGLRRFYNRRRRSLRRYP

SEQ ID NO: 26 - DNA, 1997 bp, synthetic construct
  source 1..1997: mol_type = other DNA
atgtacaggatgcaactcctgtcttgcattgcactaagtcttgcacttgtcacgaattcgggtggtcatcatcatcaccatcacggaggttctagattcagcagtactgctccagttcttcttcagtactctcatgatgctggtatgtcctggtttctggtgaaagaaggctgttacccggcttctgcaggcaaaggatgcgaaggaaactccagagaactaagtgagcccaccatgtatcacacaggggactttgaagaatggacaagaatcaccattgttattccaaggtctcttgcatccagcaagaccagattccgatggatccaggagagcagctcacagaaaaacgtgcctccatttggtttagatggagtgtacatatccgagccttgtcccagttactgcagtggccatggggactgcatttcaggagtgtgtttctgtgacctgggatatactgctgcacaaggaacctgtgtgtcaaatgtccccaatcacaatgagatgttcgataggtttgaggggaagctcagccctctgtggtacaagataacaggtgcccaggttggaactggctgtggaacacttaacgatggcaaatctctctacttcaatggccctgggaaaagggaagcccgtacggtccctctggacaccaggaatatcagacttgttcaattttatatacaaattggaagcaaaacttcaggcattacctgcatcaaaccaagaactagaaatgaagggcttattgttcagtattcaaatgacaatgggatactctggcatttgcttcgagagttggacttcatgtccttcctggaaccacagatcatttccattgacctgccacaggacgcgaagacacctgcaacggcatttcgatggtggcaaccgcaacatgggaagcattcagcccagtgggctttggatgatgttcttataggaatgaatgacagctctcaaactggatttcaagacaaatttgatggctctataggaggaggtacccttgatagtaggaaatggctgcttcacccaggaggcaccaagatgcccgtgtgtggctctactggtgatgccctggtcttcattgaaaaggccagcacccgttacgtggtcagcacagacgtcgccgtgaatgaggattccttcctacagatagacttcgctgtctcctgctcagtcacagactcttgttatgcgattgaattggaatactcagtagatcttggattgtcatggcacccattggtaagggactgtctgcctaccaatgtggaatgcagtcgctatcatctgcaacggatcctggtgtcagacactttcaacaagtggactagaatcactctgcctctccctccttataccaggtcccaagccactcgtttccgttggcatcaaccagctccttttgacaagcagcagacatgggcaatagataatgtctatatcggggatggctgcatagacatgtgcagtggccatgggagatgcatccagggaaactgcgtctgtgatgaacagtggggtggcctgtactgtgatgaccccgagacctctcttccaacccaactcaaagacaacttcaatcgagctccatccagtcagaactggctgactgtgaacggagggaaattgagtacagtgtgtggagccgtggcgtcgggaatggctctccatttcagtgggggttgtagtcgattattagtcactgtggatctaaacctcactaatgctgagttcatccaattttacttcatgtatgggtgcctgattacaccaaacaaccgtaaccaaggtgttctcttggaatattctgtcaatggaggcattacctggaacctgctcatggagattttctatgaccagtacagtaccggtggatctggagggagcactcgcaaacaaaattacatgatgaatttttcacgacaacatgggctcagacgtttctacaacagaagacgaaggtcacttaggcgatacccatg

SEQ ID NO: 27 - protein, 665 aa, synthetic construct
MYRMQLLSCIALSLALVTNSGGHHHHHHGGSRFSSTAPVLLQYSHDAGMSWFLVKEGCYPASAGKGCEGNSRELSEPTMYHTGDFEEWTRITIVIPRSLASSKTRFRWIQESSSQKNVPPFGLDGVYISEPCPSYCSGHGDCISGVCFCDLGYTAAQGTCVSNVPNHNEMFDRFEGKLSPLWYKITGAQVGTGCGTLNDGKSLYFNGPGKREARTVPLDTRNIRLVQFYIQIGSKTSGITCIKPRTRNEGLIVQYSNDNGILWHLLRELDFMSFLEPQIISIDLPQDAKTPATAFRWWQPQHGKHSAQWALDDVLIGMNDSSQTGFQDKFDGSIGGGTLDSRKWLLHPGGTKMPVCGSTGDALVFIEKASTRYVVSTDVAVNEDSFLQIDFAVSCSVTDSCYAIELEYSVDLGLSWHPLVRDCLPTNVECSRYHLQRILVSDTFNKWTRITLPLPPYTRSQATRFRWHQPAPFDKQQTWAIDNVYIGDGCIDMCSGHGRCIQGNCVCDEQWGGLYCDDPETSLPTQLKDNFNRAPSSQNWLTVNGGKLSTVCGAVASGMALHFSGGCSRLLVTVDLNLTNAEFIQFYFMYGCLITPNNRNQGVLLEYSVNGGITWNLLMEIFYDQYSTGGSGGSTRKQNYMMNFSRQHGLRRFYNRRRRSLRRYP

SEQ ID NO: 28 - DNA, 1996 bp, synthetic construct
  source 1..1996: mol_type = other DNA
atgtacaggatgcaactcctgtcttgcattgcactaagtcttgcacttgtcacgaattcgggtggtcatcatcatcaccatcacggaggttctagattcagcagtactgctccagttcttcttcagtactctcatgatgctggtatgtcctggtttctggtgaaagaaggctgttacccggcttctgcaggcaaaggatgcgaaggaaactccagagaactaagtgagcccaccatgtatcacacaggggactttgaagaatggacaagaatcaccattgttattccaaggtctcttgcatccagcaagaccagattccgatggatccaggagagcagctcacagaaaaacgtgcctccatttggtttagatggagtgtacatatccgagccttgtcccagttactgcagtggccatggggactgcatttcaggagtgtgtttctgtgacctgggatatactgctgcacaaggaacctgtgtgtcaaatgtccccaatcacaatgagatgttcgataggtttgaggggaagctcagccctctgtggtacaagataacaggtgcccaggttggaactggctgtggaacacttaacgatggcaaatctctctacttcaatggccctgggaaaagggaagcccgtacggtccctctggacaccaggaatatcagacttgttcaattttatatacaaattggaagcaaaacttcaggcattacctgcatcaaaccaagaactagaaatgaagggcttattgttcagtattcaaatgacaatgggatactctggcatttgcttcgagagttggacttcatgtccttcctggaaccacagatcatttccattgacctgccacaggacgcgaagacacctgcaacggcatttcgatggtggcaaccgcaacatgggaagcattcagcccagtgggctttggatgatgttcttataggaatgaatgacagctctcaaactggatttcaagacaaatttgatggctctataggaggaggtacccttgatagtaggaaatggctgcttcacccaggaggcaccaagatgcccgtgtgtggctctactggtgatgccctggtcttcattgaaaaggccagcacccgttacgtggtcagcacagacgtcgccgtgaatgaggattccttcctacagatagacttcgctgtctcctgctcagtcacagactcttgttatgcgattgaattggaatactcagtagatcttggattgtcatggcacccattggtaagggactgtctgcctaccaatgtggaatgcagtcgctatcatctgcaacggatcctggtgtcagacactttcaacaagtggactagaatcactctgcctctccctccttataccaggtcccaagccactcgtttccgttggcatcaaccagctccttttgacaagcagcagacatgggcaatagataatgtctatatcggggatggctgcatagacatgtgcagtggccatgggagatgcatccagggaaactgcgtctgtgatgaacagtggggtggcctgtactgtgatgaccccgagacctctcttccaacccaactcaaagacaacttcaatcgagctccatccagtcagaactggctgactgtgaacggagggaaattgagtacagtgtgtggagccgtggcgtcgggaatggctctccatttcagtgggggttgtagtcgattattagtcactgtggatctaaacctcactaatgctgagttcatccaattttacttcatgtatgggtgcctgattacaccaaacaaccgtaaccaaggtgttctcttggaatattctgtcaatggaggcattacctggaacctgctcatggagattttctatgaccagtacagtaccggtggatctggagggagcactcgcaaacaaaattacatgatgaatttttcacgacaacatgggctcagacatttctacaacagaagacgaaggtcacttaggcgatacccat

SEQ ID NO: 29 - protein, 665 aa, synthetic construct
MYRMQLLSCIALSLALVTNSGGHHHHHHGGSRFSSTAPVLLQYSHDAGMSWFLVKEGCYPASAGKGCEGNSRELSEPTMYHTGDFEEWTRITIVIPRSLASSKTRFRWIQESSSQKNVPPFGLDGVYISEPCPSYCSGHGDCISGVCFCDLGYTAAQGTCVSNVPNHNEMFDRFEGKLSPLWYKITGAQVGTGCGTLNDGKSLYFNGPGKREARTVPLDTRNIRLVQFYIQIGSKTSGITCIKPRTRNEGLIVQYSNDNGILWHLLRELDFMSFLEPQIISIDLPQDAKTPATAFRWWQPQHGKHSAQWALDDVLIGMNDSSQTGFQDKFDGSIGGGTLDSRKWLLHPGGTKMPVCGSTGDALVFIEKASTRYVVSTDVAVNEDSFLQIDFAVSCSVTDSCYAIELEYSVDLGLSWHPLVRDCLPTNVECSRYHLQRILVSDTFNKWTRITLPLPPYTRSQATRFRWHQPAPFDKQQTWAIDNVYIGDGCIDMCSGHGRCIQGNCVCDEQWGGLYCDDPETSLPTQLKDNFNRAPSSQNWLTVNGGKLSTVCGAVASGMALHFSGGCSRLLVTVDLNLTNAEFIQFYFMYGCLITPNNRNQGVLLEYSVNGGITWNLLMEIFYDQYSTGGSGGSTRKQNYMMNFSRQHGLRHFYNRRRRSLRRYP

SEQ ID NO: 30 - DNA, 2442 bp, synthetic construct
  source 1..2442: mol_type = other DNA
atgtacaggatgcaactcctgtcttgcattgcactaagtcttgcacttgtcacgaattcgggtggccaccatcatcaccatcacggaggttctagaggaggacaaaccaatgctacaagattcagactctggcaaccttataataacggtaagaaagaagaaatctggattgttgatgacttcattatcgatggaaataatgtaaacaaccctgtgatgctcttggatacatttgattttgggcccagagaagacaattggtttttctatcctggtggtaacatcggtctttattgtccatattcttcaaagggggcacctgaagaagattcagctatggtgtttgtttcaaatgaagttggtgagcattccattaccacccgtgacctaaatgtgaatgagaacaccatcatacaatttgagatcaacgttggctgttcgactgatagctcatccgcggatccagtgagactggaattttcaagggacttcggggcgacctggcaccttctgctgcccctctgctaccacagcagcagccacgtcagctctttatgctccaccgagcaccaccccagcagcacctactacgcaggaaccatgcagggctggaggagggaggtcgtgcactttgggaagctgcacctttgtggatctgtccgtttcagatggtaccagggattttaccctgccggctctcagccagtgacatgggccattgataatgtctacatcggtccccagtgtgaggagatgtgtaatggacaggggagctgtatcaatggaaccaaatgtatatgtgaccctggctactcaggtccaacctgtaaaataagcaccaaaaatcctgattttctcaaagatgatttcgaaggtcagctagaatctgatagattcttattaatgagtggtgggaaaccatctcgaaagtgtggaatcctttctagtggaaacaacctctttttcaatgaagatggcttgcgcatgttgatgacacgagacctggatttatcacatgctagatttgtgcagttcttcatgagactgggatgtggtaaaggcgttcctgaccccaggagtcaacccgtgctcctacagtattctctcaacggtggcctctcgtggagtcttcttcaggagttccttttcagcaattccagcaatgtgggcaggtacattgccctggagatacccttgaaagcccgttctggttctactcgccttcgctggtggcaaccgtctgagaatgggcacttctacagcccctgggttatcgatcagattcttattggaggaaatatttctggtaatatggtcttggaagatgatttcacaacccttgatagtaggaaatggctgcttcacccaggaggcaccaagatgcccgtgtgtggctctactggtgatgccctggtcttcattgaaaaggccagcacccgttacgtggtcagcacagacgtcgccgtgaatgaggattccttcctacagatagacttcgctgtctcctgctcagtcacagactcttgttatgcgattgaattggaatactcagtagatcttggattgtcatggcacccattggtaagggactgtctgcctaccaatgtggaatgcagtcgctatcatctgcaacggatcctggtgtcagacactttcaacaagtggactagaatcactctgcctctccctccttataccaggtcccaagccactcgtttccgttggcatcaaccagctccttttgacaagcagcagacatgggcaatagataatgtctatatcggggatggctgcatagacatgtgcagtggccatgggagatgcatccagggaaactgcgtctgtgatgaacagtggggtggcctgtactgtgatgaccccgagacctctcttccaacccaactcaaagacaacttcaatcgagctccatccagtcagaactggctgactgtgaacggagggaaattgagtacagtgtgtggagccgtggcgtcgggaatggctctccatttcagtgggggttgtagtcgattattagtcactgtggatctaaacctcactaatgctgagttcatccaattttacttcatgtatgggtgcctgattacaccaaacaaccgtaaccaaggtgttctcttggaatattctgtcaatggaggcattacctggaacctgctcatggagattttctatgaccagtacagtaagcccggatttgtgaatatccttctccctcctgatgctaaagagattgccactcgcttccgctggtggcagccaagacatgacggcctggatcagaacgactgggccattgacaatgtcctcatctcaggctctgctagcactcgcaaacaaaattacatgatgaatttttcacgacaacatgggctcagacgtttctacaacagaagacgaaggtcacttaggcgatacccatga

SEQ ID NO: 31 - protein, 813 aa, synthetic construct
MYRMQLLSCIALSLALVTNSGGHHHHHHGGSRGGQTNATRFRLWQPYNNGKKEEIWIVDDFIIDGNNVNNPVMLLDTFDFGPREDNWFFYPGGNIGLYCPYSSKGAPEEDSAMVFVSNEVGEHSITTRDLNVNENTIIQFEINVGCSTDSSSADPVRLEFSRDFGATWHLLLPLCYHSSSHVSSLCSTEHHPSSTYYAGTMQGWRREVVHFGKLHLCGSVRFRWYQGFYPAGSQPVTWAIDNVYIGPQCEEMCNGQGSCINGTKCICDPGYSGPTCKISTKNPDFLKDDFEGQLESDRFLLMSGGKPSRKCGILSSGNNLFFNEDGLRMLMTRDLDLSHARFVQFFMRLGCGKGVPDPRSQPVLLQYSLNGGLSWSLLQEFLFSNSSNVGRYIALEIPLKARSGSTRLRWWQPSENGHFYSPWVIDQILIGGNISGNMVLEDDFTTLDSRKWLLHPGGTKMPVCGSTGDALVFIEKASTRYVVSTDVAVNEDSFLQIDFAVSCSVTDSCYAIELEYSVDLGLSWHPLVRDCLPTNVECSRYHLQRILVSDTFNKWTRITLPLPPYTRSQATRFRWHQPAPFDKQQTWAIDNVYIGDGCIDMCSGHGRCIQGNCVCDEQWGGLYCDDPETSLPTQLKDNFNRAPSSQNWLTVNGGKLSTVCGAVASGMALHFSGGCSRLLVTVDLNLTNAEFIQFYFMYGCLITPNNRNQGVLLEYSVNGGITWNLLMEIFYDQYSKPGFVNILLPPDAKEIATRFRWWQPRHDGLDQNDWAIDNVLISGSASTRKQNYMMNFSRQHGLRRFYNRRRRSLRRYP

SEQ ID NO: 32 - DNA, 1963 bp, synthetic construct
  source 1..1963: mol_type = other DNA
atgtacaggatgcaactcctgtcttgcattgcactaagtcttgcacttgtcacgaattcgggtggccaccatcatcaccatcacggaggttctagaacccttgatagtaggaaatggctgcttcacccaggaggcaccaagatgcccgtgtgtggctctactggtgatgccctggtcttcattgaaaaggccagcacccgttacgtggtcagcacagacgtcgccgtgaatgaggattccttcctacagatagacttcgctgtctcctgctcagtcacagactcttgttatgcgattgaattggaatactcagtagatcttggattgtcatggcacccattggtaagggactgtctgcctaccaatgtggaatgcagtcgctatcatctgcaacggatcctggtgtcagacactttcaacaagtggactagaatcactctgcctctccctccttataccaggtcccaagccactcgtttccgttggcatcaaccagctccttttgacaagcagcagacatgggcaatagataatgtctatatcggggatggctgcatagacatgtgcagtggccatgggagatgcatccagggaaactgcgtctgtgatgaacagtggggtggcctgtactgtgatgaccccgagacctctcttccaacccaactcaaagacaacttcaatcgagctccatccagtcagaactggctgactgtgaacggagggaaattgagtacagtgtgtggagccgtggcgtcgggaatggctctccatttcagtgggggttgtagtcgattattagtcactgtggatctaaacctcactaatgctgagttcatccaattttacttcatgtatgggtgcctgattacaccaaacaaccgtaaccaaggtgttctcttggaatattctgtcaatggaggcattacctggaacctgctcatggagattttctatgaccagtacagtagcggaggaggtacccttgatagtaggaaatggctgcttcacccaggaggcaccaagatgcccgtgtgtggctctactggtgatgccctggtcttcattgaaaaggccagcacccgttacgtggtcagcacagacgtcgccgtgaatgaggattccttcctacagatagacttcgctgtctcctgctcagtcacagactcttgttatgcgattgaattggaatactcagtagatcttggattgtcatggcacccattggtaagggactgtctgcctaccaatgtggaatgcagtcgctatcatctgcaacggatcctggtgtcagacactttcaacaagtggactagaatcactctgcctctccctccttataccaggtcccaagccactcgtttccgttggcatcaaccagctccttttgacaagcagcagacatgggcaatagataatgtctatatcggggatggctgcatagacatgtgcagtggccatgggagatgcatccagggaaactgcgtctgtgatgaacagtggggtggcctgtactgtgatgaccccgagacctctcttccaacccaactcaaagacaacttcaatcgagctccatccagtcagaactggctgactgtgaacggagggaaattgagtacagtgtgtggagccgtggcgtcgggaatggctctccatttcagtgggggttgtagtcgattattagtcactgtggatctaaacctcactaatgctgagttcatccaattttacttcatgtatgggtgcctgattacaccaaacaaccgtaaccaaggtgttctcttggaatattctgtcaatggaggcattacctggaacctgctcatggagattttctatgaccagtacagtaccggtggatctggagggagcactcgcaaacaaaattacatgatgaatttttcacgacaacatgggctcagacgtttctacaacagaagacgaaggtcacttaggcgatacccat

SEQ ID NO: 33 - protein, 654 aa, synthetic construct
MYRMQLLSCIALSLALVTNSGGHHHHHHGGSRTLDSRKWLLHPGGTKMPVCGSTGDALVFIEKASTRYVVSTDVAVNEDSFLQIDFAVSCSVTDSCYAIELEYSVDLGLSWHPLVRDCLPTNVECSRYHLQRILVSDTFNKWTRITLPLPPYTRSQATRFRWHQPAPFDKQQTWAIDNVYIGDGCIDMCSGHGRCIQGNCVCDEQWGGLYCDDPETSLPTQLKDNFNRAPSSQNWLTVNGGKLSTVCGAVASGMALHFSGGCSRLLVTVDLNLTNAEFIQFYFMYGCLITPNNRNQGVLLEYSVNGGITWNLLMEIFYDQYSSGGGTLDSRKWLLHPGGTKMPVCGSTGDALVFIEKASTRYVVSTDVAVNEDSFLQIDFAVSCSVTDSCYAIELEYSVDLGLSWHPLVRDCLPTNVECSRYHLQRILVSDTFNKWTRITLPLPPYTRSQATRFRWHQPAPFDKQQTWAIDNVYIGDGCIDMCSGHGRCIQGNCVCDEQWGGLYCDDPETSLPTQLKDNFNRAPSSQNWLTVNGGKLSTVCGAVASGMALHFSGGCSRLLVTVDLNLTNAEFIQFYFMYGCLITPNNRNQGVLLEYSVNGGITWNLLMEIFYDQYSTGGSGGSTRKQNYMMNFSRQHGLRRFYNRRRRSLRRYP

SEQ ID NO: 34 - DNA, 3454 bp, synthetic construct
  source 1..3454: mol_type = other DNA
atgtacaggatgcaactcctgtcttgcattgcactaagtcttgcacttgtcacgaattcgggtggccaccatcatcaccatcacggaggttctagaggaggacaaaccaatgctacaagattcagactctggcaaccttataataacggtaagaaagaagaaatctggattgttgatgacttcattatcgatggaaataatgtaaacaaccctgtgatgctcttggatacatttgattttgggcccagagaagacaattggtttttctatcctggtggtaacatcggtctttattgtccatattcttcaaagggggcacctgaagaagattcagctatggtgtttgtttcaaatgaagttggtgagcattccattaccacccgtgacctaaatgtgaatgagaacaccatcatacaatttgagatcaacgttggctgttcgactgatagctcatccgcggatccagtgagactggaattttcaagggacttcggggcgacctggcaccttctgctgcccctctgctaccacagcagcagccacgtcagctctttatgctccaccgagcaccaccccagcagcacctactacgcaggaaccatgcagggctggaggagggaggtcgtgcactttgggaagctgcacctttgtggatctgtccgtttcagatggtaccagggattttaccctgccggctctcagccagtgacatgggccattgataatgtctacatcggtccccagtgtgaggagatgtgtaatggacaggggagctgtatcaatggaaccaaatgtatatgtgaccctggctactcaggtccaacctgtaaaataagcaccaaaaatcctgattttctcaaagatgatttcgaaggtcagctagaatctgatagattcttattaatgagtggtgggaaaccatctcgaaagtgtggaatcctttctagtggaaacaacctctttttcaatgaagatggcttgcgcatgttgatgacacgagacctggatttatcacatgctagatttgtgcagttcttcatgagactgggatgtggtaaaggcgttcctgaccccaggagtcaacccgtgctcctacagtattctctcaacggtggcctctcgtggagtcttcttcaggagttccttttcagcaattccagcaatgtgggcaggtacattgccctggagatacccttgaaagcccgttctggttctactcgccttcgctggtggcaaccgtctgagaatgggcacttctacagcccctgggttatcgatcagattcttattggaggaaatatttctggtaatatggtcttggaagatgatttcacaacccttgatagtaggaaatggctgcttcacccaggaggcaccaagatgcccgtgtgtggctctactggtgatgccctggtcttcattgaaaaggccagcacccgttacgtggtcagcacagacgtcgccgtgaatgaggattccttcctacagatagacttcgctgtctcctgctcagtcacagactcttgttatgcgattgaattggaatactcagtagatcttggattgtcatggcacccattggtaagggactgtctgcctaccaatgtggaatgcagtcgctatcatctgcaacggatcctggtgtcagacactttcaacaagtggactagaatcactctgcctctccctccttataccaggtcccaagccactcgtttccgttggcatcaaccagctccttttgacaagcagcagacatgggcaatagataatgtctatatcggggatggctgcatagacatgtgcagtggccatgggagatgcatccagggaaactgcgtctgtgatgaacagtggggtggcctgtactgtgatgaccccgagacctctcttccaacccaactcaaagacaacttcaatcgagctccatccagtcagaactggctgactgtgaacggagggaaattgagtacagtgtgtggagccgtggcgtcgggaatggctctccatttcagtgggggttgtagtcgattattagtcactgtggatctaaacctcactaatgctgagttcatccaattttacttcatgtatgggtgcctgattacaccaaacaaccgtaaccaaggtgttctcttggaatattctgtcaatggaggcattacctggaacctgctcatggagattttctatgaccagtacagtaagcccggatttgtgaatatccttctccctcctgatgctaaagagattgccactcgcttccgctggtggcagccaagacatgacggcctggatcagaacgactgggccattgacaatgtcctcatctcaggctctgctggaggtggaacgcgtgcggctggctattacccccgcttttcgcccttctttttcctgtgcacccaccacggggagctggaaggggatggggagcagggcgaggtgctcatttccctgcatattgcgggcaaccccacctactacgttccgggacaagaataccatgtgacaatttcaacaagcaccttttttgacggcttgctggtgacaggactatacacatctacaagtgttcaggcatcacagagcattggaggttccagtgctttcggatttgggatcatgtctgaccaccagtttggtaaccagtttatgtgcagtgtggtagcctctcacgtgagtcacctgcccacaaccaacctcagtttcatctggattgctccacctgcgggcacaggctgtgtgaatttcatggctacagcaacacaccggggccaggttattttcaaagatgctttagcccagcagttgtgtgaacaaggagctccaacagatgtcactgtgcacccacatctagctgaaatacatagtgacagcattatcctgagagatgactttgactcctaccaccaactgcaattaaatccaaatatatgggttgaatgtaacaactgtgagactggagaacagtgtggcgcgattatgcatggcaatgccgtcaccttctgtgaaccatatggcccacgagaactgattaccacaggccttaatacaacaacagcttctgtcctccaattttccattgggtcaggttcatgtcgctttagttattcagaccccagcatcatcgtgttatatgccaagaataactctgcggactggattcagctagagaaaattagagccccttccaatgtcagcacaatcatccatatcctctaccttcctgaggacgccaaaggggagaatgtccaatttcagtggaagcaggaaaatcttcgtgtaggtgaagtgtatgaagcctgctgggccttagataacatcttgatcatcaataccggtggatctggagggagcactcgcaaacaaaattacatgatgaatttttcacgacaacatgggctcagacgtttctacaacagaagacgaaggtcacttaggcgatacccat

SEQ ID NO: 35 - protein, 1151 aa, synthetic construct
MYRMQLLSCIALSLALVTNSGGHHHHHHGGSRGGQTNATRFRLWQPYNNGKKEEIWIVDDFIIDGNNVNNPVMLLDTFDFGPREDNWFFYPGGNIGLYCPYSSKGAPEEDSAMVFVSNEVGEHSITTRDLNVNENTIIQFEINVGCSTDSSSADPVRLEFSRDFGATWHLLLPLCYHSSSHVSSLCSTEHHPSSTYYAGTMQGWRREVVHFGKLHLCGSVRFRWYQGFYPAGSQPVTWAIDNVYIGPQCEEMCNGQGSCINGTKCICDPGYSGPTCKISTKNPDFLKDDFEGQLESDRFLLMSGGKPSRKCGILSSGNNLFFNEDGLRMLMTRDLDLSHARFVQFFMRLGCGKGVPDPRSQPVLLQYSLNGGLSWSLLQEFLFSNSSNVGRYIALEIPLKARSGSTRLRWWQPSENGHFYSPWVIDQILIGGNISGNMVLEDDFTTLDSRKWLLHPGGTKMPVCGSTGDALVFIEKASTRYVVSTDVAVNEDSFLQIDFAVSCSVTDSCYAIELEYSVDLGLSWHPLVRDCLPTNVECSRYHLQRILVSDTFNKWTRITLPLPPYTRSQATRFRWHQPAPFDKQQTWAIDNVYIGDGCIDMCSGHGRCIQGNCVCDEQWGGLYCDDPETSLPTQLKDNFNRAPSSQNWLTVNGGKLSTVCGAVASGMALHFSGGCSRLLVTVDLNLTNAEFIQFYFMYGCLITPNNRNQGVLLEYSVNGGITWNLLMEIFYDQYSKPGFVNILLPPDAKEIATRFRWWQPRHDGLDQNDWAIDNVLISGSAGGGTRAAGYYPRFSPFFFLCTHHGELEGDGEQGEVLISLHIAGNPTYYVPGQEYHVTISTSTFFDGLLVTGLYTSTSVQASQSIGGSSAFGFGIMSDHQFGNQFMCSVVASHVSHLPTTNLSFIWIAPPAGTGCVNFMATATHRGQVIFKDALAQQLCEQGAPTDVTVHPHLAEIHSDSIILRDDFDSYHQLQLNPNIWVECNNCETGEQCGAIMHGNAVTFCEPYGPRELITTGLNTTTASVLQFSIGSGSCRFSYSDPSIIVLYAKNNSADWIQLEKIRAPSNVSTIIHILYLPEDAKGENVQFQWKQENLRVGEVYEACWALDNILIINTGGSGGSTRKQNYMMNFSRQHGLRRFYNRRRRSLRRYP

SEQ ID NO: 36 - DNA, 2953 bp, synthetic construct
  source 1..2953: mol_type = other DNA
atgtacaggatgcaactcctgtcttgcattgcactaagtcttgcacttgtcacgaattcgggtggccatcatcatcaccatcacggaggtggggcggctggctattacccccgcttttcgcccttctttttcctgtgcacccaccacggggagctggaaggggatggggagcagggcgaggtgctcatttccctgcatattgcgggcaaccccacctactacgttccgggacaagaataccatgtgacaatttcaacaagcaccttttttgacggcttgctggtgacaggactatacacatctacaagtgttcaggcatcacagagcattggaggttccagtgctttcggatttgggatcatgtctgaccaccagtttggtaaccagtttatgtgcagtgtggtagcctctcacgtgagtcacctgcccacaaccaacctcagtttcatctggattgctccacctgcgggcacaggctgtgtgaatttcatggctacagcaacacaccggggccaggttattttcaaagatgctttagcccagcagttgtgtgaacaaggagctccaacagatgtcactgtgcacccacatctagctgaaatacatagtgacagcattatcctgagagatgactttgactcctaccaccaactgcaattaaatccaaatatatgggttgaatgtaacaactgtgagactggagaacagtgtggcgcgattatgcatggcaatgccgtcaccttctgtgaaccatatggcccacgagaactgattaccacaggccttaatacaacaacagcttctgtcctccaattttccattgggtcaggttcatgtcgctttagttattcagaccccagcatcatcgtgttatatgccaagaataactctgcggactggattcagctagagaaaattagagccccttccaatgtcagcacaatcatccatatcctctaccttcctgaggacgccaaaggggagaatgtccaatttcagtggaagcaggaaaatcttcgtgtaggtgaagtgtatgaagcctgctgggccttagataacatcttgatcatcaatggaggttctagaacccttgatagtaggaaatggctgcttcacccaggaggcaccaagatgcccgtgtgtggctctactggtgatgccctggtcttcattgaaaaggccagcacccgttacgtggtcagcacagacgtcgccgtgaatgaggattccttcctacagatagacttcgctgtctcctgctcagtcacagactcttgttatgcgattgaattggaatactcagtagatcttggattgtcatggcacccattggtaagggactgtctgcctaccaatgtggaatgcagtcgctatcatctgcaacggatcctggtgtcagacactttcaacaagtggactagaatcactctgcctctccctccttataccaggtcccaagccactcgtttccgttggcatcaaccagctccttttgacaagcagcagacatgggcaatagataatgtctatatcggggatggctgcatagacatgtgcagtggccatgggagatgcatccagggaaactgcgtctgtgatgaacagtggggtggcctgtactgtgatgaccccgagacctctcttccaacccaactcaaagacaacttcaatcgagctccatccagtcagaactggctgactgtgaacggagggaaattgagtacagtgtgtggagccgtggcgtcgggaatggctctccatttcagtgggggttgtagtcgattattagtcactgtggatctaaacctcactaatgctgagttcatccaattttacttcatgtatgggtgcctgattacaccaaacaaccgtaaccaaggtgttctcttggaatattctgtcaatggaggcattacctggaacctgctcatggagattttctatgaccagtacagtagcggaggaggtacccttgatagtaggaaatggctgcttcacccaggaggcaccaagatgcccgtgtgtggctctactggtgatgccctggtcttcattgaaaaggccagcacccgttacgtggtcagcacagacgtcgccgtgaatgaggattccttcctacagatagacttcgctgtctcctgctcagtcacagactcttgttatgcgattgaattggaatactcagtagatcttggattgtcatggcacccattggtaagggactgtctgcctaccaatgtggaatgcagtcgctatcatctgcaacggatcctggtgtcagacactttcaacaagtggactagaatcactctgcctctccctccttataccaggtcccaagccactcgtttccgttggcatcaaccagctccttttgacaagcagcagacatgggcaatagataatgtctatatcggggatggctgcatagacatgtgcagtggccatgggagatgcatccagggaaactgcgtctgtgatgaacagtggggtggcctgtactgtgatgaccccgagacctctcttccaacccaactcaaagacaacttcaatcgagctccatccagtcagaactggctgactgtgaacggagggaaattgagtacagtgtgtggagccgtggcgtcgggaatggctctccatttcagtgggggttgtagtcgattattagtcactgtggatctaaacctcactaatgctgagttcatccaattttacttcatgtatgggtgcctgattacaccaaacaaccgtaaccaaggtgttctcttggaatattctgtcaatggaggcattacctggaacctgctcatggagattttctatgaccagtacagtaccggtggatctggagggagcactcgcaaacaaaattacatgatgaatttttcacgacaacatgggctcagacgtttctacaacagaagacgaaggtcacttaggcgatacccat

SEQ ID NO: 37 - protein, 984 aa, synthetic construct
MYRMQLLSCIALSLALVTNSGGHHHHHHGGGAAGYYPRFSPFFFLCTHHGELEGDGEQGEVLISLHIAGNPTYYVPGQEYHVTISTSTFFDGLLVTGLYTSTSVQASQSIGGSSAFGFGIMSDHQFGNQFMCSVVASHVSHLPTTNLSFIWIAPPAGTGCVNFMATATHRGQVIFKDALAQQLCEQGAPTDVTVHPHLAEIHSDSIILRDDFDSYHQLQLNPNIWVECNNCETGEQCGAIMHGNAVTFCEPYGPRELITTGLNTTTASVLQFSIGSGSCRFSYSDPSIIVLYAKNNSADWIQLEKIRAPSNVSTIIHILYLPEDAKGENVQFQWKQENLRVGEVYEACWALDNILIINGGSRTLDSRKWLLHPGGTKMPVCGSTGDALVFIEKASTRYVVSTDVAVNEDSFLQIDFAVSCSVTDSCYAIELEYSVDLGLSWHPLVRDCLPTNVECSRYHLQRILVSDTFNKWTRITLPLPPYTRSQATRFRWHQPAPFDKQQTWAIDNVYIGDGCIDMCSGHGRCIQGNCVCDEQWGGLYCDDPETSLPTQLKDNFNRAPSSQNWLTVNGGKLSTVCGAVASGMALHFSGGCSRLLVTVDLNLTNAEFIQFYFMYGCLITPNNRNQGVLLEYSVNGGITWNLLMEIFYDQYSSGGGTLDSRKWLLHPGGTKMPVCGSTGDALVFIEKASTRYVVSTDVAVNEDSFLQIDFAVSCSVTDSCYAIELEYSVDLGLSWHPLVRDCLPTNVECSRYHLQRILVSDTFNKWTRITLPLPPYTRSQATRFRWHQPAPFDKQQTWAIDNVYIGDGCIDMCSGHGRCIQGNCVCDEQWGGLYCDDPETSLPTQLKDNFNRAPSSQNWLTVNGGKLSTVCGAVASGMALHFSGGCSRLLVTVDLNLTNAEFIQFYFMYGCLITPNNRNQGVLLEYSVNGGITWNLLMEIFYDQYSTGGSGGSTRKQNYMMNFSRQHGLRRFYNRRRRSLRRYP

SEQ ID NO: 38 - DNA, 3433 bp, synthetic construct
  source 1..3433: mol_type = other DNA
atgtacaggatgcaactcctgtcttgcattgcactaagtcttgcacttgtcacgaattcgggtggcgactacaaagacgatgacgacaagaccggtggtgcggctggctattacccccgcttttcgcccttctttttcctgtgcacccaccacggggagctggaaggggatggggagcagggcgaggtgctcatttccctgcatattgcgggcaaccccacctactacgttccgggacaagaataccatgtgacaatttcaacaagcaccttttttgacggcttgctggtgacaggactatacacatctacaagtgttcaggcatcacagagcattggaggttccagtgctttcggatttgggatcatgtctgaccaccagtttggtaaccagtttatgtgcagtgtggtagcctctcacgtgagtcacctgcccacaaccaacctcagtttcatctggattgctccacctgcgggcacaggctgtgtgaatttcatggctacagcaacacaccggggccaggttattttcaaagatgctttagcccagcagttgtgtgaacaaggagctccaacagatgtcactgtgcacccacatctagctgaaatacatagtgacagcattatcctgagagatgactttgactcctaccaccaactgcaattaaatccaaatatatgggttgaatgtaacaactgtgagactggagaacagtgtggcgcgattatgcatggcaatgccgtcaccttctgtgaaccatatggcccacgagaactgattaccacaggccttaatacaacaacagcttctgtcctccaattttccattgggtcaggttcatgtcgctttagttattcagaccccagcatcatcgtgttatatgccaagaataactctgcggactggattcagctagagaaaattagagccccttccaatgtcagcacaatcatccatatcctctaccttcctgaggacgccaaaggggagaatgtccaatttcagtggaagcaggaaaatcttcgtgtaggtgaagtgtatgaagcctgctgggccttagataacatcttgatcatcaatggttctagaggaggacaaaccaatgctacaagattcagactctggcaaccttataataacggtaagaaagaagaaatctggattgttgatgacttcattatcgatggaaataatgtaaacaaccctgtgatgctcttggatacatttgattttgggcccagagaagacaattggtttttctatcctggtggtaacatcggtctttattgtccatattcttcaaagggggcacctgaagaagattcagctatggtgtttgtttcaaatgaagttggtgagcattccattaccacccgtgacctaaatgtgaatgagaacaccatcatacaatttgagatcaacgttggctgttcgactgatagctcatccgcggatccagtgagactggaattttcaagggacttcggggcgacctggcaccttctgctgcccctctgctaccacagcagcagccacgtcagctctttatgctccaccgagcaccaccccagcagcacctactacgcaggaaccatgcagggctggaggagggaggtcgtgcactttgggaagctgcacctttgtggatctgtccgtttcagatggtaccagggattttaccctgccggctctcagccagtgacatgggccattgataatgtctacatcggtccccagtgtgaggagatgtgtaatggacaggggagctgtatcaatggaaccaaatgtatatgtgaccctggctactcaggtccaacctgtaaaataagcaccaaaaatcctgattttctcaaagatgatttcgaaggtcagctagaatctgatagattcttattaatgagtggtgggaaaccatctcgaaagtgtggaatcctttctagtggaaacaacctctttttcaatgaagatggcttgcgcatgttgatgacacgagacctggatttatcacatgctagatttgtgcagttcttcatgagactgggatgtggtaaaggcgttcctgaccccaggagtcaacccgtgctcctacagtattctctcaacggtggcctctcgtggagtcttcttcaggagttccttttcagcaattccagcaatgtgggcaggtacattgccctggagatacccttgaaagcccgttctggttctactcgccttcgctggtggcaaccgtctgagaatgggcacttctacagcccctgggttatcgatcagattcttattggaggaaatatttctggtaatatggtcttggaagatgatttcacaacccttgatagtaggaaatggctgcttcacccaggaggcaccaagatgcccgtgtgtggctctactggtgatgccctggtcttcattgaaaaggccagcacccgttacgtggtcagcacagacgtcgccgtgaatgaggattccttcctacagatagacttcgctgtctcctgctcagtcacagactcttgttatgcgattgaattggaatactcagtagatcttggattgtcatggcacccattggtaagggactgtctgcctaccaatgtggaatgcagtcgctatcatctgcaacggatcctggtgtcagacactttcaacaagtggactagaatcactctgcctctccctccttataccaggtcccaagccactcgtttccgttggcatcaaccagctccttttgacaagcagcagacatgggcaatagataatgtctatatcggggatggctgcatagacatgtgcagtggccatgggagatgcatccagggaaactgcgtctgtgatgaacagtggggtggcctgtactgtgatgaccccgagacctctcttccaacccaactcaaagacaacttcaatcgagctccatccagtcagaactggctgactgtgaacggagggaaattgagtacagtgtgtggagccgtggcgtcgggaatggctctccatttcagtgggggttgtagtcgattattagtcactgtggatctaaacctcactaatgctgagttcatccaattttacttcatgtatgggtgcctgattacaccaaacaaccgtaaccaaggtgttctcttggaatattctgtcaatggaggcattacctggaacctgctcatggagattttctatgaccagtacagtaagcccggatttgtgaatatccttctccctcctgatgctaaagagattgccactcgcttccgctggtggcagccaagacatgacggcctggatcagaacgactgggccattgacaatgtcctcatctcaggctctgctagcactcgcaaacaaaattacatgatgaatttttcacgacaacatgggctcagacgtttctacaacagaagacgaaggtcacttaggcgatacccat

SEQ ID NO: 39 - protein, 1144 aa, synthetic construct
MYRMQLLSCIALSLALVTNSGGDYKDDDDKTGGAAGYYPRFSPFFFLCTHHGELEGDGEQGEVLISLHIAGNPTYYVPGQEYHVTISTSTFFDGLLVTGLYTSTSVQASQSIGGSSAFGFGIMSDHQFGNQFMCSVVASHVSHLPTTNLSFIWIAPPAGTGCVNFMATATHRGQVIFKDALAQQLCEQGAPTDVTVHPHLAEIHSDSIILRDDFDSYHQLQLNPNIWVECNNCETGEQCGAIMHGNAVTFCEPYGPRELITTGLNTTTASVLQFSIGSGSCRFSYSDPSIIVLYAKNNSADWIQLEKIRAPSNVSTIIHILYLPEDAKGENVQFQWKQENLRVGEVYEACWALDNILIINGSRGGQTNATRFRLWQPYNNGKKEEIWIVDDFIIDGNNVNNPVMLLDTFDFGPREDNWFFYPGGNIGLYCPYSSKGAPEEDSAMVFVSNEVGEHSITTRDLNVNENTIIQFEINVGCSTDSSSADPVRLEFSRDFGATWHLLLPLCYHSSSHVSSLCSTEHHPSSTYYAGTMQGWRREVVHFGKLHLCGSVRFRWYQGFYPAGSQPVTWAIDNVYIGPQCEEMCNGQGSCINGTKCICDPGYSGPTCKISTKNPDFLKDDFEGQLESDRFLLMSGGKPSRKCGILSSGNNLFFNEDGLRMLMTRDLDLSHARFVQFFMRLGCGKGVPDPRSQPVLLQYSLNGGLSWSLLQEFLFSNSSNVGRYIALEIPLKARSGSTRLRWWQPSENGHFYSPWVIDQILIGGNISGNMVLEDDFTTLDSRKWLLHPGGTKMPVCGSTGDALVFIEKASTRYVVSTDVAVNEDSFLQIDFAVSCSVTDSCYAIELEYSVDLGLSWHPLVRDCLPTNVECSRYHLQRILVSDTFNKWTRITLPLPPYTRSQATRFRWHQPAPFDKQQTWAIDNVYIGDGCIDMCSGHGRCIQGNCVCDEQWGGLYCDDPETSLPTQLKDNFNRAPSSQNWLTVNGGKLSTVCGAVASGMALHFSGGCSRLLVTVDLNLTNAEFIQFYFMYGCLITPNNRNQGVLLEYSVNGGITWNLLMEIFYDQYSKPGFVNILLPPDAKEIATRFRWWQPRHDGLDQNDWAIDNVLISGSASTRKQNYMMNFSRQHGLRRFYNRRRRSLRRYP

SEQ ID NO: 40 - protein, 700 aa, synthetic construct
MASRSTETELQVAVKTSAKKDSRKKGQDRSEATLIKRFKGEGVRYKAKLIGIDEVSAARGDKLCQDSMMKLKGVVAGARSKGEHKQKIFLTISFGGIKIFDEKTGALQHHHAVHEISYIAKDITDHRAFGYVCGKEGNHRFVAIKTAQAAEPVILDLRDLFQLIYELKQREELEKKAQKDKQCEQAVYQTILEEDVEDPVYQYIVFEAGHEPIRDPETEENIYQVPTSQKKEGVYDVPKSQPVSNGYSFEDFEERFAAATPNRNLPTDFDEIFEATKAVTQLELFGDMSTPPDITSPPTPATPGDAFIPSSSQTLPASADVFSSVPFGTAAVPSGYVAMGAVLPSFWGQQPLVQQQMVMGAQPPVAQVMPGAQPIAWGQPGLFPATQQPWPTVAGQFPPAAFMPTQTVMPLPAAMFQGPLTPLATVPGTSDSTRSSPQTDKPRQKMGKETFKDFQMAQPPPVPSRKPDQPSLTCTSEAFSSYFNKVGVAQDTDDCDDFDISQLNLTPVTSTTPSTNSPPTPAPRQSSPSKSSASHASDPTTDDIFEEGFESPSKSEEQEAPDGSQASSNSDPFGEPSGEPSGDNISPQAGSSRGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 41 - protein, 640 aa, synthetic construct
MAPRRVRSFLRGLPALLLLLLFLGPWPAASHGGKYSREKNQPKPSPKRESGEEFRMEKLNQLWEKAQRLHLPPVRLAELHADLKIQERDELAWKKLKLDGLDEDGEKEARLIRNLNVILAKYGLDGKKDARQVTSNSLSGTQEDGLDDPRLEKLWHKAKTSGKFSGEELDKLWREFLHHKEKVHEYNVLLETLSRTEEIHENVISPSDLSDIKGSVLHSRHTELKEKLRSINQGLDRLRRVSHQGYSTEAEFEEPRVIDLWDLAQSANLTDKELEAFREELKHFEAKIEKHNHYQKQLEIAHEKLRHAESVGDGERVSRSREKHALLEGRTKELGYTVKKHLQDLSGRISRARHNELTGGSGGNSAPLERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGARTGGGGSGGGGSSTRKQNYMMNFSRQHGLRRFYNRRRRSLRRYP

SEQ ID NO: 42 - DNA, 119 bp, synthetic construct
  source 1..119: mol_type = other DNA
ttgattcttcatgggtatcgcctaagtgaccttcgtcttctgttgtagaaacgtctgagtccatgttgtcgtgaaaaattcatcatgtaattttgtttgcgagtgctgttaaaatcaaa

SEQ ID NO: 43 - RNA, 20 nt, synthetic construct
  source 1..20: mol_type = other RNA
tgaatttttcacgacaacat

SEQ ID NO: 44 - protein, 7 aa, synthetic construct
PKKKRRV

SEQ ID NO: 45 - protein, 7 aa, synthetic construct
PKKKRKV

SEQ ID NO: 46 - protein, 18 aa, synthetic construct
KRTADGSEFESPKKKRKV

SEQ ID NO: 47 - protein, 16 aa, synthetic construct
KRPAATKKAGQAKKKK

SEQ ID NO: 48 - DNA, 60 bp, synthetic construct
  source 1..60: mol_type = other DNA
tagcaataaaggatcgtttattttcattggaagcgtgtgttggttttttgatcaggcgcg

SEQ ID NO: 49 - RNA, 20 nt, synthetic construct
  source 1..20: mol_type = other RNA
ggccaaaggggctggccttc

SEQ ID NO: 50 - RNA, 20 nt, synthetic construct
  source 1..20: mol_type = other RNA
ctctgcggggctttgacgtc

SEQ ID NO: 51 - RNA, 20 nt, synthetic construct
  source 1..20: mol_type = other RNA
gtcctcgacagcgtccccgt

SEQ ID NO: 52 - DNA, 2000 bp, synthetic construct
  source 1..2000: mol_type = other DNA
acaaatcaccatttccaggcaccactcgagtctgccaattccactttcgcttaatctcattttaaaatcctctacagatgaggctctgtcgctgccgcgaggggccgggcggccgcggcgggagggacagggggcctgggtgggaagggagcgcgagggcccctagcggttcgcggcctcgggcggattcccagaacgagtgcgggccgcccggactggcagactgcgagccccgaggggcgcgggacgcagccggctcagacaaagaaaccggcgccagcggccgtccctgccgcccctctccttccctcacgcatcctcccaggaaaaacagggcacactgacggccaaaggggctggccttccccttcagacagctggacgggaggtgccctctgcggggctttgacgtccctcgcagaagagtcgcgggctcagcggtcctcgacagcgtccccgtcccgctccccggcgggcgcccctccctgtcctcccgggtgcgaaccgggcgctggccggggactccggggacgcgtgcgcccctcgccgcgcgaggtgccgccgagccagcccgagagggcggggggcgggcggggcggcgcgcgggggcgggggagcggccgggacacgtgtggcggcggcgggggggacgcggcgcccggggctttaagaaggtgtggagcggggcgggcgctttcccaggcctggccgaggggcgtcgcgcagaggcggcggcggcgcacggaggcggcagacgacgcgctctcggcgcccgcagccccggtcccgcgctcccgcggcccaaagtaactttgggagccgccgtctcccgcggaacttctcggcgggggcccgctcccaggcccgctcccgagcccgttccgctcccgtccgccttcttctcgccttctctccgcgtggctcctccgtcccggcgtctccaaaactgaatgagcgagcggcgcgtagggcgcgcggcggcggcggcggcggcggcggcatggagcgcagtggctgggcccggcagactttcctcctagcgctgttgctgggggcgacgctgagggcgcgcgcggcggctggctattacccccgcttttcgcccttctttttcctgtgcacccaccacggggagctggaaggggatggggagcagggcgaggtgctcatttccctgcatattgcgggcaaccccacctactacgttccgggacaagaataccatggtaggtaccgcagcacctccgcgctcgccgccgccgggtgcgcccgccagcgcacctttctcatccctttccttcttccaaggggccttggaaacaagaaccatgacaaccctggccccagcgatgcgcttggtccagggagcgagaataaagttagtggagaatggagtcacaagttgggattcattctccatgtccccactccccgaacgcatctcctggtaatgagctggtgcttgtgctaaacgcctgagaattcttggaggccggtgtctttccccctcactccccgtacttaccgcaggcactttaaattgaacctgcgcattttgtttctttactcctaggacttggatttcagacctttgtcctgctcccggtccccccagatgggtccccgggggggcgttgggggctgtcgctgggctttgcgggcaggctggtgacagattgaagcgcccaggtctgtggctgccccgcgccgctcgaccgcaaagttcccgctgctcaccagccaggagtccctggcatttcattcagtgtcatacagccctcctgaaagggagcggctgcttccagctccctattcttttgaaagtgtgatttaatatgtcagtaattgatgatttttctgattcctgccttacttttacatggtgcttcttgttagaattatctttgtgatgtttattaatgactttgtattttatctgcacagctttaatttccaggtagaggctggaatcccttataatttagaggtatcttaaaaat

SEQ ID NO: 53 - protein, 30 aa, synthetic construct
RKQNYMMNFSRQHGLRRFYNRRRRSLRRYP

SEQ ID NO: 54 - DNA, 19 bp, synthetic construct
  source 1..19: mol_type = other DNA
gtcccagcctttagttcct

SEQ ID NO: 55 - DNA, 21 bp, synthetic construct
  source 1..21: mol_type = other DNA
aactacacaggcactttcaac

SEQ ID NO: 56 - protein, 30 aa, synthetic construct
RKQNYMMNFSRQHGLRHFYNRRRRSLRRYP

SEQ ID NO: 57 - protein, 30 aa, synthetic construct
RKQNYMMNFSRQHGLRDFYNRRRRSLRRYP

SEQ ID NO: 58 - protein, 30 aa, synthetic construct
RKQNYMMNFSRQHGLRKFYNRRRRSLRRYP

SEQ ID NO: 59 - protein, 24 aa, synthetic construct
RKQNYMMNFSRQHGLRHFYNRRRR

SEQ ID NO: 60 - DNA, 2100 bp, synthetic construct
  source 1..2100: mol_type = other DNA
atggccagtaggtcaactgaaacggagctccaagtggcagttaaaaccagcgcgaagaaggatagtcgcaagaagggacaggacagatcagaagctactctcatcaaaaggttcaagggcgagggggtgaggtataaagcgaaattgattggtattgacgaagtatcagctgcacgaggagacaagctttgtcaagactctatgatgaaacttaaaggtgtggtagccggtgcacggtcaaagggagaacacaaacagaagatattcttgacaatttcctttgggggaataaagatttttgacgaaaagactggtgcccttcaacatcatcatgcggtccacgaaatctcttatatagcaaaggacataactgatcacagggccttcgggtatgtttgcggtaaggaaggtaaccatcgcttcgtggccattaaaactgctcaggcggcagaacccgttatactcgatcttcgcgacttgtttcaactcatctacgagttgaagcaaagagaggagcttgaaaagaaagcccagaaagataaacagtgcgaacaagctgtataccagacaatactcgaggaagacgttgaagaccctgtatatcagtacatagtttttgaggcagggcatgaaccgataagagatccagagactgaggaaaacatttatcaggtcccgacaagccaaaaaaaggagggtgtgtacgatgtgccaaagtcccagcccgtcagtaatggctacagtttcgaggacttcgaagagcggttcgccgcagctacgcccaaccgcaacctcccaaccgatttcgacgagatatttgaggcaaccaaggccgttacacagctcgaactcttcggcgatatgtctactccacctgacatcacgagccctcctacacccgcgactccgggcgacgcattcattccatcctccagtcaaaccctcccggcttccgcggacgtgttttcctctgtcccgttcggaaccgcagcggttccgtctggatatgttgcgatgggggcagtgttgccctctttttggggccaacagccactggttcaacagcagatggtcatgggcgcccagcccccggttgctcaggtcatgccaggagctcaacccatagcgtggggccagccaggactgttccccgcgactcagcaaccctggccaaccgtggctgggcaatttccaccagcggcattcatgcctacgcagaccgtcatgcctctgcccgctgctatgtttcaaggcccccttacacctctggcaacggtgcctggaacttccgactccacaagaagttccccgcagaccgataagccgcggcaaaaaatgggtaaagaaacttttaaagattttcaaatggcgcaacctccgccggtaccgagcagaaaaccggaccagccaagcctcacctgcacatctgaagccttttctagctattttaataaggtcggtgtagctcaagacacggatgactgtgatgattttgatatctcacaactcaacttgacaccagtcaccagcacaactcccagtactaacagtccgccaactcccgcgccgcgccagtcctcaccgtctaaatcttccgcttcacatgcatctgatcctacgacggacgatatcttcgaagaaggttttgaatcaccgagtaagagcgaagaacaagaagctccagatggttctcaagcgtcctccaatagcgatccttttggggaaccaagcggggagccatctggtgacaatatatcaccacaggcaggcagtagtcgcggcgtccaagttgagacgatttcccctggtgatggacggacattccccaagcggggccagacctgtgttgttcactacacgggaatgttggaagacggtaaaaaagtcgacagtagtcgcgatcggaacaagccattcaaattcatgctgggcaaacaagaagttatccggggctgggaagaaggcgtcgcacaaatgtctgtagggcaaagagcaaaactcacaataagtccagactacgcgtatggcgcaactggccaccctggtattataccgccacatgcgacgctggtcttcgacgtagaattgttgaagttggaa

SEQ ID NO: 61 - protein, 4 aa, synthetic construct
HNEL

SEQ ID NO: 62 - protein, 6 aa, synthetic construct
HHHHHH

SEQ ID NO: 63 - DNA, 119 bp, synthetic construct
  source 1..119: mol_type = other DNA
ttgattcttcatgggtatcgcctaagtgaccttcgtcttctgttgtagaaatgtctgagcccatgttgtcgtgaaaaattcatcatgtaattttgtttgcgagtgctgttaaaatcaaa

SEQ ID NO: 64 - protein, 24 aa, synthetic construct
RKQNYMMNFSRQHGLRRFYNRRRR

SEQ ID NO: 65 - protein, 32 aa, synthetic construct
STRKQNYMMNFSRQHGLRHFYNRRRRSLRRYP

SEQ ID NO: 66 - DNA, 29 bp, synthetic construct
  source 1..29: mol_type = other DNA
gacaacatgggctcagacattctacaaca

SEQ ID NO: 67 - DNA, 26 bp, synthetic construct
  source 1..26: mol_type = other DNA
acaacatgggctcagcntttctacaa

SEQ ID NO: 68 - protein, 32 aa, synthetic construct
STRKQNYMMNFSRQHGLRRFYNRRRRSLRRYP

SEQ ID NO: 69 - protein, 29 aa, synthetic construct
RKQNYMMNFSRQHGLRHFYNRRRRLRRYP

SEQ ID NO: 70 - protein, 29 aa, synthetic construct
RKQNYMMNFSRQHGLHHFYNRRRRLRRYP

SEQ ID NO: 71 - protein, 24 aa, synthetic construct
RKQNYMMNFSRQHGLHHFYNRRRR

SEQ ID NO: 72 - protein, 29 aa, synthetic construct
RKQNYMMNFSRQHGLRRFYNRRRRLRRYP

SEQ ID NO: 73 - protein, 29 aa, synthetic construct
RKQNYMMNFSRQHGLRKFYNRRRRLRRYP

SEQ ID NO: 74 - protein, 24 aa, synthetic construct
RKQNYMMNFSRQHGLRKFYNRRRR

SEQ ID NO: 75 - protein, 29 aa, synthetic construct
RKQNYMMNFSRQHGLRDFYNRRRRLRRYP

SEQ ID NO: 76 - protein, 24 aa, synthetic construct
RKQNYMMNFSRQHGLRDFYNRRRR

SEQ ID NO: 77 - protein, 30 aa, synthetic construct
RKQNYMMNFSRQHGLHHFYNRRRRSLRRYP

SEQ ID NO: 78 - DNA, 7138 bp, synthetic construct
  source 1..7138: mol_type = other DNA
  CDS 2782..2808: protein_id = 79; translation = YPYDVPDYA
  CDS 2455..2775: protein_id = 80; translation = GVQVETISPGDGRTFPKRGQTCVVHYTGMLEDGKKVDSSRDRNK PFKFMLGKQEVIRGWEEGVAQMSVGQRAKLTISPDYAYGATGHPGIIPPHATLVFDVE LLKLE
  CDS complement(5188..6060): codon_start = 1; protein_id = 81; translation = MSIQHFRVALIPFFAAFCLPVFAHPETLVKVKDAEDQLGARVGY IELDLNSGKILESFRPEERFQVEVAMMSTFKVLLCGAVLSRIDAGQEQLGRRIHYSQN DLVEYSPVTEKHLTDGMTVRELCSAAITMSDNTAANLLLTTIGGPKELTAFLHNMGDH VTRLDRWEPELNEAIPNDERDTTMPVAMATTLRKLLTGELLTLASRQQLIDWMEADKV AGPLLRSALPAGWFIADKSGAGERGSRGIIAALGPDGKPSRIVVIYTTGSQATMDERN RQIAEIGASLIKHW
acgcgttcgagctcgccccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgggggatcttggtggcgtgaaactcccgcacctcttcggccagcgaattccagaagccgccaccatggcttctagatcaactgagacagaacttcaagtagctgtgaaaaccagcgccaagaaagactccagaaagaaaggtcaggatcgcagtgaagccactttgataaagaggtttaaaggtgaaggggtccggtacaaagccaaattgatcgggattgatgaagtttccgcagctcggggagacaagttatgtcaagattccatgatgaaactcaagggcgttgttgctggcgctcgttccaaaggagaacacaaacagaaaatctttttaaccatctcctttggaggaatcaaaatctttgatgagaagacaggggcccttcagcatcatcatgctgttcatgaaatatcctacattgcaaaggacattacagatcaccgggcctttggatatgtttgtgggaaggaagggaatcacagatttgtggccataaaaacagcccaggcggctgaacctgttattctggacttgagagatctctttcaactcatttatgaattgaagcaaagagaagaattagaaaaaaaggcacaaaaggataagcagtgtgaacaagctgtgtaccagacaatattggaagaggatgttgaagatcctgtgtaccagtacattgtgtttgaggctggacacgagccaatccgtgatcccgaaacggaagaaaacatttatcaggttcccaccagccaaaagaaggaaggtgtttatgatgtgccaaaaagtcaacctgtaagtaatggctattcgtttgaggattttgaagaacggtttgctgcagccaccccgaacagaaacctgcccacagactttgatgagatttttgaggcaacgaaggctgtgacccaattagaactttttggggacatgtccacaccccctgatataacctctccccccactcctgcaactccaggtgatgcctttatcccatcttcatctcagacccttccagcgagtgcagatgtgtttagttctgtacctttcggcactgctgctgtaccctcaggttacgttgcaatgggcgctgtcctcccgtccttctggggtcagcagcccctcgtccaacagcagatggtcatgggtgcccagccaccagtcgctcaggtgatgccgggggctcagcccatcgcatggggccagccgggtctctttcctgccactcagcagccctggccaactgtggccgggcagtttccgccagccgccttcatgcccacacaaactgttatgcctttgccagctgccatgttccaaggtcccctcaccccccttgccaccgtcccaggcacgagtgactccaccaggtcaagtccacagaccgacaagcccaggcagaaaatgggcaaagaaacgtttaaggatttccagatggcccagcctccgcccgtgccctcccgcaaacccgaccagccctccctcacctgtacctcagaggccttctccagttacttcaacaaagtcggggtggcacaggatacagacgactgtgatgactttgacatctcccagttgaatttgacccctgtgacttctaccacaccatcgaccaactcacctccaaccccagcccctagacagagctctccatccaaatcatctgcatcccatgccagtgatcctaccacagatgacatctttgaagagggctttgaaagtcccagcaaaagcgaagagcaagaagctcctgatggatcacaggcctcatccaacagtgatccatttggtgagcccagtggggagcccagtggtgataatataagtccacaggccggtagctctagaggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaactagttatccgtacgacgtaccagactacgcataagaaaagtgaggatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaaggattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgaggagctttttgcaaaagccctaggcctccaaaaaagcctcttcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattgccacctccacctgaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagg

SEQ ID NO: 79 - protein, 9 aa, synthetic construct
YPYDVPDYA

SEQ ID NO: 80 - protein, 107 aa, synthetic construct
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 81 - protein, 290 aa, synthetic construct
MSIQHFRVALIPFFAAFCLPVFAHPETLVKVKDAEDQLGARVGYIELDLNSGKILESFRPEERFQVEVAMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW